(19) United States
(12) Patent Application Publication
(10) Pub. No.: US 2014/0366224 A1
(43) Pub. Date: Dec. 11, 2014

(54) PROCESS FOR MODIFYING THE ARCHITECTURE AND IMPROVING THE YIELD OF CROP PLANTS

(71) Applicant: Consejo Superior De Investigaciones Cientificas, Madrid (ES)

(72) Inventors: Enrique Rojo De La Viesca, Madrid (ES); Maria Maite Sanmartin Artinano, Madrid (ES); Jose Juan Sanchez Serrano, Madrid (ES)

(73) Assignee: Consejo Superior De Investigaciones Cientificas, Madrid (ES)

(21) Appl. No.: 14/076,739

(22) Filed: Nov. 11, 2013

Related U.S. Application Data
(63) Continuation-in-part of application No. PCT/GB2012/051146, filed on May 21, 2012.

(30) Foreign Application Priority Data
May 19, 2011 (ES) P201130812

(51) Int. Cl. C12N 15/82

(52) U.S. Cl. CPC C12N 15/8261
USPC 800/298; 536/23.6; 435/320.1; 435/419

(57) ABSTRACT

This invention identifies the plant MINIYO (IYO) gene and the AtRTR1 gene for the initiation of cell differentiation in all plant meristems and in embryogenesis. This invention relates methods for generating transgenic plants in which expression of the IYO and/or AtRTR1 genes or their orthologous genes is modified to advancing or delaying the onset of differentiation in one or more meristems of the plant.

### RELATED APPLICATIONS AND INCORPORATION BY REFERENCE

This application is a continuation-in-part application of international patent application Serial No. PCT/GB2012/051146 filed 21 May 2012, which published as PCT Publication No. WO 2012/156760 on 22 Nov. 2012, which claims benefit of Spanish patent application Serial No. P201130812 filed 19 May 2011.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

This invention lies within the technical field of agriculture. The various aspects of the invention may also be applied to the crop productivity for food and feed production as well as biomass production for the energy sector, specifically the development of transgenic plants for use in obtaining biofuels, such as in the production of bioethanol.

### BACKGROUND OF THE INVENTION

Plant architecture has a marked effect on the yield of plants. This influence is exerted through photosynthetic potential, the transfer of assimilated materials and nutrients to the different organs of the plant, and finally manifests itself in the potential growth and yield of the plant.

Cell differentiation is a fundamental process in all organisms and, together with cell proliferation, dictates the size and shape of the organisms (Ramirez-Parra et al., Int. J. Dev. Biol. 2005). Thus, modification of plant architecture and yield depend at the most fundamental level on regulating cell proliferation and differentiation. The molecular mechanisms controlling the cell cycle in plants are well known (De Veylder et al., Nat. Rev. Mo. Cell Biol. 2007) and are largely conserved relative to the mechanisms that control this cycle in other eukaryotes. Many of the methods for modifying the architecture of plants are based on controlling cell proliferation, for example by altering the dormancy/activation of buds (TCP genes) or altering the balance of proliferation in meristems or organ primordia (STM, WUS, AS1, SWP genes).

On the other hand, the mechanisms responsible for initiating cell differentiation are very poorly understood. In addition, it is not known which genetic factors initiate cell differentiation. In animals, it is assumed that the start of differentiation involves a transcriptional re-ordering which makes it possible to activate developmental programmes which are silenced in progenitor stem cells. There is evidence that silencing of these programmes is due to blocking the productive transcriptional elongation of developmental regulatory genes (Guenther et al., Cell 2007; Stock et al., Nature Cell Biol. 2007), but the factors which activate this elongation upon differentiation have not been identified.

An example of manipulating plant architecture and crop yield is disclosed in US 2009/0320163 which describes a method for obtaining transgenic plants whose plant architecture has been modified through alterations in the expression of PDR genes (Plant Developmental Regulators). These genes have similarities with phosphatidyl ethanolamide binding proteins (PEBPs), which act as inhibitors in the signalling cascades of MAP kinases, with the result that transgenic plants with a greater number of seeds, a better plant foliage architecture, stronger stems and a larger plant biomass in general are obtained.

Another specific example is transgenic Arabidopsis plants which have reduced levels of expression of AtMago mRNA (RNAi-AtMago plants). It is known that the Mago Nashi gene is involved in organisation of apical and root meristems, but not floral meristems, and also affects the formation of pollen and the development of seeds in Arabidopsis (Nam-I1 et al. Plant Science 176 (2009) 461-469). RNAi-AtMago plants generally presented delayed vegetative growth, producing a larger number of leaves of smaller size, apical meristems with excessively vacuolated cells and large intercellular spaces giving rise to shorter and branched stems, smaller root meristems and shorter lateral roots with premature differentiation of root hairs. RNAi-AtMago plants also show reduced pollen production and germination, occasionally giving rise to non-viable seeds.

At the present time, there are many methods available for modifying root and aboveground architecture and improving crop yields, including modifying the size of meristems. These methods are based on changing the expression of genes that have specific effects in each meristem, for example in shoot apical meristems through altering the expression of genes such as STM, CLV3 or WUS, in root apical meristems through altering the expression of genes such as PLT, SHY2 or RGF1, and in general modifying the synthesis, transport and signalling of auxins and cytokinins.

Citation or identification of any document in this application is not an admission that such document is available as prior art to the present invention.

### SUMMARY OF THE INVENTION

To the best of Applicants' knowledge, in the state of the art, no gene has been identified which acts directly to switch on cell differentiation. The decision as to whether to differentiate or not is shared by cells of all the meristems throughout the plant as well as in the developing embryo, which means that identification of a conserved genetic switch controlling this fate decision makes it possible to control cell differentiation throughout the plant in a targeted way. This includes, for example, control during embryogenesis and in the apical, floral and vascular meristems. Modulation of this unique switch is thus a novel mechanism, directly applicable to all the meristems of the plant and in embryos, which makes it possible to activate, block or delay the initiation of differentiation in a highly specific way.

The present invention identifies this common switch which initiates cell differentiation. The invention is aimed at methods for generating transgenic plants with altered cell differentiation and at transgenic plants obtained through such methods. Plants with altered cell differentiation are desirable tools in agriculture as they can be used to increase yield and the present invention is aimed at addressing the need for more productive crop plants.

The invention relates to methods for improving the root and aboveground architecture of plants to obtain better crop yields. According to the invention, the MINIYO and RTR1 genes are good tools for genetic manipulation to control the timing of the onset of differentiation in embryogenesis and in all of the meristems of the plant. According to the invention, MINIYO and RTR1 nucleic acid sequences can therefore be used to regulate the size and number of plant embryos, meristems and the organs generated from them. MINIYO and RTR1 interact and regulate the activity of RNA polymerase II (Pol II), and are jointly involved in the activation of transcriptional elongation and the expression of growth programmes which control the initiation of cell differentiation in the plant. The transgenic plants according to the invention differ from the parent plants in that they have an increase or decrease in the expression and/or genetic activity of MINIYO and/or RTR1, including the Arabidopsis thaliana AtMINIYO and AtRTR1 genes or their orthologues in other plant species. This gives rise to advance, delay or blocking of the initiation of differentiation in apical, floral and/or root meristems, thereby modifying the number and size of meristems and/or the number and size of the organs generated from them.

Preferably, the transgenic plants according to the invention have partly or wholly reduced expression of the MINIYO and/or RTR1 genes, including AtMINIYO and AtRTR1 or their orthologues in other plant species, in such a way that there is an improvement in their plant architecture which leads to improved crop yields in comparison with the wild plants. For example, the transgenic plants have meristems with increased size (greater stem thickness), and they also have ectopic meristems giving rise to additional inflorescences, multiple flowers and/or a large number of side roots and seeds with double embryos.

In a first aspect, the invention relates to an isolated nucleic acid sequence which may comprise a nucleotide sequence encoding for an amino acid sequence of SEQ ID NO: 5 or an orthologue thereof.

In a second aspect, the invention relates to an isolated nucleic acid sequence which may comprise a nucleotide sequence encoding for amino acid sequence of SEQ ID NO: 11 or an orthologue thereof.

In a further aspect, the invention relates to an expression vector which may comprise one or more of the isolated nucleic acid sequence(s) of the invention.

In another aspect, the invention relates to a transgenic plant wherein the activity of a MINIYO and/or RTR1 polypeptide is inactivated, repressed or down-regulated.

In an additional aspect, the invention relates to a transgenic plant wherein the activity of a MINIYO and/or RTR1 polypeptide is increased or up-regulated.

In a further aspect, the invention relates to a use of a MINIYO and/or RTR1 polypeptide to control the initiation of cell differentiation in plant apical, root and/or floral meristems.

In an additional aspect, the invention relates to a use of a MINIYO and/or RTR1 polypeptide to delay the initiation of cell differentiation in plant apical, root and floral meristems.

In another aspect, the invention relates to a method for delaying the onset of cell differentiation and increasing the number of undifferentiated cells in a plant said method which may comprise decreasing the activity of a MINIYO and/or RTR1 polypeptide.

In another aspect, the invention relates to a method for increasing cell differentiation in a plant said method which may comprise increasing the activity of a MINIYO and/or RTR1 polypeptide.

In further aspect, the invention relates to an isolated nucleic acid sequence which may comprise SEQ ID No. 48 or SEQ ID No. 49 and uses thereof to direct spatial and temporal expression of target genes.

Also included are methods of producing transgenic plants with altered activity of a MINIYO and/or RTR1 polypeptide and method of increasing yield by decreasing the activity of activity of a MINIYO and/or RTR1 polypeptide.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE FIGURES

The following detailed description, given by way of example, but not intended to limit the invention solely to the specific embodiments described, may best be understood in conjunction with the accompanying drawings.

FIG. 1. The mutant iyo-1 has delayed differentiation in the shoot (A) and root (B) apical meristems, in the procambium (C) and in the protoderm (D). (E) The expression of stem cell markers in iyo-1 plants extends beyond its normal meristematic niche. The boxes show the pattern of expression of the markers in wild plants (Wt). iyo-1 plants develop ectopic shoot apical meristems (F), multiple flowers from a single bud (G), ectopic root apical meristems (H) and additional stomas (I). (J) iyo-1/iyo-2 plants develop twin (double) embryos. (K) The double embryos are viable and germinate, developing into plants with a very low degree of differentiation. (L) iyo-1 plants also occasionally develop double embryos which develop normally.

FIG. 2. The iyo-1 mutant has a larger number of lateral roots, which develop ectopically in positions in which they do not appear in wild type plants (Wt): two lateral roots developing opposite one another or even emerging from the same point.

FIG. 3. (A) Diagram of the positional map of the mutant iyo-1. The number of recombinant chromosomes in a total of 1500 genotyped mutant plants is indicated. (B) Alignment of the MINIYO sequence and its orthologues in plants. At: Arabidopsis thaliana, SEQ ID No. 55; Mt: Medicago truncatula, SEQ ID No. 56; Vv: Vitis vinifera, SEQ ID No. 57; Os: Oryza sativa, SEQ ID No. 58; Pp: Physcomitrella patens, SEQ ID No. 59. The G/E mutation of the iyo-1 allele in the motif united to RNA RGG and conserved glycines is indicated. (C) The hypomorphic allele iyo-1 does not affect accumulation of the IYO transcript. (D) Phenotype of the null allele iyo-2.(E) Phenotype of iyo-1/iyo-2 and iyo-1/iyo-3 embryos. (F) Phenotype of iyo-1/iyo-2 plants.

FIG. 4. Expression of the β-glucuronidase (GUS) gene (A) and an IYO-GFP fusion (B) under the control of the IYO promoter in roots. (C) Detail of a root epidermis showing nuclear accumulation of IYO-GFP in cells about to begin differentiation. (D-F) Expression in shoot and root apices of IYO-GFP and RPB10-GFP (RPB-GFP) fusions under the control of the 35S promoter. Arrowheads mark nuclear accumulation of IYO-GFP in cells about to begin differentiation at the transition zone of the root.

FIG. 5. (A) Northern-blot with a specific IYO probe hybridised against samples of total RNA from different tissues and organs. Hypoc+SAM: Hypocotyl and shoot apical meristem of 7-day plants. Cotyledons: cotyledons of 7-day plants. Rosette leaves, cauline leaves, stems, inflorescence apices and flowers were from 30-day plants. (B) In situ hybridisation of inflorescence apices of 22-day plants with an antisense IYO probe. The box shows hybridisation with an IYO sense probe. (C-E) Pattern of expression of the β-glucuronidase (GUS) gene under the control of the IYO promoter. (F) Complementation of the iyo-1 mutant phenotypes with the ³⁵S::IYO-GFP construct. (G-H) Pattern of expression and subcellular accumulation of IYO-GFP under the control of the 35S promoter. (I) Pattern of expression and subcellular accumulation of GFP-IYO under the control of the 35S promoter. (J) Pattern of subcellular accumulation of GFP-IYO under the control of the IYO promoter in root apices of plants treated with 0.9 μM leptomycin B (+LMB) or with an equivalent quantity of the solvent used (-LMB) for 1 or 24 hours.

FIG. 6. (A) The majority of the 380 genes most significantly induced (p≦0.00015957) in the inflorescence apices of wild type plants relative to those of iyo-1 plants (Wt/iyo-1) are over-expressed in inflorescence apices of 35S::IYO-GFP plants relative to those of wild-type plants (IYO-GFPoe/Wt). (B) Histological sections of the apices of wild-type (Wt) and IYO-GFP over-expressing plants (IYO-GFPoe) 14 and 21 days after sowing. The arrowheads indicate prematurely differentiated cells. (C) Compaction of inflorescence in plants which over-express IYO-GFP (IYO-GFPoe).

FIG. 7. (A) Termination of the primary shoot apical meristem in plants which over-express IYO-HA under the 35S promoter (IYO-HAoe). (B) Termination of the primary root apical meristem in plants over-expressing IYO-HA under the 35S promoter (IYO-HAoe). The boxes show the meristem of wild type plants (Wt) at equivalent stages. (C) Premature differentiation of xylem vessels in lateral root primordia (arrows) and root hairs in epidermal cells prior to their elongation (asterisks).

FIG. 8. (A) IYO interacts in vitro with the Rpb3 sub-unit from RNA Polymerase II as shown by the specific pull-down of Rpb3 together with IYO-(B) IYO interacts in vivo with the Rpb3 and Rpb10 sub-units from RNA Polymerase II as shown by bimolecular reconstitution of YFP in the nucleus. (C) IYO is necessary for the activity of RNA Polymerase II in differentiating organs. Left-hand panel: Levels of the Rpb1 sub-unit phosphorylated in Serine 2 of the CTD (Ser2P) in leaf primordia (LP) and in mature leaves (ML) from wild-type (Wt) and iyo-1 mutant plants (iyo). Right-hand panel: Levels of the mRNA of RPB1 in leaf primordia (LP) or mature leaves (ML) from wild-type (Wt) and iyo-1 mutant plants (iyo) (D) Analysis of the relative expression in flowers versus undifferentiated tissues (calluses or cell cultures) of the 380 genes most significantly induced (p≦0.00015957) and the 380 most significantly repressed (p≦0.00066388) in floral meristems (and associated floral primordia) from wild-type plants relative to those from iyo-1 plants (Wt/iyo). (E) Constitutive ubiquitination of Rpb1 in iyo-1 plants. Extracts (Inputs) from wild (W) or iyo-1 (i) plants treated (+) or not (−) with MG132 were immunoprecipitated (IP) with antibodies against Rpb1 and analysed by Western blot with antibodies against Rpb1 or against ubiquitin (Ubq). (F) The over-expression of IYO increases resistance to the transcriptional elongation inhibitor 6-azauracil. Plants grown for 11 days in the presence of increasing concentrations of 6-azauracil (in μM) are shown. (G) IYO interacts in vitro with the histone acetyl transferase ELO3. (H) IYO interacts genetically with components of the Elongator complex to activate cell differentiation.

FIG. 9. Development of atrtr1-1 (rtr1-1) embryos compared to wild type embryos (Wt) at the corresponding stage in the panels above.

FIG. 10. (A) Formation of ectopic shoot apical meristems in atrtr1-2 plants causes fasciation and thickening of the stem. (B) Ectopic floral meristems give rise to duplicated flowers (and fruits) in atrtr1-2 plants. (C) Duplicated root apical meristems in atrtr1-2 plants. (D) Genetic interaction between AtRTR1 and IYO. The double iyo-1atrtr1-2 mutants grow as an undifferentiated callus. (E) Expression of the UidA (GUS) gene under the control of the AtRTR1 promoter in apical root meristems, lateral root primordia (arrow) and pericycle. (F) The fusion protein AtRTR1-GFP is excluded from the nuclei in undifferentiated cells of the meristem (arrows).

FIG. 11. (A) atrt1-2 plants (rtr1-2) develop ectopic meristemoids which give rise to clusters of stomas (arrowheads). (B) Cotyledons from iyo-1 and atrtr1-2 plants maintain cells which express the stem cell marker pTMM::TMM-GFP (arrowheads) at later stages than cotyledons from wild type plants (Wt).

FIG. 12. (A) The AtRTR1-GFP protein is excluded from the nucleus in untreated plants (arrowheads). (B) Treatments with leptomycin B, an inhibitor of Exportin1, causes nuclear accumulation of AtRTR1-GFP (arrows).

FIG. 13. IYO interacts in vivo with the AtRTR1 as shown by bimolecular reconstitution of YFP in the nucleus in two different combinations of fusions of AtRTR1 and IYO with nYFP and cYFP.

FIG. 14. AtRTR1-YFP accumulates in the nucleus when co-expressed with IYO-HA (AtRTR1-YFP+IYO-HA) but not when expressed on its own (AtRTR1-YFP).

FIG. 15. The atrtr1-2 and iyo-1 mutants have almost identical changes in the transcriptome relative to wild type plants. The majority of the 380 genes most significantly induced (p≦0.00015957) or repressed genes (p≦0.00066388) in the inflorescence apices of wild-type plants relative to those of iyo-1 plants (Wt/iyo-1) are similarly affected (induced or repressed respectively) in inflorescence apices of wild type plants relative to those of atrtr1-2 plants (Wt/art-2).

FIG. 16. Alignment of protein sequences coded by orthologous genes of MINIYO in a representative set of plants using the Clustal W program. The ordered sequences, from top to bottom, are those of: Arabidopsis thaliana, SEQ ID No. 5, Arabidopsis lyrata, SEQ ID No. 60, Brachypodium distachyon, SEQ ID No. 16, Citrus clementina, SEQ ID No. 61, Citrus sinensis, SEQ ID No. 62, Manihot sculenta, SEQ ID No. 63, Oryza sativa, SEQ ID No. 12, Populus trichocarpa, SEQ ID No. 64, Prunus persica, SEQ ID No. 65, Ricinus communis, SEQ ID No. 66, Setaria italica, SEQ ID No. 67, Sorghum bicolor, SEQ ID No. 17, Vitis vinifera, SEQ ID No. 68, Zea mays, SEQ ID No. 13, Physcomitrella patens, SEQ ID No. 69, Carica papaya, SEQ ID No. 70, Glycine max, SEQ ID No. 45, Medicago truncatula, SEQ ID No. 71 and Eucalyptus grandis, SEQ ID No. 72.

FIG. 17. Phylogenetic tree constructed from the alignment of the polypeptide sequence of the orthologues of MINIYO using the Clustal W programme.

FIG. 18. Examples of highly-conserved domains in the MINIYO proteins of plants. The sequences, in order from top to bottom, are those of: Arabidopsis thaliana, Arabidopsis lyrata, Brachypodium distachyon, Citrus clementina, Citrus sinensis, Manihot sculenta, Oryza sativa, Populus trichocarpa, Prunus persica, Ricinus communis, Setaria italica, Sorghum bicolor, Vitis vinifera, Zea mays, Carica papaya, Glycine max, Medicago truncatula, Eucalyptus grandis and Physcomitrella patens. The amino acids of the MINIYO protein of Arabidopsis thaliana corresponding to the domains shown are: (a) aa. 960-980. This domain contains the RGG motif mutated into the hypomorphic iyo-1 allele of Arabidopsis which is conserved in all the plant orthologues; Arabidopsis thaliana, SEQ ID No. 84, Arabidopsis lyrata, SEQ ID No. 85 Brachypodium distachyon, SEQ ID No. 86 Citrus clementina, SEQ ID No. 87 Citrus sinensis, SEQ ID No. 88 Manihot sculenta, SEQ ID No. 89 Oryza sativa, SEQ ID No. 90 Populus trichocarpa, SEQ ID No. 91 Prunus persica, SEQ ID No. 92 Ricinus communis, SEQ ID No. 93 Setaria italica, SEQ ID No. 94 Sorghum bicolor, SEQ ID No. 95 Vitis vinifera, SEQ ID No. 96 Zea mays, SEQ ID No. 97 Physcomitrella patens, SEQ ID No. 98 Carica papaya, SEQ ID No. 99 Glycine max, SEQ ID No. 100 Medicago truncatula SEQ ID No. 101 and Eucalyptus grandis SEQ ID No. 102 (b) aa. 209-255; Arabidopsis thaliana, SEQ ID No. 103, Arabidopsis lyrata, SEQ ID No. 104 Brachypodium distachyon, SEQ ID No. 105 Citrus clementina, SEQ ID No. 106 Citrus sinensis, SEQ ID No. 107 Manihot sculenta, SEQ ID No. 108 Oryza sativa, SEQ ID No. 109 Populus trichocarpa, SEQ ID No. 110 Prunus persica, SEQ ID No. 111 Ricinus communis, SEQ ID No. 112 Setaria italica, SEQ ID No. 113 Sorghum bicolor, SEQ ID No. 114 Vitis vinifera, SEQ ID No. 115 Zea mays, SEQ ID No. 116 Physcomitrella patens, SEQ ID No. 117 Carica papaya, SEQ ID No. 118 Glycine max, SEQ ID No. 119 Medicago truncatula SEQ ID No. 120 and Eucalyptus grandis SEQ ID No. 121. (c) aa. 317-396; Arabidopsis thaliana, SEQ ID No. 122, Arabidopsis lyrata, SEQ ID No. 123 Brachypodium distachyon, SEQ ID No. 124 Citrus clementina, SEQ ID No. 125 Citrus sinensis, SEQ ID No. 126 Manihot sculenta, SEQ ID No. 127 Oryza sativa, SEQ ID No. 128 Populus trichocarpa, SEQ ID No. 129 Prunus persica, SEQ ID No. 130 Ricinus communis, SEQ ID No. 131 Setaria italica, SEQ ID No. 132 Sorghum bicolor, SEQ ID No. 133 Vitis vinifera, SEQ ID No. 134 Zea mays, SEQ ID No. 135 Physcomitrella patens, SEQ ID No. 136 Carica papaya, SEQ ID No. 137 Glycine max, SEQ ID No. 138 Medicago truncatula SEQ ID No. 139 and Eucalyptus grandis SEQ ID No. 140. (d) aa. 350-389.; Arabidopsis thaliana, SEQ ID No. 141, Arabidopsis lyrata, SEQ ID No. 142 Brachypodium distachyon, SEQ ID No. 143 Citrus clementina, SEQ ID No. 144 Citrus sinensis, SEQ ID No. 145 Manihot sculenta, SEQ ID No. 146 Oryza sativa, SEQ ID No. 147 Populus trichocarpa, SEQ ID No. 148 Prunus persica, SEQ ID No. 149 Ricinus communis, SEQ ID No. 150 Setaria italica, SEQ ID No. 151 Sorghum bicolor, SEQ ID No. 152 Vitis vinifera, SEQ ID No. 153 Zea mays, SEQ ID No. 154 Physcomitrella patens, SEQ ID No. 155 Carica papaya, SEQ ID No. 156 Glycine max, SEQ ID No. 157 Medicago truncatula SEQ ID No. 158 and Eucalyptus grandis SEQ ID No. 159 (e) aa. 417-437; Arabidopsis thaliana, SEQ ID No. 160, Arabidopsis lyrata, SEQ ID No. 161 Brachypodium distachyon, SEQ ID No. 162 Citrus clementina, SEQ ID No. 163 Citrus sinensis, SEQ ID No. 164 Manihot sculenta, SEQ ID No. 165 Oryza sativa, SEQ ID No. 166 Populus trichocarpa, SEQ ID No. 167 Prunus persica, SEQ ID No. 168 Ricinus communis, SEQ ID No. 169 Setaria italica, SEQ ID No. 170 Sorghum bicolor, SEQ ID No. 171 Vitis vinifera, SEQ ID No. 172 Zea mays, SEQ ID No. 173 Physcomitrella patens, SEQ ID No. 174 Carica papaya, SEQ ID No. 175 Glycine max, SEQ ID No. 176 Medicago truncatula SEQ ID No. 177 and Eucalyptus grandis SEQ ID No. 178 (f) aa. 529-559; Arabidopsis thaliana, SEQ ID No. 179, Arabidopsis lyrata, SEQ ID No. 180 Brachypodium distachyon, SEQ ID No. 181 Citrus clementina, SEQ ID No. 182 Citrus sinensis, SEQ ID No. 183 Manihot sculenta, SEQ ID No. 184 Oryza sativa, SEQ ID No. 185 Populus trichocarpa, SEQ ID No. 186 Prunus persica, SEQ ID No. 187 Ricinus communis, SEQ ID No. 188 Setaria italica, SEQ ID No. 189 Sorghum bicolor, SEQ ID No. 190 Vitis vinifera, SEQ ID No. 191 Zea mays, SEQ ID No. 192 Physcomitrella patens, SEQ ID No. 193 Carica papaya, SEQ ID No. 194 Glycine max, SEQ ID No. 195 Medicago truncatula SEQ ID No. 196 and Eucalyptus grandis SEQ ID No. 197 (g) aa. 529-597; Arabidopsis thaliana, SEQ ID No. 198, Arabidopsis lyrata, SEQ ID No. 199 Brachypodium distachyon, SEQ ID No. 200 Citrus clementina, SEQ ID No. 201 Citrus sinensis, SEQ ID No. 202 Manihot sculenta, SEQ ID No. 203 Oryza sativa, SEQ ID No. 204 Populus trichocarpa, SEQ ID No. 205 Prunus persica, SEQ ID No. 206 Ricinus communis, SEQ ID No. 207 Setaria italica, SEQ ID No. 208 Sorghum bicolor, SEQ ID No. 209 Vitis vinifera, SEQ ID No. 210 Zea mays, SEQ ID No. 211 Physcomitrella patens, SEQ ID No. 212 Carica papaya, SEQ ID No. 213 Glycine max, SEQ ID No. 214 Medicago truncatula SEQ ID No. 215 and Eucalyptus grandis SEQ ID No. 216 (h) aa. 1136-1145; Arabidopsis thaliana, SEQ ID No. 217, Arabidopsis lyrata, SEQ ID No. 218 Brachypodium distachyon, SEQ ID No. 219 Citrus clementina, SEQ ID No. 220 Citrus sinensis, SEQ ID No. 221 Manihot sculenta, SEQ ID No. 222 Oryza sativa, SEQ ID No. 223 Populus trichocarpa, SEQ ID No. 224 Prunus persica, SEQ ID No. 225 Ricinus communis, SEQ ID No. 226 Setaria italica, SEQ ID No. 227 Sorghum bicolor, SEQ ID No. 228 Vitis vinifera, SEQ ID No. 229 Zea mays, SEQ ID No. 230 Physcomitrella patens, SEQ ID No. 231 Carica papaya, SEQ ID No. 232 Glycine max, SEQ ID No. 233 Medicago truncatula SEQ ID No. 234 and Eucalyptus grandis SEQ ID No. 235 (i) aa. 1144-1416 Arabidopsis thaliana, SEQ ID No. 236, Arabidopsis lyrata, SEQ ID No. 237 Brachypodium distachyon, SEQ ID No. 238 Citrus clementina, SEQ ID No. 239 Citrus sinensis, SEQ ID No. 240 Manihot sculenta, SEQ ID No. 241 Oryza sativa, SEQ ID No. 242 Populus trichocarpa, SEQ ID No. 243 Prunus persica, SEQ ID No. 244 Ricinus communis, SEQ ID No. 245 Setaria italica, SEQ ID No. 246 Sorghum bicolor, SEQ ID No. 247 Vitis vinifera, SEQ ID No. 248 Zea mays, SEQ ID No. 249 Physcomitrella patens, SEQ ID No. 250 Carica papaya, SEQ ID No. 251 Glycine max, SEQ ID No. 252 Medicago truncatula SEQ ID No. 253 and Eucalyptus grandis SEQ ID No. 254.

FIG. 19. Alignment of protein sequences coded by orthologous genes of AtRTR1 in a representative set of plants using the Clustal W programme. The sequences in order from top to bottom are those of: Arabidopsis thaliana, SEQ ID No. 11, Carica papaya, SEQ ID No. 73, Cucumis sativa, SEQ ID No. 74, Eucalyptus grandis, SEQ ID No. 75, Glycine max, SEQ ID No. 24, Mimulus guttatus, SEQ ID No. 76, Manihot esculenta, SEQ ID No. 77, Populus trichocarpa, SEQ ID No. 78, Prunus persica, SEQ ID No. 79, Ricinus communis, SEQ ID No. 80, Vitis vinifera, SEQ ID No. 81, Zea mays, SEQ ID No. 23, Sorghum bicolor, SEQ ID No. 26, Setaria italica, SEQ ID No. 82, Oryza sativa, SEQ ID No. 12, Brachypodium distachyon, SEQ ID No. 27 and Picea glauca, SEQ ID No. 83.

FIG. 20. Phylogenetic tree constructed from the alignment of the polypeptide sequences of the orthologues of AtRTR1 using the Clustal W programme.

FIG. 21. Examples of highly-conserved domains in the RTR1 proteins of plants. The sequences, in order from top to bottom, are those of: Arabidopsis thaliana, Carica papaya, Cucumis sativa, Eucalyptus grandis, Glycine max, Mimulus guttatus, Manihot esculenta, Populus trichocarpa, Prunus persica, Ricinus communis, Vitis vinifera, Zea mays, Sorghum bicolor, Setaria italica, Oryza sativa, Brachypodium distachyon and Picea glauca. The amino acids of the AtRTR1 protein of Arabidopsis thaliana corresponding to the domains shown are: (a) aa. 39-61. This domain includes catalytic cysteine C56 which is strictly conserved in all the orthologues; Arabidopsis thaliana, SEQ ID No. 255 Carica papaya, SEQ ID No. 256 Cucumis sativa, SEQ ID No. 257 Eucalyptus grandis, SEQ ID No. 258 Glycine max, SEQ ID No. 259 Mimulus guttatus, SEQ ID No. 260 Manihot esculenta, SEQ ID No. 261 Populus trichocarpa, SEQ ID No. 262 Prunus persica, SEQ ID No. 263 Ricinus communis, SEQ ID No. 264 Vitis vinifera, SEQ ID No. 265 Zea mays, SEQ ID No. 266 Sorghum bicolor, SEQ ID No. 267 Setaria italica, SEQ ID No. 268 Oryza sativa, SEQ ID No. 269 Brachypodium distachyon SEQ ID No. 270 and Picea glauca SEQ ID No. 271. (b) aa. 39-61; Arabidopsis thaliana, SEQ ID No. 272 Carica papaya, SEQ ID No. 273 Cucumis sativa, SEQ ID No. 274 Eucalyptus grandis, SEQ ID No. 275 Glycine max, SEQ ID No. 276 Mimulus guttatus, SEQ ID No. 277 Manihot esculenta, SEQ ID No. 278 Populus trichocarpa, SEQ ID No. 279 Prunus persica, SEQ ID No. 280 Ricinus communis, SEQ ID No. 281 Vitis vinifera, SEQ ID No. 282 Zea mays, SEQ ID No. 283 Sorghum bicolor, SEQ ID No. 284 Setaria italica, SEQ ID No. 285 Oryza sativa, SEQ ID No. 286 Brachypodium distachyon SEQ ID No. 287 and Picea glauca SEQ ID No. 288. (c) aa. 77-89; Arabidopsis thaliana, SEQ ID No. 289 Carica papaya, SEQ ID No. 290 Cucumis sativa, SEQ ID No. 291 Eucalyptus grandis, SEQ ID No. 292 Glycine max, SEQ ID No. 293 Mimulus guttatus, SEQ ID No. 294 Manihot esculenta, SEQ ID No. 295 Populus trichocarpa, SEQ ID No. 296 Prunus persica, SEQ ID No. 297 Ricinus communis, SEQ ID No. 298 Vitis vinifera, SEQ ID No. 299 Zea mays, SEQ ID No. 300 Sorghum bicolor, SEQ ID No. 301 Setaria italica, SEQ ID No. 302 Oryza sativa, SEQ ID No. 303 Brachypodium distachyon SEQ ID No. 304 and Picea glauca SEQ ID No. 305. (d) aa. 429-435; Arabidopsis thaliana, SEQ ID No. 306 Carica papaya, SEQ ID No. 307 Cucumis sativa, SEQ ID No. 308 Eucalyptus grandis, SEQ ID No. 309 Glycine max, SEQ ID No. 310 Mimulus guttatus, SEQ ID No. 311 Manihot esculenta, SEQ ID No. 312 Populus trichocarpa, SEQ ID No. 313 Prunus persica, SEQ ID No. 314 Ricinus communis, SEQ ID No. 315 Vitis vinifera, SEQ ID No. 316 Zea mays, SEQ ID No. 317 Sorghum bicolor, SEQ ID No. 318 Setaria italica, SEQ ID No. 319 Oryza sativa, SEQ ID No. 320 Brachypodium distachyon SEQ ID No. 321 and Picea glauca SEQ ID No. 322. (e) aa. 473-507; Arabidopsis thaliana, SEQ ID No. 323 Carica papaya, SEQ ID No. 324 Cucumis sativa, SEQ ID No. 325 Eucalyptus grandis, SEQ ID No. 326 Glycine max, SEQ ID No. 327 Mimulus guttatus, SEQ ID No. 328 Manihot esculenta, SEQ ID No. 329 Populus trichocarpa, SEQ ID No. 330 Prunus persica, SEQ ID No. 331 Ricinus communis, SEQ ID No. 332 Vitis vinifera, SEQ ID No. 333 Zea mays, SEQ ID No. 334 Sorghum bicolor, SEQ ID No. 335 Setaria italica, SEQ ID No. 336 Oryza sativa, SEQ ID No. 337 Brachypodium distachyon SEQ ID No. 338 and Picea glauca SEQ ID No. 339. (f) 552-589 Arabidopsis thaliana, SEQ ID No. 340 Carica papaya, SEQ ID No. 341 Cucumis sativa, SEQ ID No. 342 Eucalyptus grandis, SEQ ID No. 343 Glycine max, SEQ ID No. 344 Mimulus guttatus, SEQ ID No. 345 Manihot esculenta, SEQ ID No. 346 Populus trichocarpa, SEQ ID No. 347 Prunus persica, SEQ ID No. 348 Ricinus communis, SEQ ID No. 349 Vitis vinifera, SEQ ID No. 350 Zea mays, SEQ ID No. 351 Sorghum bicolor, SEQ ID No. 352 Setaria italica, SEQ ID No. 353 Oryza sativa, SEQ ID No. 354 Brachypodium distachyon SEQ ID No. 355 and Picea glauca SEQ ID No 0.356.

FIG. 22. Seed yield test of a line co-suppressed in AtRTR1. Seed yield was measured in wild type Arabidopsis plants (Col-0 plants) and in plants from a 35S::AtRTR1-GFP line displaying co-supression of the AtRTR1-GFP transgene and of the endogenous AtRTR1 gene (si-art plants). Six independent experiments were carried out. The plants were germinated in sterile MS media supplemented with 1% sucrose and transplanted to soil in individual pots after 1 week (10-16 plants per genotype in each experiment). Seeds were collected from individual plants after they were fully dried and the total seed weight measured. For comparing the results of the different experiments, Applicants calculated the relative yield of each plant as the ratio of the yield of that plant relative to the average yield of control plants (Col-0) in that particular experiment. (A) Average relative seed yield of Col-0 and si-art plants in all six experiments combined. A 12% increase in the average seed yield of si-art plants is observed, with a p-value of 0.006 in an unpaired two tailed t-test. (B) Average relative seed yield of Col-0 and si-art plants in each of the six independent experiments. In experiments 3 and 6 the yield of si-art plants is increased 32% and 27% over that of the control plants (p-value=0.006). The error bars represent standard deviation.

FIG. 23. Phenotype of 35S::IYO-GFP tomato plants. Shown are untransformed tomato plants of the variety Moneymaker (TMM) and three independent transgenic plants of the same variety expressing a 35S::IYO-GFP construct (Lines 1-3). Line 1 displays determinate growth of the primary shoot. The arrow marks the terminated primary shoot. Lines 2 and 3 display reduced apical dominance resulting in a shorter stature and increased branching. Arrowheads mark premature outgrowth of lateral buds.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

The basis of this invention is the characterisation of the AtMINIYO (At4g38440) and AtRTR1 (At5g26760) genes which may comprise a highly conserved, common molecular switch that initiates differentiation in all plants. The acronym IYO is used herein to refer to the AtMINIYO gene.

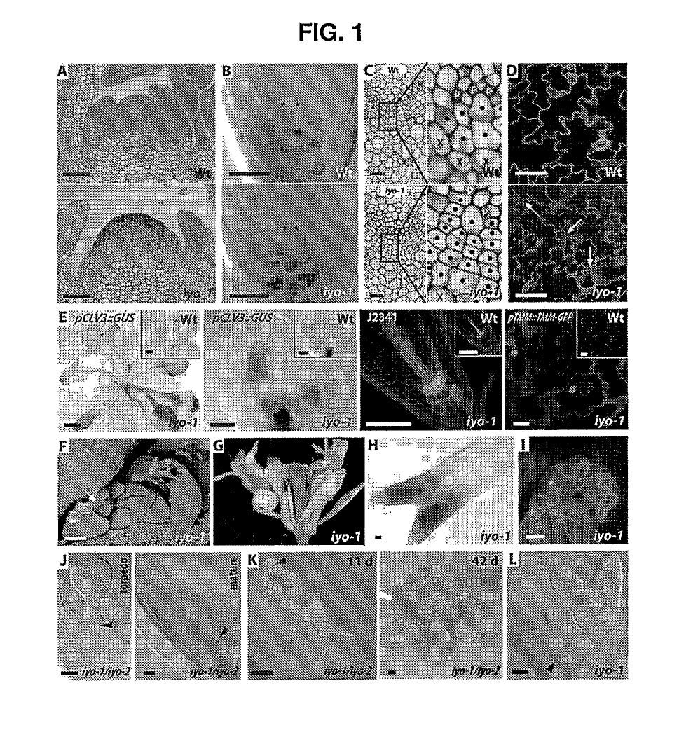

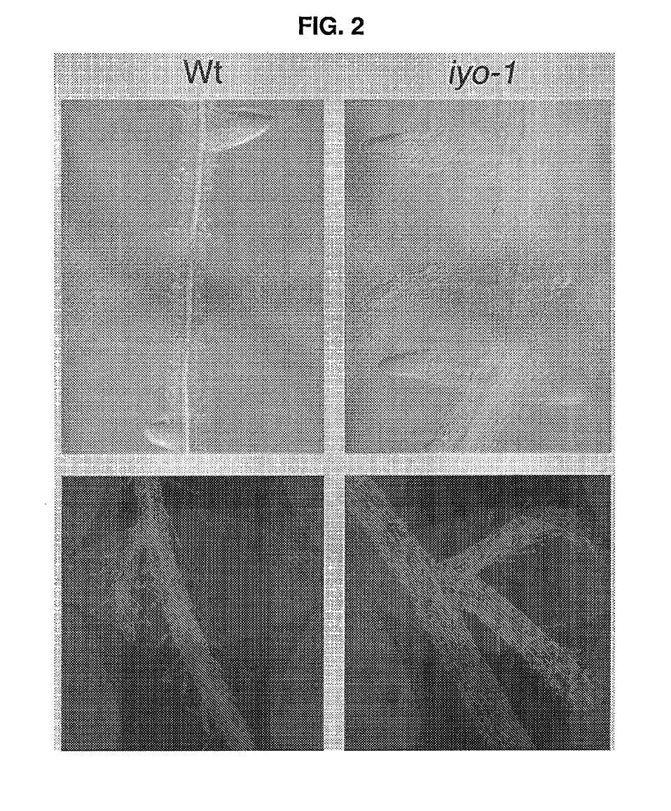

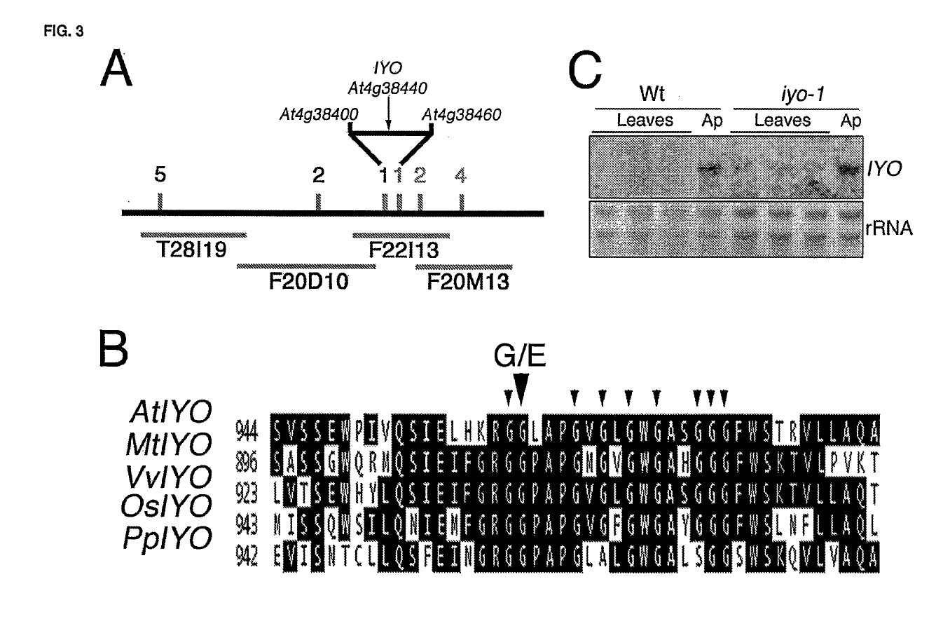

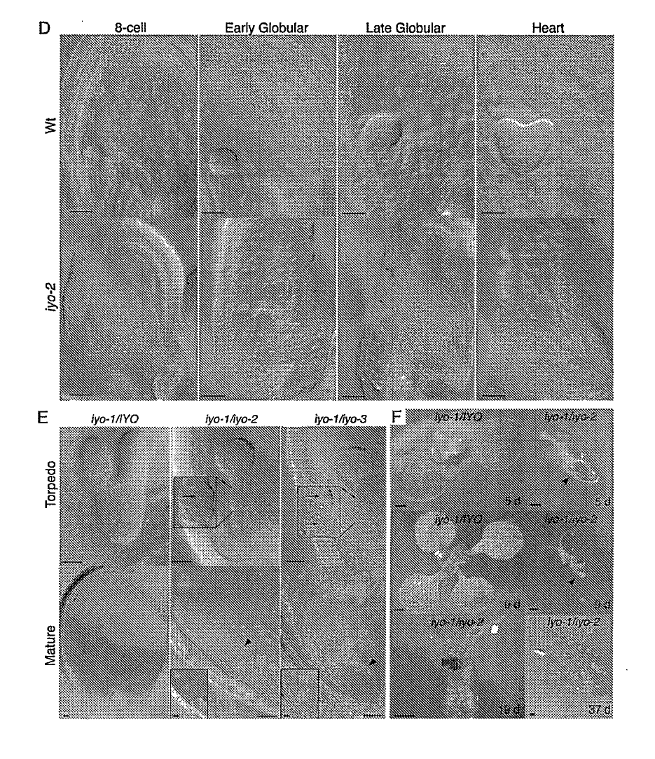

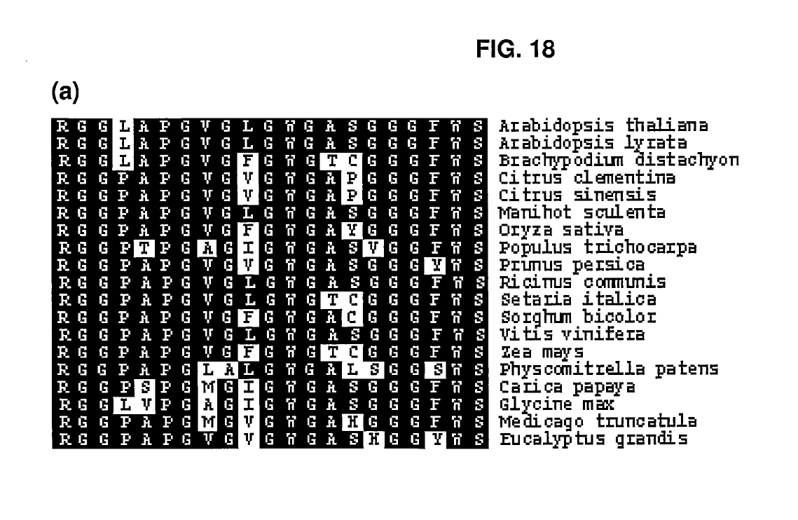

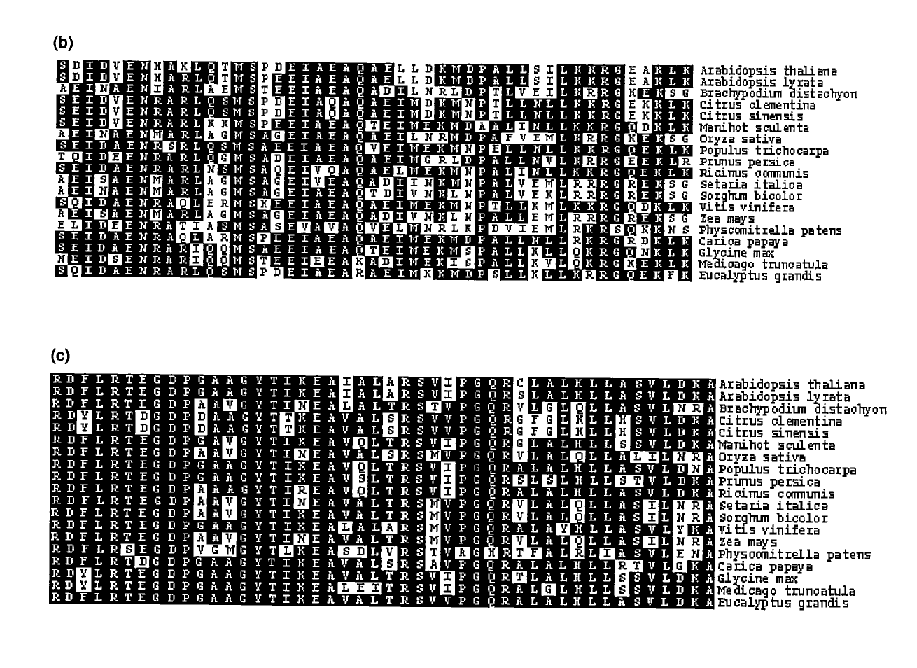

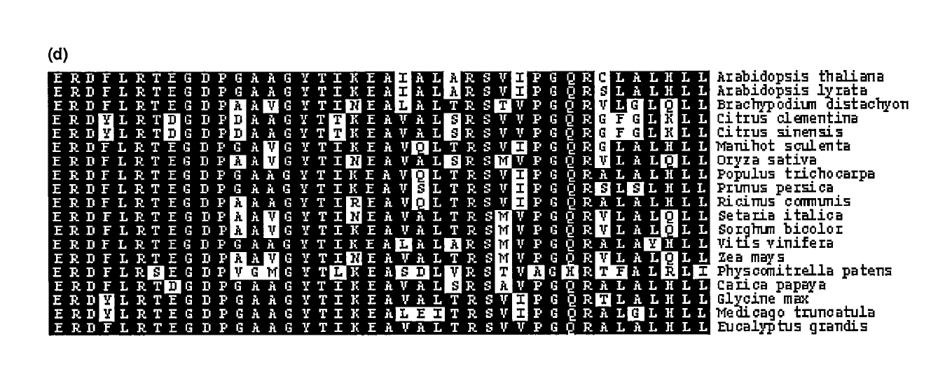

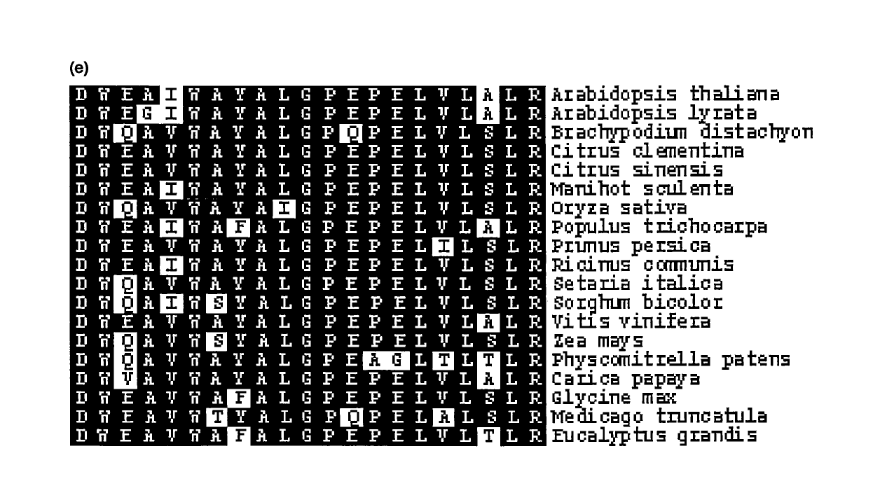

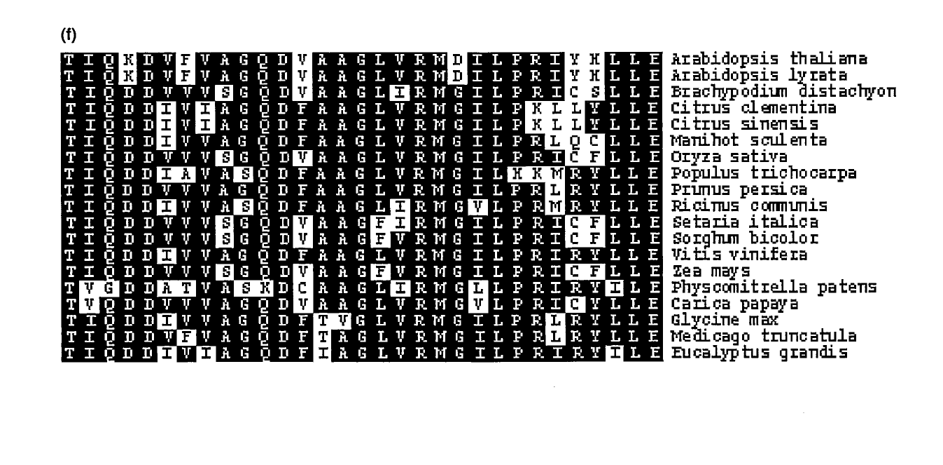

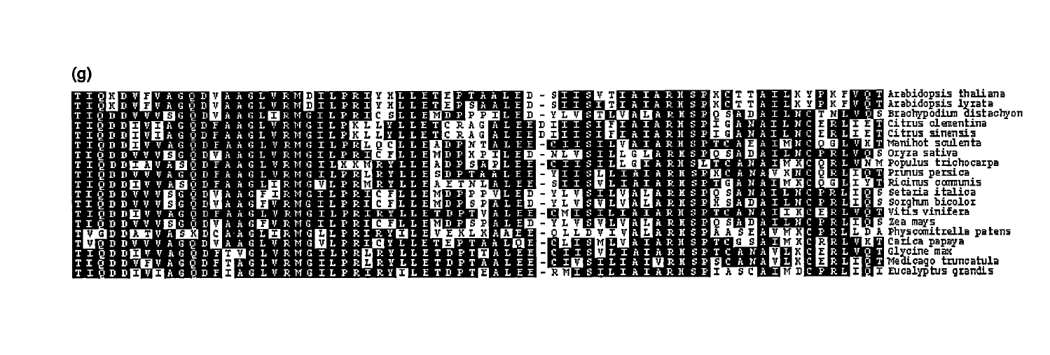

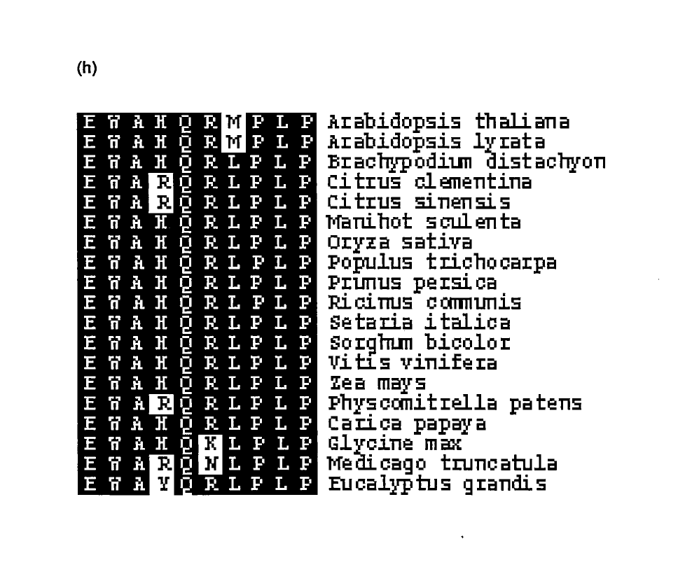

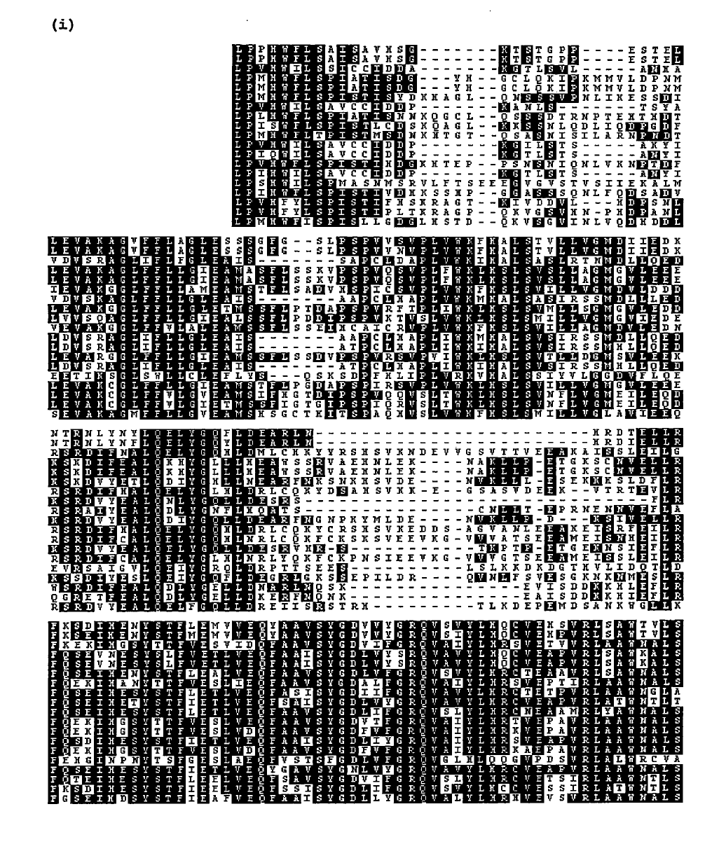

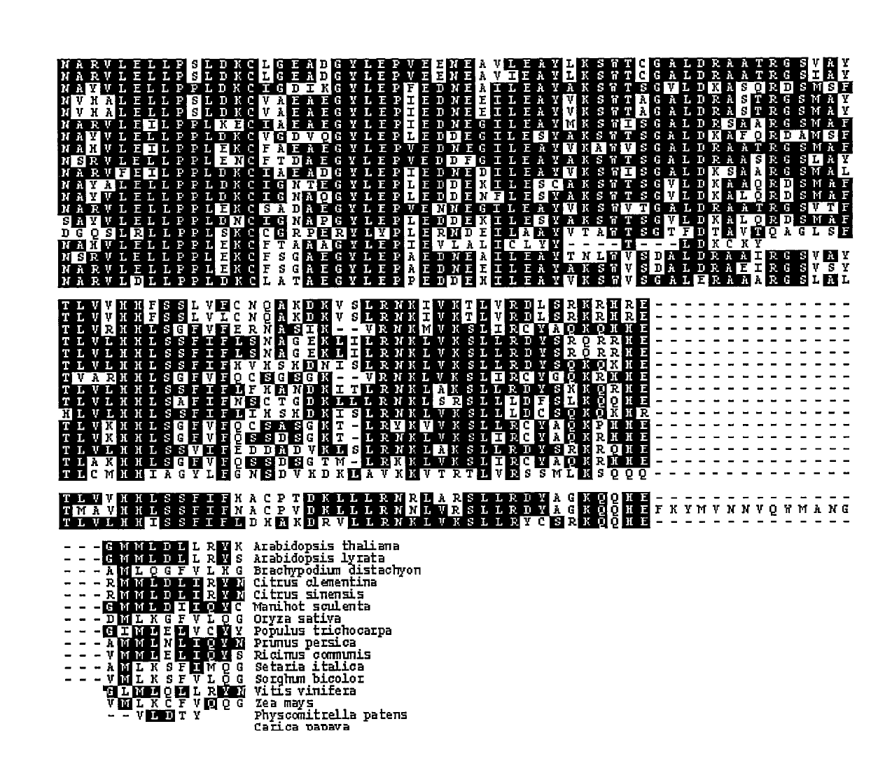

Applicants have identified and characterised the mutant miniyo-1 (iyo-1) which shows a delay in the initiation of cell differentiation (FIGS. 1 and 2). Through positional mapping, Applicants have shown that phenotypes with delayed differentiation in iyo-1 plants are due to a point mutation in the IYO gene (At4g38440, FIGS. 3A-B). The iyo-1 allele causes a change in amino acid 962 from a glycine to a glutamate residue. This disrupts an RGG RNA-binding motif which is strictly conserved in all MINIYO orthologues (FIGS. 3B and 18a). This mutation gives rise to a partial loss of activity in the IYO gene and results in delayed onset of differentiation in all the meristems of the plant (FIG. 1). As a consequence, ectopic stem cells are generated (FIGS. 1A-E, 2) and these give rise to new shoot and root meristems, new floral meristems (double and triple flowers), adjacent groups of stomas (FIG. 1F-I) and a larger number of lateral roots, including in positions in which they do not develop in wild-type plants (FIG. 2). Total blocking of IYO activity causes embryogenesis to be arrested at early stages (FIG. 3E-F).

In addition, the combination of the hypomorphic iyo-1 allele with null alleles (iyo-2 or iyo-3) gives rise to the formation of seeds with double embryos (FIGS. 1J and 3D) which mature and upon germinating give rise to plants with multiple meristem poles which generate rudimentary leaves (FIG. 1K). Also in the case of the iyo-1 allele there are cases of double embryos which develop in a similar way to wild-type embryos (FIG. 1L).

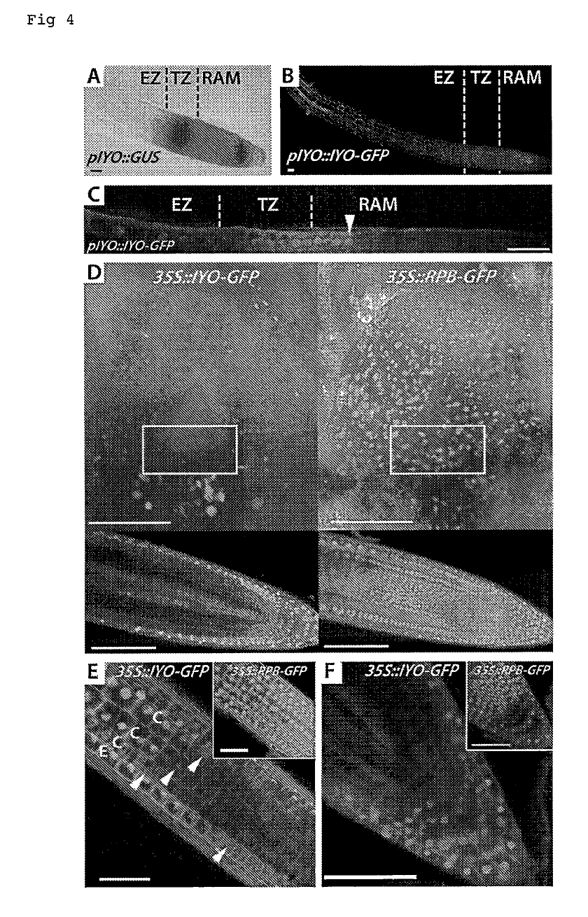

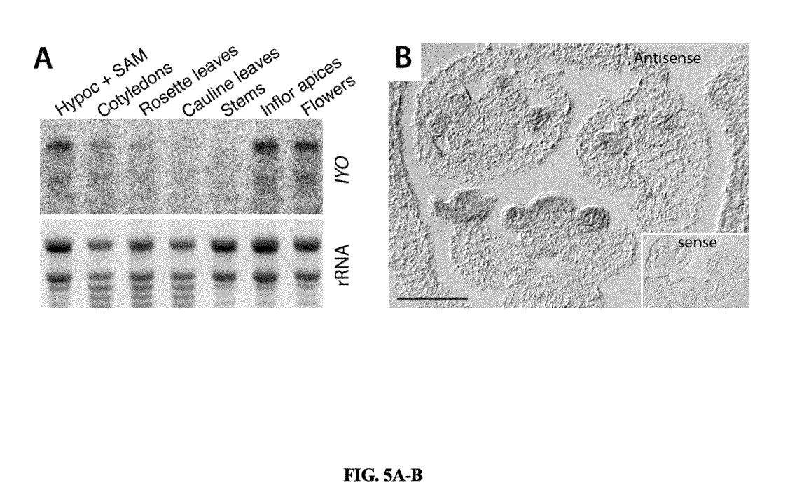

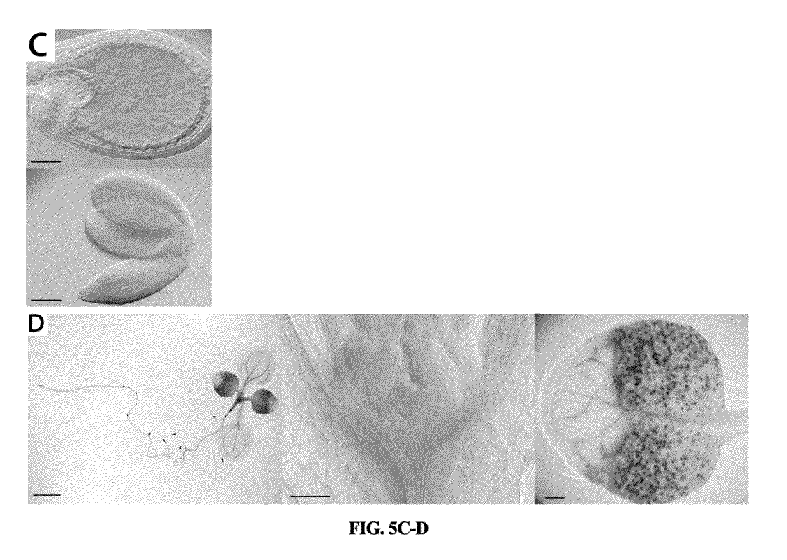

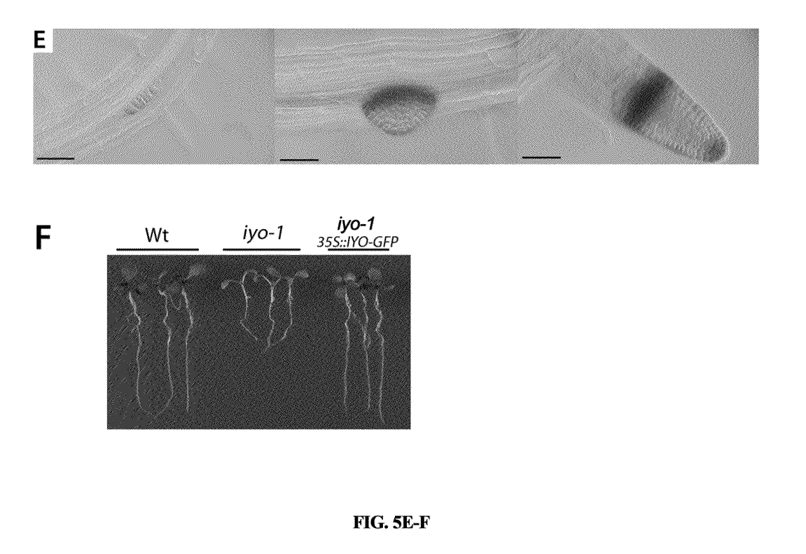

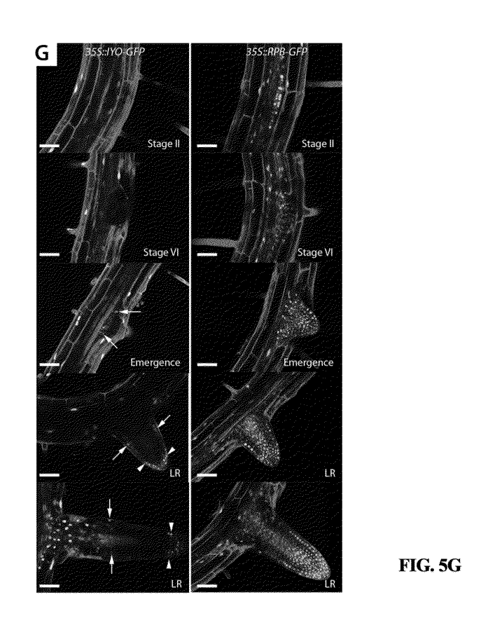

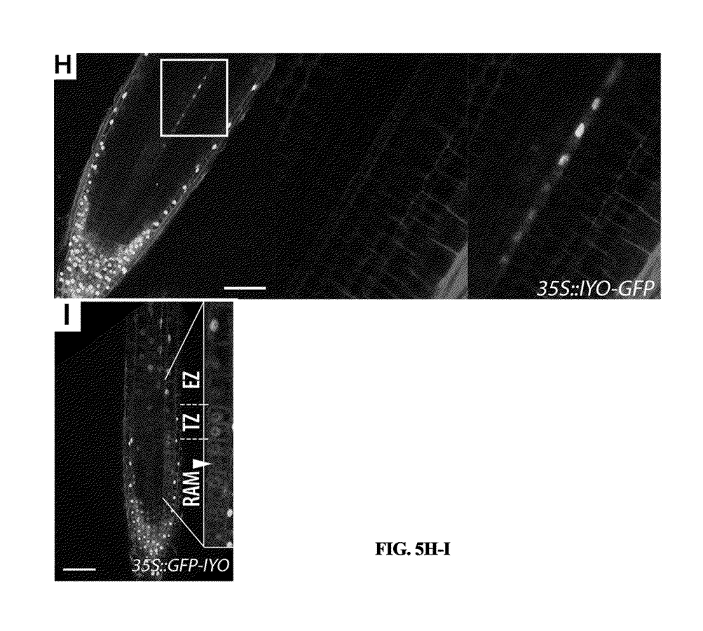

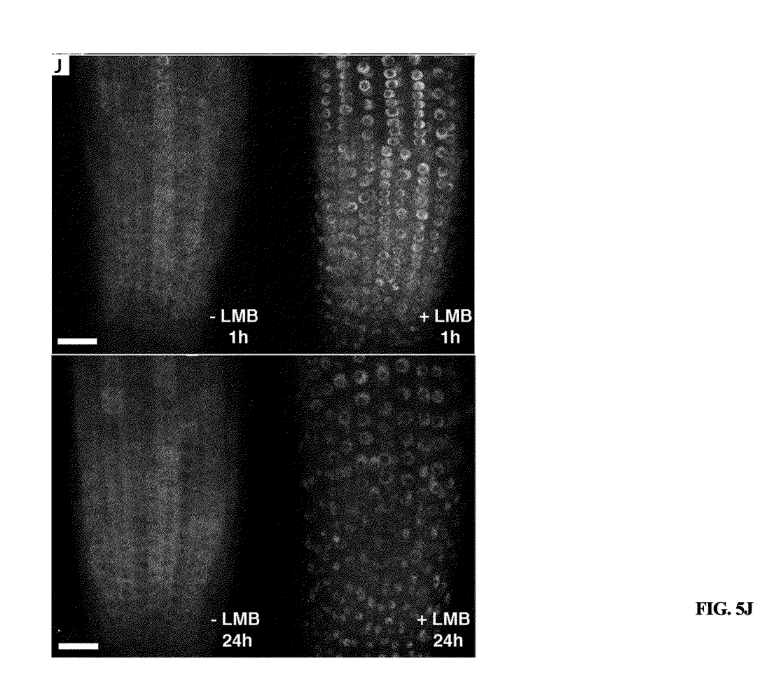

Expression of the IYO gene is regulated at the transcriptional and post-transcriptional level to direct its activity specifically to the periphery of the meristems where differentiation begins (FIGS. 4 and 5). The promoter of the IYO gene is exclusively active in developing seeds, in meristems and in cells in the early stages of differentiation (FIGS. 4A-B, 5C-E). Moreover, specific signal sequences present in the IYO protein restrict nuclear accumulation of the protein exclusively to cells which are about to begin differentiation (FIGS. 4B-F, 5G-I). The protein is excluded from the nucleus in proliferating meristematic cells through active Exportin1-dependent export, as it is inhibited by treatment with leptomycin B (FIG. 5j).

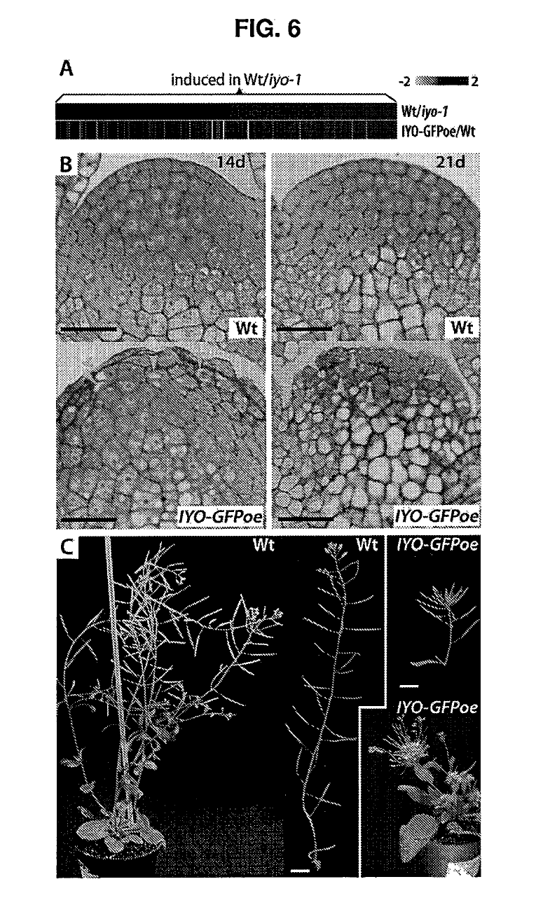

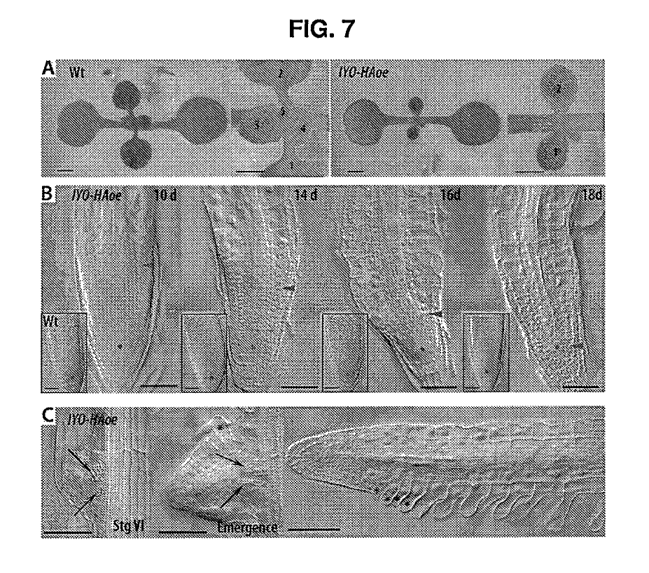

The overexpression of IYO or its fusions with proteins or peptides such as GFP, HA, FLAG under the control of the constitutive 35S promoter leads to premature differentiation in meristems, including in the rib meristem with the consequent shortening of internodes and the compaction of inflorescences (FIG. 6B-C), and even to meristem consumption (FIG. 7).

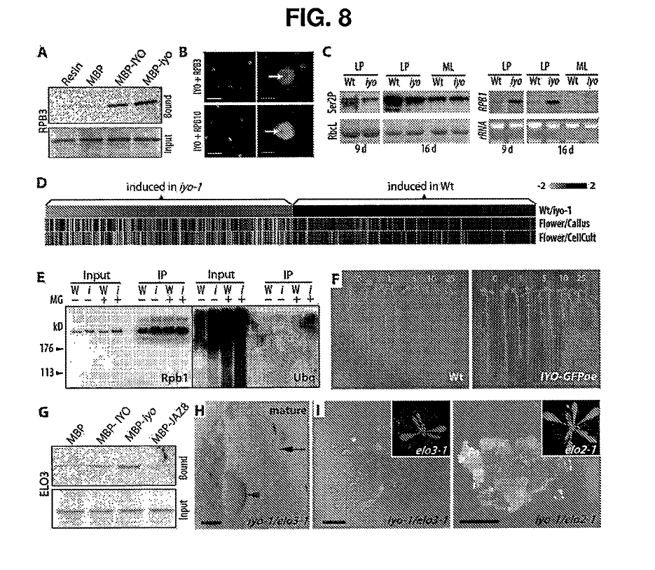

The IYO gene codes for a protein which interacts physically with RNA polymerase II (Pol II) and with elongation complexes (FIG. 8A-B, G-I) and which activates transcriptional elongation (FIG. 8C, E-F) and the expression of development programmes directing differentiation (FIGS. 8C-D, 6A).

Through the analysis of co-expressed genes, Applicants identified the gene At5g26760 (AtRTR1) which has a Pearson correlation coefficient of r=0.625 with IYO. At5g26760 codes for a protein which is highly conserved in all plant species, is homologous to the RTR1 protein from Saccharomyces cerevisae, and also has homologues in animals. RTR1 interacts with Pol II and results have recently been published which indicate that it acts as a transition phosphatase for Pol II in yeast (Mosley et al., 2009). This phosphatase is thought to be involved in the dephosphorylation of serine 5 in the C-terminal domain of the RPB 1 (CTD) sub-unit, a modification preceding phosphorylation in serine 2 which is necessary for Pol II to enter into productive elongation. Bearing in mind the homology between At5g26760 and this phosphatase which acts in the transition between initiation and elongation in transcription and its co-expression with the IYO gene, which is an activator of transcriptional elongation, Applicants postulate that At5g26760/AtRTR1 is jointly involved with the IYO gene in the activation of transcriptional elongation and the initiation of cell differentiation in plants.

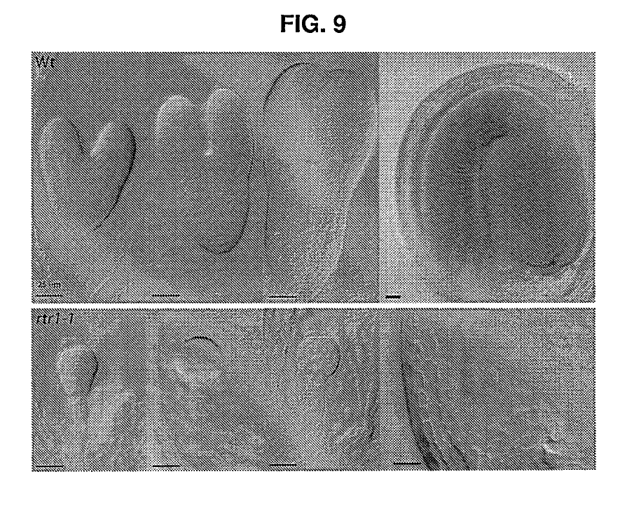

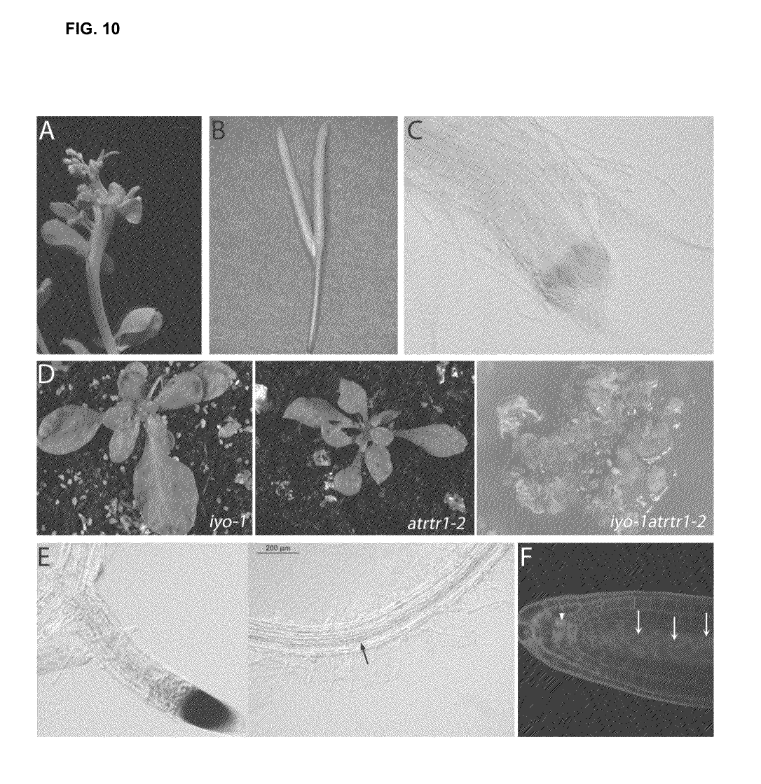

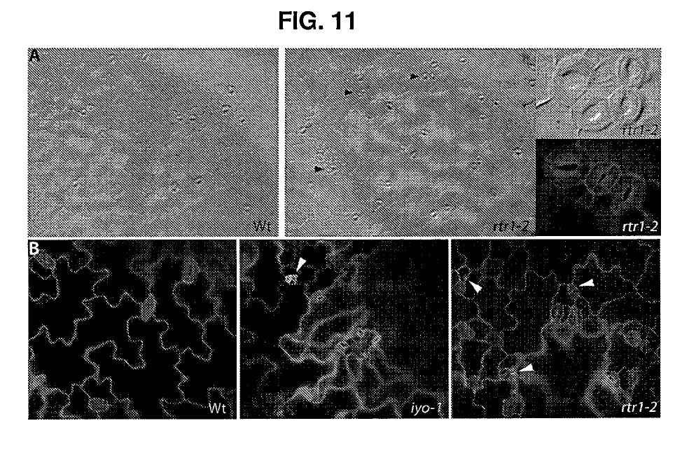

As a first step in analysing the function of AtRTR1, Applicants studied two mutant alleles in the SALK collection. The allele atrtr1-1 (SALK_{—}012339) has an insertion of T-DNA in the first exon which gives rise to the total loss of function of RTR1. Homozygous atrtr1-1 plants arrest their growth during early stages of embryogenesis (FIG. 9). The arrested embryos have a phenotype which is very similar to strong alleles of iyo, which may comprise an almost total block on cell differentiation and the production of twin embryos from suspensor cells. The atrtr1-2 allele (SALK_{—}115762) has a T-DNA insertion in the third intron of the gene, which is spliced out but with low efficiency, resulting in very low accumulation of the full-length transcript. The atrtr1-2 allele results in a partial loss of AtRTR1 activity and phenocopies almost exactly the weak allele iyo-1. atrtr1-2 plants have delayed onset of differentiation in the different meristems of the plant and therefore maintain ectopic stem cells that generate additional shoot, flower and root meristems (FIG. 10A-C) and ectopic meristemoids (FIG. 11). Ectopic embryos that germinate and give rise to cloned plants are also generated.

The phenotype of a double iyo-1atrtr1-2 mutant shows a clear interaction between the IYO and AtRTR1 genes in control of the initiation of cell differentiation. These double mutants have a total block on differentiation that gives rise to growth in the form of a mass of undifferentiated cells (FIG. 10D). In addition, Applicants have demonstrated that the over-expression of IYO in a mutant atrtr1-2 ecotype does not give rise to any phenotype, demonstrating that the RTR1 gene is necessary for the function of MINIYO.

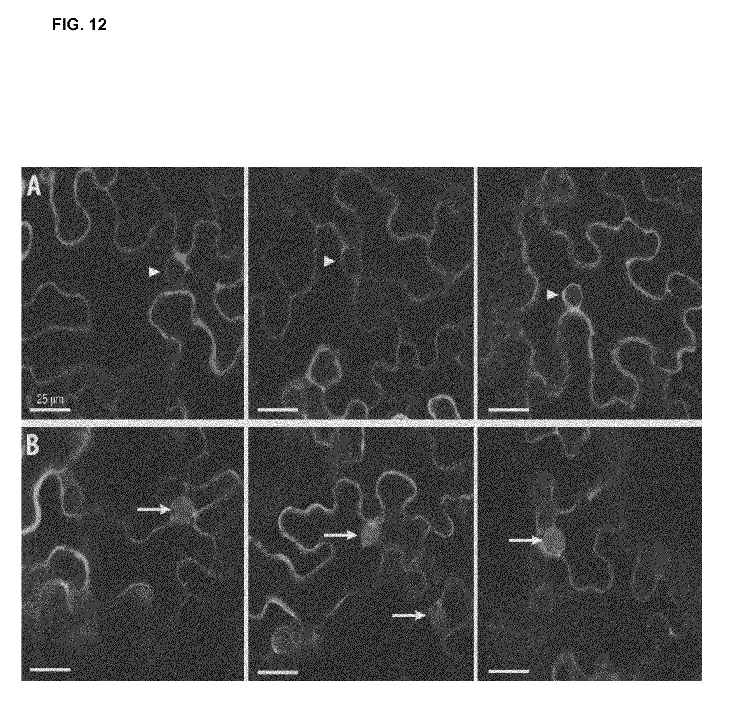

In order to study the expression of RTR1, Applicants generated transgenic plants expressing the promoter of RTR1 and the first three exons and introns of RTR1 translationally fused to the UidA (GUS) reporter gene. This construct directs the activity of GUS specifically to meristems and differentiating tissues (FIG. 10E), in a pattern which is very similar to that of plants which express GUS under the promoter of IYO, which is consistent with the high level of co-expression of the corresponding transcripts and is compatible with the fact that both genes function together and in a coordinated way. Furthermore, Applicants analysed the pattern of accumulation of AtRTR1 fused to the GFP reporter under the control of the constitutive CaMV 35S promoter . In the same way as IYO-GFP, the fusion product AtRTR1-GFP does not accumulate in the nuclei of undifferentiated cells (FIG. 10F), being excluded through active export dependent on Exportin1 (FIGS. 10F and 12).

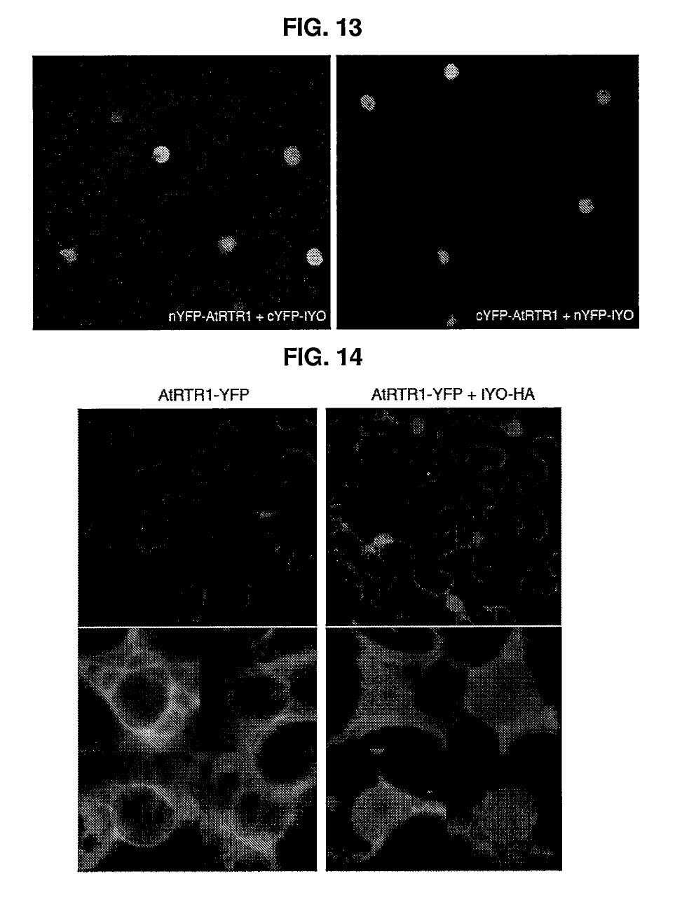

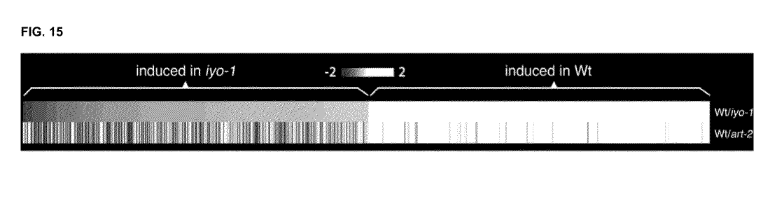

Although the bulk of AtRTR1 is present in the cytosol, we found high levels of nuclear fluorescence reconstitution when split YFP fused to IYO and AtRTR1 was expressed in Nicotiana benthamiana leaves (FIGS. 12-14). This confirms that IYO and AtRTR1 interact physically, and that they do so in the nucleus. Moreover, reconstituted YFP stabilizes complexes, and this leads to large accumulation of AtRTR1 in the nucleus, which suggest that complex formation serves to retain AtRTR1 in that compartment. To test this, Applicants analyzed the subcellular distribution of AtRTR1 in the presence or absence of co-expressed IYO. These experiments confirmed that co-expressed IYO increases the accumulation of AtRTR1 in the nucleus.

These results demonstrate that MINIYO and RTR1 form a complex in the nucleus and that they have a common and shared function in combining to initiate cell differentiation by the activation of transcriptional elongation through the interaction and modification of Pol II.

Applicants have shown that downregulation of MINIYO and/or RTR1 genes or their proteins leads to a delayed onset of differentiation, increased meristem size/number and ectopic meristems. Thus, downregulation of MINIYO and/or RTR1 can be useful in increasing plant yield. The term “yield” as described herein relates to yield-related traits. Specifically, these include an increase in biomass and/or seed yield. This can be achieved by increased growth. An increase in yield can be, for example, assessed by the harvest index, i.e. the ratio of seed yield to aboveground dry weight. Thus, according to the invention, yield may comprise one or more of: increased seed yield per plant, increased seed filling rate, increased number of filled seeds, increased harvest index, increased number of seed capsules/pods, increased seed size, increased growth or increased branching, for example inflorescences with more branches. Preferably, yield may comprise an increased number of seed capsules/pods and/or increased branching. Yield is increased relative to control plants. An increase in yield may be about 5, 10, 20, 30, 40, 50% or more compared to a control plant.

In contrast, overexpression of MINIYO and/or RTR1 genes can be used to eliminate branches of the inflorescence meristem in crops where this is useful.

The invention is therefore based on the generation of plants having either increased or reduced activity of MINIYO and/or RTR1 genes or their proteins, including AtMINIYO and AtRTR1 or their orthologues in other plant species, to advance or delay the onset of differentiation in the meristems, at the required time in each case. Thus, the activity of MINIYO and/or RTR1, including AtMINIYO and AtRTR1 or their orthologues in other plant species, may be inactivated, repressed or downregulated. In another aspect, the activity of MINIYO and/or RTR1, including AtMINIYO and AtRTR1 or their orthologues in other plant species, is increased or up-regulated. Transgenic or mutant plants which express MINIYO and/or RTR1 genes, including AtMINIYO and AtRTR1 or their orthologues in other plant species, but where the function of the protein is partly lost, may be obtained according to the various aspects of the invention.

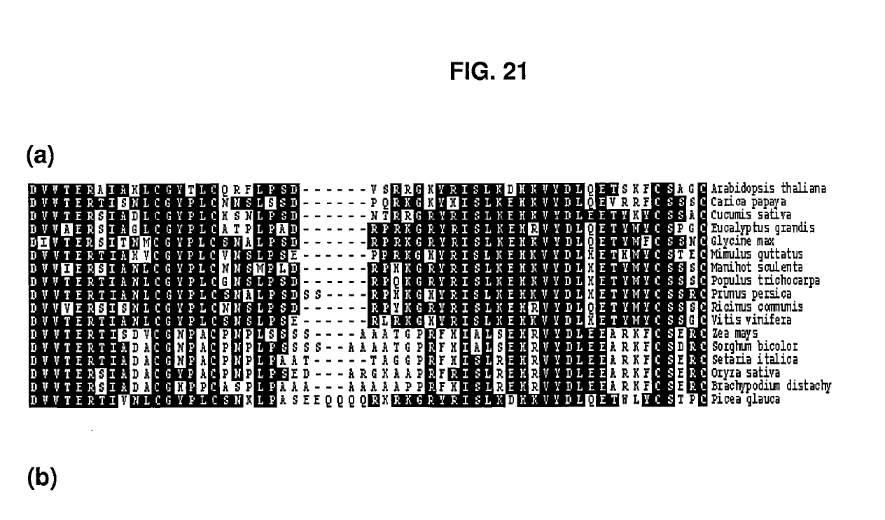

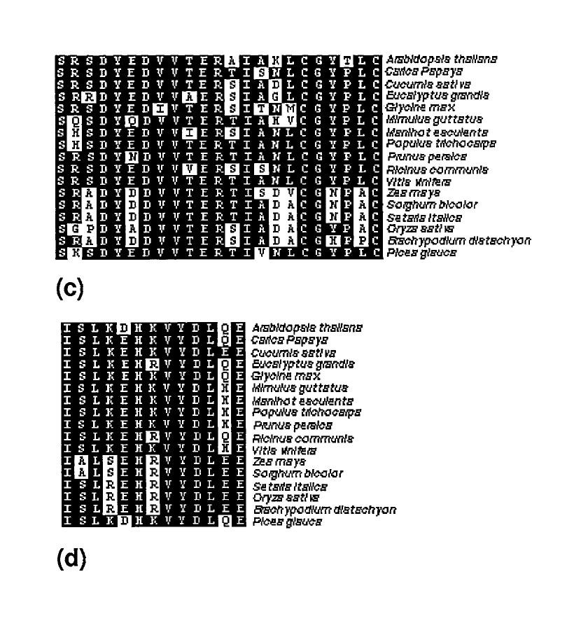

Alternatively, null mutants are obtained which are transformed with or carry attenuated or mutant versions of MINIYO or RTR1 genes, such as the iyo-1 alleles and atrtr1-2 alleles or a combination thereof, or other alleles which have mutated amino acids in regions which are highly conserved in MINIYO and RTR1 proteins, including AtMINIYO and AtRTR1 or their orthologues in other plant species (FIGS. 18 and 21). The expected phenotypes are similar to those already observed in Arabidopsis, i.e.: larger meristems and directly generated organs (thicker stems), ectopic meristems which give rise to additional inflorescences, duplicated flowers, a larger number of side roots and seeds with double embryos.

Throughout this disclosure, MINIYO and RTR1 are used to refer to the genes homologous/proteins to the Arabidopsis AtMINIYO and AtRTR1 genes/proteins respectively, as described herein.

Specifically, a skilled person would therefore understand that the invention not only relates to isolated AtMINIYO and AtRTR1 genes/proteins as defined in SEQ ID No. 1, 8, 5 and 11 and their uses in the various aspects of the invention, but that the present invention relates to methods and uses of homologues and orthologues of the AtMINIYO or AtRTR1 genes and their polypeptides in other plant species, including transgenic plants where expressing or activity of such an orthologous gene/protein is increased or decreased.

Thus, in a first aspect, the invention relates to an isolated nucleic acid molecule or sequence which may comprise a nucleic acid molecule of SEQ ID No. 1 coding for the AtMINIYO protein of SEQ ID No. 5, or its orthologue in another plant species. In another aspect, the invention relates to an isolated nucleic acid sequence or molecule which may comprise a nucleic acid of SEQ ID No. 8 coding for the AtRTR1 protein of SEQ ID No. 11 or its orthologue in another plant species.

As explained herein, said nucleic acid molecule(s) control(s) the initiation of cell differentiation in apical, root and floral meristems of the plant. Preferably, the nucleic acid molecule(s) is/are homologous to the corresponding nucleic acid molecules which code for the AtMINIYO proteins of SEQ ID No. 5 or its orthologue in another plant species, and/or the AtRTR1 protein of SEQ ID No. 11 or its orthologue in another plant species.

In a preferred embodiment of the invention, the nucleic acid molecule is characterised by interacting and/or modifying the RNA polymerase II (Pol II) involved in the activation of transcriptional elongation and the expression of developmental programmes which direct the initiation of cell differentiation in seeds and in all the apical, root and floral and other meristems of the plant. In a preferred embodiment, the protein AtMINIYO or its orthologue in other plant species, and the protein AtRTR1 or its orthologue in other plant species, interact and/or modify the RNA polymerase II (Pol II), and are jointly involved in the activation of transcriptional elongation and the expression of developmental programmes which direct the initiation of cell differentiation in seeds and in all the apical, root and floral and other meristems of the plant.

This invention also protects an isolated nucleic acid which may comprise a nucleotide sequence coding for an amino acid sequence of the protein IYO and/or the protein AtRTR1, or their orthologues in another plant species, which are at least 30% identical to the sequences coded by SEQ ID NO: 1 and/or SEQ ID NO: 8 to control the initiation of cell differentiation in seeds and in apical, root and floral and other meristems of a plant, and their uses, preferably their use to control the initiation of cell differentiation in seeds and in apical, root and floral meristems of a plant.

Accordingly, the invention relates to an isolated nucleic acid sequence which may comprise or which may consist of SEQ No. 1 or 8 or a homologue, orthologue or functional variant thereof. In one embodiment, the isolated nucleic acid sequence may comprise or consists of SEQ No. 1 or 8. In another embodiment, the isolated nucleic acid sequence may comprise or consists of a nucleic acid sequence that encodes for an orthologue of the protein identified in SEQ No. 5 or 11.

As used herein, the words “nucleic acid”, “nucleic acid sequence”, “nucleic acid molecule”, “nucleotide”, or “polynucleotide” are intended to include DNA molecules (e.g., cDNA-as is the case for SEQ ID NO: 1 and SEQ ID NO: 8- or genomic DNA), RNA molecules (e.g., mRNA), natural occurring, mutated, synthetic DNA or RNA molecules, and analogs of the DNA or RNA generated using nucleotide analogs. The skilled person will understand that where the nucleic acid according to the invention includes RNA, reference to the sequence shown should be construed as reference to the RNA equivalent, with U substituted for T. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. The sequences may also be synthetically made sequences. The nucleic acid may be wholly or partially synthetic, depending on design.

The term “functional part or functional variant” as used herein refers to a variant gene or polypeptide sequence or part of the gene or polypeptide sequence which retains the biological function of the full non-variant sequence, i.e. acts as a molecular switch to initiate cell differentiation. Variant degenerate sequences of the nucleotide sequences according to the invention whose product is a protein having the same function as the protein coded by each of the sequences SEQ ID NO: 5 and SEQ ID NO: 11 are thus included within the scope of the invention. The amino acid sequence may be coded by any nucleotide sequence which gives rise to any of the amino acid sequences according to the invention. Due to the fact that the genetic code is degenerate, the same amino acid may be coded for by different codons (triplets), and thus the same amino acid sequence may be coded for by different nucleotide sequences.

The homologue, orthologue or functional variant of SEQ ID No. 1 or 8 encodes a polypeptide that is 30%-99% identical to a sequence encoded by SEQ No. 1 or 8. For example, the polypeptide of the invention has, in increasing order of preference, at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 5 or 11, respectively, and/or represented by the AtMINIYO or AtRTR1 orthologues and paralogues shown herein.

In one embodiment, the isolated nucleic acid of the invention encodes a polypeptide that is at least 30% identical to a sequence encoded by SEQ No. 5 or 11. In another embodiment, the degree of identity between the amino acid sequences encoded by SEQ ID NO: 1 or SEQ ID NO: 8 originating from Arabidopsis thaliana and an amino acid sequences from another plant, preferably a plant belonging to the superfamily Viridiplantae, is around 90% or 95%. Furthermore, all sequences whose transcription product is substantially identical to the amino acid sequences SEQ ID NO: 5 and SEQ ID NO: 11 according to this invention are included.

The amino acid sequences which are at least 30% identical to those coded by SEQ ID NO: 1 and SEQ ID NO: 8 are homologous sequences from A. thaliana or other organisms in which the protein for which they code has an equivalent function to the protein coded by the said MINIYO and RTR1 genes of plant origin, for example from Arabidopsis. The homologous sequences in general relate to sequences from different species originating from a common ancestral sequence. Two types of homology are generally distinguished in sequence homology: orthology and paralogy. Orthologous sequences belong to species which have a common past. Paralogous sequences are those which are found in the same organism and originate from duplication of a given gene. In one embodiment, the invention relates to any homologous sequences, including both orthologous and paralogous, which are at least 30% identical to the amino acid sequences encoded by SEQ ID NO: 1 or SEQ ID NO: 8, without prejudice to whether other sequences with lower degrees of identity with MINIYO and RTR1 are also regarded as being an object of the invention.

The overall sequence identity is determined using a global alignment algorithm, for example the Needleman Wunsch algorithm in the program GAP (GCG Wisconsin Package, Accelrys), preferably with default parameters and preferably with sequences of mature proteins (i.e. without taking into account secretion signals or transit peptides).

The orthologue may be selected from a MINIYO or RTR1 gene in any other plant, preferably a plant of superfamily Viridiplantae, in particular monocotyledonous and dicotyledonous plants, including forage plants and vegetables for livestock, ornamental plants, crop plants for use in human or animal nutrition and plants for use as bioenergy. Specific plants from which the ortholgue may be derived are listed elsewhere in this application as non-limiting examples of transgenic plants. In one embodiment of the various aspects of the invention, the MINIYO gene is selected from one of the following plants:

Oryza sativa (SEQ ID No. 12 peptide sequence, SEQ ID No. 18 nucleic acid sequence), Zea mays (SEQ ID No. 13 peptide sequence, SEQ ID No. 19 nucleic acid sequence), Glycine max (SEQ ID No. 14 and 15 peptide sequences, SEQ ID No. 20, 21 nucleic acid sequences), Brachypodium distachyon (SEQ ID No. 16 peptide sequence), Sorghum bicolor (SEQ ID No. 17 peptide sequence).

In one embodiment of the various aspects of the invention, the RTR1 gene is selected from one of the following plants:

Oryza sativa (SEQ ID No. 22 peptide sequence, SEQ ID No. 28 nucleic acid sequence), Zea mays (SEQ ID No. 23 peptide sequence, SEQ ID No. 29 nucleic acid sequence), Glycine max (SEQ ID No. 24, 25 peptide sequences, SEQ ID No. 30, 31 nucleic acid sequences), Brachypodium distachyon (SEQ ID No. 27 peptide sequence), Sorghum bicolor (SEQ ID No. 26 peptide sequence).

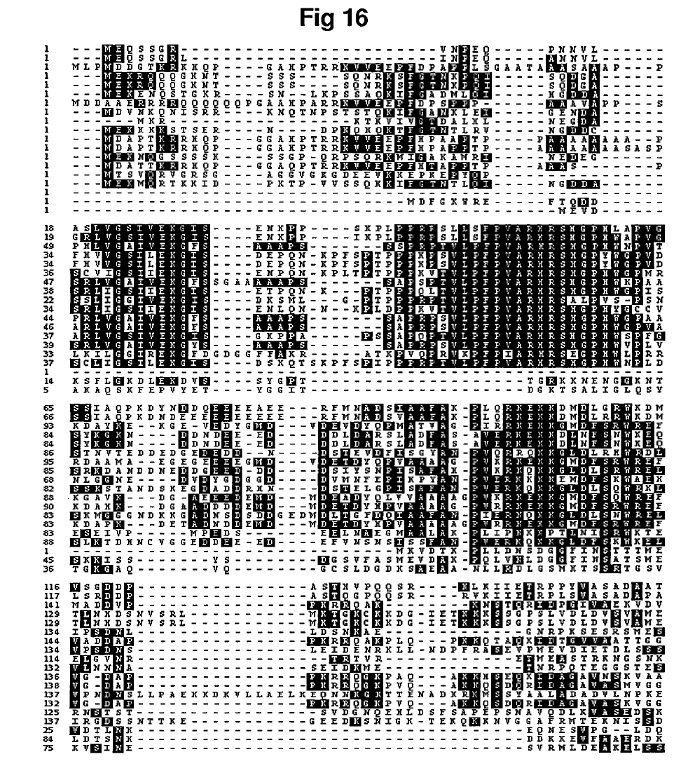

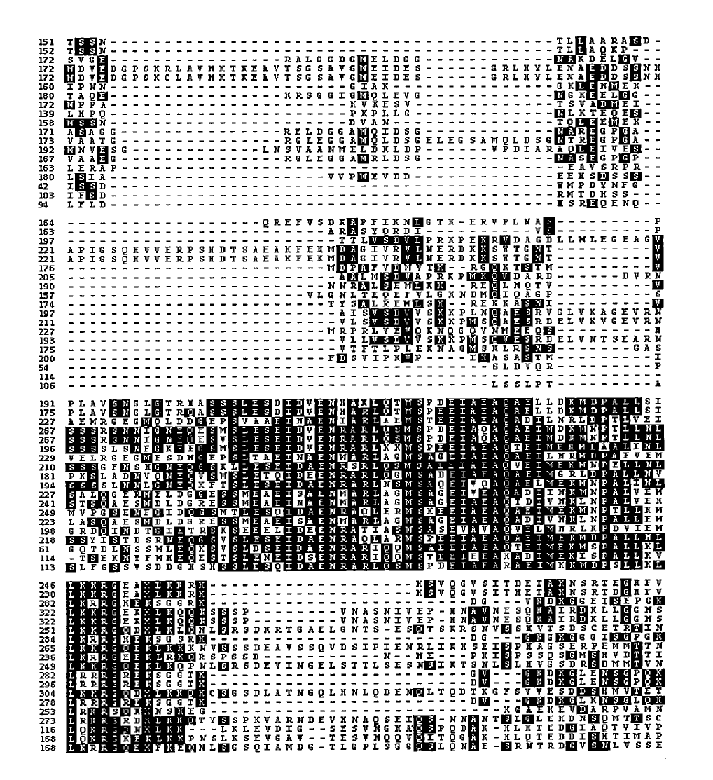

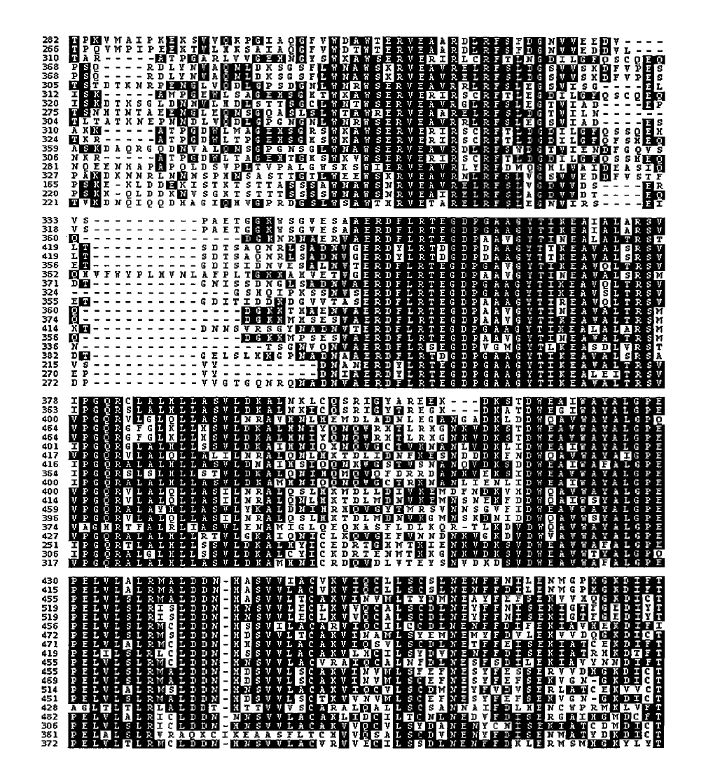

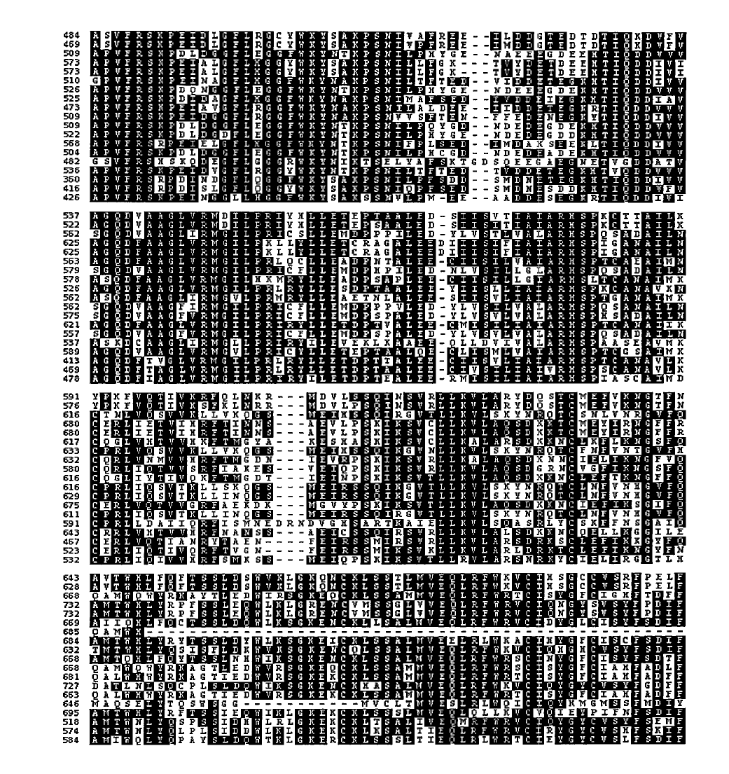

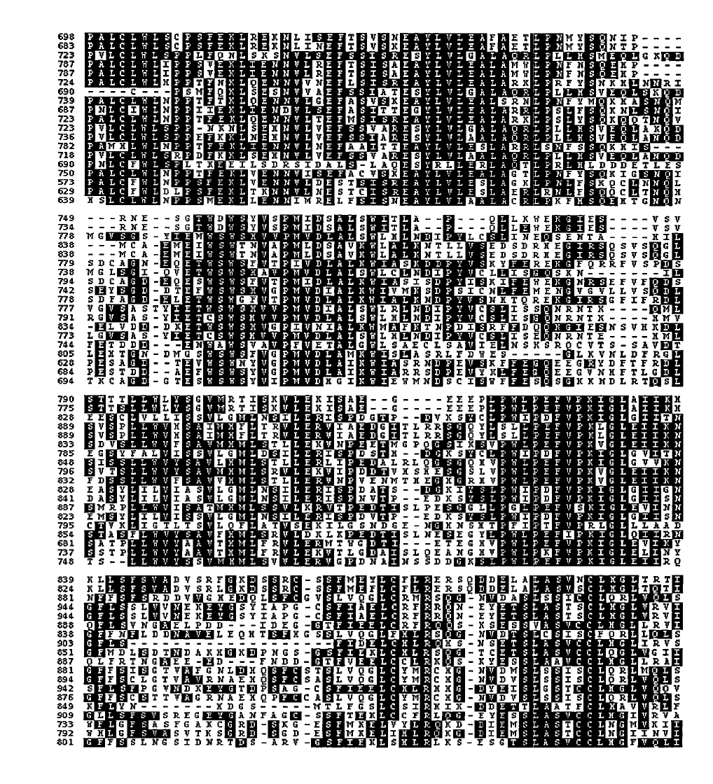

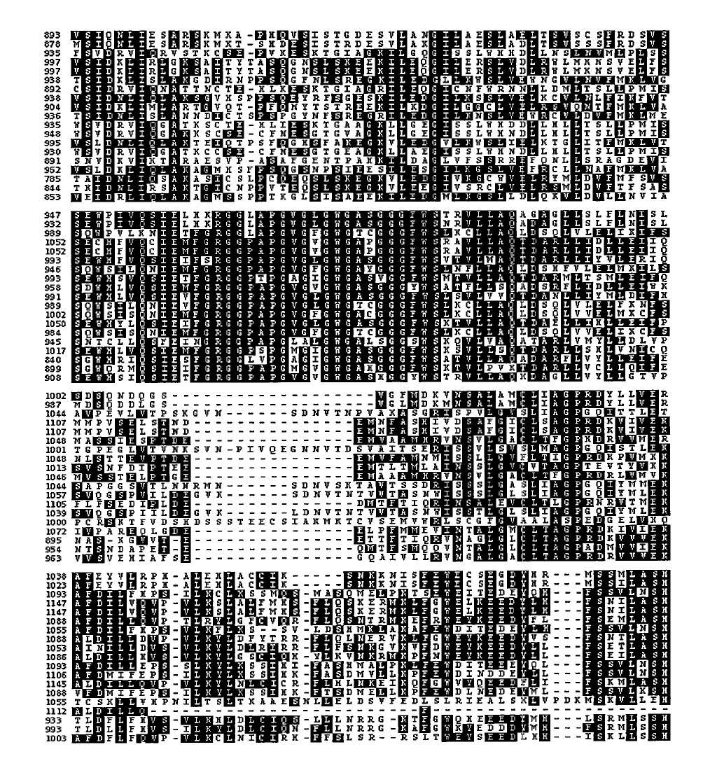

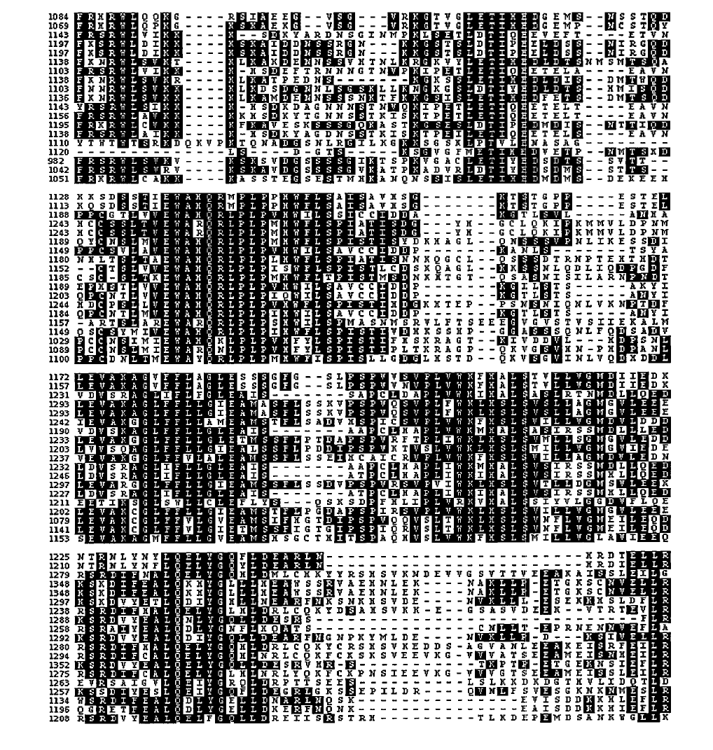

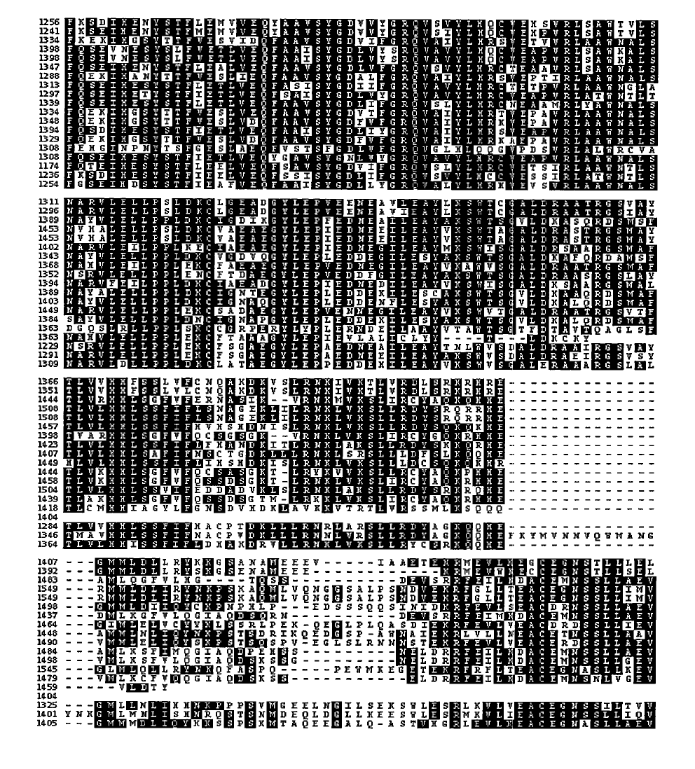

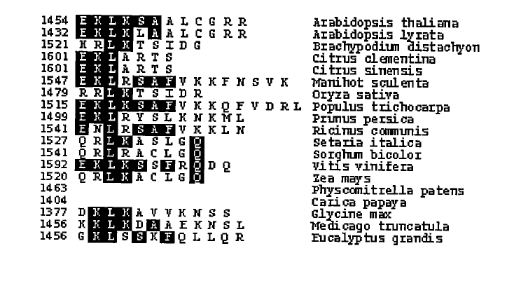

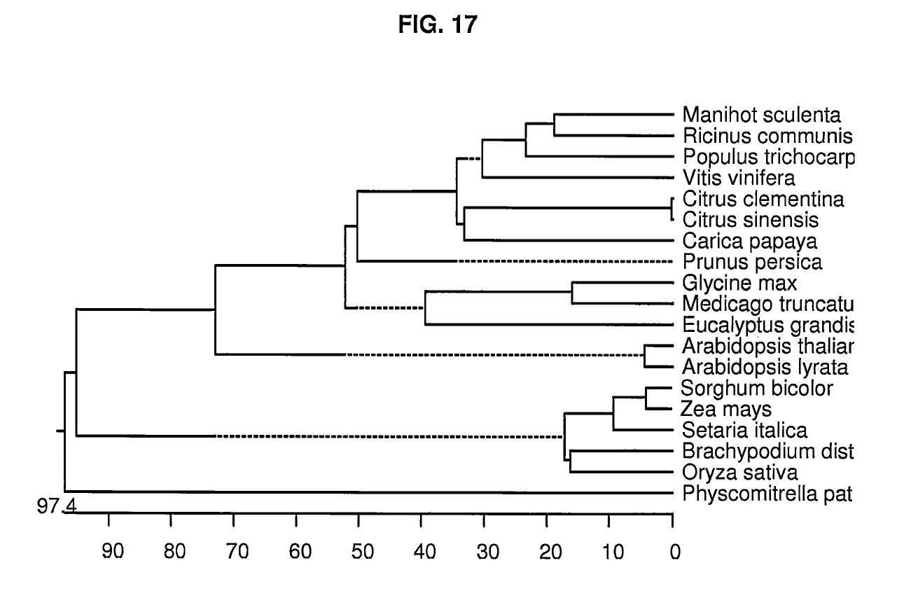

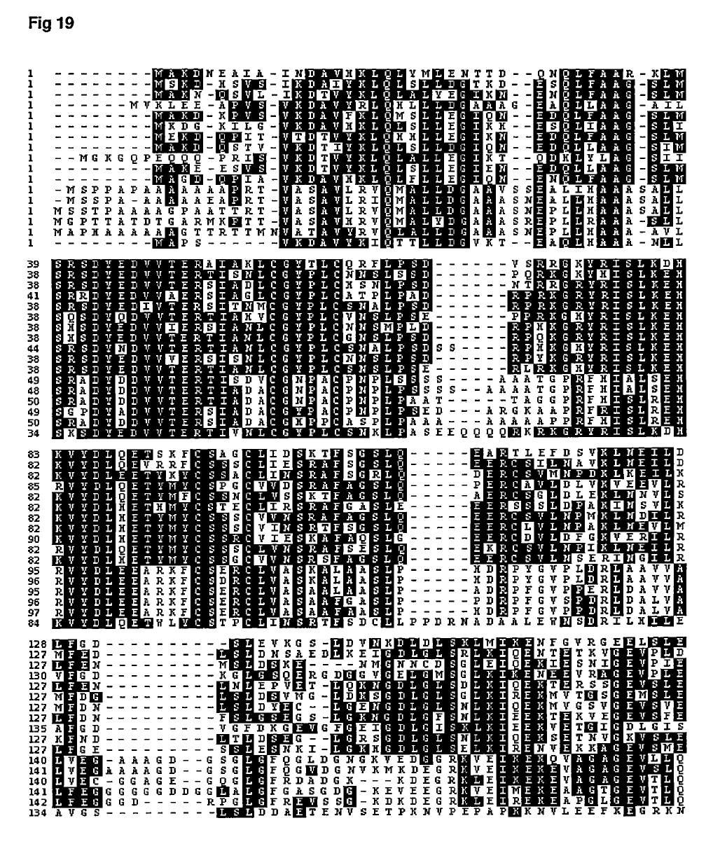

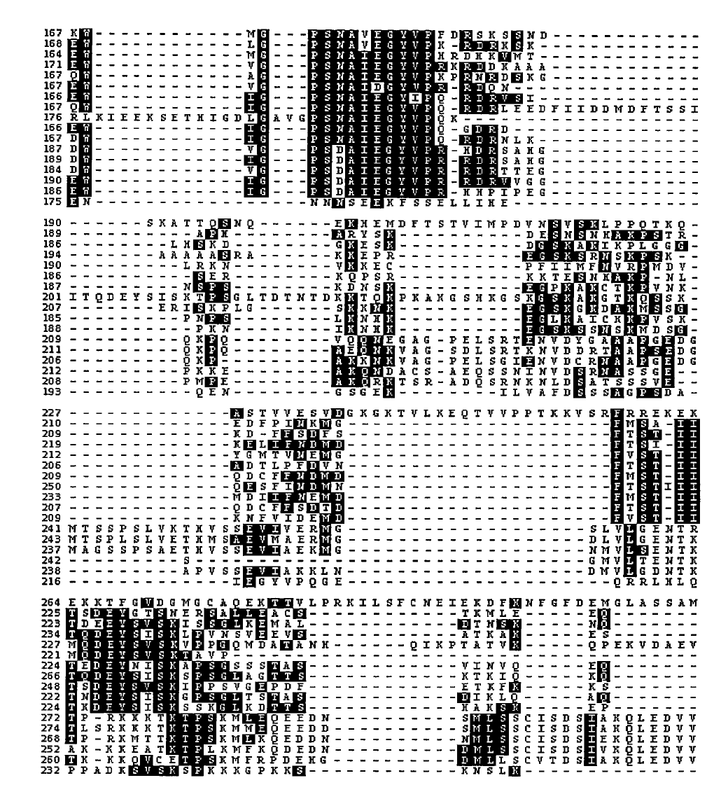

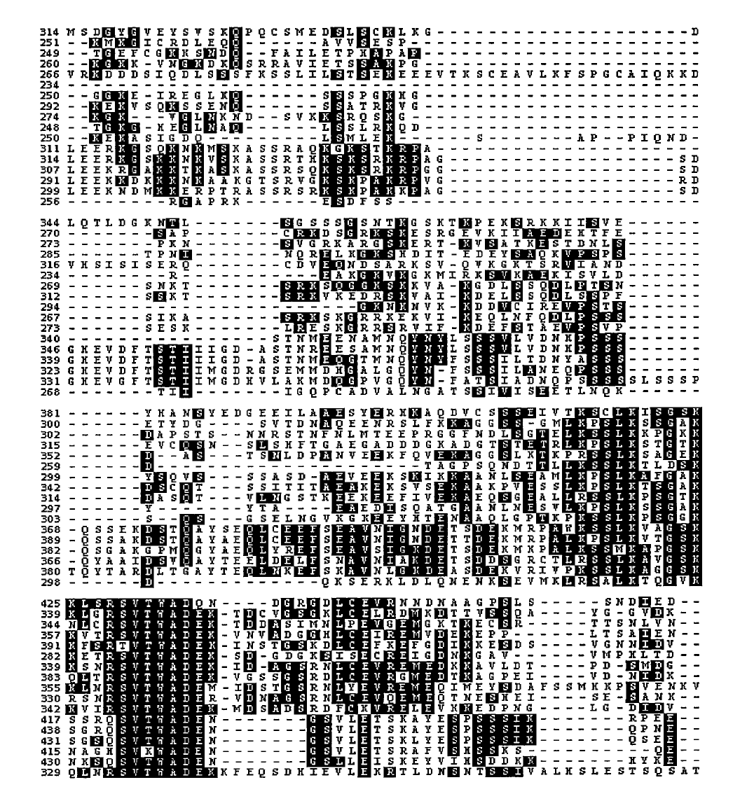

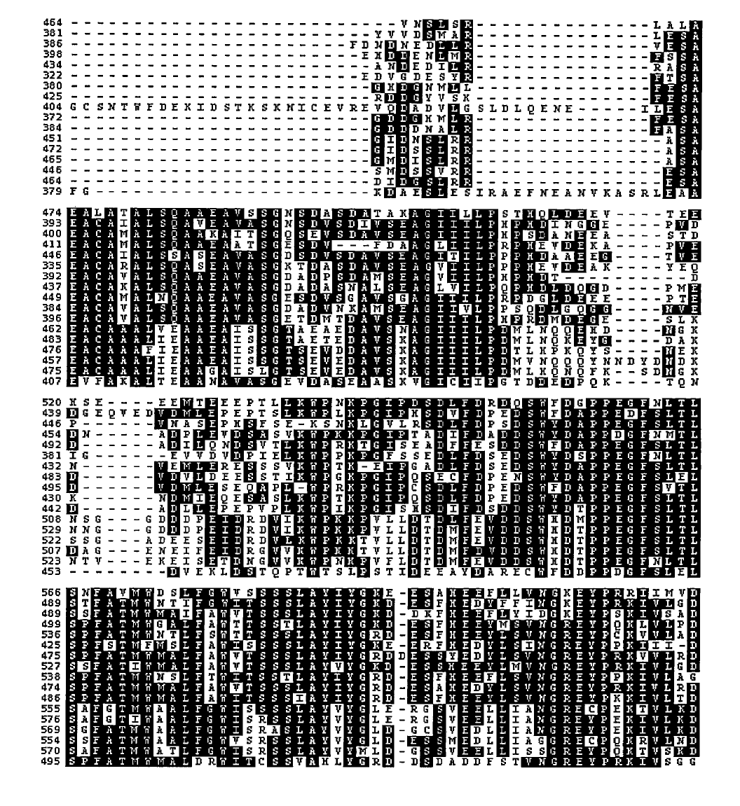

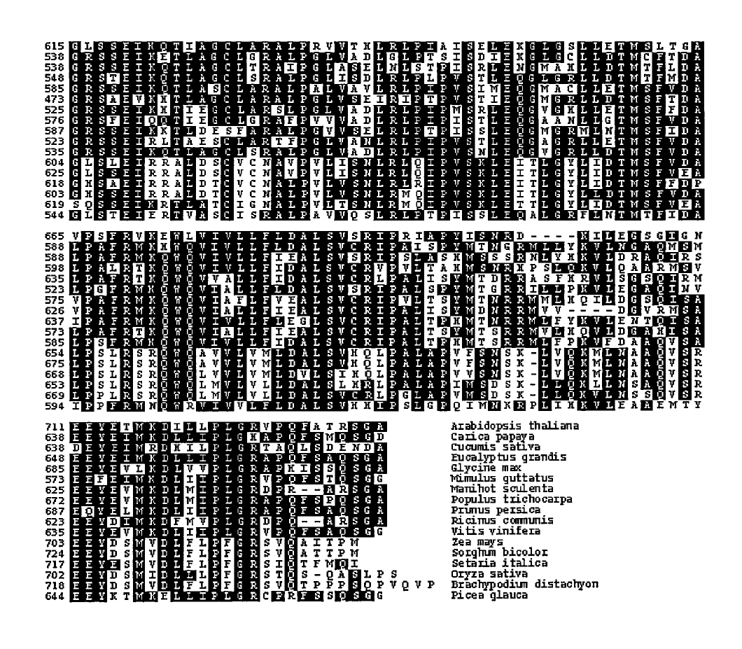

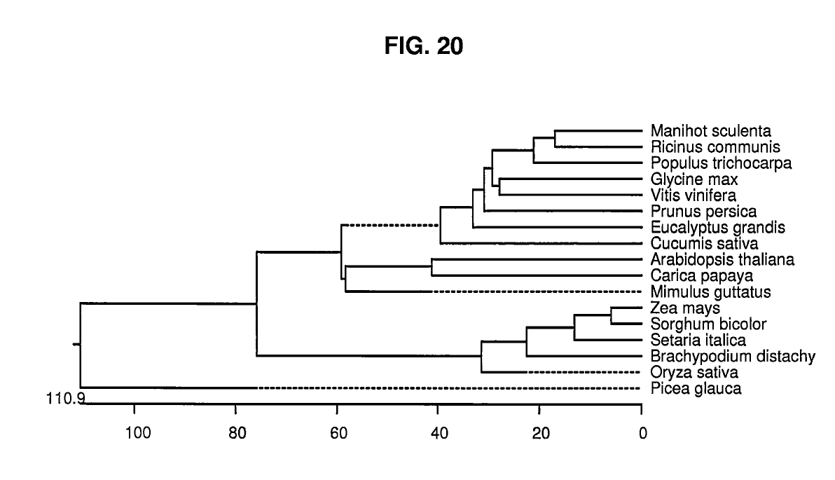

As shown in FIGS. 16 to 21, genes encoding for MINIYO and RTR1 in plants and their resulting proteins are conserved and show a number of conserved domains.

For the MINIYO protein, one of these domains is a glycine rich domain which may comprise an RGG element. This domain is located at position 960-980 in Arabidopsis AtMINIYO. Mutating G962E results in a partial loss of function mutant. Therefore, orthologues of AtMINIYO are characterised by the presence of a conserved glycine rich domain as shown in FIG. 3b for MINIYO proteins from different species. Thus, orthologues proteins may comprise a sequence which has at least 80%, 81%, 82%, 83%, 84%, 85%, 86%, 87%, 89%, 90%, 91%, 92%, 93%, 94%, 95%, 96%, 97%, 98% or 99% identity with the domain in Arabidopsis SEQ ID No. 32: RGGLAPGVGLGWGASGGGFWS. FIG. 18 (a).

In addition, as shown in FIG. 18, orthologues are characterised by the presence of one or more further conserved domains which show a high degree of sequence identity, preferably at least 80%, 81%, 82%, 83%, 84%, 85%, 86%, 87%, 89%, 90%, 91%, 92%, 93%, 94%, 95%, 96%, 97%, 98% or 99% to the following domains in Arabidopsis:

amino acids 209-255
SEQ ID No. 33
SDIDVENHAKLQTMSPDEIAEAQAELLDKMDPALLSILKKRGEAKLK.
FIG. 18 (b);
amino acids 317-396
SEQ ID No. 34
RDLRFSFDGNVVEEDVVSPAETGGKWSGVESAAERDFLRTEGDPGAAGY
TIKEAIALARSVIPGQRCLALHLLASVLDKA. FIG. 18 (c);
amino acids 350-389
SEQ ID No. 35
ERDFLRTEGDPGAAGYTIKEAIALARSVIPGQRCLALHLL. FIG. 18
(d);
amino acids 417-437
SEQ ID No. 36
DWEAIWAYALGPEPELVLALR. FIG. 18 (e).;
amino acids 529-559
SEQ ID No. 37
TIQKDVFVAGQDVAAGLVRMDILPRIYHLLEE. FIG. 18 (f);
amino acids 529-597
SEQ ID No. 38
TIQKDVFVAGQDVAAGLVRMDILPRIYHLLEEPTAALEDSIISVTIAIAR
HSPKCTTAILKYPKFVQT. FIG. 18 (g);
amino acids 1136-1145
SEQ ID No. 39
EWAHQRMPLP. FIG. 18 (h);
and/or
amino acids 1144-1416
SEQ ID No. 40
LPPHWFLSAISAVHSGKTSTGPPESTELLEVAKAGVFFLAGLESSSGFGS
LPSPVVSVPLVWKFHALSTVLLVGMDIIEDKNTRNLYNYLQELYGQFLDE
ARLNHRDTELLRFKSDIHENYSTFLEMVVEQYAAVSYGDVVYGRQVSVYL
HQCVEHSVRLSAWTVLSNARVLELLPSLDKCLGEADGYLEPVEENEAVLE
AYLKSWTCGALDRAATRGSVAYTLVVHHFS SLVFCNQAKDKVSLRNKIV
KTLVRDLSRKRHREGMMLDLLRYK. FIG. 18 (i)

For the RTR1 protein, there is a conserved domain (DUF408) with a zinc-finger like motif located at the N-terminus of the protein that is found in all the orthologues from plants, animals and fungi. This domain is located at position 45-98 in Arabidopsis AtRTR1. The zinc-finger-like-motif has been implicated in interaction with the RNA Polymerase II C-terminal domain (CTD) and the Integrator complex in humans and is required for CTD-phosphatase activity in yeast and humans (Mosley et al., 2009; Egloff et al., 2011). Interestingly, this motif is also required for interaction of RTR1 with IYO. Substituting the putative zinc coordinating cysteine residues (C56A/C61A or C94A/C98A) in the full-length AtRTR1 protein for alanine abrogates interaction with IYO. Intriguingly, however, both the truncated N-terminal and the C-terminal halves of RTR1 can interact with IYO, suggesting that although RTR1 binds at both ends of the protein to IYO, it requires an intact zinc-finger-like motif in the context of the full length protein for binding.

A consensus sequence for the zinc-finger like motif derived from sequences from multicellular eukaryotes is (the putative Zinc-coordinating cysteines are highlighted in bold):

D[IV]V[TDEV]ER[ASTF]I[AVIS][KND][LAV]CGY[TP][LRA]
CXXXLX₇₋₁₅[YF][RK]IS[LT][KSR][TAED][HKN][KR]VYD
[IL][THEQ]EXXX[FY]CXXXC

A blast search against the non-redundant protein sequence database at NCBI with the corresponding sequence from Arabidopsis DVVTERAIAKLCGYTLCQRFLPSDVSRRGKYRISLKDHKVYDLQETSKFCSAGC SEQ ID No. 41 retrieved the RTR1 orthologues from plants, animals and fungi with a low E-value (<10⁻⁶).

Therefore, orthologues of AtRTR1 from plants animals and fungi are characterised by the presence of a conserved zinc-finger like motif as shown in FIG. 21. Thus, orthologues proteins may comprise a sequence which has at least 80%, 81%, 82%, 83%, 84%, 85%, 86%, 87%, 89%, 90%, 91%, 92%, 93%, 94%, 95%, 96%, 97%, 98%, or 99% identity with the domain in Arabidopsis SEQ ID No. 42:

DVVTERAIAKLCGYTLCQRFLPSDVSRRGKYRISLKDHKVYDLQETSKFC
SAGC. FIG. 21 (a).

In addition, orthologues to the AtRTR1 Arabidopsis protein are characterised by the presence of one or more further conserved domains as shown in FIG. 21, which show a high degree of sequence identity, preferably at least 80%, 81%, 82%, 83%, 84%, 85%, 86%, 87%, 89%, 90%, 91%, 92%, 93%, 94%, 95%, 96%, 97%, 98%, or 99% to the following domains in Arabidopsis:

amino acids 39-61
SEQ ID No. 43
SRSDYEDVVTERAIAKLCGYTLC. This domain includes
catalytic cysteine C56 which is strictly conserved
in all the orthologues. FIG. 21 (b).
amino acids. 77-89
SEQ ID No. 44
ISLKDHKVYDLQE. FIG. 21 (c);
amino acids. 429-435
SEQ ID No. 45
SVTWADQ. FIG. 21 (d);;
amino acids 473-507
SEQ ID No. 46
AEALATALSQAAEAVSSGNSDASDATAKAGIILLP. FIG. 21 (e);
and/or
amino acids 552-589.
SEQ ID No. 47
SWFDGPPEGFNLTLSNFAVMWDSLFGWVSSSSLAYIYG. FIG. 21
(f)

Thus, a skilled person would understand that MINIYO and RTR1 are highly conserved in plants and characterised by the presence of the conserved domains above. Accordingly, the MINIYO and RTR1 proteins according to the invention can be defined and identified through the presence of these domains set out herein, in particular with reference to FIGS. 18 and 21. A skilled person would therefore be able to identify orthologues to AtMINIYO and ATRTR1 by reference to these domains through routine methods.

Another aspect of the invention relates to an expression vector which may comprise one or more isolated nucleic acid molecule(s) of the invention. The invention also relates to the use of an expression vector as described herein to control the initiation of cell differentiation in the apical, root floral and/or other meristems of a plant.

The term “vector” refers to a fragment of DNA which has the ability to replicate in a given host and, as the term indicates, it can act as a vehicle to multiply another DNA fragment which has been fused to it (“insert”). “Insert” refers to a fragment of DNA fused to the vector; in the case of this invention the vector may comprise any of the sequences described in accordance with the aspects of the invention which, when fused to the same, can replicate in a suitable host. The vectors may be plasmids, cosmids, bacteriophages or lentiviral vectors suitable for transforming or transfecting fungal or animal cells, without excluding other kinds of vectors which correspond to the definition of vector provided.

Expression of the said nucleic acid molecules may be under the control of a promoter sequence. The promoter used in the gene constructs of the vectors described above to express MINIYO or RTR1 may be an endogenous MINIYO or RTR1 promoter, for example the AtMINIYO or AtRTR1 promoter (SEQ Id No 48 and 49) or a MINIYO or RTR1 promoter from a AtMINIYO or AtRTR1 orthologue.

Alternatively, the promoter may regulate overexpression of the gene. Overexpression according to the invention means that the transgene is expressed at a level that is higher than expression of endogenous counterparts (MINIYO or RTR1) driven by their endogenous promoters. For example, overexpression may be carried out using a strong promoter, such as the cauliflower mosaic virus promoter (CaMV35S), the rice actin promoter or the maize ubiquitin promoter or any promoter that gives enhanced expression.

Alternatively, an inducible expression system may be used, where expression is driven by a promoter induced by environmental stress conditions (for example the pepper pathogen-induced membrane protein gene CaPIMPI or promoters that may comprise the dehydration-responsive element (DRE), the promoter of the sunflower HD-Zip protein gene Hahb4 or Hahb1, which is inducible by water stress, high salt concentrations and ABA, or a chemically inducible promoter (such as steroid- or ethanol-inducible promoter system). Such promoters are described in the art. Other suitable promoters and inducible systems are also known to the skilled person.

As a skilled person will know, the expression vector may also comprise a selectable marker which facilitates the selection of transformants, such as a marker that confers resistance to antibiotics, such as kanamycin.

In any of the expression vectors described herein, wild type sequences that encode MINIYO or RTR1 polypeptides can be included, but in one embodiment, variant sequence or fragments may also be used, provided such sequences encode a polypeptide that has the same biological activity as the wild type sequence. Sequence variations in the wild type sequence include silent base changes that do not lead to a change in the encoded amino acid sequence and/or base changes that affect the amino acid sequence, but do not affect the biological activity of the polypeptide. Changes may be conservative amino acid substitutions, i.e. a substitution of one amino acid residue where the two residues are similar in properties. Thus, variant/mutant polypeptides encoded by such sequences retain the biological activity of the wild type polypeptide and act on cell differentiation.

In another embodiment, mutant sequence or fragments may also be used, which encode a polypeptide that has a different biological activity as the wild type sequence. These modifications are described below.

A sequence or vector described herein encoding for the MINIYO or RTR1 protein is introduced as a transgene into the plant. This can be carried out by various methods as known in the field of plant genetic engineering, for example using transformation with Agrobacterium or particle bombardment.

Another embodiment of the invention relates to a host cell which may comprise the expression vector of the invention.

The term “cell” as understood in this invention relates to a prokaryotic or eukaryotic cell. The cell may be a bacterium capable of replicating a transformed foreign DNA such as for example any of the strains of the species Escherichia coli. Preferably cell refers to a eukaryotic fungal, plant or animal cell. Thus, in the case where the cell is a fungus, the term cell may comprise at least an individual cell of a yeast, a mycelium of a filamentous fungus, or other fungal cell of any type, whether germinal (spore) or vegetative, differentiated or undifferentiated. In the case of an animal cell it may be any normal or tumour cell line, from any tissue or organ, adult or embryonal, multipotent (undifferentiated) or differentiated. Likewise a protoplast (a fungal cell without a cell wall) is also included in this definition.

The invention also includes a method for generating of transgenic plants which constitutively or conditionally express or over-express a nucleic acid of the invention, that is a nucleic acid that encodes for a plant MINIYO and/or RTR1 protein, throughout the plant or in specific meristems, to advance the onset of differentiation, reducing the size of the meristems or eliminating them, depending upon the level of over-expression obtained. It also includes the over-expression of mutated versions of MINIYO and/or RTR1 which are not excluded from the nucleus in undifferentiated cells. In one aspect of the invention, mutated constructs of MINIYO and/or RTR1 that are retained in the cytosol or in the nucleus are expressed under constitutive, inducible, tissue-specific or developmental-stage-specific promoters, to modify specifically cell proliferation or cell differentiation rates in different meristems and during embryogenesis. These constructs are described below.

The invention also includes a method for generating transgenic plants in which a nucleic acid of the invention that encodes for a plant MINIYO and/or RTR1 protein is expressed throughout the plant or in specific meristems, to delay the onset of differentiation. Such nucleic acids include mutated constructs of MINIYO and/or RTR1 as described herein.

These methods include introducing an nucleic acid of the invention into said plant by means of recombinant DNA technology and expressing said transgene in the plant.

In another aspect, the invention relates to a transgenic plant wherein the activity of a MINIYO polypeptide as described herein is inactivated, repressed or down-regulated. As described above, said MINIYO protein is at least 30% identical to the sequences coded by SEQ ID NO:1. In one embodiment, the MINIYO protein may comprise or consists of SEQ ID No. 5. Thus, in another aspect, the invention relates to a transgenic plant wherein the activity of a RTR1 polypeptide as described herein is inactivated, repressed or down-regulated. As described above, said RTR1 protein is at least 30% identical to the sequences coded by SEQ ID NO:8. In one embodiment, the RTR1 protein may comprise or consists of SEQ ID No. 11.

In one embodiment, the transgenic plant may be characterised in that activity of both a MINIYO and RTR1 polypeptide as described herein is inactivated, repressed or down-regulated.

In another embodiment, RNA-mediated gene suppression or RNA silencing may be used to achieve silencing of the MINIYO or RTR1 gene. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining. The term should not therefore be taken to require complete “silencing” of expression.

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post transcriptional gene silencing and both may be used according to the methods of the invention.

RNA-mediated gene suppression or RNA silencing according to the methods of the invention includes co-suppression wherein over-expression of the MINIYO or RTR1 gene sense RNA or mRNA leads to a reduction in the level of expression of the genes concerned. RNAs of the transgene and homologous endogenous gene are co-ordinately suppressed.

Other techniques used in the methods of the invention include antisense RNA to reduce transcript levels of the endogenous MINIYO and/or RTR1 gene in a plant. In this method, RNA silencing does not affect the transcription of a gene locus, but only causes sequence-specific degradation of target mRNAs. An “antisense” nucleic acid sequence may comprise a nucleotide sequence that is complementary to a “sense” nucleic acid sequence encoding a MINIYO and/or RTR1 protein, or a part of a MINIYO and/or RTR1 protein, i.e. complementary to the coding strand of a double-stranded cDNA molecule or complementary to an mRNA transcript sequence. The antisense nucleic acid sequence is preferably complementary to the endogenous MINIYO and/or RTR1 gene to be silenced. The complementarity may be located in the “coding region” and/or in the “non-coding region” of a gene. The term “coding region” refers to a region of the nucleotide sequence which may comprise codons that are translated into amino acid residues. The term “non-coding region” refers to 5′ and 3′ sequences that flank the coding region that are transcribed but not translated into amino acids (also referred to as 5′ and 3′ untranslated regions).

The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art.

Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded.

Thus, a plant may be transformed to introduce a RNAi, snRNA, dsRNA, siRNA, miRNA, ta-siRNA or cosuppression molecule that has been designed to target the expression of the MINIYO and/or RTR1 gene and selectively decreases or inhibits the expression of the gene or stability of its transcript. Preferably, the RNAi, snRNA, dsRNA, siRNA, miRNA, ta-siRNA or cosuppression molecule used in the methods of the invention may comprise a fragment of at least 17 nt, preferably 22 to 26 nt and can be designed on the basis of the information shown in SEQ ID No. 1 and/or 8. Guidelines for designing effective siRNAs are known to the skilled person.

siNA molecules may be double stranded. In one embodiment, double stranded siNA molecules may comprise blunt ends. In another embodiment, double stranded siNA molecules may comprise overhanging nucleotides (e.g., 1-5 nucleotide overhangs, preferably 2 nucleotide overhangs). In some embodiments, the siRNA is a short hairpin RNA (shRNA); and the two strands of the siRNA molecule may be connected by a linker region (e.g., a nucleotide linker or a non-nucleotide linker). The siNAs of the invention may contain one or more modified nucleotides and/or non-phosphodiester linkages. Chemical modifications well known in the art are capable of increasing stability, availability, and/or cell uptake of the siNA. The skilled person will be aware of other types of chemical modification which may be incorporated into RNA molecules. In one embodiment, recombinant DNA constructs as described in U.S. Pat. No. 6,635,805, incorporated herein by reference, may be used.

Silencing of the MINIYO and/or RTR1 gene may also be achieved using virus-induced gene silencing.

For example, the transgenic plant having reduced activity of the MINIYO and/or RTR1 polypeptides may be characterised in that in comparison with the wild phenotype plant said plant has a reduction of between 50% and 100% in the expression of a gene encoding for an amino acid sequence of the MINIYO and/or RTR1 protein as described herein.

In one embodiment, the endogenous MINIYO or RTR1 gene carries a functional mutation.

In another embodiment, the transgenic plant expresses a transgene said transgene which may comprise a modified MINIYO or RTR1 nucleic acid sequence when compared to a wild type sequence.

For example, said modification/functional mutation of the MINIYO nucleic acid sequence results in a polypeptide which may comprise a substitution of the second conserved G in the RGG motif (SEQ ID No. 32).

With reference to the Arabidopsis sequence, this is G962E. The numbering of the amino acid residues as used in this disclosure is based on the numbering of the Arabidopsis AtMINIYO. Because the MINIYO or RTR1 amino acid sequence in other species may comprise fewer or more amino acids, the position of the second conserved G in the RGG motif residue may not be 962, but may between position 947 to 1067 (FIG. 16). The positions of the targeted residues according to the invention in some exemplified plant species are shown in FIG. 3B.

In another embodiment, the modification is a substitution or deletion of one or more residues within the nuclear localisation signals present in the MINIYO and/or RTR1 protein. In another embodiment, it is an insertion.

MINIYO and RTR1 are required for cell proliferation in meristematic cells, where they accumulate primarily in the cytosol, albeit shuttling through the nucleus, as evidenced by the fast nuclear accumulation when export is blocked with leptomycin B. Moreover, in the meristem periphery, MINIYO accumulates in the nucleus and together with RTR1 switches on cell differentiation. The shuttling between the cytosol and the nucleus implies that MINIYO and RTR1 have domains responsible for nuclear import and for nuclear export. Those signals can be identified through a blind genetic search, testing the localization of mutant versions of the proteins (i.e: a deletion series), or by a directed bioinformatic search for nuclear localization signals (NLS) and nuclear export signals (NES) in the protein sequences. Those skilled in the art will be aware that by mutating the domains required for nuclear import or nuclear export, it is possible to generate MINIYO and RTR1 constructs such that the encoded proteins are retained in the cytosol or in the nucleus, respectively. In this way, the two activities of MINIYO and RTR1 (promoting cell proliferation and promoting cell differentiation) may be uncoupled. Constructs that cause retention of MINIYO and RTR1 in the cytosol may specifically promote proliferation without affecting the timing of differentiation, whereas the constructs that cause MINIYO and RTR1 to be retained in the nucleus will specifically promote cell differentiation.

The predicted NLS in AtMINIYO are located at aa. 250-262 (GEAKLKKRKHSVQ, SEQ ID No. 50) and at aa. 1397-1420 (RDLSRKRHREGMMLDLLRYKKGSA, SEQ ID No. 51).

Thus, the invention relates to a nucleic acid construct which may comprise a MINIYO nucleic acid sequence which encodes for a polypeptide that has a mutation in one of both of the NLS of the resulting MINIYO polypeptide. The mutation may be a substitution or deletion of one or more residues in the NLS, preferably all residues. In one embodiment, residues aa. 250-262 and/or aa. 1397-1420 in AtMINIYO or corresponding residues in orthologues are deleted. Said construct may be introduced and expressed in a transgenic plant according to the methods of the invention to exclude the MINIYO polypeptide from the cell nucleus and thus block cell differentiation and stimulate cell proliferation. Using inducible promoters, the nucleic acid may be included in an expression vector as described herein so that the timing of the expression can be specifically determined.

In one embodiment of the invention, an MINIYO protein impaired in nuclear import is expressed under the control of an embryo specific promoter (such as the Arabidopsis cruciferin promoter, the Brassica napus Napin A promoter, the rice glutelin promoter, the maize 19 Kda zein promoter, the wheat SPA promoter or the pea legumin promoter) or an endosperm-specific promoter (such as the wheat gliadin promoter, the rice prolamin promoter, or the maize END promoter) to increase cell proliferation, seed size and yield in a seed crop.

In another embodiment of the invention, an MINIYO protein impaired in nuclear import is expressed under the control of a shoot meristem promoter (such as KNOX gene promoters from Brassica, rice or maize) to increase cell proliferation, meristem size, meristem number, production of aerial organs and crop yield (leaves, flowers).

In one embodiment of the invention, an MINIYO protein impaired in nuclear import is expressed under the control of axillary bud specific promoter (BRC1 promoter from Arabidopsis, TB1 promoter from maize, OSTB1 promoter from rice, ATC085 promoter from tobacco, S1BRC1a and S1BRC1b promoters from tomato) to increase branching and yield.

In one embodiment of the invention, an MINIYO protein impaired in nuclear import is expressed under the control of a root meristem specific promoter (RCH1 promoter, the brassica G1-3b promoter) to increase cell proliferation, root growth, nutrient uptake and plant yield.

In one embodiment of the invention, an MINIYO protein impaired in nuclear import is expressed under the control of the IYO promoter that is active in embryos and in plant meristems, to increase seed size, meristem size, plant growth and improve yields in target crops.

The predicted NLS in AtRTR1 is located at aa. 340-368. LKGDLQTLDGKNTLSGSSSGSNTKGSKTK, SEQ ID No. 52.

Thus, the invention relates to a nucleic acid construct which may comprise a RTR1 nucleic acid sequence which encodes for a polypeptide that has a mutation in the NLS of the resulting RTR1 polypeptide. The mutation may be a substitution or deletion of one or more residues in the NLS, preferably all residues. Said construct may be introduced and expressed in a transgenic plant according to the methods of the invention to exclude the RTR1 polypeptide from the cell nucleus and thus block cell differentiation and stimulate cell proliferation. Using inducible promoters, the nucleic acid may be included in a an expression vector as described herein so that the timing of the expression can be specifically determined.

In one embodiment of the invention, an RTR1 protein impaired in nuclear import is expressed under the control of an embryo specific promoter (such as the Arabidopsis cruciferin promoter, the Brassica napus Napin A promoter, the rice glutelin promoter, the maize 19 Kda zein promoter, the wheat SPA promoter or the pea legumin promoter) or an endosperm-specific promoter (such as the wheat gliadin promoter, the rice prolamin promoter, or the maize END promoter) to increase cell proliferation, seed size and yield in a seed crop.

In another embodiment of the invention, an RTR1 protein impaired in nuclear import is expressed under the control of a shoot meristem promoter (such as KNOX gene promoters from Brassica, rice or maize) to increase cell proliferation, meristem size, meristem number, production of aerial organs and crop yield (leaves, flowers).

In one embodiment of the invention, an RTR1 protein impaired in nuclear import is expressed under the control of a root meristem specific promoter (RCH1 promoter, the brassica G1-3b promoter) to increase cell proliferation, root growth, nutrient uptake and plant yield.

In one embodiment of the invention, an RTR1 protein impaired in nuclear import is expressed under the control of the MINIYO promoter, active in embryos and in plant meristems, to increase seed size, meristem size, plant growth and improve yields in target crops.

Also within the scope of the invention are transgenic plants wherein both the MINIYO protein and the RTR1 are impaired in nuclear import. Combinations of the manipulations of the NLS in MINIYO protein and the RTR1 as set out above can be used to achieve this.

The activity of RTR1 may also be decreased by manipulating the interaction between MINIYO and RTR1 proteins. This can achieved by manipulating certain residues in the MINIYO and/or RTR1 polypeptide sequences. For example, substituting the putative zinc coordinating cysteine residues for alanines (C56A/C61A or C94A/C98A in the Arabidopsis sequence) in the full-length AtRTR1 protein abrogates the interaction with MINIYO.

Another aspect of the invention refers to a transgenic plant wherein the activity of a MINIYO polypeptide is increased or up-regulated. Another aspect of the invention refers to a transgenic plant wherein the activity of a RTR1 polypeptide is increased or up-regulated.

In one embodiment, the transgenic plant is characterised in that the activity of both a MINIYO and a RTR1 polypeptides is increased or up-regulated in the same plant.

For example, said plant overexpresses a nucleic acid encoding for a MINIYO protein that is at least 30% identical to the sequences coded by SEQ ID NO:1. In another embodiment, said plant overexpresses a nucleic acid encoding for a RTR1 protein that is at least 30% identical to the sequences coded by SEQ ID NO:8.

In another embodiment, said plant expresses a transgene said transgene which may comprise a modified MINIYO and a RTR1 nucleic acid sequence when compared to a wild type sequence.

For example, said modification is a substitution or deletion of one or more residues within the nuclear export signal present in the MINIYO or RTR1 protein.

Preferably, over-expression will be between 2 and 100 times the expression of the endogenous mRNA.

One way of increasing the activity of MINIYO or RTR1 is to retain the protein in the nucleus.

The predicted NES in AtMINIYO is located at 432-440. LVLALRMAL SEQ ID No. 53.

Thus, the invention relates to a nucleic acid construct which may comprise a MINIYO nucleic acid sequence which encodes for a polypeptide that has a mutation in the NES of the resulting MINIYO polypeptide. The mutation may be a substitution or deletion of one or more, preferably all residues of the NES. In one embodiment, residues 432-440 in AtMINIYO or corresponding residues in orthologues are deleted. Said construct may be introduced and expressed in a transgenic plant according to the methods of the invention to retain the MINIYO polypeptide in the cell nucleus and stimulate cell differentiation. Using inducible promoters, the nucleic acid may be included in a an expression vector as described herein so that the timing of the expression of the mutated nucleic acid can be specifically determined.

In one embodiment of the invention, a MINIYO protein impaired in nuclear export is expressed under the control of axillary bud specific promoter (BRC 1 promoter from Arabidopsis, TB1 promoter from maize, OSTB1 promoter from rice, ATC085 promoter from tobacco, S1BRC1a and S1BRC1b promoters in tomato) to reduce branching and increase yield. This is particularly important for forestry applications, for instance for growing closely packed trees used for pulp production in the paper or biofuel industry.

In one embodiment of the invention, a MINIYO protein impaired in nuclear export is expressed under the control of an inflorescence meristem specific promoter (such as the LFY promoter) to terminate the inflorescence meristem in crops that are cultivated for their vegetative organs and in which flowering reduces the harvest (lettuce, spinach, sugar beet, potato, and others).

The predicted NES in RTR1 is located at 340-349 (LKGDLQTLDG, SEQ ID No. 54).

Thus, the invention relates to a nucleic acid construct which may comprise a RTR1 nucleic acid sequence which encodes for a polypeptide that has a mutation in the NES of the resulting RTR1 polypeptide. The mutation may be a substitution or deletion of one or more, preferably all residues of the NES. In one embodiment, residues 432-440 are deleted. Said construct may be introduced and expressed in a transgenic plant according to the methods of the invention to retain the RTR1 polypeptide in the cell nucleus and stimulate cell differentiation. Using inducible promoters, the nucleic acid may be included in an expression vector as described herein so that the timing of the expression of the mutated nucleic acid can be specifically determined.

Also within the scope of the invention are transgenic plants wherein both the MINIYO protein and the RTR1 are impaired in nuclear import. Combinations of the manipulations of the NLS in MINIYO protein and the RTR1 as set out above can be used to achieve this.

In one aspect, the invention relates to transgenic plants wherein both, MINIYO and RTR1 have been manipulated. As shown in the examples, MINIYO and RTR1 are jointly responsible for the control of cell differentiation, supporting a close functional interaction. Differentiation in the iyo-1atrtr1-2 double mutants was almost completely blocked and the plants eventually developed as a friable callus of undifferentiated cells. This phenotype is much stronger than the sum of the phenotypes of the single mutants. Thus, transgenic plants according to the invention may have reduced or increased activity for both, MINIYO and RTR1 by manipulating activity of MINIYO and RTR1 as explained herein.

For the purposes of the invention, “transgenic”, “transgene” or “recombinant” means with regard to, for example, a nucleic acid sequence, an expression cassette, gene construct or a vector which may comprise the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)

are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 incorporated by reference.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

Transgenic plants according to the invention display altered cell differentiation and proliferation compared to a control plant. A control plant according to the invention is a plant that is not modified in the same way as the transgenic plant of the invention with respect to MINIYO and/or RTR1 expression or polypeptide activity. In one embodiment, this plant is a wild type plant. In another embodiment, this plant is a parent plant that may comprise additional modifications through expression of other transgene of interest that modify desired pathways, for example stress resistance.

The MINIYO or RTR1 genes according to the different aspects of the invention may be an exogenous gene, such as Arabidopsis AtMINIYO or AtRTR1, overexpressed in a different plant species. Alternatively, the MINIYO or RTR1 may be an endogenous plant gene, i.e. a gene that is endogenous to the plant in which it is introduced via recombinant methods and (over)-expressed.

In a preferred embodiment of the invention, the transgenic plant is characterised in that it is selected from the group which may comprise: plants for particular use in the methods according to the invention include all the plants belonging to the superfamily Viridiplantae, in particular monocotyledonous and dicotyledonous plants including forage plants and vegetables for livestock, ornamental plants, crop plants for use in human or animal nutrition, plants for use as bioenergy, trees, and bushes selected from the list which may comprise: Acer spp., Actinidia spp., Abelmoschus spp., Agropyron spp., Allium spp., Amaranthus spp., Ananas comosus, Annona spp., Apium graveolens, Arabidopsis thaliana, Arachis spp, Artocarpus spp., Asparagus officinalis, Avena sativa, Averrhoa carambola, Benincasa hispida, Bertholletia excelsea, Beta vulgaris, Brachypodium spp., Brassica spp., Cadaba farinosa, Camellia sinensis, Camelina spp., Canna indica, Capsicum spp., Carex elata, Carica papaya, Carissa macrocarpa, Carya spp., Carthamus tinctorius, Castanea spp., Cichorium endivia, Cinnamomum spp., Citrullus lanatus, Citrus spp., Cocos spp., Coffea spp., Colocasia esculenta, Cola spp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Eleusine coracana, Eriobotrya japonica, Eucalyptus spp., Eugenia uniflora, Fagopyrum spp., Fagus spp., Ficus carica, Fortunella spp., Fragaria spp., Ginkgo biloba, Glycine spp., Gossypium hirsutum, Helianthus spp., Hemerocallis fulva, Hibiscus spp., Hordeum spp., Ipomoea batatas, Juglans spp., Jatropha spp., Lactuca sativa, Lathyrus spp., Lens culinaris, Linum usitatissimum, Litchi chinensis, Lotus spp., Luffa acutangula, Lupinus spp., Luzula sylvatica,Macrotyloma spp., Malus spp., Malpighia emarginate, Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Ornithopus spp., Oryza spp., Panicum miliaceum, Passiflora edulis, Pastinaca sativa, Persea spp., Petroselinum crispum, Phaseolus spp., Phoenix spp., Physalis spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Saccharum spp., Sambucus spp., Secale cereale, Sesamum spp., Sinapis sp., Solanum spp., Sorghum bicolor, Spinacia spp., Syzygium spp., Tamarindus indica, Theobroma cacao, Trifolium spp., Triticosecale rimpaui, Triticum spp., Tropaeolum minus, Tropaeolum majus, Vaccinium spp., Vicia spp., Vigna spp., Viola odorata, Vitis spp., Zea mays, Zizania palustris, Ziziphus spp., among others.

According to a preferred embodiment of the invention, the crop plant is a plant such as tomato, potato, pepper, fruiting plants of the prunus and citrus genuses, Jatropha curcas, soya, sunflower, rape, alfalfa, canola, cotton, brassica genuses or tobacco. Even more preferably, the plant is a monocotyledonous one such as sugar cane, and even more preferably a cereal such as rice, maize, wheat, rye, barley, millet, sorghum or oats. Most preferred plants are maize, rice, wheat, sorghum, canola and cotton.

Another preferred embodiment of the invention relates to a product obtained from the transgenic plant as described above, the said product being selected from seeds, stones, leaves, flowers, roots, flour and fruit. In a more preferred embodiment the said product is a transgenic seed. In one embodiment the products produced by said methods of the invention are plant products such as, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. In another embodiment the inventive methods for the production are used to make agricultural products such as, but not limited to, plant extracts, proteins, amino acids, carbohydrates, fats, oils, polymers, vitamins, and the like. It is possible that a plant product consists of one ore more agricultural products to a large extent.

The invention also relates to a method for generating a transgenic plant with altered cell differentiation and cell proliferation which may comprise altering the activity of a gene encoding a MINIYO polypeptide. This may be achieved by expressing a MINIYO transgene in a plant so that activity is altered. In another aspect, the invention relates to a method for generating a transgenic plant with altered cell differentiation and cell proliferation which may comprise altering the activity of a gene encoding a RTR1 polypeptide as defined herein. In another aspect, plants are generated where the activity of both, MINIYO and RTR1, is manipulated. As explained elsewhere, alteration of the activity of MINIYO and/or RTR1 means that the activity may be increased or decreased. This may be achieved by manipulating the NLS/NES sequences and introducing constructs that express MINIYO and/or RTR1 proteins modified in this way as explained herein. Other ways of manipulating the activity of MINIYO and RTR1, such as gene silencing or the generation of partial loss of function mutants, are also set out herein.

In another aspect, the invention relates to a plant obtained/obtainable by said methods.

In another aspect, the invention relates to a method for altering localisation of MINIYO and/or RTR1 in a plant by manipulating the NLS/NES sequences of MINIYO and/or RTR1 as described herein. In this way, MINIYO and/or RTR1 may be retained or excluded from the nucleus in one or more meristems. The NLS/NES sequences can be manipulated to achieve this as set out herein and transgenes carrying such manipulations can be introduced and expressed in a plant.

In another aspect, the invention also relates to a method for improving the architecture and yield of plants through genetic changes to the MINIYO (SEQ ID No 1), AtRTR1 (SEQ ID No 8) genes or their orthologues in other plants. The expression of improving architecture refers to the non-exclusive list of altering the size/number of one or more meristems, altering the number of side branches, altering inflorescence, altering thickness of the stems, modify thickness of the stems and increasing plant yield.

In a preferred embodiment of the method for improving the architecture and yield of plants, the method is used to alter the size of one or more meristems, including increasing or decreasing the activity of the MINIYO and RTR1 genes.

In a preferred embodiment of the method, it is used to increase the size of the meristems by delaying the onset of differentiation and consequently increasing the number of undifferentiated cells brought about through the loss of function of MINIYO and/or RTR1. Preferably, to obtain ectopic shoot apical meristems through delaying the onset of differentiation and the consequent increase in the number of undifferentiated cells caused by the loss of function of MINIYO and/or RTR1.

In another preferred embodiment of the improving the architecture and yield of plants, the method is to obtain ectopic floral meristems through delaying the onset of differentiation and the consequent increase in the number of undifferentiated cells caused by the loss of function of MINIYO and/or RTR1.

In another preferred embodiment of the method, it is used to obtain ectopic root meristems through delaying the onset of differentiation and the consequent increase in the number of undifferentiated cells caused by the loss of function of MINIYO and/or RTR1.

In another preferred embodiment, the method is to obtain ectopic embryos through delaying the onset of differentiation in the suspensor cells caused through the loss of function of MINIYO and/or RTR1.

In a preferred embodiment, the method is used to reduce or eliminate meristems through delaying the onset of differentiation caused by the increased activity of MINIYO and/or RTR1. Preferably, to reduce the number of side branches in crops through increasing the activity of MINIYO and/or RTR1, specifically in axillary buds.

In another preferred embodiment, the method is used to compact inflorescence through increasing the activity of MINIYO and/or RTR1 in reproductive meristems.

In another preferred embodiment, the thickness of the stems of herbaceous plants is increased.

In another preferred embodiment, secondary growth in shrubs is modified.

In another more aspect, the invention includes a method to increase plant yield by decreasing or downregulating the activity of MINIYO and/or RTR1 in a transgenic plant. This may be achieved as described elsewhere, including through manipulating of the NLS sequences, creating mutant proteins that lead to partial loss of function or gene silencing.

Another preferred embodiment of the invention relates to the development of transgenic plants for use in obtaining biofuels, such as in the production of bioethanol.

The invention also relates to the use of a polypeptide having at least 30% sequence identity to a polypeptide encoded by SEQ ID NO. 1 or 8 in altering cell differentiation, cell proliferation, meristem formation/growth and/or increasing crop yield.

In another aspect, the invention relates to manipulating the interaction between MINIYO and RTR1 proteins. This can achieved by manipulating certain residues in the MINIYO and/or RTR1 polypeptide sequences. For example, substituting the putative zinc coordinating cysteine residues for alanines (C56A/C61A or C94A/C98A in the Arabidopsis sequence) in the full-length AtRTR1 protein abrogates the interaction with MINIYO.

In another embodiment, the invention relates to an isolated nucleic acid sequence which may comprise or which may consist of SEQ ID No. 48 (AtMINIYO promoter). In another embodiment, the invention relates to an isolated nucleic acid sequence which may comprise or which may consist of SEQ ID No. 49 (RTR1 promoter).

Such promoter sequences may be fused to any gene of interest to direct spatial and temporal expression of the target gene. The invention also relates to the use of these promoter sequence in directing expression at sites of active cell proliferation and differentiation (for example shoot apical meristem (SAM), in leaf and flower primordia, in unfertilized ovules and in developing embryos, but not in mature organs).

The invention also relates to methods for screening for loss of function mutants of MINIYO and/or RTR1 in plants. These methods may comprise generating a mutant population by using mutagens known in the art. Specifically included are modifications of the endogenous locus by mutagenesis, including chemical mutagenesis, leading to a deletion, insertion or substitution in the endogenous locus. The mutagen may be fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde.

In one embodiment, the method used to create and analyse mutations is targeting induced local lesions in genomes (TLLING).

A skilled person will know that different approaches can be used to generate such mutants. In one embodiment, insertional mutagenesis is used. In this embodiment, as discussed in the examples, T-DNA may used as an insertional mutagen which disrupts MINIYO or RTR1 gene expression. These plants thus do not carry a transgene to alter expression of the endogenous locus, but the endogenous locus is modified by mutagenesis. The methods also involve analyzing cell proliferation/differentiation compared to control wild type plants. If cell proliferation/differentiation is delayed, then this may be due to a mutation in MINIYO and/or RTR1.

In one embodiment, methods that solely rely on essentially biologically processes are specifically disclaimed.

Through the description and the claims the word “comprises” and its variants is not intended to exclude other technical features, additives, components or steps. To those skilled in the art other objects, advantages and characteristics of the invention will be apparent partly from the description and partly from the practice of the invention. The following figures and examples are provided by way of illustration, and are not intended to restrict the invention.

The disclosure of all references cited is incorporated.

### REFERENCES

Clark et al. Development 121, 2057-2067, 1995.
De Veylder et al., Nat. Rev. Mo. Cell Biol. 2007.
Guenther et al., Cell 130, 77-88. 2007.
Stock et al. Nature Cell. Bio1.9, 1428, 1435. 2007.
Mosley et al. Mol. Cell. 34, 168-179. 2009.
Nam-II et al. Plant Science 176, 461-469. 2009.
Ramirez-Parra et al. Int. J. Dev. Biol. 2005
Kosugi et al. Proc. Natl. Acad. Sci. USA 106, 10171-6. 2009.
Kosugi et al. J. Biol. Chem. 284, 478-485. 2009.
Thompson et al., J. Biol. Chem. 280, 21854-7. 2005.
Zhang et al., Proc. Natl. Acad. Sci. USA 97, 12577-82. 2000.
Yeung et al., J. Cell Biochem. 103, 456-70. 2008.
Egloff et al., Mol. Cell. 45, 111-122. 2011.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

The following specific examples provided in this patent document serve to illustrate the nature of this invention. These examples are included purely for illustrative purposes and should not be interpreted as limiting the invention claimed here. Therefore the examples described below illustrate the invention without limiting the scope of its application.

### Example 1

1. Identification of the miniyo-1 (iyo-1) mutant. The iyo-1 mutant is identified in a population mutagenised by ethylmethane sulphonate in the Landsberg erecta ecotype which brought about a change of amino acid (G962E) in the coded protein.
2. Positional map of the iyo-1 mutant to provide evidence of point mutations in the IYO gene. The mutant was crossed with accession Col-0 and it was proved that the iyo-1 phenotype is inherited in a recessive way. F2 plants with a mutant phenotype were used to clone the mutation through positional mapping. The analysis of more than 1500 mutant plants made it possible to establish the position of the mutation to 6 genes in chromosome 4. Sequencing of the 6 genes showed that there was a single mutation in the At4g38440 gene in iyo-1 mutants. This was a point mutation (G to A).
3. Generation of ectopic stem cells in an iyo-1 mutant. It was proved by microscope studies and by analysing the expression of markers that one of the phenotypes of the iyo-1 mutant was the formation of ectopic stem cells in all meristems of the plant.
4. Total blocking of IYO activity in iyo-2 and iyo-3 mutants. Mutants were obtained that had a T-DNA insertion in the At4g38440 gene and it was proved that they cause blocking of endosperm development and early arrest of embryogenesis. iyo-2 has T-DNA insertion at nucleic acid position 234 (Salk 099873). iyo-2 has T-DNA insertion at nucleic acid position 1357 (Salk 0692_g12).
5. Combination of the hypomorphic allele iyo-1 with the null alleles (iyo-2 or iyo-3). The allele combination iyo-1/iyo-2 or iyo-1/iyo-3 caused almost total blocking of differentiation of the embryo and the formation of ectopic embryos from the suspensor. The embryos were viable and after germination the plants grew as a callus-like structure with multiple meristematic poles and producing only rudimentary leaves.
6. Double embryos in an iyo-1 mutant. Although with low penetrance, Applicants observed that the iyo-1 mutant can also form double embryos from the suspensor, which apparently developed normally.
7. Transcriptional and post-transcriptional regulation of the expression of IYO.
7.1. Monitoring of activity of the IYO promoter.
   Through a Northern type analysis, in situ hybridisation and studies of the promoter Applicants have demonstrated that the IYO gene is expressed specifically during embryogenesis in meristem cells and in cells adjacent to the meristem which are initiating differentiation.
7.2. Nuclear accumulation of IYO protein
   Confocal microscopic studies of functional fusions of IYO protein with GFP which complement the iyo-1 and iyo-2 mutants have shown that IYO is excluded from the nucleus in undifferentiated cells in a manner which is dependent on Exportin1, while it accumulates in the nucleus in cells which are initiating differentiation.
8. Over-expression of IYO or its fusions with proteins or peptides such as GFP, HA, FLAG under the control of the 35S promoter.
   Col-0 Arabidopsis plants were transformed with constructs which expressed fusions of the epitopes GFP, 3XHA or FLAG at the C-terminal of IYO under the control of the 35S promoter. The lines which accumulated larger quantities of the transgene transcript and the transgenic protein had gain of function phenotypes, from premature differentiation in the shoot apical meristem and compaction of inflorescence but no apparent effect on the root meristem to termination of the shoot and root apical meristems in lines with the maximum levels of accumulation.
9. Physical interactions between IYO protein and RNA Polymerase II (Pol II) and with elongation complexes. Through pull-down studies with proteins synthesised in bacteria and in vitro, studies of bimolecular complementation of YFP, measurements of accumulation of different phosphorylated and ubiquitinised forms of the RPB 1 sub-unit and analysis of genetic interactions Applicants have proved that IYO interacts with Pol II and with the elongator complex and is required for maintaining the global levels of elongating Pol II in differentiating tissues.

### Example 2

1. Identification of the gene At5g26760 (atrtr1). Through the analysis of co-expression Applicants discovered the At5g26760 gene which is highly co-expressed with IYO and codes for a protein homologous to RTR1, a yeast protein which functions as a transition phosphatase that dephosphorylates Ser5 in the CTD from the RPB1 sub-unit of Pol II to promote the shift from initiation to productive elongation.
2. Analysis of AtRTR1 function in the mutant alleles atrtr1-1 (SALK_{—}012339, T-DNA insertion at nucleic between nucleic acid 414 and 415) and atrtr1-2 (SALK_{—}115762, T-DNA insertion at nucleic between nucleic acid 864 and 865). Applicants obtained homozygous mutants of atrtr1-1, showing by Nomarski microscopy that it caused blocking of differentiation in the embryos, the formation of ectopic embryos from the suspensor and early embryo arrest. The homozygous mutant atrtr1-2 has phenotypes very similar to the iyo-1 mutant: enlarged shoot apical meristems, thicker stems, formation of ectopic shoot, floral and root meristems, delayed differentiation of the protodermis and the formation of clusters of stomas.
3. Generation of ectopic stem cells in atrtr1-2 mutants. Microscopic studies and analysis of marker expression reveal that ectopic stem cells form in all meristems in the atrtr1-2 mutants.
4. Investigation of gene interaction between IYO and AtRTR1. The double mutant iyo-1atrtr1-2 was obtained and it was shown that cell differentiation was completely blocked, the plants growing as calluses of undifferentiated cells, demonstrating clear gene interaction.
5. Investigation into the over-expression of IYO in an atrtr1-2 mutant context. Heterozygous plants from IYO-3XHA over-expressing lines displaying termination of shoot and root meristems were crossed with homozygous atrtr1-2 plants. In the F2, homozygous plants for the IYO-3XHA construct were recovered that were homozygous for atrtr1-2 or were wild type for the AtRTR1 locus. It was proved that while IYO-3XHA over-expression in wild type plants produced shoot and root meristem termination, in the atrtr1-2 background it did not cause any phenotype, demonstrating that AtRTR1 activity is required for IYO function.
6. Investigation into the expression of AtRTR1 in transgenic plants which express an AtRTR1 promoter construct and the first three exons and introns of AtRTR1 translationally fused to the UidA (GUS) gene. Histological studies of GUS activity in transgenic plants expressing the UidA gene under the control of the AtRTR1 promoter show that this is specifically located in embryos, meristem cells and cells adjacent to the meristem which are beginning to differentiate.
7. Investigation into the accumulation of RTR1 fused to GFP under the control of the constitutive 35S promoter and the AtRTR1 promoter. Confocal microscopy studies of plants expressing stable functional fusions of AtRTR1 protein to GFP which complement atrtr1-1 mutants have demonstrated that AtRTR1 is excluded from the nucleus in undifferentiated cells in a form dependent on Exportin1.

### Example 3

### Bioinformatics Analysis

To search for candidate nuclear localization signals (NLS), the Arabidopsis IYO and AtRTR1 protein sequences were submitted to the cNLS mapper server (http://nls-mapperlab.keio.ac.jp). This tool searches for classical NLS (cNLS) recognized by the nuclear receptor importin. cNLSs detected with this tool have been validated in a number of yeast proteins (Kosugi et al., PNAS 2009). The server predicted in IYO a monopartite NLS (aa. 250-262) and a bipartite NLS (aa. 1397-1420), both with a score of 10, the maximum possible. When a GUS-GFP reporter protein was fused with NLSs having a score between 8-10 they accumulated in the nucleus (Kosugi et al., PNAS 2009, JBC 2009). In the case of AtRTR1, the server predicted a bipartite NLS with a long linker and a score of 5.2 in the middle of the protein (aa. 340-368). GUS-GFP proteins fused with NLSs with a score 3-7 were localized in the cytosol and the nucleus (Kosugi et al., PNAS 2009, JBC 2009). Moreover, when bi-partite NLSs have long linkers they are functional normally in unstructured regions, which frequently coincide with the N- and C-terminal ends of the proteins. Thus, for IYO, two high confidence cNLSs were predicted, while for AtRTR1, no clear cNLSs were detected. It is possible that AtRTR1 is imported through an importin α-independent pathway, as is the case for the majority of nuclear proteins. In this regard, Applicants have shown that nuclear IYO leads to AtRTR1 accumulation in the nucleus, which could be by mediating its import.

To search for candidate nuclear export signals (NES), the Arabidopsis IYO and AtRTR1 protein sequences were submitted to the NetNES server (http://www.cbs.dtu.dk/services/NetNES). This tool searches for leucine-rich NES, and predictions have been validated (for example for BRCA1, Thompson et al., JBC 2005). The server predicted a NES in IYO (aa. 432-440) and in AtRTR1 (aa 340-349). Consistent with these predictions, inhibition of the NES receptor CRM1 with leptomycin B, leads to nuclear accumulation of IYO and AtRTR1 in Arabidopsis and Nicotiana cells, suggesting that their nuclear export is NES-dependent. Moreover the truncated C-terminal half of AtRTR1 (aa 305-735) that contains the predicted NES is exported from the nucleus.

To test for altered localization, mutated versions of the proteins are fused to GFP and their subcellular distribution by transient expression in Nicotiana benthamiana leaves is analysed (where wild type IYO accumulates primarily in the nucleus and AtRTR1 in the cytosol). Analysis is also carried out in in Arabidopsis cell cultures untreated (where both proteins are localized in the cytosol) or treated with leptomycin B (where both proteins are localized in the nucleus). In the case of IYO constructs are designed lacking each predicted NLSs and one combining mutations in both NLSs, as it has been shown in other proteins with multiple NLSs that only after mutating all of them is their nuclear import blocked (i.e. Zhang et al., PNAS 2000; Yeung et al., J. Cell Biochem. 2008).

Homology Analysis

Alignment by clustalW of the whole polypeptide sequence shows that the % identity between IYO orthologues from embryophytes (A thaliana, A lyrata, Brachypodium, Carica, citrus, Eucalyptus, Manihot, Medicago, Oryza, Physcomitrella, Populus, Prunus, Ricinus, Selaginella, Sorghum, Vitis, Zea) is higher than 30%. If only angiosperm sequences are aligned the overall identity is higher than 39%.

Alignment by clustalW of the whole polypeptide sequence shows that the % identity between IYO orthologues from embryophytes (A thaliana, A lyrata, Brachypodium, Carica, citrus, Eucalyptus, Manihot, Medicago, Oryza, Physcomitrella, Populus, Prunus, Ricinus, Picea, Sorghum, Vitis, Zea) is higher than 25%. If only spermatophyte sequences are aligned the overall identity is higher than 32%.

An Interpro scan of the polypeptide sequence of IYO reveals two conserved domains IPR013929 (PF08620) and IPR013930 (PF08621) in the N-terminus of the protein (aa 209-255 and 317-396, respectively), which are found in orthologues from plants, animals and fungi. Moreover, blast searches reveal two other domains highly conserved in IYO orthologues from multicellular eukaryotes (aa 529-597 and 1144-1416). In addition, IYO has a glycine rich domain with an RGG motif (aa 960-980) that is strictly conserved in orthologues from plants. Glycine rich domains and RGG boxes have been linked to nucleic acid binding (Gendra et al., Plant Journal 2004). Moreover, the iyo-1 allele is a missense mutation that changes the motif from RGG to RGE and reduces the transcriptional activity of the protein. This indicates that this domain contacts the DNA or the nascent transcript to facilitate transcription. This domain is not clearly identifiable in the animal orthologues of IYO, but alignment of their sequences reveals a high number of conserved glycines in this region.

AtRTR1 contains a conserved domain (DUF408) with a zinc-finger like motif located at the N-terminus of the protein that is found in all the orthologues from plants, animals and fungi. A consensus sequence for that motif derived from sequences from multicellular eukaryotes is (in bold the putative Zinc-coordinating cysteines):

D[IV]V[TDEV]ER[ASTF]I[AVIS][KND][LAV]CGY[TP][LRA]
CXXXLX₇₋₁₅[YF][RK]IS[LT][KSR][TAED][HKN][KR]VYD
[IL][THEQ]EXXX[FY]CXXXC

A blast search against the non-redundant protein sequence database at NCBI with the corresponding sequence from Arabidopsis DVVTERAIAKLCGYTLCQRFLPSDVSRRGKYRISLKDHKVYDLQETSKFCSAGC retrieved the AtRTR1 orthologues from plants and animals and fungi with a low E-value (<10⁻⁶).

The zinc-finger-like-motif has been implicated in interaction with the RNA Polymerase II C-terminal domain (CTD) and the Integrator complex in humans and is required for CTD-phosphatase activity in yeast and humans (Mosley et al., 2009; Egloff et al., 2011). Interestingly, this motif is also required for interaction of AtRTR1 with IYO. Substituting for alanine the putative zinc coordinating cysteine residues (C56A/C61A or C94A/C98A) in the full-length AtRTR1 protein abrogates interaction with IYO. Intriguingly, however, both the truncated N-terminal and the C-terminal halves of AtRTR1 can interact with IYO, suggesting that although AtRTR1 binds at both ends of the protein to IYO, it requires an intact zinc-finger-like motif in the context of the full length protein.

### Example 4

Applicants analyzed the expression pattern of an AtRTR1 promoter construct driving the GUS reporter gene (pART::GUS). This same promoter driving an AtRTR1 cDNA fully complements atrtr1 mutant phenotypes, indicating that it reproduces the activity of the endogenous gene. In roots, pART::GUS was strongly expressed in root apical meristem (RAM) and in transition cells, in the pericycle layer and in lateral root primordia. In the aerial part of the plant, pAtRTR1::GUS was expressed in the shoot apical meristem (SAM), in leaf and flower primordia, in unfertilized ovules and in developing embryos, but not in mature organs. These results suggest that AtRTR1 is exclusively expressed at sites of active cell proliferation and differentiation, in a pattern highly similar to that of IYO.

To determine the subcellular distribution of AtRTR1Applicants analyzed a translational fusion to GFP. Under the control of the constitutive 35S promoter (35S::AtRTR1-GFP) Applicants only obtained transgenic lines expressing low levels of the tagged protein that complemented partially the atrtr1-1 null mutation (i.e: atrtr1-1 plants transgenic for this construct were viable but resembled the hypomorphic atrtr1-2 plants). These results suggest that expressing high levels of AtRTR1 protein in a constitutive manner may be deleterious for plant development. Applicants then transformed plants with AtRTR1-GFP driven by its own promoter (pAtRTR1::AtRTR1-GFP). The resulting lines had higher levels of expression and complemented fully the atrtr1-1 null mutation. This indicates that pAtRTR1::AtRTR1-GFP reproduces the activity of the endogenous gene and can be used as a proxy for localization of ART. pAtRTR1::AtRTR1-GFP fluorescence in the root was restricted to the tip, consistent with the pattern of expression found in pAtRTR1-GUS lines. Importantly, the fluorescence was found in the cytosol and strongly excluded from the nucleus (FIG. 6A-B). Similarly, in Nicotiana benthamiana leaf cells transiently expressing a 35S::ATRTR1-GFP construct, fluorescence was confined to the cytosol. In yeast and mammalian cells, the orthologues of ATRTR1, RTR1 and RPAP2, are also localized primarily in the cytosol, but they redistribute partially to the nucleus upon inhibition of the XPO1 nuclear export receptor with leptomycin B (LMB), which also inhibits the Arabidopsis receptor (Kudo et al., 1999, Haasen et al., 1999). After treating Arabidopsis with LMB, nuclear accumulation of ATRTR1-GFP was observed in cells of the root transition zone and in Nicotiana benthamiana leaf epidermal cells. These results suggest that, at least in differentiating and mature cells, AtRTR1 is imported into the nucleus, although the higher rate of nuclear export leads to its steady state accumulation in the cytosol. The remarkable conservation in nuclear-cytoplasmic shuttling of RTR1 homologues in all eukaryotic lineages indicates that it constitutes an important regulatory mechanism for this family of phosphatases.

Applicants tested for the in vivo interaction between IYO and AtRTR1 through a bimolecular fluorescence complementation assay in epidermal cells from Nicotiana benthamiana leaves. YFP complementation was observed with different combinations of split YFP fused at the N- or C-terminus of the respective proteins and not in any of the multiple negative controls tested. Interestingly, the reconstituted fluorescence was localized in the nucleus, suggesting that these proteins interact specifically in this compartment, possibly to regulate transcription. Moreover, Applicants found that both the DUF408-containing N-terminal half and the C-terminal half of AtRTR1 interact with IYO, suggesting that the two proteins bind through at least two sites. Unexpectedly, substituting the putative zinc coordinating cysteine residues for alanines (C56A/C61A or C94A/C98A) in the full-length AtRTR1 protein abrogates the interaction with IYO, suggesting that although AtRTR1 binds at both ends of the protein to IYO, it requires an intact zinc-finger-like motif for binding in the context of the full length protein.

Considering that AtRTR1-GFP expressed in Nicotiana benthamiana cells is found exclusively in the cytosol, it was surprising to find AtRTR1 strongly interacting with IYO in the nucleus. Applicants reasoned that when bound to IYO, AtRTR1 is retained in the nucleus. To test this Applicants expressed AtRTR1-GFP together with IYO-HA or an empty vector. Importantly, co-expression with IYO-HA led to nuclear AtRTR1-GFP accumulation in Nicotiana cells, confirming that IYO retains AtRTR1 in the nucleus. The levels of nuclear fluorescence were much lower than in the split YFP assays, where the IYO-AtRTR1 complex is stabilized through the irreversible reconstitution of YFP. This suggest that the IYO-AtRTR1 association is very transient, explaining why nuclear AtRTR1 accumulation cannot detected in transition cells of the meristem, even though IYO is present in the nucleus of those cells.

To test for genetic interaction between IYO and ATRTR1, Applicants combined the atrtr1-2 with the iyo-1 hypomorphic mutations. Differentiation in the iyo-1atrt1-2 double mutants was almost completely blocked and the plants eventually developed as a friable callus of undifferentiated cells. This phenotype is much stronger than the sum of the phenotypes of the single mutants, and indicates a strong genetic interaction of IYO and AtRTR1 in the control of cell differentiation, supporting a close functional interaction. Transcriptome analysis of iyo-1 mutants supports that IYO functions as a global transcriptional regulator of developmental programs. In inflorescence meristems, IYO was required for proper expression of flower development programs, including activating the expression of the homeotic flower organ identity genes, which are the master regulators of organogenesis in those meristems. Applicants performed a similar analysis in the atrtr1-2 mutant and Applicants found a very high overlap (>80%) in the up-regulated and down-regulated genes in iyo-1 and atrtr1-2 inflorescences meristems relative to wild type. These results indicate that IYO and AtRTR1 regulate as a complex transcription of developmental programs. Consistent with their functioning together, Applicants found that a functional AtRTR1 gene is required for IYO activity in cell differentiation. Over expression of IYO-HA provokes premature differentiation and termination of the root and shoot apical meristems. Importantly, in an atrtr1-2 background or in AtRTR1 co-suppressed line, these effects of IYO-HA over expression are eliminated, demonstrating that IYO requires AtRTR1 for its activity.

### Example 5

### Yield Analysis in Arabidopsis

Applicants have measured seed yield in a line co-suppressed in AtRTR1, which was chosen because it had a weak loss of function phenotype. This line was characterized by a few extra shoot meristems, but otherwise normal development.

Seed Yield Test

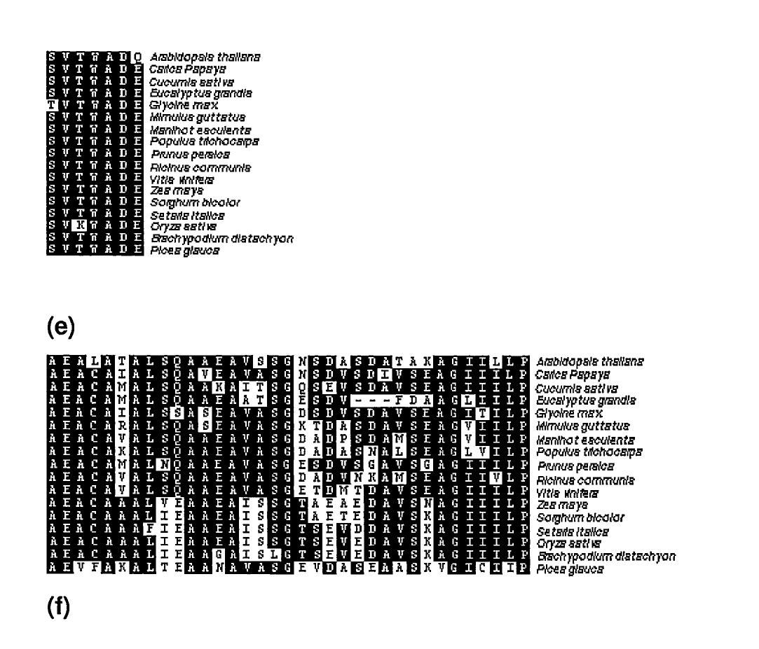

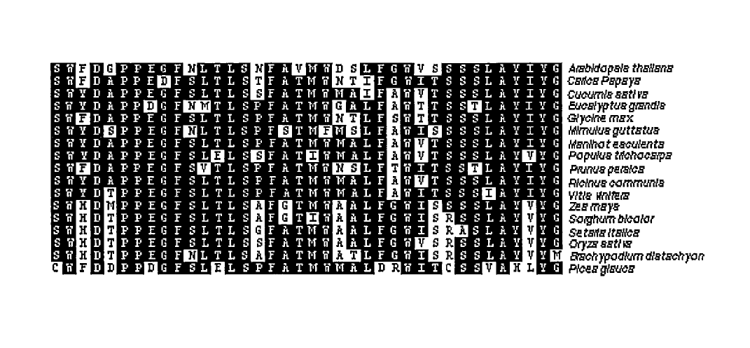

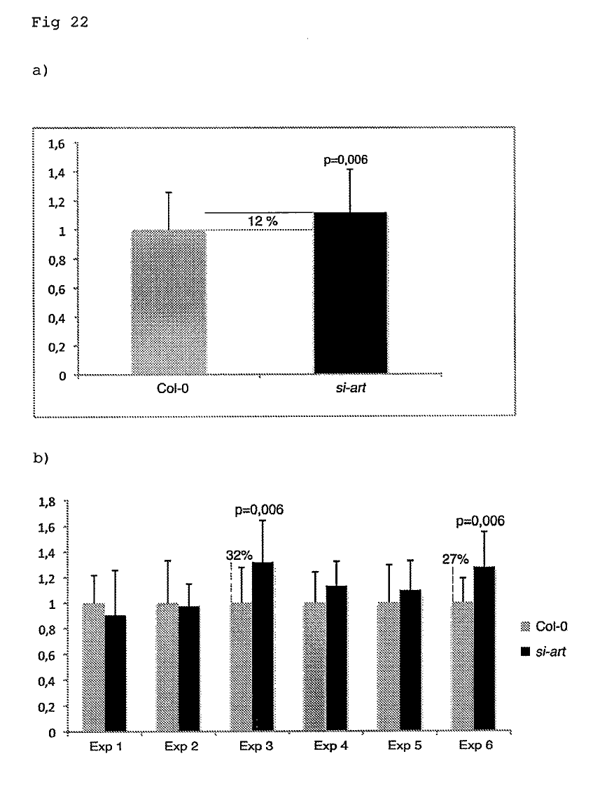

During the generation of lines transgenic for a 35S::AtRTR1-GFP construct, Applicants isolated a line (si-art line) showing co-suppression of the transgene and of the endogenous AtRTR1 gene. The phenotype of si-art plants is weaker than that of the hypomorphic atrtr1-2 allele, forming some ectopic shoot apical meristems (SAMs) that give rise to split primary shoots but otherwise developing very similarly to wild type plants. To test if the formation of ectopic SAMs affects yield, Applicants measured seed production in si-art plants and in the corresponding wild type background (Col-0). Applicants carried out six independent experiments (experiments 1-3 in the greenhouse, 4-6 in a growth chamber) and harvested seeds after the plants were fully dried. In each experiment, Applicants measured the seed yield (in weight) of individual plants, and then calculated their yield relative to the average yield of control plants (Col-0) in that particular experiment. Combining in this way the data from all six experiments, Applicants found a 12% increase in the average seed yield of si-art plants, with a p-value of 0.006 in an unpaired two tailed t-test. The results are shown in FIG. 22. If the individual experiments are analyzed separately, Applicants find significant differences (p-value<0.05) between the average yields in experiments 3 and 6, in which the yield of si-art plants is increased 32% and 27%, respectively over that of the control plants (p-value=0.006).

### Example 6

### Experiments in Tomato

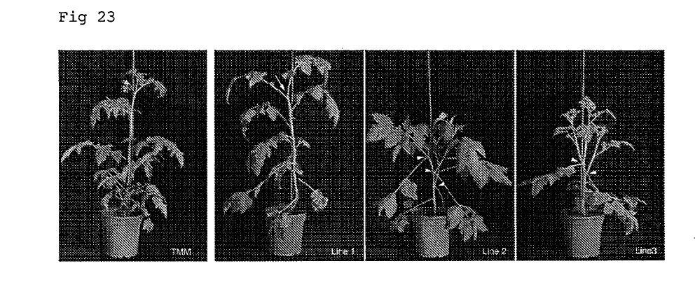

Tomato plants of the cultivar Moneymaker were transformed by co-cultivation with Agrobacterium tumefaciens with a sequence coding for the Arabidopsis IYO protein fused to GFP under the control of the 35S promoter. This construct fully complements the phenotypes of weak (iyo-1) and null (iyo-2) alleles in Arabidopsis. Plants were regenerated from independent transformed calli and transplanted to soil. Applicants analyzed roots from those lines in the confocal microscope and observed accumulation of GFP fluorescence in nuclei of differentiated cells, demonstrating that they are transgenic for the construct and that subcellular localization of the IYO protein in tomato is the same as in Arabidopsis. The development of the transgenic lines reveals that the Arabidopsis protein is functional in tomato and that its overexpression provokes premature onset of cell differentiation as it does in Arabidopsis. Some of the transgenic lines (e.g. Line 1, FIG. 23) show a determinate growth pattern, in contrast to untransformed Moneymaker plants that are indeterminate (FIG. 23). In other lines (e.g. Lines 2 and 3, FIG. 23) the branching pattern is altered (FIG. 23). The transgenic lines are fertile and produces fruits with viable seeds.

SEQUENCE LISTING

SEQ ID NO: 1 MINIYO cDNA Arabidopsis thaliana
ATGGAGCAAAGTAGCGGGAGAGTCAATCCGGAACAGCCGAACAACGTCT
TGGCGAGCCTTGTCGGGAGCATCGTGGAGAAAGGAATATCGGAGAATAA
GCCTCCAAGCAAGCCGCTTCCCCCAAGGCCCTCCCTTCTTTCCTTCCCCGT
CGCTCGTCATCGTTCTCACGGACCCCATTTGGCTCCTGTGGGAAGCAGCAT
AGCACAACCTAAGGATTACAATGACGATCAGGAAGAAGAAGAAGCAGAA
GAACGTTTCATGAATGCAGACTCCATTGCTGCTTTTGCTAAACCGCTTCAA
AGAAAAGAGAAGAAAGACATGGACCTCGGGAGGTGGAAAGATATGGTCT
CTGGGGATGATCCTGCATCCACACATGTCCCTCAGCAATCAAGGAAACTT
AAGATCATTGAAACGAGACCGCCCTATGTTGCTTCAGCCGATGCGGCCAC
TACATCCAGCAACACTTTACTGGCTGCCAGGGCATCAGACCAGAGAGAGT
TTGTTTCTGATAAAGCACCGTTTATTAAAAATTTGGGAACCAAGGAAAGG
GTTCCTTTAAACGCTTCTCCTCCCCTAGCTGTTTCGAATGGACTTGGGACT
CGACACGCGTCTTCGTCTCTTGAAAGTGATATTGATGTTGAGAACCATGC
AAAGTTGCAGACAATGTCACCCGACGAGATTGCTGAGGCTCAGGCTGAGT
TATTGGACAAGATGGATCCTGCACTACTCTCCATTTTGAAGAAACGAGGT
GAGGCAAAATTGAAGAAGCGAAAGCATTCTGTGCAGGGGGTTTCCATCAC
CGATGAAACAGCAAAGAATTCAAGAACTGAGGGTCATTTTGTCACTCCTA
AAGTGATGGCAATACCGAAAGAAAAAAGTGTGGTGCAAAAGCCAGGGAT
AGCCCAAGGATTCGTGTGGGATGCATGGACTGAGAGGGTTGAGGCAGCC
AGAGACTTGAGATTTTCTTTTGACGGGAATGTTGTTGAGGAAGATGTTGTC
TCGCCAGCTGAAACTGGTGGAAAGTGGTCTGGTGTTGAATCTGCTGCCGA
ACGTGATTTCTTGAGAACCGAGGGGGATCCTGGGGCCGCAGGTTACACTA
TCAAAGAAGCTATTGCTCTTGCACGAAGTGTGATTCCCGGGCAGAGATGT
CTTGCTTTGCATCTGCTTGCATCTGTACTCGACAAAGCTTTGAACAAACTT
TGTCAAAGCAGAATAGGCTACGCAAGGGAAGAAAAAGATAAATCCACTG
ACTGGGAAGCCATCTGGGCTTATGCCCTTGGACCGGAACCTGAGCTTGTC
TTAGCATTGAGGATGGCTCTTGATGACAACCATGCCTCTGTTGTTATAGCA
TGTGTAAAAGTGATTCAGTGTCTACTGAGCTGTTCTCTTAACGAGAATTTC
TTTAATATTCTGGAGAACATGGGACCACACGGGAAAGATATCTTCACGGC
CTCGGTGTTCAGGAGTAAGCCGGAAATTGATCTTGGCTTCCTCCGTGGTTG
CTACTGGAAGTACAGCGCTAAACCCTCCAATATTGTTGCGTTCCGTGAAG
AAATCTTGGATGACGGGACAGAAGATACGGATACTATTCAGAAAGATGTT
TTTGTAGCCGGACAAGATGTTGCTGCTGGTCTCGTCAGAATGGATATCCTT
CCAAGAATTTATCACCTTCTGGAGACAGAACCAACAGCAGCGCTTGAGGA
CAGCATAATCTCTGTTACTATTGCGATAGCAAGGCATTCTCCAAAATGCA
CAACTGCAATCTTGAAGTATCCCAAATTTGTGCAAACAATTGTGAAAAGA
TTCCAATTGAACAAAAGAATGGACGTTCTTTCTTCTCAGATCAACTCTGTC
CGCCTCTTAAAGGTGTTGGCCCGGTATGATCAAAGTACTTGCATGGAATTT
GTGAAGAATGGGACTTTCAATGCGGTCACATGGCATTTGTTTCAGTTCACC
TCATCTCTTGACTCATGGGTGAAGCTAGGGAAGCAGAACTGCAAGCTTTC
ATCTACCTTGATGGTTGAACAGCTCCGGTTTTGGAAGGTCTGTATCCATAG
TGGCTGTTGCGTATCTCGCTTCCCAGAGCTATTCCCAGCTCTGTGTCTGTG
GTTGAGTTGTCCATCATTCGAAAAGCTCAGGGAGAAAAATCTCATCAGCG
AGTTTACTTCTGTGTCAAACGAGGCCTACCTGGTCCTTGAGGCTTTTGCCG
AGACACTTCCTAATATGTACTCACAAAACATTCCACGGAATGAATCTGGG
ACATGGGACTGGAGCTATGTTAGCCCTATGATTGATTCAGCACTGAGTTG
GATAACATTGGCCCCGCAATTACTCAAGTGGGAGAAAGGAATCGAAAGT
GTCTCTGTATCAACTACTACTCTGTTGTGGTTGTATTCAGGTGTCATGCGT
ACAATTTCCAAAGTCCTTGAGAAAATCTCTGCGGAGGGAGAGGAAGAAC
CTCTACCATGGCTACCGGAGTTTGTTCCAAAGATTGGCCTTGCCATTATCA
AGCACAAGCTTCTTAGTTTTTCTGTTGCAGACGTAAGTAGGTTTGGAAAA
GACTCTTCCAGGTGTTCCTCTTTTATGGAGTATTTGTGTTTTCTAAGAGAA
CGATCTCAAGATGACGAACTAGCATTAGCTTCTGTGAATTGTCTTCATGGG
TTAACACGGACTATCGTGTCCATCCAAAATCTGATAGAATCTGCTAGATC
CAAGATGAAAGCTCCGCATCAGGTAAGTATTTCCACTGGAGATGAATCTG
TGCTTGCAAATGGGATACTGGCAGAGTCTCTGGCTGAGCTAACATCTGTG
TCGTGCTCTTTTAGAGATTCTGTTTCATCAGAATGGCCCATCGTGCAATCA
ATTGAGCTACATAAACGAGGCGGATTGGCCCCCGGCGTTGGACTTGGTTG
GGGAGCTAGCGGTGGTGGGTTTTGGTCAACCAGAGTTCTGTTGGCACAGG
CTGGTGCCGGTCTTCTGAGTCTCTTTCTTAACATCTCTCTGAGCGACTCGC
AGAATGATCAGGGATCTGTTGGCTTTATGGATAAAGTAAACTCCGCTTTA
GCTATGTGTTTGATTGCAGGTCCAAGGGATTATTTACTCGTGGAAAGAGC
CTTTGAATATGTCCTTAGACCGCATGCTTTAGAACACCTGGCCTGCTGTAT
CAAGTCAAACAAAAAAAACATATCGTTTGAATGGGAATGCAGCGAAGGG
GACTATCATCGTATGAGCAGTATGCTTGCTTCTCACTTCAGACATAGATGG
TTACAGCAAAAGGGAAGATCGATAGCCGAGGAAGGGGTCAGTGGGGTAA
GGAAGGGCACAGTTGGTCTGGAGACTATTCATGAGGACGGTGAAATGTCA
AATAGTTCAACTCAGGATAAAAAATCAGACTCCTCGACCATAGAGTGGGC
TCACCAGAGAATGCCCCTACCTCCACACTGGTTTCTCAGCGCCATCTCAGC
AGTCCACAGTGGTAAAACCTCAACAGGGCCACCAGAATCCACAGAGTTG
CTTGAAGTTGCAAAAGCTGGAGTTTTCTTTCTTGCAGGACTTGAGTCATCG
TCTGGTTTTGGATCGCTTCCCTCTCCTGTTGTGAGTGTACCGTTGGTTTGGA
AGTTTCACGCTTTGTCTACCGTATTGCTTGTTGGAATGGACATCATCGAAG
ACAAGAACACTAGGAACTTGTACAATTATCTGCAGGAGCTTTATGGGCAG
TTTCTTGATGAAGCGAGACTAAATCACCGTGACACTGAGCTTCTGAGGTT
CAAGTCAGACATTCATGAGAACTACTCTACTTTTCTGGAGATGGTGGTGG
AGCAGTATGCTGCGGTGTCATATGGTGATGTAGTGTATGGCCGGCAGGTC
TCGGTTTACCTGCATCAATGCGTGGAACACTCTGTTCGGCTTTCGGCATGG
ACAGTGCTCTCCAATGCCCGTGTTCTCGAGCTTCTGCCGAGTCTAGACAA
GTGCTTGGGAGAAGCGGATGGTTACCTCGAACCTGTTGAGGAAAATGAGG
CCGTCCTTGAGGCCTACCTGAAGTCATGGACTTGTGGGGCATTGGACAGA
GCTGCGACGCGTGGATCAGTAGCCTATACGCTGGTTGTGCATCACTTTTCA
TCTTTAGTCTTTTGCAACCAAGCCAAGGATAAAGTATCCCTGCGGAATAA
GATTGTCAAGACTCTTGTCAGGGATTTATCAAGAAAGCGGCATCGTGAGG
GGATGATGTTAGATCTCCTGCGGTATAAGAAAGGGTCTGCGAACGCCATG
GAAGAAGAAGTGATAGCAGCGGAGACAGAGAAAAGAATGGAGGTGTTG
AAAGAGGGTTGCGAAGGGAACTCCACCCTCCTCTTGGAACTGGAGAAGCT
GAAATCAGCCGCTCTCTGTGGAAGAAGGTGA
SEQ ID NO: 2 nucleic acid sequence mutant iyo-1 Arabidopsis
thaliana
ATGGAGCAAAGTAGCGGGAGAGTCAATCCGGAACAGCCGAACAACGTCT
TGGCGAGCCTTGTCGGGAGCATCGTGGAGAAAGGAATATCGGAGAATAA
GCCTCCAAGCAAGCCGCTTCCCCCAAGGCCCTCCCTTCTTTCCTTCCCCGT
CGCTCGTCATCGTTCTCACGGACCCCATTTGGCTCCTGTGGGAAGCAGCAT
AGCACAACCTAAGGATTACAATGACGATCAGGAAGAAGAAGAAGCAGAA
GAACGTTTCATGAATGCAGACTCCATTGCTGCTTTTGCTAAACCGCTTCAA
AGAAAAGAGAAGAAAGACATGGACCTCGGGAGGTGGAAAGATATGGTCT
CTGGGGATGATCCTGCATCCACACATGTCCCTCAGCAATCAAGGAAACTT
AAGATCATTGAAACGAGACCGCCCTATGTTGCTTCAGCCGATGCGGCCAC
TACATCCAGCAACACTTTACTGGCTGCCAGGGCATCAGACCAGAGAGAGT
TTGTTTCTGATAAAGCACCGTTTATTAAAAATTTGGGAACCAAGGAAAGG
GTTCCTTTAAACGCTTCTCCTCCCCTAGCTGTTTCGAATGGACTTGGGACT
CGACACGCGTCTTCGTCTCTTGAAAGTGATATTGATGTTGAGAACCATGC
AAAGTTGCAGACAATGTCACCCGACGAGATTGCTGAGGCTCAGGCTGAGT
TATTGGACAAGATGGATCCTGCACTACTCTCCATTTTGAAGAAACGAGGT
GAGGCAAAATTGAAGAAGCGAAAGCATTCTGTGCAGGGGGTTTCCATCAC
CGATGAAACAGCAAAGAATTCAAGAACTGAGGGTCATTTTGTCACTCCTA
AAGTGATGGCAATACCGAAAGAAAAAAGTGTGGTGCAAAAGCCAGGGAT
AGCCCAAGGATTCGTGTGGGATGCATGGACTGAGAGGGTTGAGGCAGCC
AGAGACTTGAGATTTTCTTTTGACGGGAATGTTGTTGAGGAAGATGTTGTC
TCGCCAGCTGAAACTGGTGGAAAGTGGTCTGGTGTTGAATCTGCTGCCGA
ACGTGATTTCTTGAGAACCGAGGGGGATCCTGGGGCCGCAGGTTACACTA
TCAAAGAAGCTATTGCTCTTGCACGAAGTGTGATTCCCGGGCAGAGATGT
CTTGCTTTGCATCTGCTTGCATCTGTACTCGACAAAGCTTTGAACAAACTT
TGTCAAAGCAGAATAGGCTACGCAAGGGAAGAAAAAGATAAATCCACTG
ACTGGGAAGCCATCTGGGCTTATGCCCTTGGACCGGAACCTGAGCTTGTC
TTAGCATTGAGGATGGCTCTTGATGACAACCATGCCTCTGTTGTTATAGCA
TGTGTAAAAGTGATTCAGTGTCTACTGAGCTGTTCTCTTAACGAGAATTTC
TTTAATATTCTGGAGAACATGGGACCACACGGGAAAGATATCTTCACGGC
CTCGGTGTTCAGGAGTAAGCCGGAAATTGATCTTGGCTTCCTCCGTGGTTG
CTACTGGAAGTACAGCGCTAAACCCTCCAATATTGTTGCGTTCCGTGAAG
AAATCTTGGATGACGGGACAGAAGATACGGATACTATTCAGAAAGATGTT
TTTGTAGCCGGACAAGATGTTGCTGCTGGTCTCGTCAGAATGGATATCCTT
CCAAGAATTTATCACCTTCTGGAGACAGAACCAACAGCAGCGCTTGAGGA
CAGCATAATCTCTGTTACTATTGCGATAGCAAGGCATTCTCCAAAATGCA
CAACTGCAATCTTGAAGTATCCCAAATTTGTGCAAACAATTGTGAAAAGA
TTCCAATTGAACAAAAGAATGGACGTTCTTTCTTCTCAGATCAACTCTGTC
CGCCTCTTAAAGGTGTTGGCCCGGTATGATCAAAGTACTTGCATGGAATTT
GTGAAGAATGGGACTTTCAATGCGGTCACATGGCATTTGTTTCAGTTCACC
TCATCTCTTGACTCATGGGTGAAGCTAGGGAAGCAGAACTGCAAGCTTTC
ATCTACCTTGATGGTTGAACAGCTCCGGTTTTGGAAGGTCTGTATCCATAG
TGGCTGTTGCGTATCTCGCTTCCCAGAGCTATTCCCAGCTCTGTGTCTGTG
GTTGAGTTGTCCATCATTCGAAAAGCTCAGGGAGAAAAATCTCATCAGCG
AGTTTACTTCTGTGTCAAACGAGGCCTACCTGGTCCTTGAGGCTTTTGCCG
AGACACTTCCTAATATGTACTCACAAAACATTCCACGGAATGAATCTGGG
ACATGGGACTGGAGCTATGTTAGCCCTATGATTGATTCAGCACTGAGTTG
GATAACATTGGCCCCGCAATTACTCAAGTGGGAGAAAGGAATCGAAAGT
GTCTCTGTATCAACTACTACTCTGTTGTGGTTGTATTCAGGTGTCATGCGT
ACAATTTCCAAAGTCCTTGAGAAAATCTCTGCGGAGGGAGAGGAAGAAC
CTCTACCATGGCTACCGGAGTTTGTTCCAAAGATTGGCCTTGCCATTATCA
AGCACAAGCTTCTTAGTTTTTCTGTTGCAGACGTAAGTAGGTTTGGAAAA
GACTCTTCCAGGTGTTCCTCTTTTATGGAGTATTTGTGTTTTCTAAGAGAA
CGATCTCAAGATGACGAACTAGCATTAGCTTCTGTGAATTGTCTTCATGGG
TTAACACGGACTATCGTGTCCATCCAAAATCTGATAGAATCTGCTAGATC
CAAGATGAAAGCTCCGCATCAGGTAAGTATTTCCACTGGAGATGAATCTG
TGCTTGCAAATGGGATACTGGCAGAGTCTCTGGCTGAGCTAACATCTGTG
TCGTGCTCTTTTAGAGATTCTGTTTCATCAGAATGGCCCATCGTGCAATCA
ATTGAGCTACATAAACGAGGCGAATTGGCCCCCGGCGTTGGACTTGGTTG
GGGAGCTAGCGGTGGTGGGTTTTGGTCAACCAGAGTTCTGTTGGCACAGG
CTGGTGCCGGTCTTCTGAGTCTCTTTCTTAACATCTCTCTGAGCGACTCGC
AGAATGATCAGGGATCTGTTGGCTTTATGGATAAAGTAAACTCCGCTTTA
GCTATGTGTTTGATTGCAGGTCCAAGGGATTATTTACTCGTGGAAAGAGC
CTTTGAATATGTCCTTAGACCGCATGCTTTAGAACACCTGGCCTGCTGTAT
CAAGTCAAACAAAAAAAACATATCGTTTGAATGGGAATGCAGCGAAGGG
GACTATCATCGTATGAGCAGTATGCTTGCTTCTCACTTCAGACATAGATGG
TTACAGCAAAAGGGAAGATCGATAGCCGAGGAAGGGGTCAGTGGGGTAA
GGAAGGGCACAGTTGGTCTGGAGACTATTCATGAGGACGGTGAAATGTCA
AATAGTTCAACTCAGGATAAAAAATCAGACTCCTCGACCATAGAGTGGGC
TCACCAGAGAATGCCCCTACCTCCACACTGGTTTCTCAGCGCCATCTCAGC
AGTCCACAGTGGTAAAACCTCAACAGGGCCACCAGAATCCACAGAGTTG
CTTGAAGTTGCAAAAGCTGGAGTTTTCTTTCTTGCAGGACTTGAGTCATCG
TCTGGTTTTGGATCGCTTCCCTCTCCTGTTGTGAGTGTACCGTTGGTTTGGA
AGTTTCACGCTTTGTCTACCGTATTGCTTGTTGGAATGGACATCATCGAAG
ACAAGAACACTAGGAACTTGTACAATTATCTGCAGGAGCTTTATGGGCAG
TTTCTTGATGAAGCGAGACTAAATCACCGTGACACTGAGCTTCTGAGGTT
CAAGTCAGACATTCATGAGAACTACTCTACTTTTCTGGAGATGGTGGTGG
AGCAGTATGCTGCGGTGTCATATGGTGATGTAGTGTATGGCCGGCAGGTC
TCGGTTTACCTGCATCAATGCGTGGAACACTCTGTTCGGCTTTCGGCATGG
ACAGTGCTCTCCAATGCCCGTGTTCTCGAGCTTCTGCCGAGTCTAGACAA
GTGCTTGGGAGAAGCGGATGGTTACCTCGAACCTGTTGAGGAAAATGAGG
CCGTCCTTGAGGCCTACCTGAAGTCATGGACTTGTGGGGCATTGGACAGA
GCTGCGACGCGTGGATCAGTAGCCTATACGCTGGTTGTGCATCACTTTTCA
TCTTTAGTCTTTTGCAACCAAGCCAAGGATAAAGTATCCCTGCGGAATAA
GATTGTCAAGACTCTTGTCAGGGATTTATCAAGAAAGCGGCATCGTGAGG
GGATGATGTTAGATCTCCTGCGGTATAAGAAAGGGTCTGCGAACGCCATG
GAAGAAGAAGTGATAGCAGCGGAGACAGAGAAAAGAATGGAGGTGTTG
AAAGAGGGTTGCGAAGGGAACTCCACCCTCCTCTTGGAACTGGAGAAGCT
GAAATCAGCCGCTCTCTGTGGAAGAAGGTGA
SEQ ID NO: 3 Arabidopsis thaliana nucleic acid sequence
mutant iyo-2
ATGGAGCAAAGTAGCGGGAGAGTCAATCCGGAACAGCCGAACAACGTCT
TGGCGAGCCTTGTCGGGAGCATCGTGGAGAAAGGAATATCGGAGAATAA
GCCTCCAAGCAAGCCGCTTCCCCCAAGGCCCTCCCTTCTTTCCTTCCCCGT
CGCTCGTCATCGTTCTCACGGACCCCATTTGGCTCCTGTGGGAAGCAGCAT
AGCACAACCTAAGGATTACAATGACGATCAGGAAGAAGAAGAAGCAGAA
GAACGTTTCATGAATGCAGACTCC[T-DNA, Salk 099873-]
TTGCTGCTTTTGCTAAACCGCTTCAAAGAAAAGAGAAGAAAGACATGGA
CCTCGGGAGGTGGAAAGATATGGTCTCTGGGGATGATCCTGCATCCACAC
ATGTCCCTCAGCAATCAAGGAAACTTAAGATCATTGAAACGAGACCGCCC
TATGTTGCTTCAGCCGATGCGGCCACTACATCCAGCAACACTTTACTGGCT
GCCAGGGCATCAGACCAGAGAGAGTTTGTTTCTGATAAAGCACCGTTTAT
TAAAAATTTGGGAACCAAGGAAAGGGTTCCTTTAAACGCTTCTCCTCCCC
TAGCTGTTTCGAATGGACTTGGGACTCGACACGCGTCTTCGTCTCTTGAAA
GTGATATTGATGTTGAGAACCATGCAAAGTTGCAGACAATGTCACCCGAC
GAGATTGCTGAGGCTCAGGCTGAGTTATTGGACAAGATGGATCCTGCACT
ACTCTCCATTTTGAAGAAACGAGGTGAGGCAAAATTGAAGAAGCGAAAG
CATTCTGTGCAGGGGGTTTCCATCACCGATGAAACAGCAAAGAATTCAAG
AACTGAGGGTCATTTTGTCACTCCTAAAGTGATGGCAATACCGAAAGAAA
AAAGTGTGGTGCAAAAGCCAGGGATAGCCCAAGGATTCGTGTGGGATGC
ATGGACTGAGAGGGTTGAGGCAGCCAGAGACTTGAGATTTTCTTTTGACG
GGAATGTTGTTGAGGAAGATGTTGTCTCGCCAGCTGAAACTGGTGGAAAG
TGGTCTGGTGTTGAATCTGCTGCCGAACGTGATTTCTTGAGAACCGAGGG
GGATCCTGGGGCCGCAGGTTACACTATCAAAGAAGCTATTGCTCTTGCAC
GAAGTGTGATTCCCGGGCAGAGATGTCTTGCTTTGCATCTGCTTGCATCTG
TACTCGACAAAGCTTTGAACAAACTTTGTCAAAGCAGAATAGGCTACGCA
AGGGAAGAAAAAGATAAATCCACTGACTGGGAAGCCATCTGGGCTTATG
CCCTTGGACCGGAACCTGAGCTTGTCTTAGCATTGAGGATGGCTCTTGATG
ACAACCATGCCTCTGTTGTTATAGCATGTGTAAAAGTGATTCAGTGTCTAC
TGAGCTGTTCTCTTAACGAGAATTTCTTTAATATTCTGGAGAACATGGGAC
CACACGGGAAAGATATCTTCACGGCCTCGGTGTTCAGGAGTAAGCCGGAA
ATTGATCTTGGCTTCCTCCGTGGTTGCTACTGGAAGTACAGCGCTAAACCC
TCCAATATTGTTGCGTTCCGTGAAGAAATCTTGGATGACGGGACAGAAGA
TACGGATACTATTCAGAAAGATGTTTTTGTAGCCGGACAAGATGTTGCTG
CTGGTCTCGTCAGAATGGATATCCTTCCAAGAATTTATCACCTTCTGGAGA
CAGAACCAACAGCAGCGCTTGAGGACAGCATAATCTCTGTTACTATTGCG
ATAGCAAGGCATTCTCCAAAATGCACAACTGCAATCTTGAAGTATCCCAA
ATTTGTGCAAACAATTGTGAAAAGATTCCAATTGAACAAAAGAATGGACG
TTCTTTCTTCTCAGATCAACTCTGTCCGCCTCTTAAAGGTGTTGGCCCGGT
ATGATCAAAGTACTTGCATGGAATTTGTGAAGAATGGGACTTTCAATGCG
GTCACATGGCATTTGTTTCAGTTCACCTCATCTCTTGACTCATGGGTGAAG
CTAGGGAAGCAGAACTGCAAGCTTTCATCTACCTTGATGGTTGAACAGCT
CCGGTTTTGGAAGGTCTGTATCCATAGTGGCTGTTGCGTATCTCGCTTCCC
AGAGCTATTCCCAGCTCTGTGTCTGTGGTTGAGTTGTCCATCATTCGAAAA
GCTCAGGGAGAAAAATCTCATCAGCGAGTTTACTTCTGTGTCAAACGAGG
CCTACCTGGTCCTTGAGGCTTTTGCCGAGACACTTCCTAATATGTACTCAC
AAAACATTCCACGGAATGAATCTGGGACATGGGACTGGAGCTATGTTAGC
CCTATGATTGATTCAGCACTGAGTTGGATAACATTGGCCCCGCAATTACTC
AAGTGGGAGAAAGGAATCGAAAGTGTCTCTGTATCAACTACTACTCTGTT
GTGGTTGTATTCAGGTGTCATGCGTACAATTTCCAAAGTCCTTGAGAAAAT
CTCTGCGGAGGGAGAGGAAGAACCTCTACCATGGCTACCGGAGTTTGTTC
CAAAGATTGGCCTTGCCATTATCAAGCACAAGCTTCTTAGTTTTTCTGTTG
CAGACGTAAGTAGGTTTGGAAAAGACTCTTCCAGGTGTTCCTCTTTTATGG
AGTATTTGTGTTTTCTAAGAGAACGATCTCAAGATGACGAACTAGCATTA
GCTTCTGTGAATTGTCTTCATGGGTTAACACGGACTATCGTGTCCATCCAA
AATCTGATAGAATCTGCTAGATCCAAGATGAAAGCTCCGCATCAGGTAAG
TATTTCCACTGGAGATGAATCTGTGCTTGCAAATGGGATACTGGCAGAGT
CTCTGGCTGAGCTAACATCTGTGTCGTGCTCTTTTAGAGATTCTGTTTCAT
CAGAATGGCCCATCGTGCAATCAATTGAGCTACATAAACGAGGCGGATTG
GCCCCCGGCGTTGGACTTGGTTGGGGAGCTAGCGGTGGTGGGTTTTGGTC
AACCAGAGTTCTGTTGGCACAGGCTGGTGCCGGTCTTCTGAGTCTCTTTCT
TAACATCTCTCTGAGCGACTCGCAGAATGATCAGGGATCTGTTGGCTTTAT
GGATAAAGTAAACTCCGCTTTAGCTATGTGTTTGATTGCAGGTCCAAGGG
ATTATTTACTCGTGGAAAGAGCCTTTGAATATGTCCTTAGACCGCATGCTT
TAGAACACCTGGCCTGCTGTATCAAGTCAAACAAAAAAAACATATCGTTT
GAATGGGAATGCAGCGAAGGGGACTATCATCGTATGAGCAGTATGCTTGC
TTCTCACTTCAGACATAGATGGTTACAGCAAAAGGGAAGATCGATAGCCG
AGGAAGGGGTCAGTGGGGTAAGGAAGGGCACAGTTGGTCTGGAGACTAT
TCATGAGGACGGTGAAATGTCAAATAGTTCAACTCAGGATAAAAAATCAG
ACTCCTCGACCATAGAGTGGGCTCACCAGAGAATGCCCCTACCTCCACAC
TGGTTTCTCAGCGCCATCTCAGCAGTCCACAGTGGTAAAACCTCAACAGG
GCCACCAGAATCCACAGAGTTGCTTGAAGTTGCAAAAGCTGGAGTTTTCT
TTCTTGCAGGACTTGAGTCATCGTCTGGTTTTGGATCGCTTCCCTCTCCTGT
TGTGAGTGTACCGTTGGTTTGGAAGTTTCACGCTTTGTCTACCGTATTGCT
TGTTGGAATGGACATCATCGAAGACAAGAACACTAGGAACTTGTACAATT
ATCTGCAGGAGCTTTATGGGCAGTTTCTTGATGAAGCGAGACTAAATCAC
CGTGACACTGAGCTTCTGAGGTTCAAGTCAGACATTCATGAGAACTACTC
TACTTTTCTGGAGATGGTGGTGGAGCAGTATGCTGCGGTGTCATATGGTG
ATGTAGTGTATGGCCGGCAGGTCTCGGTTTACCTGCATCAATGCGTGGAA
CACTCTGTTCGGCTTTCGGCATGGACAGTGCTCTCCAATGCCCGTGTTCTC
GAGCTTCTGCCGAGTCTAGACAAGTGCTTGGGAGAAGCGGATGGTTACCT
CGAACCTGTTGAGGAAAATGAGGCCGTCCTTGAGGCCTACCTGAAGTCAT
GGACTTGTGGGGCATTGGACAGAGCTGCGACGCGTGGATCAGTAGCCTAT
ACGCTGGTTGTGCATCACTTTTCATCTTTAGTCTTTTGCAACCAAGCCAAG
GATAAAGTATCCCTGCGGAATAAGATTGTCAAGACTCTTGTCAGGGATTT
ATCAAGAAAGCGGCATCGTGAGGGGATGATGTTAGATCTCCTGCGGTATA
AGAAAGGGTCTGCGAACGCCATGGAAGAAGAAGTGATAGCAGCGGAGAC
AGAGAAAAGAATGGAGGTGTTGAAAGAGGGTTGCGAAGGGAACTCCACC
CTCCTCTTGGAACTGGAGAAGCTGAAATCAGCCGCTCTCTGTGGAAGAAG
GTGA
SEQ ID NO: 4 Arabidopsis thaliana nucleic acid sequence
mutant iyo-3
ATGGAGCAAAGTAGCGGGAGAGTCAATCCGGAACAGCCGAACAACGTCT
TGGCGAGCCTTGTCGGGAGCATCGTGGAGAAAGGAATATCGGAGAATAA
GCCTCCAAGCAAGCCGCTTCCCCCAAGGCCCTCCCTTCTTTCCTTCCCCGT
CGCTCGTCATCGTTCTCACGGACCCCATTTGGCTCCTGTGGGAAGCAGCAT
AGCACAACCTAAGGATTACAATGACGATCAGGAAGAAGAAGAAGCAGAA
GAACGTTTCATGAATGCAGACTCCATTGCTGCTTTTGCTAAACCGCTTCAA
AGAAAAGAGAAGAAAGACATGGACCTCGGGAGGTGGAAAGATATGGTCT
CTGGGGATGATCCTGCATCCACACATGTCCCTCAGCAATCAAGGAAACTT
AAGATCATTGAAACGAGACCGCCCTATGTTGCTTCAGCCGATGCGGCCAC
TACATCCAGCAACACTTTACTGGCTGCCAGGGCATCAGACCAGAGAGAGT
TTGTTTCTGATAAAGCACCGTTTATTAAAAATTTGGGAACCAAGGAAAGG
GTTCCTTTAAACGCTTCTCCTCCCCTAGCTGTTTCGAATGGACTTGGGACT
CGACACGCGTCTTCGTCTCTTGAAAGTGATATTGATGTTGAGAACCATGC
AAAGTTGCAGACAATGTCACCCGACGAGATTGCTGAGGCTCAGGCTGAGT
TATTGGACAAGATGGATCCTGCACTACTCTCCATTTTGAAGAAACGAGGT
GAGGCAAAATTGAAGAAGCGAAAGCATTCTGTGCAGGGGGTTTCCATCAC
CGATGAAACAGCAAAGAATTCAAGAACTGAGGGTCATTTTGTCACTCCTA
AAGTGATGGCAATACCGAAAGAAAAAAGTGTGGTGCAAAAGCCAGGGAT
AGCCCAAGGATTCGTGTGGGATGCATGGACTGAGAGGGTTGAGGCAGCC
AGAGACTTGAGATTTTCTTTTGACGGGAATGTTGTTGAGGAAGATGTTGTC
TCGCCAGCTGAAACTGGTGGAAAGTGGTCTGGTGTTGAATCTGCTGCCGA
ACGTGATTTCTTGAGAACCGAGGGGGATCCTGGGGCCGCAGGTTACACTA
TCAAAGAAGCTATTGCTCTTGCACGAAGTGTGATTCCCGGGCAGAGATGT
CTTGCTTTGCATCTGCTTGCATCTGTACTCGACAAAGCTTTGAACAAACTT
TGTCAAAGCAGAATAGGCTACGCAAGGGAAGAAAAAGATAAATCCACTG
ACTGGGAAGCCATCTGGGCTTATGCCCTTGGACCGGAACCTGAGCTTGTC
TTAGCATTGAGGATGGCTCTTGATGACAACCATGCCTCTGTTGTTATAGCA
TGTGTA[T-DNA
salk692_g12]AAAGTGATTCAGTGTCTACTGAGCTGTTCTCTTAACGAGAATT
TCTTTAATATTCTGGAGAACATGGGACCACACGGGAAAGATATCTTCACG
GCCTCGGTGTTCAGGAGTAAGCCGGAAATTGATCTTGGCTTCCTCCGTGGT
TGCTACTGGAAGTACAGCGCTAAACCCTCCAATATTGTTGCGTTCCGTGA
AGAAATCTTGGATGACGGGACAGAAGATACGGATACTATTCAGAAAGAT
GTTTTTGTAGCCGGACAAGATGTTGCTGCTGGTCTCGTCAGAATGGATATC
CTTCCAAGAATTTATCACCTTCTGGAGACAGAACCAACAGCAGCGCTTGA
GGACAGCATAATCTCTGTTACTATTGCGATAGCAAGGCATTCTCCAAAAT
GCACAACTGCAATCTTGAAGTATCCCAAATTTGTGCAAACAATTGTGAAA
AGATTCCAATTGAACAAAAGAATGGACGTTCTTTCTTCTCAGATCAACTCT
GTCCGCCTCTTAAAGGTGTTGGCCCGGTATGATCAAAGTACTTGCATGGA
ATTTGTGAAGAATGGGACTTTCAATGCGGTCACATGGCATTTGTTTCAGTT
CACCTCATCTCTTGACTCATGGGTGAAGCTAGGGAAGCAGAACTGCAAGC
TTTCATCTACCTTGATGGTTGAACAGCTCCGGTTTTGGAAGGTCTGTATCC
ATAGTGGCTGTTGCGTATCTCGCTTCCCAGAGCTATTCCCAGCTCTGTGTC
TGTGGTTGAGTTGTCCATCATTCGAAAAGCTCAGGGAGAAAAATCTCATC
AGCGAGTTTACTTCTGTGTCAAACGAGGCCTACCTGGTCCTTGAGGCTTTT
GCCGAGACACTTCCTAATATGTACTCACAAAACATTCCACGGAATGAATC
TGGGACATGGGACTGGAGCTATGTTAGCCCTATGATTGATTCAGCACTGA
GTTGGATAACATTGGCCCCGCAATTACTCAAGTGGGAGAAAGGAATCGAA
AGTGTCTCTGTATCAACTACTACTCTGTTGTGGTTGTATTCAGGTGTCATG
CGTACAATTTCCAAAGTCCTTGAGAAAATCTCTGCGGAGGGAGAGGAAGA
ACCTCTACCATGGCTACCGGAGTTTGTTCCAAAGATTGGCCTTGCCATTAT
CAAGCACAAGCTTCTTAGTTTTTCTGTTGCAGACGTAAGTAGGTTTGGAAA
AGACTCTTCCAGGTGTTCCTCTTTTATGGAGTATTTGTGTTTTCTAAGAGA
ACGATCTCAAGATGACGAACTAGCATTAGCTTCTGTGAATTGTCTTCATGG
GTTAACACGGACTATCGTGTCCATCCAAAATCTGATAGAATCTGCTAGAT
CCAAGATGAAAGCTCCGCATCAGGTAAGTATTTCCACTGGAGATGAATCT
GTGCTTGCAAATGGGATACTGGCAGAGTCTCTGGCTGAGCTAACATCTGT
GTCGTGCTCTTTTAGAGATTCTGTTTCATCAGAATGGCCCATCGTGCAATC
AATTGAGCTACATAAACGAGGCGGATTGGCCCCCGGCGTTGGACTTGGTT
GGGGAGCTAGCGGTGGTGGGTTTTGGTCAACCAGAGTTCTGTTGGCACAG
GCTGGTGCCGGTCTTCTGAGTCTCTTTCTTAACATCTCTCTGAGCGACTCG
CAGAATGATCAGGGATCTGTTGGCTTTATGGATAAAGTAAACTCCGCTTT
AGCTATGTGTTTGATTGCAGGTCCAAGGGATTATTTACTCGTGGAAAGAG
CCTTTGAATATGTCCTTAGACCGCATGCTTTAGAACACCTGGCCTGCTGTA
TCAAGTCAAACAAAAAAAACATATCGTTTGAATGGGAATGCAGCGAAGG
GGACTATCATCGTATGAGCAGTATGCTTGCTTCTCACTTCAGACATAGATG
GTTACAGCAAAAGGGAAGATCGATAGCCGAGGAAGGGGTCAGTGGGGTA
AGGAAGGGCACAGTTGGTCTGGAGACTATTCATGAGGACGGTGAAATGTC
AAATAGTTCAACTCAGGATAAAAAATCAGACTCCTCGACCATAGAGTGGG
CTCACCAGAGAATGCCCCTACCTCCACACTGGTTTCTCAGCGCCATCTCAG
CAGTCCACAGTGGTAAAACCTCAACAGGGCCACCAGAATCCACAGAGTT
GCTTGAAGTTGCAAAAGCTGGAGTTTTCTTTCTTGCAGGACTTGAGTCATC
GTCTGGTTTTGGATCGCTTCCCTCTCCTGTTGTGAGTGTACCGTTGGTTTGG
AAGTTTCACGCTTTGTCTACCGTATTGCTTGTTGGAATGGACATCATCGAA
GACAAGAACACTAGGAACTTGTACAATTATCTGCAGGAGCTTTATGGGCA
GTTTCTTGATGAAGCGAGACTAAATCACCGTGACACTGAGCTTCTGAGGT
TCAAGTCAGACATTCATGAGAACTACTCTACTTTTCTGGAGATGGTGGTG
GAGCAGTATGCTGCGGTGTCATATGGTGATGTAGTGTATGGCCGGCAGGT
CTCGGTTTACCTGCATCAATGCGTGGAACACTCTGTTCGGCTTTCGGCATG
GACAGTGCTCTCCAATGCCCGTGTTCTCGAGCTTCTGCCGAGTCTAGACA
AGTGCTTGGGAGAAGCGGATGGTTACCTCGAACCTGTTGAGGAAAATGAG
GCCGTCCTTGAGGCCTACCTGAAGTCATGGACTTGTGGGGCATTGGACAG
AGCTGCGACGCGTGGATCAGTAGCCTATACGCTGGTTGTGCATCACTTTTC
ATCTTTAGTCTTTTGCAACCAAGCCAAGGATAAAGTATCCCTGCGGAATA
AGATTGTCAAGACTCTTGTCAGGGATTTATCAAGAAAGCGGCATCGTGAG
GGGATGATGTTAGATCTCCTGCGGTATAAGAAAGGGTCTGCGAACGCCAT
GGAAGAAGAAGTGATAGCAGCGGAGACAGAGAAAAGAATGGAGGTGTT
GAAAGAGGGTTGCGAAGGGAACTCCACCCTCCTCTTGGAACTGGAGAAG
CTGAAATCAGCCGCTCTCTGTGGAAGAAGGTGA
SEQ ID NO: 5 Arabidopsis thaliana Protein MINIYO
MEQSSGRVNPEQPNNVLASLVGSIVEKGISENKPPSKPLPPRPSLLSFPVARHR
SHGPHLAPVGSSIAQPKDYNDDQEEEEAEERFMNADSIAAFAKPLQRKEKKD
MDLGRWKDMVSGDDPASTHVPQQSRKLKIIETRPPYVASADAATTSSNTLLA
ARASDQREFVSDKAPFIKNLGTKERVPLNASPPLAVSNGLGTRHASSSLESDI
DVENHAKLQTMSPDEIAEAQAELLDKMDPALLSILKKRGEAKLKKRKHSVQ
GVSITDETAKNSRTEGHFVTPKVMAIPKEKSVVQKPGIAQGFVWDAWTERVE
AARDLRFSFDGNVVEEDVVSPAETGGKWSGVESAAERDFLRTEGDPGAAGY
TIKEAIALARSVIPGQRCLALHLLASVLDKALNKLCQSRIGYAREEKDKSTDW
EAIWAYALGPEPELVLALRMALDDNHASVVIACVKVIQCLLSCSLNENFFNIL
ENMGPHGKDIFTASVFRSKPEIDLGFLRGCYWKYSAKPSNIVAFREEILDDGT
EDTDTIQKDVFVAGQDVAAGLVRMDILPRIYHLLETEPTAALEDSIISVTIAIA
RHSPKCTTAILKYPKFVQTIVKRFQLNKRMDVLSSQINSVRLLKVLARYDQST
CMEFVKNGTFNAVTWHLFQFTSSLDSWVKLGKQNCKLSSTLMVEQLRFWK
VCIHSGCCVSRFPELFPALCLWLSCPSFEKLREKNLISEFTSVSNEAYLVLEAF
AETLPNMYSQNIPRNESGTWDWSYVSPMIDSALSWITLAPQLLKWEKGIESV
SVSTTTLLWLYSGVMRTISKVLEKISAEGEEEPLPWLPEFVPKIGLAIIKHKLLS
FSVADVSRFGKDSSRCSSFMEYLCFLRERSQDDELALASVNCLHGLTRTIVSI
QNLIESARSKMKAPHQVSISTGDESVLANGILAESLAELTSVSCSFRDSVSSEW
PIVQSIELHKRGGLAPGVGLGWGASGGGFWSTRVLLAQAGAGLLSLFLNISLS
DSQNDQGSVGFMDKVNSALAMCLIAGPRDYLLVERAFEYVLRPHALEHLAC
CIKSNKKNISFEWECSEGDYHRMSSMLASHFRHRWLQQKGRSIAEEGVSGVR
KGTVGLETIHEDGEMSNSSTQDKKSDSSTIEWAHQRMPLPPHWFLSAISAVHS
GKTSTGPPESTELLEVAKAGVFFLAGLESSSGFGSLPSPVVSVPLVWKFHALS
TVLLVGMDIIEDKNTRNLYNYLQELYGQFLDEARLNHRDTELLRFKSDIHEN
YSTFLEMVVEQYAAVSYGDVVYGRQVSVYLHQCVEHSVRLSAWTVLSNAR
VLELLPSLDKCLGEADGYLEPVEENEAVLEAYLKSWTCGALDRAATRGSVA
YTLVVHHFSSLVFCNQAKDKVSLRNKIVKTLVRDLSRKRHREGMMLDLLRY
KKGSANAMEEEVIAAETEKRMEVLKEGCEGNSTLLLELEKLKSAALCGRR
SEQ ID NO: 6 Arabidopsis thaliana, protein allele iyo-1
MEQSSGRVNPEQPNNVLASLVGSIVEKGISENKPPSKPLPPRPSLLSFPVARHR
SHGPHLAPVGSSIAQPKDYNDDQEEEEAEERFMNADSIAAFAKPLQRKEKKD
MDLGRWKDMVSGDDPASTHVPQQSRKLKIIETRPPYVASADAATTSSNTLLA
ARASDQREFVSDKAPFIKNLGTKERVPLNASPPLAVSNGLGTRHASSSLESDI
DVENHAKLQTMSPDEIAEAQAELLDKMDPALLSILKKRGEAKLKKRKHSVQ
GVSITDETAKNSRTEGHFVTPKVMAIPKEKSVVQKPGIAQGFVWDAWTERVE
AARDLRFSFDGNVVEEDVVSPAETGGKWSGVESAAERDFLRTEGDPGAAGY
TIKEAIALARSVIPGQRCLALHLLASVLDKALNKLCQSRIGYAREEKDKSTDW
EAIWAYALGPEPELVLALRMALDDNHASVVIACVKVIQCLLSCSLNENFFNIL
ENMGPHGKDIFTASVFRSKPEIDLGFLRGCYWKYSAKPSNIVAFREEILDDGT
EDTDTIQKDVFVAGQDVAAGLVRMDILPRIYHLLETEPTAALEDSIISVTIAIA
RHSPKCTTAILKYPKFVQTIVKRFQLNKRMDVLSSQINSVRLLKVLARYDQST
CMEFVKNGTFNAVTWHLFQFTSSLDSWVKLGKQNCKLSSTLMVEQLRFWK
VCIHSGCCVSRFPELFPALCLWLSCPSFEKLREKNLISEFTSVSNEAYLVLEAF
AETLPNMYSQNIPRNESGTWDWSYVSPMIDSALSWITLAPQLLKWEKGIESV
SVSTTTLLWLYSGVMRTISKVLEKISAEGEEEPLPWLPEFVPKIGLAIIKHKLLS
FSVADVSRFGKDSSRCSSFMEYLCFLRERSQDDELALASVNCLHGLTRTIVSI
QNLIESARSKMKAPHQVSISTGDESVLANGILAESLAELTSVSCSFRDSVSSEW
PIVQSIELHKRGELAPGVGLGWGASGGGFWSTRVLLAQAGAGLLSLFLNISLS
DSQNDQGSVGFMDKVNSALAMCLIAGPRDYLLVERAFEYVLRPHALEHLAC
CIKSNKKNISFEWECSEGDYHRMSSMLASHFRHRWLQQKGRSIAEEGVSGVR
KGTVGLETIHEDGEMSNSSTQDKKSDSSTIEWAHQRMPLPPHWFLSAISAVHS
GKTSTGPPESTELLEVAKAGVFFLAGLESSSGFGSLPSPVVSVPLVWKFHALS
TVLLVGMDIIEDKNTRNLYNYLQELYGQFLDEARLNHRDTELLRFKSDIHEN
YSTFLEMVVEQYAAVSYGDVVYGRQVSVYLHQCVEHSVRLSAWTVLSNAR
VLELLPSLDKCLGEADGYLEPVEENEAVLEAYLKSWTCGALDRAATRGSVA
YTLVVHHFSSLVFCNQAKDKVSLRNKIVKTLVRDLSRKRHREGMMLDLLRY
KKGSANAMEEEVIAAETEKRMEVLKEGCEGNSTLLLELEKLKSAALCGRR
SEQ ID No. 7 MINIYO nucleic acid sequence Arabidopsis
thaliana
AAAGAGTTTTCCGTTTTGCTGAGCGGAGGCGAGAGAGGGTTTAGAGTGAT
GGAGCAAAGTAGCGGGAGAGTCAATCCGGAACAGCCGAACAACGTCTTG
GCGAGCCTTGTCGGGAGCATCGTGGAGAAAGGAATATCGGAGAATAAGC
CTCCAAGCAAGCCGCTTCCCCCAAGGCCCTCCCTTCTTTCCTTCCCCGTCG
CTCGTCATCGTTCTCACGGACCCGTAAGCCAATCCAATCCTCTAGTGCGTG
CTTTTTAGGTTTCCATCTTCCTTTTGTTGCCTTCTTCTAGATTTTAAGCACC
TTCTACTGTTGTTTAGTACTTGGGACTCCACAATTTTTCACCGTGCCTGAC
CTTGTAATTCAGCTTTCTGAGACATCTAATTTTTGTTTCTCATGTTTGATTT
TGTAGCATTTGGCTCCTGTGGGAAGCAGCATAGCACAACCTAAGGATTAC
AATGACGATCAGGAAGAAGAAGAAGCAGAAGAACGTTTCATGAATGCAG
ACTCCATTGCTGCTTTTGCTAAACCGCTTCAAAGAAAAGAGAAGAAAGAC
ATGGACCTCGGGAGGTGGAAAGATATGGTCTCTGGGGATGATCCTGCATC
CACACATGTCCCTCAGCAATCAAGGAAACTTAAGATCATTGAAACGAGAC
CGCCCTATGTTGCTTCAGCCGATGCGGCCACTACATCCAGCAACACTTTAC
TGGCTGCCAGGGCATCAGACCAGAGAGAGTTTGTTTCTGATAAAGCACCG
TTTATTAAAAATTTGGGAACCAAGGAAAGGGTTCCTTTAAACGCTTCTCCT
CCCCTAGCTGTTTCGAATGGACTTGGGACTCGACACGCGTCTTCGTCTCTT
GAAAGTGATATTGATGTTGAGAACCATGCAAAGTTGCAGACAATGTCACC
CGACGAGATTGCTGAGGCTCAGGCTGAGTTATTGGACAAGATGGATCCTG
CACTACTCTCCATTTTGAAGAAACGAGGTGAGGCAAAATTGAAGAAGCGA
AAGCATTCTGTGCAGGGGGTTTCCATCACCGATGAAACAGCAAAGAATTC
AAGAACTGAGGGTCATTTTGTCACTCCTAAAGTGATGGCAATACCGAAAG
AAAAAAGTGTGGTGCAAAAGCCAGGGATAGCCCAAGGATTCGTGTGGGA
TGCATGGACTGAGAGGGTTGAGGCAGCCAGAGACTTGAGATTTTCTTTTG
ACGGGAATGTTGTTGAGGAAGATGTTGTCTCGCCAGCTGAAACTGGTGAG
TAGAACAATACAACTGAAACACATGACAATCTTAGGTTGCTTACACTTTG
ACTGTACAGGTGGAAAGTGGTCTGGTGTTGAATCTGCTGCCGAACGTGAT
TTCTTGAGAACCGAGGGGGATCCTGGGGCCGCAGGTTACACTATCAAAGA
AGCTATTGCTCTTGCACGAAGTGTGGTATGTATGATTGCCACATATTTTAA
TTTTGATGCTAATTAATGGTTAAATTCTTTTTTCCCTCCATTTTGGCTTTAG
CTGAACAAAACCTGTAGGCTGAGACTGCGTTTTTTTCGTTATCACTGCTCA
TTGATTTGTATGTATTATTGATATATATATCAGATTCCCGGGCAGAGATGT
CTTGCTTTGCATCTGCTTGCATCTGTACTCGACAAAGCTTTGAACAAACTT
TGTCAAAGCAGAATAGGCTACGCAAGGGAAGAAAAAGATAAATCCACTG
ACTGGGAAGCCATCTGGGCTTATGCCCTTGGACCGGAACCTGAGCTTGTC
TTAGCATTGAGGTAATTTCCTGATGGGTGTAATTTTGAGACTTATTTGTGA
AGTTGTCACTCATAAATCATAAATTGTTTGTTCTTATCAATATAAGTTTCTT
TTCTTCTTTAGGATGGCTCTTGATGACAACCATGCCTCTGTTGTTATAGCA
TGTGTAAAAGTGATTCAGTGTCTACTGAGCTGTTCTCTTAACGAGAATTTC
TTTAATATTCTGGAGGTATAGTTGATTTTTCTCACTCCTAAGAAGTTATAG
TCCTCATAGAACGTGATTATACATGTTCAAACTGATAAAACCCATTTCTAT
TTCCAGAACATGGGACCACACGGGAAAGATATCTTCACGGCCTCGGTGTT
CAGGAGTAAGCCGGAAATTGATCTTGGCTTCCTCCGTGGTTGCTACTGGA
AGTACAGCGCTAAACCCTCCAATATTGTTGCGTTCCGTGAAGAAATCTTG
GATGACGGGACAGAAGATACGGATACTATTCAGAAAGATGTTTTTGTAGC
CGGACAAGATGTTGCTGCTGGTCTCGTCAGAATGGATATCCTTCCAAGAA
TTTATCACCTTCTGGAGGTGAGATCACTATCTATGTGTAACTCAGCAAGTA
AAATCATTCTTTTTGTGTCGTTGCTTAGTTTTCTGGTTTTTTTTTAATGTTCA
TGATTTCAGACAGAACCAACAGCAGCGCTTGAGGACAGCATAATCTCTGT
TACTATTGCGATAGCAAGGCATTCTCCAAAATGCACAACTGCAATCTTGA
AGTATCCCAAATTTGTGCAAACAATTGTGAAAAGATTCCAATTGAACAAA
AGAATGGACGTTCTTTCTTCTCAGATCAACTCTGTCCGCCTCTTAAAGGTA
ATACTGGTCCGCTCATACAAAATTATCTTGGGGTCGTTATATTCATTCGTC
TTTGATGTTTTTTTTACAGAACCTGATGATTCGAGTTTGTTAAGCTATCAA
TTCTCAGAGCTATTGTAACCTTCGTTCTTCTTTCTCTCTTTTTAATTTCACT
AAGGTGTTGGCCCGGTATGATCAAAGTACTTGCATGGAATTTGTGAAGAA
TGGGACTTTCAATGCGGTCACATGGCATTTGTTTCAGTTCACCTCATCTCT
TGACTCATGGGTGAAGCTAGGGAAGCAGAACTGCAAGCTTTCATCTACCT
TGATGGTTGAACAGCTCCGGTTTTGGAAGGTCTGTATCCATAGTGGCTGTT
GCGTATCTCGCTTCCCAGAGCTATTCCCAGCTCTGTGTCTGTGGTTGAGTT
GTCCATCATTCGAAAAGCTCAGGGAGAAAAATCTCATCAGCGAGTTTACT
TCTGTGTCAAACGAGGCCTACCTGGTCCTTGAGGCTTTTGCCGAGACACTT
CCTAATATGTACTCACAAAACATTCCACGGAATGAATCTGGGACATGGGA
CTGGAGCTATGTTAGCCCTATGATTGATTCAGCACTGAGTTGGATAACATT
GGCCCCGCAATTACTCAAGTGGGAGAAAGGAATCGAAAGTGTCTCTGTAT
CAACTACTACTCTGTTGTGGTTGTATTCAGGTGTCATGCGTACAATTTCCA
AAGTCCTTGAGAAAATCTCTGCGGAGGGAGAGGAAGAACCTCTACCATG
GCTACCGGAGTTTGTTCCAAAGATTGGCCTTGCCATTATCAAGCACAAGC
TTCTTAGTTTTTCTGTTGCAGACGTAAGTAGGTTTGGAAAAGACTCTTCCA
GGTGTTCCTCTTTTATGGAGTATTTGTGTTTTCTAAGAGAACGATCTCAAG
ATGACGAACTAGCATTAGCTTCTGTGAATTGTCTTCATGGGTTAACACGG
ACTATCGTGTCCATCCAAAATCTGATAGAATCTGCTAGATCCAAGATGAA
AGCTCCGCATCAGGTAAGTATTTCCACTGGAGATGAATCTGTGCTTGCAA
ATGGGATACTGGCAGAGTCTCTGGCTGAGCTAACATCTGTGTCGTGCTCTT
TTAGAGATTCTGTTTCATCAGAATGGCCCATCGTGCAATCAATTGAGCTAC
ATAAACGAGGCGGATTGGCCCCCGGCGTTGGACTTGGTTGGGGAGCTAGC
GGTGGTGGGTTTTGGTCAACCAGAGTTCTGTTGGCACAGGCTGGTGCCGG
TCTTCTGAGTCTCTTTCTTAACATCTCTCTGAGCGACTCGCAGAATGATCA
GGGATCTGTTGGCTTTATGGATAAAGTAAACTCCGCTTTAGCTATGTGTTT
GATTGCAGGTCCAAGGGATTATTTACTCGTGGAAAGAGCCTTTGAATATG
TCCTTAGACCGCATGCTTTAGAACACCTGGCCTGCTGTATCAAGTCAAAC
AAAAAAAACATATCGTTTGAATGGGAATGCAGCGAAGGGGACTATCATC
GTATGAGCAGTATGCTTGCTTCTCACTTCAGACATAGATGGTTACAGCAA
AAGGGAAGATCGATAGCCGAGGAAGGGGTCAGTGGGGTAAGGAAGGGC
ACAGTTGGTCTGGAGACTATTCATGAGGACGGTGAAATGTCAAATAGTTC
AACTCAGGATAAAAAATCAGACTCCTCGACCATAGAGTGGGCTCACCAG
AGAATGCCCCTACCTCCACACTGGTTTCTCAGCGCCATCTCAGCAGTCCAC
AGTGGTAAAACCTCAACAGGGCCACCAGAATCCACAGAGTTGCTTGAAGT
TGCAAAAGCTGGAGTTTTCTTTCTTGCAGGACTTGAGTCATCGTCTGGTTT
TGGATCGCTTCCCTCTCCTGTTGTGAGTGTACCGTTGGTTTGGAAGTTTCA
CGCTTTGTCTACCGTATTGCTTGTTGGAATGGACATCATCGAAGACAAGA
ACACTAGGAACTTGTACAATTATCTGCAGGAGCTTTATGGGCAGTTTCTTG
ATGAAGCGAGACTAAATCACCGTGACACTGAGCTTCTGAGGTTCAAGTCA
GACATTCATGAGAACTACTCTACTTTTCTGGAGATGGTGGTGGAGCAGTA
TGCTGCGGTGTCATATGGTGATGTAGTGTATGGCCGGCAGGTCTCGGTTTA
CCTGCATCAATGCGTGGAACACTCTGTTCGGCTTTCGGCATGGACAGTGCT
CTCCAATGCCCGTGTTCTCGAGCTTCTGCCGAGTCTAGACAAGTGCTTGGG
AGAAGCGGATGGTTACCTCGAACCTGTTGAGGTAATTTACAAAAATAATA
AAATGTTGATAGTGGTGAATAATGGCATCTTGAGCATCCAACTAAGATAA
AATGGTGAATTGATATTGCAGGAAAATGAGGCCGTCCTTGAGGCCTACCT
GAAGTCATGGACTTGTGGGGCATTGGACAGAGCTGCGACGCGTGGATCAG
TAGCCTATACGCTGGTTGTGCATCACTTTTCATCTTTAGTCTTTTGCAACCA
AGCCAAGGATAAAGTATCCCTGCGGAATAAGATTGTCAAGACTCTTGTCA
GGGATTTATCAAGAAAGCGGCATCGTGAGGTAACTTGAAATCCCTCATCT
CTTGTTCAATGTCATTCTGGGGACTGAGGATGATAATGAAACGTGGAAAT
AATGTTTCAGGGGATGATGTTAGATCTCCTGCGGTATAAGAAAGGGTCTG
CGAACGCCATGGAAGAAGAAGTGATAGCAGCGGAGACAGAGAAAAGAA
TGGAGGTGTTGAAAGAGGGTTGCGAAGGGAACTCCACCCTCCTCTTGGAA
CTGGAGAAGCTGAAATCAGCCGCTCTCTGTGGAAGAAGGTGAACGAGAG
AATGAGAGGAAAGAAAGTCTGTGTGTTTCTTTCTCTGTTTTGAGGTTCTCT
TACAGATGAAAAGCTGTGTAATTAAAAATCGATGTTCTTCTTCTGTTCTTG
TAAGATTTTGGATTTTTCCAATTTCTGACAAAGTTCAATTAAAAAACTTGA
CTGACATTTTGAAA
SEQ ID NO: 8 Arabidopsis thaliana, nucleic acid sequence
AtRTR1.
ATGGCAAAGGATAATGAAGCAATCGCCATTAACGATGCGGTTCACAAGCT
TCAGCTCTATATGCTCGAAAATACCACTGATCAGAACCAGCTCTTCGCGG
CGAGGAAGTTAATGTCTCGATCAGATTACGAAGATGTCGTCACTGAACGA
GCAATCGCTAAGCTCTGTGGTTATACTCTTTGCCAGAGATTTCTCCCTTCC
GATGTTTCTAGAAGAGGGAAGTATCGGATTTCGTTGAAGGACCATAAGGT
TTACGATTTACAGGAGACGAGCAAGTTTTGCTCCGCTGGTTGTTTAATTGA
TAGCAAAACGTTTTCGGGGAGTTTGCAAGAGGCTCGTACATTGGAGTTTG
ATTCGGTGAAGTTGAATGAGATTTTGGATTTGTTTGGTGATTCTTTGGAAG
TGAAAGGTTCTTTGGATGTGAATAAGGATTTGGATTTGTCTAAGCTTATGA
TTAAGGAGAATTTTGGAGTTAGAGGTGAAGAATTGTCTTTAGAGAAGTGG
ATGGGTCCTTCTAATGCTGTTGAAGGTTATGTTCCTTTTGATCGAAGCAAA
TCAAGTAATGATTCCAAGGCTACTACTCAAAGTAATCAAGAGAAGCATGA
GATGGATTTCACTAGCACAGTAATTATGCCTGATGTTAATAGTGTTTCAAA
GCTTCCACCGCAAACCAAGCAAGCTTCTACTGTTGTGGAATCTGTTGATG
GCAAAGGGAAAACAGTTCTGAAAGAGCAAACTGTAGTTCCTCCCACCAA
AAAAGTTTCGAGATTTCGTCGTGAGAAAGAAAAGGAGAAGAAGACTTTC
GGGGTTGATGGGATGGGTTGTGCCCAGGAAAAAACTACAGTTCTCCCCAG
AAAAATATTGAGTTTTTGTAATGAAATAGAGAAGGATTTTAAGAATTTTG
GGTTTGATGAGATGGGTCTTGCGAGTTCTGCTATGATGAGTGATGGATAC
GGCGTAGAATATAGTGTGTCTAAGCAGCCACAATGTTCGATGGAAGATTC
TCTTAGTTGCAAGCTAAAAGGAGATCTTCAGACTTTGGACGGGAAAAATA
CCCTATCAGGATCCTCTTCTGGTTCTAATACGAAGGGCTCGAAGACAAAA
CCAGAGAAATCAAGAAAGAAAATTATTTCTGTTGAATACCATGCTAATTC
TTATGAAGATGGTGAAGAAATCCTTGCAGCTGAATCGTATGAAAGACATA
AAGCTCAGGATGTGTGTTCATCAAGTGAAATCGTCACTAAATCATGCCTT
AAAATTTCTGGCTCGAAGAAGCTTAGTCGTTCAGTTACTTGGGCCGATCA
GAATGATGGCCGTGGTGATCTTTGTGAGGTTAGAAACAATGATAACGCAG
CAGGTCCTAGCCTGTCTTCTAATGATATAGAGGATGTCAATAGTTTATCAC
GCCTTGCATTAGCAGAAGCCCTTGCTACGGCATTGAGCCAGGCTGCCGAA
GCTGTTTCTTCGGGAAATTCAGATGCAAGTGATGCCACTGCAAAAGCTGG
AATCATTTTGTTGCCCAGCACACATCAACTTGACGAAGAGGTTACTGAGG
AACATAGTGAGGAGGAAATGACTGAAGAGGAACCAACTCTTCTCAAGTG
GCCAAATAAGCCCGGGATTCCAGATTCTGATTTGTTTGACCGTGATCAATC
GTGGTTTGATGGACCTCCAGAGGGCTTCAATCTCACATTATCAAATTTCGC
TGTGATGTGGGATTCACTGTTTGGCTGGGTATCATCGTCCTCTCTGGCATA
CATATATGGGAAGGAAGAATCTGCTCATGAGGAGTTCTTATTGGTTAACG
GGAAGGAGTACCCCCGGAGGATTATCATGGTAGATGGGCTTTCCTCAGAG
ATCAAGCAGACAATTGCTGGGTGCCTTGCCAGAGCTTTACCGAGAGTCGT
CACTCATCTCAGGCTGCCAATAGCGATATCCGAGTTAGAAAAGGGACTGG
GAAGCTTGTTGGAGACAATGTCGTTGACAGGAGCAGTTCCATCATTTAGG
GTAAAAGAATGGCTAGTGATTGTTCTTCTTTTCTTGGATGCGTTGTCTGTA
TCACGTATCCCTCGGATTGCACCTTATATATCCAACAGAGACAAGATTTTG
GAAGGAAGTGGAATTGGAAATGAAGAGTATGAGACAATGAAGGATATCC
TGTTACCACTTGGCCGTGTTCCTCAGTTTGCTACCCGAAGCGGGGCGTAG
SEQ ID NO: 9 Arabidopsis thaliana, Nucleic acid sequence
mutant atrtr1-1
ATGGCAAAGGATAATGAAGCAATCGCCATTAACGATGCGGTTCACAAGCT
TCAGCTCTATATGCTCGAAAATACCACTGATCAGAACCAGCTCTTCGCGG
CGAGGAAGTTAATGTCTCGATCAGATTACGAAGATGTCGTCACTGAACGA
GCAATCGCTAAGCTCTGTGGTTATACTCTTTGCCAGAGATTTCTCCCTTCC
GATGTTTCTAGAAGAGGGAAGTATCGGATTTCGTTGAAGGACCATAAGGT
TTACGATTTACAGGAGACGAGCAAGTTTTGCTCCGCTGGTTGTTTAATTGA
TAGCAAAACGTTTTCGGGGAGTTTGCAAGAGGCTCGTACATTGGAGTTTG
ATTCGGTGAAGTTGAATGAGATTTTGGATTTGTTTGGTGATTCTTTGGAAG
TGAAAGGTTCT[T-DNA Salk
012339]TTGGATGTGAATAAGGATTTGGATTTGTCTAAGCTTATGATTAAGG
AGAATTTTGGAGTTAGAGGTGAAGAATTGTCTTTAGAGAAGTGGATGGGT
CCTTCTAATGCTGTTGAAGGTTATGTTCCTTTTGATCGAAGCAAATCAAGT
AATGATTCCAAGGCTACTACTCAAAGTAATCAAGAGAAGCATGAGATGG
ATTTCACTAGCACAGTAATTATGCCTGATGTTAATAGTGTTTCAAAGCTTC
CACCGCAAACCAAGCAAGCTTCTACTGTTGTGGAATCTGTTGATGGCAAA
GGGAAAACAGTTCTGAAAGAGCAAACTGTAGTTCCTCCCACCAAAAAAGT
TTCGAGATTTCGTCGTGAGAAAGAAAAGGAGAAGAAGACTTTCGGGGTTG
ATGGGATGGGTTGTGCCCAGGAAAAAACTACAGTTCTCCCCAGAAAAATA
TTGAGTTTTTGTAATGAAATAGAGAAGGATTTTAAGAATTTTGGGTTTGAT
GAGATGGGTCTTGCGAGTTCTGCTATGATGAGTGATGGATACGGCGTAGA
ATATAGTGTGTCTAAGCAGCCACAATGTTCGATGGAAGATTCTCTTAGTTG
CAAGCTAAAAGGAGATCTTCAGACTTTGGACGGGAAAAATACCCTATCAG
GATCCTCTTCTGGTTCTAATACGAAGGGCTCGAAGACAAAACCAGAGAAA
TCAAGAAAGAAAATTATTTCTGTTGAATACCATGCTAATTCTTATGAAGAT
GGTGAAGAAATCCTTGCAGCTGAATCGTATGAAAGACATAAAGCTCAGG
ATGTGTGTTCATCAAGTGAAATCGTCACTAAATCATGCCTTAAAATTTCTG
GCTCGAAGAAGCTTAGTCGTTCAGTTACTTGGGCCGATCAGAATGATGGC
CGTGGTGATCTTTGTGAGGTTAGAAACAATGATAACGCAGCAGGTCCTAG
CCTGTCTTCTAATGATATAGAGGATGTCAATAGTTTATCACGCCTTGCATT
AGCAGAAGCCCTTGCTACGGCATTGAGCCAGGCTGCCGAAGCTGTTTCTT
CGGGAAATTCAGATGCAAGTGATGCCACTGCAAAAGCTGGAATCATTTTG
TTGCCCAGCACACATCAACTTGACGAAGAGGTTACTGAGGAACATAGTGA
GGAGGAAATGACTGAAGAGGAACCAACTCTTCTCAAGTGGCCAAATAAG
CCCGGGATTCCAGATTCTGATTTGTTTGACCGTGATCAATCGTGGTTTGAT
GGACCTCCAGAGGGCTTCAATCTCACATTATCAAATTTCGCTGTGATGTGG
GATTCACTGTTTGGCTGGGTATCATCGTCCTCTCTGGCATACATATATGGG
AAGGAAGAATCTGCTCATGAGGAGTTCTTATTGGTTAACGGGAAGGAGTA
CCCCCGGAGGATTATCATGGTAGATGGGCTTTCCTCAGAGATCAAGCAGA
CAATTGCTGGGTGCCTTGCCAGAGCTTTACCGAGAGTCGTCACTCATCTCA
GGCTGCCAATAGCGATATCCGAGTTAGAAAAGGGACTGGGAAGCTTGTTG
GAGACAATGTCGTTGACAGGAGCAGTTCCATCATTTAGGGTAAAAGAATG
GCTAGTGATTGTTCTTCTTTTCTTGGATGCGTTGTCTGTATCACGTATCCCT
CGGATTGCACCTTATATATCCAACAGAGACAAGATTTTGGAAGGAAGTGG
AATTGGAAATGAAGAGTATGAGACAATGAAGGATATCCTGTTACCACTTG
GCCGTGTTCCTCAGTTTGCTACCCGAAGCGGGGCGTAG
SEQ ID NO: 10 Arabidopsis thaliana, nucleic acid sequence
mutant atrtr1-2
ATGGCAAAGGATAATGAAGCAATCGCCATTAACGATGCGGTTCACAAGCT
TCAGCTCTATATGCTCGAAAATACCACTGATCAGAACCAGCTCTTCGCGG
CGAGGAAGTTAATGTCTCGATCAGATTACGAAGATGTCGTCACTGAACGA
GCAATCGCTAAGCTCTGTGGTTATACTCTTTGCCAGAGATTTCTCCCTTCC
GATGTTTCTAGAAGAGGGAAGTATCGGATTTCGTTGAAGGACCATAAGGT
TTACGATTTACAGGAGACGAGCAAGTTTTGCTCCGCTGGTTGTTTAATTGA
TAGCAAAACGTTTTCGGGGAGTTTGCAAGAGGCTCGTACATTGGAGTTTG
ATTCGGTGAAGTTGAATGAGATTTTGGATTTGTTTGGTGATTCTTTGGAAG
TGAAAGGTTCTTTGGATGTGAATAAGGATTTGGATTTGTCTAAGCTTATGA
TTAAGGAGAATTTTGGAGTTAGAGGTGAAGAATTGTCTTTAGAGAAGTGG
ATGGGTCCTTCTAATGCTGTTGAAGGTTATGTTCCTTTTGATCGAAGCAAA
TCAAGTAATGATTCCAAGGCTACTACTCAAAGTAATCAAGAGAAGCATGA
GATGGATTTCACTAGCACAGTAATTATGCCTGATGTTAATAGTGTTTCAAA
GCTTCCACCGCAAACCAAGCAAGCTTCTACTGTTGTGGAATCTGTTGATG
GCAAAGGGAAAACAGTTCTGAAAGAGCAAACTGTAGTTCCTCCCACCAA
AAAAGTTTCGAGATTTCGTCGTGAGAAAGAAAAGGAGAAGAAGACTTTC
GGGGTTGATGGGATGGGTTGTGCCCAGGAAAAAACTACAGTTCTCCCCAG
AAAAATATTGA[T-
DNA, Salk115762]GTTTTTGTAATGAAATAGAGAAGGATTTTAAGAATTTTG
GGTTTGATGAGATGGGTCTTGCGAGTTCTGCTATGATGAGTGATGGATAC
GGCGTAGAATATAGTGTGTCTAAGCAGCCACAATGTTCGATGGAAGATTC
TCTTAGTTGCAAGCTAAAAGGAGATCTTCAGACTTTGGACGGGAAAAATA
CCCTATCAGGATCCTCTTCTGGTTCTAATACGAAGGGCTCGAAGACAAAA
CCAGAGAAATCAAGAAAGAAAATTATTTCTGTTGAATACCATGCTAATTC
TTATGAAGATGGTGAAGAAATCCTTGCAGCTGAATCGTATGAAAGACATA
AAGCTCAGGATGTGTGTTCATCAAGTGAAATCGTCACTAAATCATGCCTT
AAAATTTCTGGCTCGAAGAAGCTTAGTCGTTCAGTTACTTGGGCCGATCA
GAATGATGGCCGTGGTGATCTTTGTGAGGTTAGAAACAATGATAACGCAG
CAGGTCCTAGCCTGTCTTCTAATGATATAGAGGATGTCAATAGTTTATCAC
GCCTTGCATTAGCAGAAGCCCTTGCTACGGCATTGAGCCAGGCTGCCGAA
GCTGTTTCTTCGGGAAATTCAGATGCAAGTGATGCCACTGCAAAAGCTGG
AATCATTTTGTTGCCCAGCACACATCAACTTGACGAAGAGGTTACTGAGG
AACATAGTGAGGAGGAAATGACTGAAGAGGAACCAACTCTTCTCAAGTG
GCCAAATAAGCCCGGGATTCCAGATTCTGATTTGTTTGACCGTGATCAATC
GTGGTTTGATGGACCTCCAGAGGGCTTCAATCTCACATTATCAAATTTCGC
TGTGATGTGGGATTCACTGTTTGGCTGGGTATCATCGTCCTCTCTGGCATA
CATATATGGGAAGGAAGAATCTGCTCATGAGGAGTTCTTATTGGTTAACG
GGAAGGAGTACCCCCGGAGGATTATCATGGTAGATGGGCTTTCCTCAGAG
ATCAAGCAGACAATTGCTGGGTGCCTTGCCAGAGCTTTACCGAGAGTCGT
CACTCATCTCAGGCTGCCAATAGCGATATCCGAGTTAGAAAAGGGACTGG
GAAGCTTGTTGGAGACAATGTCGTTGACAGGAGCAGTTCCATCATTTAGG
GTAAAAGAATGGCTAGTGATTGTTCTTCTTTTCTTGGATGCGTTGTCTGTA
TCACGTATCCCTCGGATTGCACCTTATATATCCAACAGAGACAAGATTTTG
GAAGGAAGTGGAATTGGAAATGAAGAGTATGAGACAATGAAGGATATCC
TGTTACCACTTGGCCGTGTTCCTCAGTTTGCTACCCGAAGCGGGGCGTAG
SEQ ID NO: 11 Arabidopsis thaliana, Protein sequence AtRTR1
MAKDNEAIAINDAVHKLQLYMLENTTDQNQLFAARKLMSRSDYEDVVTER
AIAKLCGYTLCQRFLPSDVSRRGKYRISLKDHKVYDLQETSKFCSAGCLIDSK
TFSGSLQEARTLEFDSVKLNEILDLFGDSLEVKGSLDVNKDLDLSKLMIKENF
GVRGEELSLEKWMGPSNAVEGYVPFDRSKSSNDSKATTQSNQEKHEMDFTS
TVIMPDVNSVSKLPPQTKQASTVVESVDGKGKTVLKEQTVVPPTKKVSRFRR
EKEKEKKTFGVDGMGCAQEKTTVLPRKILSFCNEIEKDFKNFGFDEMGLASS
AMMSDGYGVEYSVSKQPQCSMEDSLSCKLKGDLQTLDGKNTLSGSSSGSNT
KGSKTKPEKSRKKIISVEYHANSYEDGEEILAAESYERHKAQDVCSSSEIVTKS
CLKISGSKKLSRSVTWADQNDGRGDLCEVRNNDNAAGPSLSSNDIEDVNSLS
RLALAEALATALSQAAEAVSSGNSDASDATAKAGIILLPSTHQLDEEVTEEHS
EEEMTEEEPTLLKWPNKPGIPDSDLFDRDQSWFDGPPEGFNLTLSNFAVMWD
SLFGWVSSSSLAYIYGKEESAHEEFLLVNGKEYPRRIIMVDGLSSEIKQTIAGC
LARALPRVVTHLRLPIAISELEKGLGSLLETMSLTGAVPSFRVKEWLVIVLLFL
DALSVSRIPRIAPYISNRDKILEGSGIGNEEYETMKDILLPLGRVPQFATRSGA
SEQ ID No: 12 MINIYO Oryza sativa ssp. Japonica, Os06g37640.1
MDDAAERRRRQQQQQQPGAAHPARRKVVEEPFDPSPPPAAAVAPPSSRLVG
AIVEKGFSSGAAAAAPSSAPSPTVLPFPVARHRSHGPHWKPAARDAAMAEGE
GEEEEGMDVDETDYQPVAAAAGPVKRKEKKGMDFSRWREFVADDAPPKRR
QAKPLQPKKQTAQKIDTGVVAATTGGTAQEKRSGGIGMQLEVGNGKEELGG
AALMSDVAPRKPMKQVDARDDVRNVELRGEGMESDNGEPSLTAEINAENM
ARLAGMSAGEIAEAQAEILNRMDPAFVEMLKRRGKEKSGSRKDGGKGKGG
GISGPGKISKAMPGEWLSAGEHSGHTWKAWSERVERIRSCRFTLEGDILGFQS
CQEQQHVFWYPLHVNLAFPLTGKKAHVETVGERDFLRTEGDPAAVGYTINE
AVALSRSMVPGQRVLALQLLALILNRALQNLHKTDLIDNFKESNDDDKFND
WQAVWAYAIGPEPELVLSLRMSLDDNHDSVVLTCAKVINAMLSYEMNEMY
FDVLEKVVDQGKDICTAPVFRSKPDQNGGFLEGGFWKYNTKPSNILPHYGEN
DEEEGDEKHTIQDDVVVSGQDVAAGLVRMGILPRICFLLEMDPHPILEDNLVS
ILLGLARHSPQSADAILNCPRLVQSVVKLLVKQGSMEIHSSQIKGVNLLKVLS
KYNRQTCFNFVNTGVFHQAMWHWYRKAYTLEDWIRSGKEHCKLTSALMVE
QLRFWRTCISYGFCITHFTDFFPILCLWLSPSMFQKLSESNVVAEFSSIATESYL
VLGALAQRLPLLHSVEQLSKQDMGLSGIQVETWSWSHAVPMVDLALSWLCL
NDIPYVCLLISGQSKNILEGSYFALVISSVLGMLDSILERISPDSTHDGKSYCLP
WIPDFVPKIGLGVITNGFFNFLDDNAVELEQHTSFHGSSLVQGLFHLRSQGNV
DTSLCSISCFQRLLQLSCSIDRVIQNATTNCTEHLKESKTGIAGRILEQGICNFW
RNNLLDMLTSLLPMISSQWSILQNIEMFGRGGPAPGVGFGWGAYGGGEWSL
NFLLAQLDSHFVLELMKILSTGPEGLVTVNKSVNPIVQEGNNVTDSVAITSERI
SSVLSVSLMAGPGQISTLEKAFDILFHPSVLKFLKSSVLDSHMKLAKAFEWDI
TEDEYLHFSSVLNSHFRSRWLVIKKKHSDEFTRNNNGTNVPKIPETLETIQEET
ELAEAVNPPCSVLAVEWAHQRLPLPVHWILSAVCCIDDPKANLSTSYAVDVS
KAGLFFLLGLEAISAAPCLHAPLVWKMHALSASIRSSMDLLLEDRSRDIFHAL
QELYGLHLDRLCQKYDSAHSVKKEGSASVDEEKVTRTEVLRFQEKIHANYTT
FVESLIEQFAAVSYGDALFGRQVAIYLHRSVEPTIRLAAWNALSNAYVLELLP
PLDKCVGDVQGYLEPLEDDEGILESYAKSWTSGALDKAFQRDAMSFTVARH
HLSGFVFQCSGSGKVRNKLVKSLIRCYGQKRHHEDMLKGFVLQGIAQDSQR
NDEVSRRFEIMKDACEMNSSLLAEVRRLKTSIDR
SEQ ID No. 13 MINIYO Zea mays, GRMZM2G156818_T01
MDATTKRRHQPGGAQPTRRKVVEEPFHTAPPTPAAASPSRLVGAIVEKGYSA
AAPSSAPRPSVLPFPVARHRSHGPHWVPLVKDAPKDETADNDDEMDMDETD
YHPVAAAAAGPVRRKEKKGMDFSRWREFVGDAPPKRRQGKPVQAKKQSDQ
RIDAGAVASKVGGVAAEGRGLEGGAMRLDSGNASEGPGPVLLVSDVVSKKP
MSQVESRDELVNTSEARNLASQAESMDLDGRESSMEAEISAENMARLAGMS
AGEIAEAQADIVNKLNPALLEMLRRRGREKSGGTKDVGKDKGLKNSGLQKN
KRATPGDWLTAGEHTGHSWKVWSERVERIRSCRFTLDGDILGFQSSHEQQD
GKKMPSESVAERDFLRTEGDPAAVGYTINEAVALTRSMVPGQRVLALQLLA
SILNRALQSLHKTDLMDNVKGMNSKDNIDDWQAVWSYALGPEPELVLSLRM
ALDDNHDSVVLSCTKVVNVMLSCEFNESYFEFSEKVGNGKDICTAPVFRSKP
DLDGGFLEGGFWKYNTKPSNILPHCGDNDEDEADEKHTIQDDVVVSGQDVA
AGFVRMGILPRICFLLEMDPSPALEDYLVSVLVALARHSPQSADAILNCPRLIQ
SVTKLLINQGSMEIRSSQIRGVTLLKVLSKYNRQTCLNFVNHGVFQQALWHW
YRKAGTIEDWVRSGKEKCKLSSAMMVEQLRFWRTCISYGFCIAHFADFFPVL
CLWLSRPDFKKLSEHNVLVEFSSVARESYLVLAALAQRLPLLHSVEQLANQD
LGVSASYIETCSWSHVVPMVDLALSWLHLNDIPYVCSLISEQNRNTEHMLEM
SYLILVISSVLGMLNSILERISPDVTPEDKSYSLPWIPDFVPKIGLGIISNGFFSCS
TTVAGRNAEHQPFCCASLVQGLCYMRCHGNVDVSLSSISCLQRLVQLSWSV
DRVIQGATKCCSECFNESGTGEAGKLLAEGISSLWHNDLLHLLTSLLPMISSQ
WSISQNIEMFGRGGPAPGVGFGWGTCGGGFWSLKCLLAQLDSQLVVELIKCF
SSVQGSPIILDEGVKLDNVTNTVVTASNWISSTLGLSLIAGPGQIYMLEKVFD
MIFEPSILKYLKSSIHKFTSDMELLKPFEWDLNEDEYMLFSSVLKSHFRSRWL
AIKKKHSDKYAGDNSSTKISKTPEILETIQEETELSEAVNQPCNTLMVEWAHQ
RLPLPIHWILSAVCCIDDPKGTLSTSANYILDVSRAGLIFLLGLEAISATPCLHA
PLIWKIHALSVSIRSSMHLLQEDRSRDIFCALQELYGLHLNRLYQKFCKPNSIE
EVKGVVVGTSEEAMEISSLEILRFQEKIHGSYTTFVESLVDQFAAVSYGDFVF
GRQVAIYLHRKAEPAVRLAAWNALSSAYVLELLPPLDNCIGNAPGYLEPLED
DEKILESYAKSWTSGVLDKALQRDSMAFTLAKHHLSGFVFQSSDSGTMLRK
KLVKSLIRCYAQKRHHEVMLKCFVQQGIAQDSKSSELDRRFEILKDACEMNS
NLVGEVQRLKACLGQ
SEQ ID No: 14 MINIYO Glycine max, Glyma01g08040.1
MTKNENKVDKSVDWEAVWAFALGPEPELVLSLRICLDDNHNSVVLACTKV
VQSVLSYDANENYCDMSEIATCDMDICTAPVFRSRPDINDGFLQGGFWKYSA
KPSNILPFSDDSMDNETEGKHTIQDDIVVAAQDFTVGLVRMGILPRLRYLLEK
DPTTALEECIISILIAIARHSPTCANAVLKCERLVQTIVNRFTADNFELRSSMTK
SVKLLKVFARLDQKTCLEFIKKGYFQAMTWNLYQSPSSVDHWLRLGKEKCK
LTSALIVEQMRFWRVCIQYGYCVSYFLEMFPALCFWLNPPSFEKLVENDVLD
ESTSISREAYLVLESLAGRLPNLFSKQCLNNQLPESAGDTEVWSWNYVGPMV
DLAIKWIASRSDPEVSKFFEGQKEGRCDFPFRDLSATPLLWVYAAVTRMLFR
VLERMTWGDTISSFETEGHVPWLPEFVPKIGLELIKYWFLGFSASFGAKFGRD
SEGESFMKELVYLRQKDDIEMSLASTCCLNGMVKIITTIDNLILSAKAGICSLP
RQEQSLSKEGKVLEDGIVNGCLVELRYMLDAFMFSVSSGWHHIQSIESFGRG
GPVPGAGIGWGAPSGGFWSATFLLAQIDAKFLVSLLEIFENASKGVVTEETTFI
IQRVNAGLGLCLTAGPREKVVVEKALDLLFHVSVLKNLDLCIHNFLFNRRGR
TFGWQHEEEDYMHLRRMLSSHFRSRWLSVKVKSKSVDGSSSSGIKTSPKVG
ACLETIYEDSDMSSMTSPCCNSLMIEWAHQKLPLPVHFYLSPISTIFHSKRAGT
KKVDDVLHDPSYLIEVAKCGLFFVLGVEAMSIFHGTDIPSPVEQVSLTWKLHS
LSVNFLVGMEILEQDRSRVTFEALQDLYGELLDKARLNQSKEVISNDKKHLE
FLRFQTEIHESYSTFLEELVEQFSAVSYGDVIFGRQVSLYLHRYVETSIRLAAW
NTLSNARVLELLPPLEKCFSGAEGYLEPAEDNEAILEAYTKSWVSDALDRAAI
RGSVAYTLVVHHLSSFIFHACPMDKLLLRNRLARSLLRDYAGKQQHEGMLL
NLIHHNKPPPSVMGEELNGGVLSERNWLESRLKVLVEACEGNSSLLIVVEKL
KAAVEKSS
SEQ ID No: 15 MINIYO Glyma02g13360.1
MKVDTKPLLDNSDGGFINSTTTMEVDTLNKEQNESVPGLDQISSDWMPDYN
FGSLDVQRPGQTDLNSSMLEQKSVSLDSEIDAENRARIQQMSAEEIAEAQTEI
MEKMSPALLKLLQKRGQNKLKKLKLEVDIGSESVNGHAQSPQDAKHLHTED
GIAQTVIVPPSKEKLDDEKISTKTSTTASSSAWNAWSNRVEAVRELRFSLVGD
VVDSERVSVYDNANERDYLRTEGDPGAAGYTIKEAVALTRSVIPGQRTLALH
LLSSVLDKALHYICEDRTGHMTKIENKVDKSVDWEAVWAFALGPEPELVLSL
RICLDDNHNSVVLACAKVVQCVLSYDANENYCNISEKIATCDMDICTAPVFR
SRPDINDGFLQGGFWKYSAKPSNILPFSDDSMDNETEGKHTIQDDIVVAGQDF
TVGLVRMGILPRLRYLLETDPTTALEECIISVLIAIARHSPTCANAVLKCERLV
QTIANRYTAENFEIRSSMIRSVRLLKVLARSDRKSCLEFIKKGYFQAMTWNLY
QSPSSIDHWLRLGKEKCKLTSALIVEQMRFWRVCIQYGYCVSYFSEMFPALC
FWLNPPSFEKLVENNVLDESTSISREAYLVLESLAGKLPNLFSKQCLNNQLPE
SAGDTEVWSWNYVGPMVDLAIKWIASRNDPEVSKFFEGQEEGRYDFTFRDL
SATPLLWVYAAVTHMLFRVLERMTWGDTIETEGHVPWLPEFVPKIGLEVIKY
WFLGFSASFGAKCGRDSKGESFMKELVYLRQKDDIEMSLASTCCLNGMVKII
TAIDNLIQSAKASICSLPCQEQSLSKEGKVLEDGIVKGCWVELRYMLDVFMFS
VSSGWHRIQSIESEGRGGLVPGAGIGWGASGGGFWSATVLLAQADARFLVYL
LEIFENASKGVVTEETTFTIQRVNAGLGLCLTAGPRDKVVVEKTLDFLFHVSV
LKHLDLCIQSLLLNRRGKTFGWQHEEEDYMHLSRMLSSHFRSRWLSVKVKS
KSVDGSSSSGIKTSPKVGACLETIYEDSDTSSVTTPCCNSIMIEWAHQKLPLPV
HFYLSPISTIFHSKRAGTKIVDDVLHDPSNLLEVAKCGLFFVLGVEAMSIFHGT
DIPSPVQQVSLTWKLHSLSVNFLVGMEILEQDWSRDIFEALQDLYGELLDNA
RLNQSKEVISDDKKHLEFLRFQTEIHESYSTFLEELVEQFSAVSYGDVIFGRQV
SLYLHRCVETSIRLAAWNTLSNSRVLELLPPLEKCFSGAEGYLEPAEDNEAILE
AYTNLWVSDALDRAAIRGSVAYTLVVHHLSSFIFHACPTDKLLLRNRLARSL
LRDYAGKQQHEGMLLNLIHHNKPPPSVMGEELNGILSEKSWLESRLKVLVEA
CEGNSSILTVVDKLKAVVKNSS
SEQ ID No: 16 MINIYO Brachypodium distachyon, BD1G37370
MLPMDDGTKRKHQPGAHPTRRKVVEEPFDPAPPLSGAATAAASAAAPPPHL
VGAIVEKGFSAAAPSSSPRPTVLPFPVARHRSHGPHWNPVTKDAYKEKGEVE
DYGMDVDEVDYQPMATVAGPIRRKEKKGMDFSRWREFMADDVPPKRRQA
KKNSTQRIDPGIVAEKVDVSVGERALGGDGMELDGGNAKDELGVTTLVSDV
LPRKPEKRVDAGDLLMLEGEAGVAEMRGEGMQLDDGEPSVAAEINAENIAR
LAEMSTEEIAEAQADILNRLDPTLVEILKRRGKEKSGGRKDGVKDKGGEISEP
GKTARATPGARLVVGEHNGYSWKAWSERVERIRLCRFTLNGDILGFQSCQE
QQDGKNRNAERVAERDFLRTEGDPAAVGYTINEALALTRSTVPGQRVLGLQ
LLASVLNRAVHNLHEMDLADNLEGANGADKLDDWQAVWAYALGPQPELV
LSLRMALDDNHASVVLTCAKVINVMLTYDMNEAYFEFSEKVVHQGKDICTA
PVFRSKPDLDGGFLEGGFWKYNTKPSNILPHYGENAEEEGDEEHTIQDDVVV
SGQDVAAGLIRMGILPRICSLLEMDPPPILEDYLVSTLVALARHSPQSADAILN
CTNLVQSVVKLLVKQGSMEIHSSQIRGVTLLKVLSKYNRQTCSNLVNRGVFQ
QAMWQWYRKAYTLEDWIRSGKEQCKLSSAMMVEQLRFWRTCISYGFCIGH
FTDFFPVLCLWLSPPLFQNLSKSNVLSEFSSISRESYLVLGALAQRLPLLHSME
QLGKQDMGVSGSYIEMWSWSHVVPMVDLALSWLHLNDIPYLCSLINEQSEN
TAHILEESCLVLLISSVLGMLNSILERISPDGTPDVKSYCLPWIPDFVPKIGLGII
TNNFFSFSRDDVVGHEDQLSFCGVSLVQGLCRMRSQGNVDASLSSICCLQRL
VQLSFSVDRVIQRVSTKCSEPVKESKTGIAGKILGQGISSLWHHDLLNSLNVM
LPLSSSQWPVLKNIETFGRGGLAPGVGFGWGTCGGGFWSLKCLLAQLDSQL
VLELIKIFSAVPEVLVTPSKGVNSDNVTNPVAKASGRISPVLGVSLIAGPGQIT
TLETAFDILFHPSILKCLKSSMQSMASQMELPKTSEWEITEDEYQHFSSVLNSH
FRSRWLVIKKKSDKYARDNSGINMPKLSETLDTIQEEVEFTETVNPPCGTLVV
EWAHQRLPLPVHWILSSICCIDDAKGTLSVLANHAVDVSRAGLIFLFGLEAISS
APCLDAPLVWKIHALSASLRTNMDLLQEDRSRDIFNALQELYGQHLDMLCH
KYYRSHSVKNDEVVGSVTTVEEAKAISSLEILGFKEKIHGSYTTFVESVIDQFA
AVSYGDVIFGRQVAIYLHRSVETVVRLAAWNALSNAYVLELLPPLDKCIGDI
KGYLEPFEDNEAILEAYAKSWTSGVLDKASQRDSMSFTLVRHHLSGFVFERN
ASIKVRNKMVKSLIRCYAQKQHHEAMLQGFVLHGTQSSDEVSRRFEILKDAC
EMNSSLLAEVHRLKTSIDG
SEQ ID No: 17 MINIYO Sorghum bicolor, Sb10g022700
MDAPTKRRHQPGGAHPTRRKVVEEPFHPAPPTPAAAAAAAASASPARLVGAI
VEKGFSAAAPSSAPRPSVLPFPVARHRSHGPHWGPVAKDAHKDGAADDDDE
MDMDETDYHPVAAAAGPVRRKEKKGMDFSRWREFVGDAPPKRRQGKPVQ
AKKQSDQRIDAGAVASMVGGVAATGRGLEGGAMQLDSGELEGSAMQLDSG
NTREGPGAVLSVSDVVSKKPMSQAESRDELVKVGEVRNSTSQAESMDLDGR
ESSMEAEINAENMARLAGMSAGEIAEAQTDIVNKLNPALVEKLRRRGREKSG
GTKDVGKDKGLENSGPQKTKRATPGDWLTPGEHSGHSWKAWSERVERIRSC
RFTLDGDILGFQFSHEQQDGKKMHSESVAERDFLRTEGDPAAVGYTIKEAVA
LTRSMVPGQRVLALQLLASILNRALQNLHKTDLMDNVKEMNSNEKFDDWQ
AIWSYALGPEPELVLSLRMALDDNHDSVVLSCAKVINVMLSCEFNESYFEFSE
KVGNGKDICTAPVFRSKPDLDGDFLEGGFWKYNTKPSNILPHYGENDEDEGD
DKHTIQDDVVVSGQDVAAGFVRMGILPRICFLLEMDPSPALEDYLVSVLVAL
ARHSPHSADAILNCPRLIQSVTKLLINQGSMEIRSSQIKGVTLLKVLSKYNRQT
CLNFVNHGVFQQALWHWYRKAGTIEDWVRSGKEKCKLSSAMMVEQLRFW
RTCISYGFCIAHFADFFPVLCLWLSPPEFKKLNEHNVLVEFSSIARESYLVLAA
LAQRLPLLHSVEQLANQDRGVSASYIETCSWSHVVPMVDLALSWLHLNDIPY
VCSLISGQNRNTKHMVDASYLILVIASVLGMLNSILERISPNVTPEDKSYSLPW
IPDFVPKIGLGIISNGFFSCLGTVAVRNAEHQSFCSASLVQGLCYMRCHGNVD
VSLSSISCLQRLVQLSWSVDRVIQGAKKSCSECFNESGTGVAGKLLGEGISSL
WHNDLLHLLTSLLPMISSQWSISQNIEMFGRGGPAPGVGFGWGACGGGFWS
LKCLLAQLDSQLVVELMKCFSSVQGSPVILDEGVKSDNVTNTVVTASNWISS
SLGLSLIAGPGQIYMLEKAFDMIFEPSILKYLKSSIHKFASDMVLLKPFEWDIN
DDEYLLFSSVLNSHFRSRWLAVKKKKHSDKYTGNNSSTKISKTPETLETIQEE
TELTEAVNQPCNTLVVEWAHQRLPLPIQWILSAVCCIDDPKGTLSTSANYILD
VSRAGLIFLLGLEAISATPCLHAPLIWKIHALSVSIRSSMHLLQEDRSRDIFCAL
QELYGQHLNRLCQKFCKSKSVEEVKGVVVATSEEAMEISNHEILRFQEKIHGS
YTTFVESLVDQFAAVSYGDFVFGRQVAIYLHRKVEPAVRLAAWNALSNAYV
LELLPPLDKCIGNAQGYLEPLEDDENFLESYAKSWTSGVLDKALQRDSMAFT
LVKHHLSGFVFQSSDSGKTLRNKLVKSLIRCYAQKRHHEVMLKSFVLQGIAQ
DSKSSGNELDRRFEILKDACEMNSSLLGEVQRLRACLGQ
SEQ ID No: 18 MINIYO Oryza sativa ssp. japonica , >OS06G37640,
Gene
ATGGACGACGCGGCGGAGCGGAGGCGGCGGCAGCAGCAGCAGCAGCAG
CCAGGCGCCGCCCACCCCGCGCGCCGCAAGGTCGTGGAGGAGCCCTTCGA
CCCCTCCCCTCCCCCGGCCGCCGCCGTGGCGCCGCCTTCCTCCCGCCTCGT
CGGCGCCATCGTCGAGAAGGGCTTCTCCTCCGGCGCGGCCGCCGCCGCGC
CCTCCTCCGCCCCGAGTCCCACCGTCCTCCCCTTCCCCGTCGCCCGCCACC
GCTCCCACGGCCCCGTAAGCCGCCTACGCCTCCTCCGCCGCCGCCCTCTAT
CTCGGAACCCTAGGTTTGATGTGGTGCTTTGGTTTTGTACTCCCTGACTGA
CTATCTGCTCTTCCGCAGCACTGGAAACCGGCGGCGAGGGATGCTGCCAT
GGCGGAGGGGGAGGGCGAGGAGGAGGAAGGGATGGATGTGGACGAGAC
GGACTACCAGCCCGTGGCCGCCGCAGCTGGGCCCGTTAAGAGGAAGGAG
AAGAAGGGCATGGATTTCAGCAGGTGGCGGGAGTTCGTCGCTGACGATGC
GCCCCCGAAGCGAAGGCAGGCAAAGCCGTTGCAGCCGAAGAAACAGACT
GCGCAGAAAATTGACACCGGGGTCGTGGCTGCAACGACGGGTGGCACCG
CACAGGAGAAGCGCTCCGGGGGAATTGGTATGCAGCTGGAAGTTGGAAA
TGGTAAGGAAGAATTGGGTGGAGCTGCTTTGATGTCTGATGTGGCGCCAA
GGAAGCCGATGAAACAGGTTGATGCTAGAGATGATGTGAGGAATGTGGA
ATTGCGAGGAGAGGGTATGGAATCGGATAATGGGGAACCATCTCTTACCG
CAGAGATTAATGCGGAGAACATGGCTAGGCTGGCAGGGATGTCAGCTGG
GGAGATTGCAGAGGCACAGGCAGAGATCCTGAATAGGATGGACCCGGCA
TTTGTGGAGATGCTGAAACGACGGGGGAAGGAGAAGTCTGGGAGCAGGA
AAGATGGGGGAAAGGGCAAGGGTGGGGGGATTTCAGGCCCAGGGAAGAT
CTCGAAGGCTATGCCTGGAGAATGGTTGTCAGCTGGTGAGCATAGTGGAC
ACACTTGGAAGGCATGGAGTGAGAGAGTAGAGCGGATCAGGTCTTGTAG
GTTCACATTGGAAGGAGATATTTTGGGGTTTCAATCTTGTCAGGAGCAAC
AACATGGTAAATCATTTTTCTTTGCTCTTGTGTTGCTTTTAGCTTCTAGTGT
TCTGGTACCCGTTACATGTCAACCTGGCTTTCCCACTTACAGGCAAGAAA
GCACATGTGGAAACTGTAGGTGAGCGTGATTTTCTTCGAACAGAGGGAGA
TCCCGCAGCTGTTGGGTACACAATTAATGAAGCAGTGGCACTTAGCAGGA
GCATGGTTTGTTACTTTCTTTTGTTGTTCAGTGTGAAAATGGACTTTTGAGT
AAAGTTCAAGGGCTAAAAATGGACTTTACCCAATGCAATACAACATCTAG
TTTCTCTTTAGTATTTTTAGAGATATTAACCTTTGCATGTGAATGGATTTTG
TTGTTTTTTTTTTTAAATGTCATATCATGCATAACTGAGATACACCATCATC
GCTTAATGTTTTTCTTACATCATTTCTAAAGTGTGCCTCCAAAATATTGCA
AGATAAATAAATGTAATAATTCAGTTTTTACGTTCAAACCATAGGTTCCTG
GACAGCGCGTGCTTGCGCTTCAGCTCCTTGCTTTGATTCTTAATAGGGCCT
TGCAGAACCTACATAAGACGGATCTAATTGATAACTTTAAAGAATCAAAT
GATGATGACAAGTTTAATGACTGGCAAGCGGTTTGGGCATATGCCATCGG
ACCTGAACCTGAGTTGGTTCTCTCTCTAAGGTAAACTGGTTGTTTTCAAAC
TATAATAATAATTTTAATTTGGATTTGCTTATTCCATGCAAGAGTTTATTA
GTTACCAAAGTGAAGAGTACTTAACTGAAATATACCGGACAATTCTATTA
AGACTAAATAATCAAGAAACCTTAACACATGCTTTAGTGTCTGCACCTAT
GCTTAAAATTGTATGCCTGAAAATTTGGGCTGCCACATCTTGTATTTTAGT
CATGAATTCATGACAGCAGGCAAGCATTAACAGCATAAGGTCATCTTGTC
CATTAGCTATCAGTTAGCACTTGTAAGAAGATGTATCCAGTTAACTTAGG
CCATGTCTAGTGTAGGTGAAGCTAAGGTTGTCACATCACAGCCATGTGAT
ACACAGGGTGGATAATTTTTCTTCTCTAGTGTGCTAGCATCGATTTTAGTT
TTTGAGTGGACTCCACTAGAAATTTTAAAAACTGGCTTTCCTAGTGTATGC
AAATAAAACATTTTGCTTCGCATCAAACCATATATGCATCGATTTTGTTCC
TAGCATCTCCTCTTCTAGTGCCTAGAATCATTACTAAGCCTTCAAATCCGC
TTGCTAGCATCGGTCAAGGTGTGCACTAGACATGCCCTTATTGTCTTTCTT
TTTGCAGTCACGTTGGCTGTTTCATGTATAATAAGTTAGGAAAATTCCATC
TATAGCACAAAGTTTTCATAGGTACTAATAAATACCACAATTTTCAACCCT
TCCAGAAATGCCACAATGTGACACCACACGTAAAAAATACCACAAAATTT
TACAGAACTGAACGTATGACAAATTGAACTATTACCATGGACAAAATTGC
CCCTGGCCTTTCTTCAATCTGCATTCAGTAGTCTCCCCGTGATTCCACTCG
TCTCCTCTCTCTCTCTCTCTCTCTCTCTCTCTCTCTCTCTCTCTCTCGGCGAG
ATGAAGTGAAGCTGGTGGCCACGAATTCCGGCAATGAAGCACGGTCCTAC
CTACAATCCGTCCTCGAGGACTAGCGACCGCCGCCAGCCGCGGAGGCCGC
CAGCGCCGAGGCCGCTGAGGCGTTGGCTCTCGAGGTGAAGTCTTCCGCAC
TCCTCCCGTAACCGATGAAGGTCTGGCAACCATGGCCATGCCTCTGACCT
GCTCTAGCGTGATCCAATCACTTGGTCTCACAGGTCTCCATGATGCTCTTA
TTGCCGGCGGCCATTGCAACATCCACACATGCATCCGAATCATATGTCCC
CAAATCCGAACGCCTAAATCCATTTCCTCCTTGTGTTATTCTCTTGGCCAC
AGAAAACACCTCGTTTTGGCAGGATTTGCTGCGAATCAAGCCAAATAATC
CATTGCCTTCGTGGTAATCTGGGCATTTTCTTTGTAAAATGGCACTGTCCA
TCCCCGCCTTGTTCTGTTCTGAGTTGTGGTATTTCTGAAAGGGTGGAAAAA
TCCATGGTATTATTAGGTACCAGTGAAAACTTTGTGCTATAGATGGAATTT
TCCCTAATAAGTTATAATCTCAGAAAACTGCGGTGTGACCTAATTAACTGT
TGTCTATAGGTTTTTTTTATTTAGTTCTCTCATATGCCATCTAATTAGTTTC
TTTGCTTATATGAGTTAATTGCACCAGTGTTAGACCTTAAAATGGCCTATG
CTTTTTTCATTGTTTATTCTACTTATGTGTCATCACTAACTGGAATATCTGC
AGGATGTCATTGGATGATAACCATGATTCTGTAGTTTTGACTTGTGCTAAA
GTTATCAATGCTATGCTGAGCTATGAAATGAATGAGATGTATTTCGATGTT
TTAGAGGTAACTTTACTAATTTATCTTCTAATTTGTTTTTCGTTGAGAACTT
TATCCTTAAATGGCTGATTTTCAACATTACAGAAAGTAGTAGATCAAGGG
AAGGATATCTGTACAGCTCCTGTTTTCCGTAGCAAACCTGATCAGAATGG
GGGTTTTCTTGAAGGAGGTTTCTGGAAATACAACACAAAACCATCCAATA
TACTCCCACATTATGGTGAGAATGATGAGGAAGAAGGTGATGAGAAACA
TACCATTCAAGATGATGTGGTTGTATCAGGTCAAGATGTTGCTGCTGGTCT
TGTTAGAATGGGAATACTTCCACGGATCTGCTTCCTTTTGGAGGTGAGGGT
CTGGCCATCTGCTCTCCCTCATCCCCTATCTGCCATTGTTTCTTTCAGAATC
CCACTGCCATGCATGCACCCAGAATCCCACCGCCATGCATGCACAATATG
GGTGCATTAGGTCATCTTTTGTTTACTATATCCGTGAATTGTCTGGTTCCTT
GATGTTCAATCCTGTCTAGTACTTGCCATCCATCCTTGAAATGGCAGTGAA
ATGCCTCCTTACCTGGTTGACACATCACTTGTTTTCACCAGGAAATCTGGT
TTGCAGCAGAATTTGTGGCTGGCTTTGATTGTTCTTTTACCATCGGAAAAT
TACAATTTTGATATTCAACAACCAAACTATGATAAATTGTGTATAGCCTTT
CATTTTTAGCAATATTTTTTATTTCCTGAAGCTATGGGCCTTATCCATTTGC
AGTTTTAAGTATTTTGTAGTTCTATGATGTCATGTCATCTGATACCCGTAA
TCTTTCAAACTTCTTATCTGGTTATTGGACTGTCTCGTAGATTGTTCTGTTC
ACCCTTGGTGCTCTTGCTTGTTGACTGTTTGTTTTATAGTGATAGCTTTATA
TTGCAGTTCTATTACTTCACTAGTAAACATCATTTGAGCTAGTTGGTCTTC
TTTTTATTTATGTTTTCATTTCCTATTCTACGAGAAAGTGATGGTCATGATT
ACGGATCTTAATGGTCAAGTTTTTGTTGATTCAGATGGACCCACATCCAAT
TCTAGAAGATAATCTTGTTTCAATTCTTTTGGGATTAGCGAGACACTCTCC
ACAATCTGCTGATGCTATCTTGAACTGCCCAAGGCTCGTTCAAAGTGTTGT
TAAGTTGTTAGTCAAGCAAGGATCAATGGAAATTCACTCCTCGCAAATTA
AAGGAGTCAATCTCTTGAAGGTATCTTCTGGTGATATAAATAATCTTTTAT
TATAGGAGTATGCTGATTTTGTGTAGTTTTGGAAGTTGCAGATTATTTATT
ATATAGTTTTTCTTCTATATCATTGGTTGATAATAACTTGATCTAGTTATCC
TTCAGAAGGATTCCTTCTGAAGGATAACTAGATCAAGTCTGCCAGTAATA
CCACTTGCAATTGCTGATTGTACATCTGAAATGGTTTCCTTTTGTTGGTAA
TTAGCTACCAACAGCATCATTATGAGTTTTTTTTTTGTTTGCTGCTTCTGAT
ATCTTAACACATAGCAATTGTATTAGCCATGCCTGCTGTTATCTGAATCAA
TCACACTCCTTGCACAATGCCTTCACTGTCAACACACAACTGTTTGGTCCC
CCCCCCTCCTGCGGGCACTTTAGTTGCCCCATTAATTCATTATTATGGTAA
GATGAGAAACACCATAGTCATAAGATTTGAATGTTCAGATTATGCTAAAA
AAAATCATTACTTCTATTTCTAAATTTGTCAAAGATTTTTTTTTCACTAGCA
TCTAATTTCTTGTTCCTTTTGCCTAGGTTTTGTCCAAGTACAACAGGCAAA
CATGCTTCAATTTTGTGAACACTGGAGTTTTTCATCAGGCTATGTGGCACT
GGTACAGAAAAGCCTATACTCTTGAGGATTGGATAAGATCTGGAAAGGA
GCACTGCAAGCTTACTTCAGCATTAATGGTTGAGCAGCTGCGGTTTTGGA
GGACCTGTATCTCTTACGGGTTTTGTATAACGCATTTCACAGATTTCTTTC
CTATTTTATGCCTGTGGCTTAGCCCTTCGATGTTTCAGAAGCTAAGTGAAA
GCAATGTTGTAGCTGAATTTAGTTCCATTGCAACAGAGAGTTATCTTGTAT
TAGGAGCTCTGGCTCAAAGGCTTCCACTTCTTCATTCAGTTGAGCAGCTTA
GCAAGCAAGACATGGGACTTTCTGGTATCCAAGTTGAGACATGGTCGTGG
AGCCATGCAGTTCCAATGGTAGATTTGGCGCTATCTTGGCTATGCCTGAAT
GATATTCCTTATGTGTGTTTGCTAATCAGTGGGCAAAGTAAGAATATACTA
GAGGGAAGCTACTTTGCTTTGGTTATTTCTTCTGTGCTAGGCATGCTTGAT
TCAATACTAGAAAGGATATCTCCAGATAGTACACATGATGGTAAAAGTTA
CTGCTTGCCTTGGATACCTGACTTTGTACCCAAAATTGGCCTGGGAGTTAT
TACTAATGGATTTTTCAACTTCTTGGATGATAATGCCGTTGAACTTGAGCA
ACATACATCTTTCCATGGTTCATCGTTGGTGCAGGGACTTTTTCATTTGAG
ATCCCAGGGTAATGTTGACACATCATTGTGTTCAATTAGTTGCTTCCAAAG
GTTATTGCAGCTATCTTGCTCTATTGACAGAGTAATCCAGAACGCCACAA
CAAATTGTACAGAGCATCTGAAAGAATCAAAAACAGGGATAGCTGGCAG
GATACTAGAACAAGGTATTTGCAATTTCTGGCGTAATAACTTGTTGGACA
TGCTAACTTCATTGTTGCCAATGATTTCCTCACAGTGGTCCATATTACAAA
ACATAGAGATGTTTGGTAGAGGAGGACCAGCACTGGTGTTGGTTTTGGT
TGGGGAGCATATGGTGGAGGATTTTGGTCTTTGAATTTCCTTCTTGCACAA
TTGGATTCACATTTTGTTCTAGAATTGATGAAAATCTTGTCCACGGGGCCA
GAAGGCCTTGTCACCGTCAATAAAAGTGTGAATCCAATTGTGCAAGAAGG
AAATAATGTGACTGATTCAGTTGCCATCACTTCAGAGAGAATCAGTTCTG
TCCTCAGTGTATCTTTGATGGCAGGACCTGGGCAGATATCTACACTAGAG
AAAGCCTTTGATATCCTCTTCCACCTTTCTGTTCTGAAGTTTCTCAAATCTT
CAGTACTAGACTCACACATGAAATTAGCAAAAGCTTTTGAATGGGACATA
ACTGAAGATGAGTATCTCCATTTTAGCAGTGTACTGAATTCACATTTCAGA
TCCAGATGGTTGGTCATCAAGAAGAAGCATTCAGATGAATTTACAAGAAA
TAACAATGGCACAAATGTGCCAAAAATACCAGAGACATTGGAGACGATT
CAAGAAGAAACAGAGTTAGCAGAAGCTGTAAATCCACCTTGCAGTGTGTT
AGCTGTAGAGTGGGCACACCAGAGATTGCCCCTTCCTGTGCACTGGATTC
TAAGTGCAGTCTGTTGCATTGATGATCCAAAAGCCAACCTATCAACCAGT
TATGCTGTTGATGTTTCAAAAGCTGGTCTCTTCTTTCTTTTAGGTCTGGAA
GCCATTTCAGCGGCTCCATGTCTTCATGCTCCTTTGGTTTGGAAGATGCAT
GCACTTTCTGCCTCTATCCGCAGTAGCATGGATTTGCTTCTAGAAGACAGA
AGTAGGGATATTTTTCATGCTTTGCAAGAACTGTATGGCCTGCATTTGGAT
AGATTATGCCAGAAATATGACAGTGCTCACTCTGTCAAGAAAGAAGGATC
AGCCTCTGTGGACGAAGAAAAGGTGACCAGGACTGAAGTTCTCAGATTTC
AGGAGAAAATCCATGCAAACTATACTACTTTTGTTGAGAGCCTTATTGAG
CAATTTGCAGCTGTCTCATATGGTGATGCTCTTTTTGGTCGACAAGTAGCC
ATTTATTTGCACCGGAGTGTTGAACCCACAATTCGGCTTGCGGCCTGGAA
CGCTCTGTCTAATGCTTATGTGCTTGAACTGTTACCTCCACTAGACAAATG
CGTCGGTGATGTTCAAGGGTACTTGGAGCCTCTTGAGGTATTGTCTCTTTA
GTTTCTTGTATGCTTGTCTTTTGGTTGGCATTTTGTAGTAACAATGTAAATT
TGGGTCTAGAAGTTTGCTTTATACTACTGCTTTTGGGGTTGCACATTACAT
AGAGTATGCAAAGAGTAAACAATGCATGCTATTTTTCTTAACTATTTGATT
TCGGTACATACATGAGCACATGCCATGATTCTTTGTAAGCATGTTCCCACC
TGCTTGTACTTTGTTGCATCTGTGTGATAAAACAGTTTCAGATTTTCTGATT
ATTATTTTCACAATAATTTTGTAGGATGACGAAGGAATTTTGGAGTCTTAT
GCTAAATCATGGACTTCTGGTGCCCTTGACAAAGCTTTTCAGCGGGATGC
AATGTCTTTCACAGTAGCAAGGCATCACCTTTCTGGCTTCGTCTTCCAGTG
CAGCGGTTCTGGCAAAGTGCGGAATAAGCTGGTTAAATCGCTTATCCGGT
GCTATGGCCAGAAGCGCCATCACGAGGTAATTCCCTCGTGCTTTCCGGAA
TATTAGTCATATATTTCATAAATCATGAACCATCCTTGATGTTTGGAAAAT
TCTATACCGCAGGATATGCTCAAGGGTTTTGTTCTACAAGGCATTGCGCA
AGATTCACAACGTAATGATGAAGTTAGCCGACGATTTGAAATCATGAAGG
ATGCTTGTGAGATGAATTCTTCTCTTTTAGCTGAGGTTCGGAGACTGAAGA
CATCAATTGATAGATAAATGGCCCCAAAAGCATTTCACATGCACTCAGAG
AAGGAGCAATTTTTTTGGTGATTGTAAATAGTAACATGTGTCAATGACAA
ATGGCTAGAGAATAGTTGCATTGTTCGAGTTTACTTTGCATTACAAACCAA
CCAAAGGAAAACATCAGTGTGTGTAGGAGTGCTCCCGGTTGTGGCTTGTG
GGTTACATTTTAAAGGACAATGAAACAGACTGGTGTTACAGAGATGCTAA
CTCCAACCTTATGCTCCAGTATCCAACATTTTCTCC
SEQ ID No: 19 MINIYO Zea mays, >GRMZM2G156818, Gene
ATGGACGCGACGACGAAGCGGCGGCACCAGCCCGGCGGCGCGCAGCCCA
CACGCCGTAAGGTCGTGGAGGAACCCTTCCACACCGCACCCCCTACACCT
GCCGCGGCGTCCCCCTCCCGCCTCGTCGGCGCCATCGTCGAGAAGGGCTA
CTCCGCCGCCGCACCCTCCTCGGCGCCCCGACCTTCCGTCCTCCCCTTCCC
CGTCGCCCGCCACCGCTCCCACGGCCCCGTAAGCAGACGCCGCCCTCCCA
AACAAACCCTAGGCTCCTAACCCGCTAACCTCGATTTGCTTCGCCGCTGA
CCAACTACTACTTCCCGTTTCGCAGCACTGGGTTCCCTTGGTGAAGGACGC
CCCCAAGGATGAGACCGCGGACAACGACGACGAGATGGACATGGATGAG
ACAGACTACCATCCTGTGGCAGCTGCTGCAGCTGGTCCTGTGAGGAGGAA
GGAGAAGAAGGGCATGGATTTTAGCCGGTGGCGTGAGTTTGTTGGCGATG
CTCCCCCCAAGCGGAGGCAGGGGAAGCCAGTGCAGGCCAAGAAACAGAG
CGATCAGAGAATTGATGCTGGGGCTGTAGCTTCTAAGGTAGGTGGTGTGG
CAGCTGAGGGGAGAGGATTGGAGGGAGGTGCTATGCGGCTTGACAGTGG
AAATGCTAGCGAAGGGCCAGGTCCCGTGCTTTTGGTTTCTGATGTCGTGTC
CAAGAAGCCAATGAGTCAGGTTGAATCGAGAGATGAATTGGTGAACACA
AGTGAGGCTAGGAATTTGGCATCACAAGCAGAGAGTATGGACCTCGACG
GCAGGGAGTCATCTATGGAAGCAGAGATCAGCGCAGAGAACATGGCTAG
GTTGGCTGGGATGTCTGCAGGGGAGATTGCAGAGGCCCAAGCAGATATTG
TAAATAAGCTGAACCCTGCATTGCTGGAGATGCTGAGGCGGCGGGGAAG
GGAGAAGTCTGGAGGCACGAAGGATGTGGGTAAGGACAAGGGTCTGAAA
AACTCAGGGCTGCAGAAGAATAAAAGGGCTACACCAGGGGATTGGTTGA
CTGCTGGTGAACATACTGGGCACTCCTGGAAGGTGTGGAGCGAAAGAGTG
GAGCGGATCAGGTCTTGTAGGTTCACATTAGATGGAGACATATTGGGGTT
CCAATCTTCTCACGAGCAACAAGATGGTATACCACATGGTTTTGCTCATAT
GAGCTACATTTGCTTCTGTTGGTGTGGTATCCTTTAGATGACAACCTATAT
CTCCCACTTTCAGGCAAGAAGATGCCTTCAGAAAGTGTTGCTGAGCGTGA
TTTCCTTAGAACAGAAGGTGATCCTGCAGCTGTTGGGTACACAATCAATG
AGGCAGTGGCACTTACCAGGAGCATGGTTTGTTTTTTTTATACACCCTCCA
TTTCGAATTTTATTTGTCCATTTACTTGGGAATTGATTTTCCTGTCGAATAC
GAATGAGAGCTGTTTCATTGCATTTAGAAGAAAAACCAGAATACAATGCA
CCCACAATATGTGCCACATCCACACACCAATCTCTTAGTTACAATCGCTTC
AGTTACATAAAGAACATGAGCCGACCTGAACACTATCAAGACAACTATGA
GAAAGTTGTTGAAGTGTATAAGTGGATTGGCTACCTTCTCCCATCAGCTTA
AGCTTTTGGGTTGAACTGGTTAGTGCGTCCATTCTAACATGGTATCAAAGC
CAGAGGTCTCGAGTTCGAATCCTGGCAGAGGCTTTATTTGTGCCTCCACCC
ATTTATTTCCACGTTTGCGCCTTTCTCTCTGGCTGCGTTCGCGCCTTTCTCT
CTGGCTGCACGTGAGTGGGGGTGTTGAAGTGTATAAGTGGATTGGCTACC
TTCTCCCATCAGCTTAAGCTTTTGGGTTGAACTGGTTAGTGCGTCCATTCT
AACAAAAGTAACCAGGCAATGCGTTCAACCTGCCTTAGCCCTTAGCAGCA
GCTGCATTGCATATATTTATATGGGCACATGGCCATTGTTACAGACTAGG
AGTTGACCATTTAGGGATGAAAATGGATACTTATTCGGATATCAATTTTTT
GATCTTTTTTCTTTGATTGTGAATAAATAGGATATAAAATCTGTTATGTAA
ATTCATATTCTTGTTTTTAGCATTGAGCTTGTAAAGTTACATAAAATCTTA
AAAAGTAAACCTCAAATTTATCATATATCTTCTCAAGTGATAGATATAAA
ATTTGGATACAATTCAGATTTGGACACCAATAATTTTTTTTACCTTTTTTGT
TGTAGAGAGCAAATAATGTATAAAACAGTTCATGCAAAATTTTATTCTTA
TCTGTAATAATGTGCTTGATAACACAAAAAAAGATTAACATCAAGGTGCT
AGGCGAGGTAATGAGTTTTTTTCCCACAAATTTGGTTTGTATAGAGCGTTA
GCTATTGAATTGGCTATATGGCAAGTTCAATATTCTAGTCTATTTTATACG
TATTTGGTGAAGGTAGTAAATAGTAACCGTTTTTTCTCGAACGCGCAAGA
AAAATGCACATCATTAATTCATTATATTAAGAAGATGTAGAAAAAGGTCC
ATGAGGACCAGTACAAAGTCAGGCACCCCTTACGGTGGCCAAAAACAGG
AATACACAAAAGAATCCACAAACATTCTAGCAAACGAAACTCTACTCAA
GACCAGGGATTGGGGCTGTTAGGAGGTCAGAGGAGTATCCGAGGAAGAC
ACAGTGAGTGGAGCGGGGGGCTAGTTTATGAGGAGCAGTGGAGGCGAGA
TTAGGGTAGCAAGCGCACCCGAAAACACAAAGATGGTTATAGGAGGGGT
GGATGCCGAAAAGAGCGAAGTAAGGAGTGGGGTGGGTAACCGCCTTGGT
GGGAAGACAGTTGAGGAGGTAGGTGGCAGTGGCGAGGGCCTCAGCCCAG
TAGTGAGCAGGAAGGGAAGCCTGAAAGAGTAGAGAGCGCACAACATCGT
TCGTCGTGCGAATCATACGTTCAGCCCTGCTGTTTTGAGGAGACGTGTAG
GGGCATGACATGCAGAGGTGGATGCCGCGAGAGAGGAAGAAGTCACGGG
ACACATTGTTATAGAACTCACGCTCGTTGTCACACTGGATGCTCCGGATG
GTGCGACCGAACTGAGTAGAGACCCAGGCGAAGAAGTGAGACAGGGTGG
GAAAAGTGTCCGACTTCTGACGTAAAGGAAAAGTCCACAAATAATGCTAG
AAGTCATCGAGAATGAGTAAATAATACTCATAGCCAGAAATGCTGGTTAC
ATGGGATGTCAATACATCACAGTGTATCAGATCAAAAATGCCTGCTGCTC
TAGAGGAGGAGGTGGGGAATGGGAGCCTAACATGGTGACCTAACTGACA
AGCATGATAGAGGTGCTCAAAAGATCCCCTACAACCAGAAACAGAAGTA
CTACTGGAGAGCTTGGACATCACATCACGCCCGGGGTGGCCAAGACGAC
GATGCCAAGTCGCAGAAGAGGCGGTCGCTGCAAGAACATGTGGGGCAGA
GGTCGAAGTAGTGGGAGCAGGTAGACGAAGCGTGTAGAGGGGCCCGGGG
CTGTCACACCGAGCGAGAAGAGTCCTGGTGGCAAGATCCTTCACAGAAA
GACCAAACGGGTCAAACTCCATGGGACAAGAGTTGTCAGTAGTGAACTG
ACGTACGAAAAGAAGATTTTGAATGATGTGGGGGGCAACAAGGACGTTG
GTTAAGCGGAAAGGACCGGGAAGGACCGCGTCACCTACTGAGGTGACGG
GAAGGGCGGACCCGTTTCCGACAATGATGGAAGAGGGAAGAGAAGGGTG
GACGGGGGAGGTGGAAGATAGAATACATGCGTCCGGGGTGGTGTGGTAG
GAGGCGCCGGAATCCGCCACCCAGTCAGAGACTGAGGTGGGCGGGGCCA
GCGTCATCATGGAGAAGGAGCTGGCGAGGGATTGCGCGTCCCACCCGTTA
GTCCAAGGCCCCCACATGGACTGAGCCGGAGACCCCGGGAGTGGCAGAA
GACCTTGCTGAGGCTGGGGAGGAGCCGAGGGTGCCGCCGGGGGTGCGGC
GGCAAAGAAGAACTGCTGAGAGGCGGTGGGGCAAGGGGCCGAGGCACCC
GTGGACCGCCCGAGCCACATGTGAATGGTGCCAGTCCACGGGTTGTAGAA
GGATGGCCACTGAGAGCCGCCCGAGGAACCACCACGGCCTCCCGACGGG
CCATCGCCACTGCGTCCGCCCTTGCGACCACGGCCGCGACCGCGGCTGCG
AGCCCCCCCGAAGCCAGCAGTCTGTCGCGAAGCTCCAGAGGGCAGCGGG
GCTGGAGTGCGGGGAGCAGGAACCCCCCCCGGAGTCGGAGGGGGCAGCC
TGAGGTGCGCTGTAGAGGGCCGTGGCGGGAGCGGTGGTTGCAGCGGCGG
AGAGGCGAAGCTCCTCGAGAAGCAGATCGTTCTTAGCCTCCGCAAAGGTG
GGAAACGGCTTCATGCGGGTGAGGATGGGGGCCACGCGATCGAAGCGAG
TACTCAGACCACAGAGGAGGTTGAGCACGAGGCTCTCATCGGTCATGGGA
GCCCCGAGGGTGCGGAGAGTGTCTGCCATCGTCTTCATCTGACGGCAGTA
CTCGTCCATAGAGAGATCGCCCTGAACAAGCTGGCGGAAGGCGGTCTCTA
GGTACAGGATCCGAGTCTGGCGGTTGTCGAGGAACTGAGCCTCAAGGGCA
CCCCAGATACGGTGAGCAGTATCGTCCGGCACTCGAACAATGTCTTGAAG
TTCGGCGGTGAGGGTGCCATGGATCCAAGAGAGCACCACTTCGTCCATCA
GGAGCCAATCTTCCGTCGGTGCAGCGACCGTGGGGAGGACGTGGTCGGC
GAGGGCGTAGCGGCGGAGGGTCTGCAGGACCTGGTCGCGCCACCGAGGA
TACTGAGTGGAGTCCGGCGCGAGAACGTCGGTGACGGTTGCCCGGATGCT
GGTGAGGGCCGCCGCCTGGGCGTGAAGAGAGGCGCGAAGGGTCGAGGTT
GGGACGGGCGCGGGCTCGAAAGTGTCGGCGCTGTGACGAGCGGACATGG
GAGAACCATCTGAGACGCCCTTGCCAGCGAGGCGACGATAGGTCTCGGC
GTACTGGCGCTCGATGGCGTCGACAACCGCCTGTTCCTGCTCAAGGGCAC
GAGCGGCGTCCTGGGCCCACTGGCGAGCCGCCGCAACGGCAGCACGGGT
GGCAGCGAGGATGGCTGCAGCGTCATCCGAAGGGGAAGGAGCACGCAGT
GTAGAGGTGATAGCGGACGCCTGGCTATAGACCGTCGTGCCAATGGTGCC
ACTGGAAAAAACCAGGGGCAACAACACGGCGTCGTCGTGGCCGAGCACG
GTGGTCTGGGACGCGTGAGGAAGGGAGATCGGCGCGGGACAGACACCGG
CAAAGCCGGAGCCGGCCGGACTCGGCAGCGGCACGGGCGAAGGAAGCGC
CGGCGGCGCAGGGGGCGCAGGAGGAGGGGCAGCGAGGCCGGGGGCGGG
CAGACCCGGCGGCAGAGGTGTGGCCGACACGGGCTGTGCGCCGGCGGGG
CCGGAGCCGGAGTCGCTGGAGGGCGGTGGCGGCGCGGGGCTCCCGGCCA
TGAGGGCCAGCCCGGCGGGTGGTGGCGCGGCCGACGCGGGCCCGATCCA
GCGGAGGGAGGCCGGCGGCACGGGATGCCCGGCCAGAGTCGCGCCGCGC
GCGGGAGGAGAGGGCGCGGCCAACGCAGCCCTGTAGGAGAGAGGGAGA
GGAGGGAAGAGCTCGCCGGAGGAGGGGAGAGGGGGCCGGCCAGCCATG
GGGCGGCGGCTGGAGGAAGGGAGGGGTGGCGGCGGCGGCTGGAACAGTC
AAGGCTCTGGTACCATGTGGGAATGTCTGCTGTTACAGAAACCCTGGCTA
CAGTGCGGCTGCGGCTACAGTACTAGTAAACCCTAGCTACAGTATTAGTG
ACCTAATGGGCCAGGCCCATGTACATATATCTGTAACAAGTTCTATTGTTC
CTTATTCTCTTGGGTACTACAGCAAAAAGTCTAGGAGCAAGATCCTCAAT
CCTTTTTCTTTGAAGCCAGCGGTCAGTCCAGAACAAGGTTGAGGAGCCAT
TACCAATTTCAGTGATTACAGCAATGGAAAAGAATGCTTTCACTGTTTCA
GGGAGCTGCATAGGGAACAAGATCCAGGGTTTTGAAGGATCAGTTTTTGC
TAACCACAGCCATCTCACTCTTAGAGCCCAGCCAAGTTCTTTTTGGGTTTC
AAAGAATACATTTTTACCATCATCTGTTAAAATGTACTTCTAAATTTTTAG
AAGTTGCAATATCATGACATATGATATAATTCAGCTTCCTCAAATTATAGG
TTCCGGGGCAGCGTGTGCTTGCACTGCAACTTCTTGCTTCTATTCTGAATA
GGGCATTGCAGAGCCTTCATAAGACCGATCTAATGGATAATGTTAAAGGA
ATGAATTCCAAGGATAACATTGATGACTGGCAAGCAGTTTGGTCCTATGC
CCTTGGGCCTGAACCAGAATTGGTACTCTCCCTAAGGTAAATTTACCTTTC
TTAAATACCCCTTTTATTATCAATGCAAACCCATTTTCTTGGGAGTTAGCT
TCACAATTTTATCTACTGAGGAAGTGAGTGGAGCTATTGCATGGTTTCTAC
TCTTTACCGATATCTAGTGCTGACACTGGCATTCTATCTACTGAGTGTTTG
TGCTATGTGAGTTTGTGATCTAAACGTCATTTTTTATTTATTGGATTTAGTG
CTTCCTTGTTACATGCACGGAGTAATATTATGCATTCAGATTTACAATGCA
TATAAATGAAGAGAACCATAAATGGAACATTTGAATAGTTTTGATCAGTA
GTAAACATGTAAATACATTCATGGACCTCTCATATTTGTGTTGTCCCTTAG
CTGTGTATTTTAAATTTAAAGGTGGCATGTGTGTTGGTGTTTGTGTGTGCA
TCCACCTTGTAATCTAAAATACATGGTTGCATTTTTTTTGTTTGACCCAAA
GACAACTACTTGGATTTGAGACCTTGTGATGCCATTTTCTTTTCTTTCCTAT
TTTCTACTCTGCACTCTGTCACTAACTGATGCATTTGCAGGATGGCTTTGG
ATGATAACCATGATTCTGTAGTTTTGAGTTGTACTAAAGTAGTTAATGTTA
TGCTGAGCTGTGAGTTCAATGAATCCTATTTTGAATTTTCAGAGGTAATTT
TGTCCTTTTTGGGCAGTTCTAGTCTATTTTCGTTGCTAGTTTGAGCCTCATT
TTGTCATTGTTATACCTGCAGAAAGTAGGTAATGGAAAAGATATCTGCAC
AGCTCCTGTGTTTCGTAGCAAACCTGACTTAGATGGAGGCTTTCTTGAAGG
TGGGTTCTGGAAATACAACACAAAACCTTCAAATATACTCCCACATTGTG
GTGATAATGACGAAGACGAAGCTGATGAGAAGCATACAATTCAGGATGA
TGTTGTTGTGTCTGGCCAAGATGTTGCTGCTGGTTTTGTTAGGATGGGAAT
ACTTCCACGAATCTGTTTTCTGTTGGAGGTCAGTTTGCCTTATTCTTTGATT
TGTTGTCGTTTCTTTGAAAATACTTCAAACATGCGGAAGTTACATTGACTT
TTGTGGTTTCTTATGTTCAATAATACATAACCCACTCGATGTTATTTGCAC
TTACTCATGCCCCCACACTATTGAGTGATGTCAGGGGTGGTTTGAAAGTTT
GACTAATTTTTAATGGCCAATGCCGCTATTGGGCCACCTGGTCCTCACCAG
CAAATATCATTTTGGTCACACCATGTCAATATTTGTTGCTAGTGAAAGTGT
AATAAGATAATTATGATTAACTTTTGAATTTTTAGCACCAACCTTGGGAAT
TGGGAAATTTTAGAACATATTGTGGTGCTAGGTGTCCTATAATTATGACTC
CGTTATCAATTACTCACTCGTGTACAGCTTTGTTGTTGTTGCTTGCCCCATC
CTGTGGGGCTAGTATCTAGGGCATTTCTATTCCTCTTAATTTATGCTCGGA
TCCCCTACTATTTTTCTTTTGAAAAATAATTGCCATATAATTTTAACTGGAT
GTACCTATTAATTTATTATATCTTTTATCCAACATCTGCATATAATTTTGAA
TGTATACCCTGGCTTGTGTGCTGTTCTCTTGAACTGTCTTGATATTTGTTCC
ACTATGTATTTTGTAGTGATAACTGATAACGGTGCACTGCACTTGTACATT
TCTAGTGAGCCCTGTATCAGCTAATGACTAGTTGACTCATTTTTTTGCTAA
TATTTTTGTTCTTTTCATTCAAAAAACTGTATTCATTATGGGTGTTAAACTG
AACCTTTTCTTTTCCATTGGTTCAGATGGATCCATCTCCTGCTTTAGAAGA
TTATCTTGTATCGGTTCTTGTAGCACTAGCCAGGCACTCCCCACAATCTGC
TGATGCAATCTTGAATTGTCCAAGACTTATTCAAAGTGTTACTAAGCTGTT
GATAAATCAAGGATCAATGGAAATTCGCTCCTCACAGATCAGAGGGGTTA
CTCTCTTGAAGGTATTTTTTTGGTTCTTTTGACACCCCCCGTGTTATAGAGT
TATCATGATAGATAGTTGGAATCATCGATTGACATATATTGCACACATGTT
GATAGTTTGATGCTGAATAATTCCTGTACGTGCAGATTATTATTAGACAGA
TGCTTCTCTTGGTCATCCTTGCTATTTTACTGACCGGGAGATGTTTTAATAT
TCCCAGTAATGTGTGGTACTCCGTACTCGTCTGAATTATTAGTTGAGCTCA
AATGATTTTCTTTGTATGCAAGTGCATTAATAGTTCACATTGAATTGCCAT
ATGATATACATGTTCCATGAATCGGTTCCTTTCTAGTTCAGGATGCTTGCT
CCTTGTCTCCCTCAATCACACTGTTCTCCACATTTTATGTACCCACACATA
CTAATTATGTGTTTTGATATTTTATAGTCTACATATTCACTTATATGTTCCA
GTATTATTATTATTTGAAAATACAAGCATCTTGAAAGCTACTGCAATTGAG
TCATATTCCAGAAAACATGTGCAATTTTCTTTGTTTTTCTGCCAAGCAGGA
CTTCTTTTGACCATCTAAAGCCACCTATTCTGTTTTGATCAGGTTTTGTCCA
AATACAACAGACAAACCTGCTTGAATTTTGTGAATCATGGAGTTTTCCAG
CAGGCATTGTGGCACTGGTACAGAAAAGCTGGTACTATTGAGGACTGGGT
AAGATCTGGAAAGGAAAAATGCAAGCTTAGTTCAGCAATGATGGTTGAG
CAGCTGCGGTTTTGGAGAACCTGCATCTCCTATGGGTTTTGTATAGCTCAC
TTTGCTGATTTCTTTCCTGTTTTGTGTCTGTGGCTTAGTCGTCCTGATTTTA
AGAAACTAAGTGAACACAATGTTCTTGTTGAGTTTAGTTCCGTTGCTAGA
GAGTCATATCTTGTCTTAGCTGCTCTGGCACAAAGGTTACCACTTCTTCAT
TCAGTGGAGCAGCTTGCCAATCAAGATCTGGGAGTTTCTGCCAGTTATATT
GAGACATGTTCTTGGAGCCATGTTGTCCCGATGGTTGACTTAGCTTTATCT
TGGTTACATCTTAATGATATTCCCTATGTATGTTCACTAATCAGCGAGCAG
AATAGGAATACAGAGCACATGTTAGAGATGAGTTATCTGATTTTGGTGAT
TTCTTCTGTGCTAGGCATGCTTAATTCAATTTTGGAAAGAATATCACCAGA
TGTCACTCCTGAGGATAAAAGTTACAGCTTGCCCTGGATACCTGATTTTGT
CCCCAAAATTGGCCTGGGCATAATTAGTAATGGTTTTTTCAGCTGTTCGAC
CACTGTTGCTGGTAGAAATGCAGAACATCAGCCTTTTTGCTGTGCATCTTT
GGTGCAGGGACTTTGTTATATGAGATGCCATGGTAATGTTGATGTATCATT
GTCTTCCATTAGCTGCCTTCAAAGATTGGTGCAGCTATCCTGGTCTGTCGA
CAGAGTGATCCAGGGAGCCACAAAATGTTGTTCCGAGTGTTTCAATGAGT
CTGGAACAGGTGAAGCTGGCAAACTACTAGCTGAAGGTATCTCCAGTTTA
TGGCATAATGATTTGCTACACTTGCTGACTTCGCTTTTGCCAATGATTTCA
TCACAATGGTCCATATCACAGAACATAGAGATGTTTGGTAGAGGAGGACC
AGCTCCTGGTGTTGGGTTTGGCTGGGGGACATGTGGTGGAGGGTTTTGGT
CTCTTAAATGCCTACTTGCACAATTGGATTCACAATTGGTTGTAGAATTGA
TCAAATGTTTCTCTTCAGTTCAAGGAAGTCCTATCATCCTCGATGAAGGTG
TGAAGTTAGATAATGTGACTAACACAGTTGTGACAGCTTCAAATTGGATC
AGTTCTACCCTAGGGTTGTCTCTGATTGCTGGACCTGGACAAATCTATATG
TTGGAGAAGGTTTTCGATATGATTTTTGAACCTTCCATTCTGAAGTATCTC
AAATCATCTATACATAAATTTACCTCTGACATGGAATTACTGAAACCTTTT
GAATGGGACTTAAATGAAGATGAATATATGCTCTTCAGCAGTGTTCTCAA
ATCACATTTCAGATCCAGATGGTTAGCCATCAAGAAGAAGCATTCAGATA
AATATGCAGGAGATAATAGCAGCACCAAGATTTCAAAAACACCAGAGAT
ATTGGAGACAATTCAAGAAGAAACAGAGTTGTCAGAAGCTGTAAATCAA
CCTTGCAACACATTAATGGTAGAGTGGGCGCATCAGAGACTGCCTCTTCC
TATCCACTGGATTCTAAGTGCAGTTTGCTGCATTGATGATCCAAAAGGCA
CACTCTCAACATCAGCCAACTATATTCTTGATGTCTCAAGGGCTGGTCTTA
TCTTCCTTTTAGGTCTGGAGGCCATTTCAGCTACCCCGTGCCTTCATGCTC
CTTTGATCTGGAAAATTCATGCACTTTCGGTCTCTATCCGCTCTAGCATGC
ATTTGCTACAGGAAGACAGAAGTAGGGATATTTTCTGTGCTTTACAGGAA
CTGTATGGCCTGCATCTGAACAGGTTATACCAAAAATTCTGTAAACCAAA
CTCTATCGAGGAAGTTAAGGGCGTTGTAGTGGGCACTTCAGAGGAAGCGA
TGGAGATCAGTAGCCTTGAAATTCTCAGGTTTCAGGAGAAAATTCATGGA
AGCTATACTACTTTTGTTGAGAGCCTGGTTGATCAATTTGCAGCTGTCTCA
TATGGAGATTTTGTTTTTGGTCGGCAAGTGGCCATTTATCTGCATAGAAAG
GCTGAGCCAGCAGTACGTCTTGCAGCATGGAATGCACTGTCTAGTGCGTA
TGTGCTTGAACTGTTACCCCCGCTAGACAATTGCATTGGCAACGCCCCAG
GATACTTGGAGCCTCTTGAGGTACATTTTCTTTAATTTATTTTGCATTTCTC
TTCCAGAGAAACCTTTTCATGGAGTAACTATGTGAGTGATTTATTTTGCAA
CTTGACCATGTACTCTTGTCTCTGTGTGTTGTAGAGCCTAGAATATGCAAT
AGGCACATCATGATGCATTACTATTTTGCTGACATTTATTTTGGTTTCTTTA
TGTATGAAATCCATCCACCATTTAGTATGATGAACTTCTTTTGTTGGCTGC
TTTTCCATGTTCAAAGGACAGTCTCACTTTGTCAAATTTTTACAATACTTCT
GCAGGATGATGAAAAAATTTTGGAATCTTATGCTAAATCATGGACGTCTG
GTGTCCTGGACAAAGCTTTACAGCGTGATTCCATGGCCTTCACATTGGCA
AAGCATCACCTTTCAGGCTTTGTCTTCCAGTCCAGCGATTCTGGCACAATG
CTGCGAAAAAAACTGGTCAAATCGCTTATCCGGTGCTATGCACAGAAGCG
GCATCATGAGGTAGTTGGTCGCTCATGTTTCTTTGTTTGCTTGGTCCACGG
CAATTCCTTCCACGCCACTGTCTGAGTGTCTGTTGAATTCTGTACAACAGG
TTATGCTTAAGTGCTTCGTTCAGCAAGGCATCGCACAGGATTCCAAGAGC
AGTGAGCTTGACCGGAGATTTGAAATCTTGAAGGATGCTTGTGAGATGAA
CTCCAACCTCGTAGGTGAAGTCCAGAGACTGAAGGCATGTCTTGGCCAAT
GAAGCCAGATATTTAAGTGTGTCATTAACTTGGCAGCGTTAGTTGTTGGG
AAAGCTCGACAAAGTGGCCCAAAATATGCAACTGAGGAAACTCACGGTC
GGGGCTAGTGGTTACTTTTAATTTTGATGAAGGGAACGCCGAGCACGCAG
CAACCCTGAGTCTAACCTGGCTATGGTTAGGCTGCTCTTAGCAGAGCATG
CATATGGTCATGTAAACAGTAGACCACACTGTCTATTGAGTGAAGTTTGA
AATAGACTTTATATATAGCATTAGGCTTCCTTTGATTCC
SEQ ID No: 20 MINIYO Glycine max, >GM01G08040, Gene
ATTCCTGGACAAAGAGCCCTTGCATTGCATCTCCTTTCGTCTGTGCTTGAT
AAGGCATTACACTATATTTGCAAAGACAGAACAGGGTATATGACAAAAA
ATGAGAACAAAGTTGACAAATCAGTTGACTGGGAAGCTGTTTGGGCTTTT
GCACTGGGCCCAGAACCTGAGCTTGTGTTGTCACTTAGGTAAAGTTTGCTT
TTTGGTCCAAGCTGTTGGTATTATAAACTACCTGCTATAACTGAGTGAAGA
TGCATGGTTTTCTTTTTCCTCTTTTAAGAAAATGAATAATGTTGTTTATATT
GCTGTTAATCATTAAGCATACAAGTTGATTGGTTTGGATGTAAGTAAAATT
CCAGCTAGGTTTTATGTGTGTAATTTTAATATCACTTCCATCTGGCGTGTG
ACATATATCTGGTTCCATTACTGAGTATTTACATGTCTTGCACCCTAGAAG
CCAGCCTTATTTGTGGTAAAATGTTTTATAATATTATTATGCCTTAGAATA
CTTTAGAGTATCTTAGTTAATCTCGAGAGTGGTTTCCATATTTAATTATTTT
TGATGATTTTTTAATTATTAGGAATTTAGGATTTGGAACCTTGTCTTAAGG
ATGAGGGTTAGTGCTTTGTCTTTTGTAAAATATCATTACACACCAATGAAG
ATAAAATTCTCTCCTATTCTCTTTATTTATTTAAATCCCCATAATATGAACC
TGGTTCCGAAGTGATCCCATCATCTGTGATTTGACCCCATCACTTGCTGCC
TTGTTCTCTAAACGGGTGCTCATCTTTGTTTCTGTGTTTTTTTTTTTCTTTTA
ATTTTTGGTCGCTGTGTCCTTAGTCCTTGAGGATTCTTATCCTCTTATATTT
GTATCTTTCTCTTCTATCTTAATATATTCTTTGCTTATATAAGCTTTGTTGA
TTATTATGCTTTGGGATGAGCTCTTGTGTATACTGGCTTTTTCTAATCTGTG
CCCTTTTTTTGTGGTCTTCATCCTAAAACTGTGATGATCATCATATGTTAA
AATGGATGCTGGTTGGCCCTTGGACCCTTAGGGAATTTTACTTCCTGCATA
ACTGTTTCTAAAATTCAATTGGAAGAGTGGGAATCTAGATTGCATACTTG
ACTCACACCTTCTGTCCTCTTGTATTTAATAATATGGAGTGTTGGTGAGGC
TATAAAGATTTTTTATTATGAATTATAAATTACTAAATGCAAATAAATGTC
ATCCCAAATAGATGGCTATATATCTTGTCAATATGTTTGAATTTTGCCTAT
AGATGGTATTATTATGTTGTAATCCATGTGGTATTTTGAGTTGCTTATGAT
AAATATACTGATGAATACTTTAATTCTTTCAAATTGTATTAGAGTTTAATG
CCCATGCATTGGTAGTGTAAAAAAGTTTGACACTGTCATCCAATAACAAA
TCAATGTTTGAATTACTTTAAAATAATTATTTTATAAGTCAAAAAACTTAC
CATTCATTGTGGGTTGTGATTGGATAACTGTAAAACTTTTTACACTGTCAG
TACATAATCCTTTTTCTCATTGTATTATCATAAATTTATTTTTTTCCTCATT
ATATGGGCATCTTTTGACGGCAGTTGTGCTCTAATTTTGTAGGATATGTCT
TGATGATAACCACAATTCTGTAGTTCTGGCCTGCACAAAAGTTGTTCAATC
TGTATTGAGTTATGATGCAAATGAGAACTACTGTGATATGTCAGAGGTAA
CTGGATTTTTATTATCTTTTAATTTGACAATATTATTATGTTTAAAATTAGA
TTCATCTGATTTTTTATTTTAATTCCAGAAGATAGCAACTTGTGACATGGA
TATTTGCACGGCTCCAGTTTTTAGGAGCAGACCAGATATTAATGATGGATT
CCTTCAAGGGGGTTTCTGGAAGTACAGTGCCAAACCTTCTAATATTCTTCC
TTTCAGCGATGATTCAATGGATAATGAGACTGAAGGAAAACATACTATTC
AAGACGATATAGTGGTTGCTGCGCAAGATTTTACTGTAGGTCTAGTTCGC
ATGGGAATCCTTCCTAGGCTTCGTTATCTTTTGGAGGTAAAACATAATTAA
TCATTGATCATGCTGAAACATTTGATGTCATGAGACTCTATCTACAAAGAT
TAAGGATATTTTTGTTTTATGATGTTTGAACTTCCTTGTCAAATTCATTATT
CTCTAATGGTTGCAGAAAGATCCTACAACAGCTTTAGAAGAATGTATCAT
TTCTATACTGATTGCTATAGCGAGGCATTCACCTACATGTGCTAATGCTGT
ACTGAAATGTGAAAGACTTGTTCAGACAATTGTGAATAGATTTACTGCTG
ACAATTTTGAACTTCGATCTTCTATGACTAAATCTGTAAAACTCTTGAAGG
TGAGTGGTAATTTTCCATTTTATTTTATAGGAAGTGATTTCTACATCAGCA
GCCAGAGAATCATTCCTAATGTTTCATTTGATTTTGGAAAAGGTGTTTGCT
CGGTTAGACCAGAAAACTTGTTTAGAGTTTATAAAGAAAGGGTATTTTCA
GGCTATGACTTGGAATTTATATCAAAGTCCTTCCTCCGTTGACCACTGGCT
AAGGCTAGGGAAGGAAAAATGTAAACTCACATCTGCTCTGATTGTTGAAC
AAATGCGTTTCTGGAGGGTCTGCATTCAATATGGATATTGTGTGTCTTACT
TCTTGGAAATGTTCCCTGCCTTGTGTTTTTGGTTGAATCCACCTTCATTTGA
AAAACTTGTTGAAAACGATGTTTTGGATGAATCTACTTCCATCTCTAGGGA
GGCATACCTTGTTCTGGAGTCTTTGGCTGGAAGGCTTCCTAATCTATTTTC
AAAGCAGTGTCTAAACAACCAACTTCCAGAGTCTGCTGGTGACACCGAGG
TATGGTCTTGGAATTATGTTGGTCCAATGGTTGACTTAGCCATAAAGTGGA
TAGCAAGCAGAAGTGATCCAGAAGTATCCAAGTTCTTTGAGGGACAGAA
AGAAGGAAGATGTGACTTTCCTTTCCGAGATCTTTCTGCAACTCCTTTGTT
GTGGGTGTATGCTGCTGTGACTCGCATGCTTTTCAGAGTGCTTGAAAGAAT
GACATGGGGGGATACTATCAGCTCTTTTGAAACTGAAGGGCATGTGCCAT
GGCTTCCAGAATTTGTACCTAAGATTGGACTTGAGTTGATCAAATATTGGT
TTTTGGGCTTTTCTGCATCCTTTGGGGCAAAATTTGGAAGAGATTCTGAAG
GTGAATCTTTTATGAAGGAACTAGTTTATTTGAGGCAGAAGGATGATATT
GAAATGTCTTTAGCTTCCACCTGTTGTCTAAATGGAATGGTCAAGATTATT
ACTACAATTGATAATCTGATACTGTCTGCCAAAGCTGGCATCTGTAGTCTC
CCACGCCAAGAACAAAGTCTCTCAAAAGAAGGAAAAGTGCTTGAGGACG
GCATTGTTAATGGGTGTTTGGTTGAGTTAAGGTATATGCTTGATGCTTTCA
TGTTTTCAGTTTCTTCAGGGTGGCACCACATACAGTCCATTGAGTCATTTG
GCAGAGGAGGACCAGTTCCAGGGGCAGGAATTGGATGGGGTGCCCCAAG
TGGAGGATTTTGGTCAGCAACATTTTTATTGGCACAAATAGATGCAAAAT
TTCTCGTCTCTTTGCTGGAAATTTTTGAAAATGCGTCTAAAGGTGTTGTGA
CTGAAGAAACAACCTTCATTATCCAAAGGGTGAATGCTGGCTTGGGATTG
TGTTTAACTGCAGGACCTAGAGAAAAGGTTGTTGTAGAAAAGGCATTGGA
TCTCTTGTTCCATGTCTCTGTTTTGAAGAACCTTGATCTCTGCATACATAAT
TTTCTCTTCAATAGAAGGGGTAGAACTTTTGGCTGGCAACATGAAGAAGA
GGATTACATGCACTTAAGAAGAATGTTATCATCTCATTTCAGGAGCAGAT
GGTTGTCTGTAAAGGTGAAGTCTAAATCTGTGGATGGCAGTAGTTCCTCT
GGCATTAAGACCTCTCCAAAGGTTGGTGCTTGTTTGGAAACCATATATGA
GGATTCAGACATGTCTTCCATGACAAGTCCGTGCTGTAATTCTTTAATGAT
AGAATGGGCTCACCAGAAACTACCACTTCCAGTTCACTTTTACCTTAGTCC
AATCTCAACAATTTTCCATAGCAAGCGGGCTGGTACTAAAAAAGTTGATG
ATGTACTACATGATCCATCTTATCTGATTGAAGTTGCCAAATGTGGACTTT
TCTTTGTTTTAGGTGTTGAAGCAATGTCCATTTTTCATGGCACTGACATTC
CTTCTCCTGTTGAACAAGTATCATTGACATGGAAGTTACATTCCTTATCTG
TCAATTTCCTTGTTGGAATGGAGATACTTGAACAAGATCGGAGCAGGGTT
ACTTTTGAAGCTTTGCAGGATCTTTATGGTGAGCTTCTTGATAAGGCAAGG
TTAAACCAAAGTAAAGAAGTTATTTCAAATGATAAAAAGCATCTTGAGTT
TCTGAGGTTCCAAACTGAGATTCATGAAAGTTACTCAACTTTTCTTGAAGA
ACTTGTGGAGCAGTTTTCTGCTGTTTCTTATGGTGATGTTATTTTTGGCCGG
CAAGTTTCACTTTATCTACACCGTTATGTTGAAACTTCCATTCGACTTGCT
GCCTGGAATACACTGTCCAATGCTCGTGTTCTTGAGCTTTTGCCACCTTTA
GAAAAATGCTTTTCTGGTGCTGAAGGATACCTTGAACCTGCTGAGGTAAA
AAAAATAATCATTAACTACCAGCTATTTTACTCTGGAGCATTATTTGCAGA
GGATATTGATTGAGTACTTGTTCTTTTGATACTTTTTTCCTTTCTATGTTGA
TGATATTGGGTTGAATATATAAATGTCACTTATATTTTATATTTTATTATA
ATTTTGTAAATTTTCAAAAAGTGGTTGTTTAAAATGCGCTTAAAAACAGA
AAAGCGTATGCTACAAAGTACAGCTAATGTTGCAGAGTTTAACTGTGTTA
CAAAATTTACAATACCTTTGACATTTATTGGTTGTATTGTAGCATCTGTCA
TGAATTTTGAGGAAGCTTTTGAGAGCACAGGAAATACCAAAATAAGCCAT
ACTGATACTGGGTCCTTTTATTTTTTATATGATGCATAATAAAGTGAAGTT
CCTAATTGGAATTTTGGATATAGTGAAGGATCTTATACTTCCAGCAATATA
ACAGATAGGGTCACTTAGCACCAAACATGCACATATTTTTTACAATGACA
TGTTTTTAAAGCTTGTCTTTAGTAGGGAAGAAATGTAAGGTTGTATCTTTG
ATACTTTGAAGTTAGAAACTATCACGATGGCTTTGTTGTGTAAGTTACCGT
GAGGAATGAGGATTGTTGTTCATCAAGTTTCCTTATCTAAATTTTTTTATTT
TCCATTTTTTGTTAATATCTATAACTTCTGTGAATAGCAACAATTGGAAAT
GGGTAGGTGATAATTGTTTACTACTGTATTATCTGTGGAGGGGTGCACTTG
CCACCTGTGTGGTTGCACCAGCAATGCATACTAGTCATTGGCTCAGAATA
CACCATATATCTATCAACATTAATGGTGTACTGTATCTCTGCAGGATAATG
AAGCCATTTTGGAGGCTTATACGAAGTCGTGGGTTTCTGATGCCCTTGACA
GGGCTGCAATTCGAGGATCAGTTGCATATACTCTTGTTGTCCATCATCTTT
CCTCCTTTATATTTCATGCCTGTCCCATGGATAAGTTATTGCTGCGAAACA
GACTTGCTAGGTCTCTGTTGCGAGATTATGCTGGGAAACAGCAACACGAG
GTAACCAAAGTCTTAAAAGCTTTTATCCTGATGGACAAATGGTAGTTTAA
GCAAATCACCATTCAGAGCTTGTGCTGCCATTCATTCTCCAATTAGTTTTA
TCCCATCTGATTATGGAAATCAAATGTTATATGTGAATGTGTGCTGTGCAC
GTGTGTGCTTGTTCTGTCATCACGTTGTTTGGAATGTATCAGCTAGGACTT
TGGCCTACCTAATAAAATTTTAAGAGTGTGCAGTGAGACATCACTGTTTA
GGTAAATCCTAGTTGCAGTATTTTTATTACCAAGAATTTGAGGCCATGGA
AGAAGTATAAGATTTTGTCCTATCTTTTTCATCCTAATTTAATTTGCATTGT
TGCGTCTGTATTTACGATTATATAGCCATAATGTTGGTGTAGCCAGACATT
ACTAATGGAACCCTTGTTGCAGGGTATGTTACTGAACCTCATTCACCATAA
CAAGCCGCCACCATCTGTCATGGGAGAGGAGCTGAATGGTGGTGTACTTT
CTGAAAGGAATTGGTTAGAGTCCAGATTAAAAGTATTGGTTGAGGCTTGT
GAGGGAAATTCCTCTCTTTTGATAGTAGTAGAGAAGTTAAAGGCTGCTGT
AGAAAAGAGTTCATAGTGAGGTAACTAATATATCGAATGCTTGTAAATAT
ATTCGAGATATACGATGAGTACAACTAAGGTTTATATATAGATTTTTGCA
ATGGGGTCTCAAATGCATTAATTGGAGCATACATGGCCACTGATATTGAT
ATTTTTTGTTAGAATGGAGTCTGCTGATTTTTTAATCGAGTCCTAAGGCAT
GCAATGTACATAATGAAGGAATACAAGTATTTTAGTCGGGTTATTATAAA
ATACACTGTTTCACTCC
SEQ ID No: 21 MINIYO Glycine max, >GM02G13360, Gene
GAGTTTGAAAAAGTTTCAGCCTTTGCCAAGCCGGTACAGAGGAGGAGGA
AAAAGGGTTTGGATTTTAGAAAATGGAAAGAGATCACTCGGGATGATAGT
TCTTCCTTCGGGAAGGAATCAGAGAAGGATGTGTCAAGCTTTAGCCAAAC
TACTGGGAAAAAGAAGAATGAAAAGGGCAGTAAGAGCACATACAAGAA
AACCTCATCTTTGGATGATAATGTCATTTCTCCAATGAAAGTGGATACAA
AACCACTGTTAGATAACTCAGATGGTGGGTTTATCAATTCAACTACCACT
ATGGAAGTAGATACATTAAATAAGGTAGATCATGAGGAAAAAGTTAAAC
ACGCCAGAATTTATGATGACAAGGAGCAAAATGAATCTGTGCCTGGATTG
GACCAAATTTCTTCTGATTGGATGCCTGATTACAATTTTGGATCCCTGGAT
GTGCAAAGGCCAGGGCAAACTGACTTGAATTCAAGCATGCTGTCTTGTTC
TAGTTCCAATAGTATTAGAAGTGAACAAAAGTCCGTGTCTCTTGATAGTG
AAATTGATGCTGAGAATCGAGCTCGGATTCAGCAAATGTCAGCTGAGGAG
ATTGCAGAAGCCCAGACTGAGATAATGGAGAAGATGAGCCCTGCATTACT
AAAATTACTGCAGAAGAGGGGGCAGAATAAATTGAAGAAACTAAAATTA
GAAGTGGATATTGGCTCAGAATCTGTGAATGGACATGCTCAGAGTCCTCA
GGATGCAAAACATCTACACACAGAGGATGGGATCGCTCAAACAGTGATT
GTGCCACCATCCAAAGAAAAGCTAGATGATGAGAAAATTAGCACGAAGA
CTTCAACCACCGCTAGTAGTAGTGCATGGAATGCTTGGAGCAATAGAGTT
GAGGCTGTTAGGGAGCTACGATTTTCCTTGGTTGGGGATGTTGTTGATTCT
GAACGTGTATCAGTTTATGGTATGCGTTTCTATTCTTCTTATGTTTCTTTAC
TATCTTCTGGTTCTAATATCAAGATGATGCTTTGTATGATCCTTGCATATG
TATATATATTAGAGTATCTCCTTGTACCATTCTGGACTCTGTAGTTAACTA
ACAAGTGCCGGCTAAGAATTCTGTTATTAGTCAGTTGTAACTGACAGATT
AATTTCAGCTAGGCTATCTCTATATATCTCAGACATACCTCTTCTATTTGC
GATCAATGAATAAACATTTTATCAGTAATCACTCTGAATATCTCAGTGCTC
TCTTTCTCTCTCTGCTCGATGAATCCTAACAATATATGGGTTCTTCTGTATT
TAGTGTTTTGCATATGTAGGTGCTTGGTTAGTGATAGTAATAGTATTGTCT
TCTTAATGTAGATATATGCTCTTCTGTAATTTTTTTTTCTTTATTGGGTATG
TGGTAAAGCTAGTGATAGTTTGGGTTGCTAATGTTTGAATACTTTGCTTCA
TGAAACATCAACCATTGTTTATAGATTGTGATTTGTATTCAATATTATACA
TTATACTACCTCCAGACTAAAATATAAGCAACAAAAAATCAATGTGTTTG
GGTTAAAATATAAACAAATTTTAACTAACTCTCTCCTATTTAATGATAATA
TCTCCAAAATACCCTTCATTTAATTAGAGTTTTATTTCCAATAAACTTCTCT
TTTTTGTCCTATGCAATTAATGTAAATGGTACTTTAGGAAATAAATTAACT
TTTTTTATTGAGACTAACAAAATTAAATAATGCTAACTAATTTTATTGATG
AGTGTGAATTAGTTTTTTTTGCTTATATTTCAGTCCGGAGGGAGTAGTATT
TTTTGTTCCCAGCAATTGATGATCTTTTTTTGGTTGACTACATTTCTAAATG
CACATGAACCAAAAAAAACACTTACTGTTTGTTTAACTTATTTGGAGAAA
TAATCACATTTTTTCTGGTTTCTGAGAGAGATTTTGAAAAATTGGCGATTA
TTAATCCAAATTTAATCTTTATCAAACATTTTAGCATTTCCATGTGTCATGT
AGGCAATATATTGCTGGTAAATACAAATACAGGAAAGTTAAGATCAATGA
TGTTGCTATATCATGGCTTGTCTGATAGCACTGAGTAGATTATGCTCCCTG
CCCCCCTGCATTTGCCACAAATTACAATAATGTTCTGGTTCATGCCTAGAC
TGAAATTTCTGTATGGTTTCCTTTGCTTCCAAATGTTTCCAAGTTCATAGA
CTTGATTAGATGGCTTCTGTTGTATATTCAGCATATTAGATGTTAGCATTT
GCATATAACAGAACGGAGGTGCAAATATTGATTTAAATAATGAATATTCA
TGGATATCCATTGATATCCTGATTATTCTGTTAGCACTGATATCCATCAAA
TCTTAGCATTTGTTGTATATTACTATATTCATTGATATCCTGTTTATTTTGG
AAATAGGATGGAGGTTTAAATATAAATTTTGAATTTTCTTTATGTGACAAT
TCTTTGTCATGATACAGACAATGCCAATGAACGTGACTATCTACGGACTG
AGGGAGATCCTGGTGCTGCCGGTTATACAATTAAAGAAGCAGTGGCACTC
ACTAGAAGTGTGGTATGTTATTTGTTGTAACTGATGTATATTTCAGTTGGA
TGCCTGGACCTGGAACAGTTTGGATGGCTTTACAATTATGAATATAAAAT
ATATAAATTTATAGCATTCCCCTTTAGTAACACAGCGTCTGCCAAATGTTT
TGTTTTTTTAGATTCCTGGACAAAGGACCCTTGCATTGCATCTCCTTTCATC
TGTGCTTGATAAGGCATTACACTATATTTGCGAAGACAGAACAGGGCATA
TGACAAAAATTGAGAACAAAGTTGACAAATCAGTTGACTGGGAGGCTGTT
TGGGCTTTTGCACTCGGCCCAGAACCTGAGCTTGTGTTGTCACTTAGGTAA
AGTTTACTTTTTGGTCCAAGCTGTTGGTATTATAAACTACCTGCTATGACT
GAGTGAAGATGCATGGGTTTCTTTTTCTTCTTTTAAGAAAACGAATATTGT
TGTTTATATTGCTGTTAATCATTAATCATACAAGTTGATAGGTTTGGATGT
AAGTAAAATTCCAGCTTGGTTATGTGTGTAATTTTAATATCACTTTCATCT
GGCATGTGAGATGGACTCAATGAAGCTTGAGATCACATATCTATCTGTTT
ACATTACTGATTATTTACATGTCTTGCACTCTTGAAGCCAGCCTTTTTTGTG
GTAAAATGTTTAATATTATTATGCCTTAGAATACTTTAGAGTATCTTAGTT
AATCTCTTGAGAGTGGTTTCCATATTTAATTATTTTTCATGATTTCTTAATT
AATTAGGATTTGGAATCTTTTCTTAAGGATGAGGATTAGTGCTTTGTCTTT
TGTCACATATCATTACACACCGATGAAGATAGCATTCTCTCCTATTCTCTT
TATTTAATTAAATCCCTATAATATTAACCTGGTTTCAAAGTGGTCCCATCC
TCTGTGATTTGACCCCATCACCTGCTGCCTAGTTCTCTAAACTGGTGCTCA
TCTTTGTTTCTGTGGTTTTTTTCTTCCTTCCAGTTAGCTGTCTTTCCTTCTGC
CATTGTCTATCTTTTCTTCTTTGTATGTAAAGATTTTGATCTGTCAAATTTG
ATGCCTAATTGCCTTATCTCCTAGTTACTGCATGATGTCCAGATTGAGCTA
TGGTTCTGGTTCTCATTTGTTCTTACATTGCCCAGCAGTTTCCTTTCTCTGG
AATAATTTGTTTAGTGTTTTCATAGAATATGGGGTTCGTCCCTAGAATCTT
CAGCACTTCCTGTATGTTAATTTTGGAGGATTTGGTACTAGCTGAGAATCC
AGAAAGTTTTGGTGTTCTTCTCTGTGTGAAGTTCTTTGGGTATATTCAGTT
AGAAATCATCTTCTGTGGATGATAGTCTTCTAATGAGTTCATTCGGGTTCA
GCTAGTTTTTCTAGCTTCTTTGTGGTGTTCTGCTTATGGTCTTTATATATAA
GATTTTATTTTTGTTTGATTGATTGTGTGTTTCTTAATCTTTGTTTTTTTCCT
TTAATTTTTGGTTGTCGTGTCTTTAGTCCTTGAGGATTCTTATCCTCTTATA
TTTGTATCTTTCTCTTCTATCTTAATAAATTCTTTGCTTATATAAGCTTTGTT
GATTGTTATGTTTTGGGATGAGCTCTTATGTATACTGGCTTTTCCTAATCTG
TGCCCTTTTTTGTGGTTTCTTCCTAAAACTGTGATGAGCATCATATGTTAA
AACAGAAGCTGGTTGGCCCTTGGACCCTTGGGTAATTTTACTACCTGCATA
GCTGTTTCTAAAATTCAATTGGAAGAGTGGGAATGTAGATTGCATACTTG
ATTGACTCATAATACCTTCTGTCCTCTTGTATTTAATAACATGGAGTGTTG
GTGAGGCTAGAAAGATATTTTATTTTGAATTATAAATTACTAAATGCAAA
TAAATGTCATCCCAAATAGATGGCTATATATCTTGTCAATATGTTTGAATT
TTGCCAATAGATGGTATTATTATGTTGTGACTTGTGATCCTTGTGCTATTTT
GACTAGCTTATGATAAATATACTGATGAATACTTTAATTCTTTCAAATTGT
ATTTTTATAATTTTTTTCCTCATTATATGGCCATCTTTTGACAGCAGTATTG
TGCTCTGATTTTTTGTAGGATATGTCTTGATGATAACCACAATTCTGTAGT
TCTGGCCTGTGCAAAAGTTGTTCAATGTGTATTGAGTTATGATGCAAATGA
GAACTATTGTAATATCTCAGAGGTAACTGGATTTTTATTATCTTTTAATTT
GACAATATTGTTGCGTTTAAAATTAGATTCATCTGGTTTTTATTTTAATTC
CAGAAGATAGCAACTTGTGACATGGATATTTGCACGGCTCCAGTTTTTAG
GAGCAGACCTGATATTAATGATGGATTCCTTCAAGGGGGTTTCTGGAAGT
ACAGTGCCAAACCTTCTAATATTCTTCCTTTCAGTGATGATTCAATGGATA
ATGAGACCGAAGGAAAACATACTATTCAAGACGATATAGTGGTTGCTGGG
CAAGATTTTACTGTAGGTCTAGTTCGCATGGGAATCCTTCCTAGGCTTCGT
TATCTTTTGGAGGTAAAACATAATTAATCATTCATCATGCTGAAACATTTG
ATGTCATGAGATTCTGTCTACAAAGATTAAGGATATTTTTGTnTACAAGG
TTTGAACTTTCATGTCAAATTCATTATTCTCTAATGGTTGCAGACAGATCC
TACAACAGCTTTAGAAGAATGTATTATTTCCGTACTGATTGCTATAGCGAG
GCATTCACCTACATGTGCTAATGCTGTACTGAAATGTGAAAGACTTGTTCA
GACAATTGCAAATAGATATACTGCTGAAAATTTTGAAATTCGATCTTCTAT
GATTAGATCTGTAAGACTCTTGAAGGTGAGTGGTAATTTTCCATTTTATTT
TACAGGAAGTTATTTCTGCATCATCAGCTAGAGAATCATTCCTAATGTTTC
ATTTGATTTTGGAAAAGGTTTTAGCTCGGTCGGACCGGAAATCTTGTTTAG
AGTTTATAAAGAAAGGGTATTTTCAGGCTATGACTTGGAATTTATATCAA
AGCCCTTCCTCCATTGACCACTGGCTAAGGTTAGGGAAGGAAAAATGTAA
ACTCACATCTGCTCTGATTGTTGAACAAATGCGTTTCTGGAGGGTCTGCAT
TCAATATGGATATTGTGTGTCTTACTTCTCGGAAATGTTCCCTGCCTTGTG
TTTTTGGTTGAATCCGCCTTCATTTGAAAAACTTGTTGAAAACAATGTTTT
GGATGAATCTACTTCCATCTCTAGGGAGGCTTACCTTGTTCTGGAGTCTTT
GGCTGGAAAACTTCCAAACCTATTTTCAAAGCAGTGCCTAAACAATCAAC
TTCCAGAGTCTGCTGGTGACACAGAGGTATGGTCTTGGAATTATGTTGGTC
CAATGGTTGACTTAGCCATAAAGTGGATAGCAAGCAGAAATGATCCAGA
AGTATCTAAGTTCTTTGAGGGACAGGAAGAAGGAAGATATGACTTTACTT
TCCGAGATCTTTCTGCAACTCCTTTGTTGTGGGTGTATGCTGCTGTGACTC
ACATGCTTTTCAGAGTGCTTGAAAGGATGACATGGGGGGATACTATTGAA
ACTGAAGGGCATGTGCCATGGCTTCCAGAATTTGTACCTAAGATTGGACT
TGAGGTAATCAAATATTGGTTTTTGGGCTTTTCTGCATCTTTTGGGGCAAA
ATGTGGAAGAGATTCTAAAGGCGAATCTTTTATGAAGGAACTAGTTTATT
TGAGGCAGAAGGATGATATTGAAATGTCTTTAGCTTCCACCTGTTGTCTAA
ATGGAATGGTTAAGATTATTACTGCAATTGATAATCTGATACAGTCTGCC
AAGGCTAGCATCTGTAGTCTCCCATGCCAAGAACAAAGTCTCTCAAAAGA
AGGAAAAGTGCTTGAGGATGGCATCGTTAAAGGGTGTTGGGTTGAATTAA
GGTATATGCTTGATGTTTTCATGTTTTCAGTTTCTTCAGGGTGGCACCGCA
TACAGTCCATTGAGTCATTTGGCAGAGGAGGACTGGTTCCAGGGGCAGGA
ATTGGATGGGGTGCCTCAGGTGGAGGATTTTGGTCAGCAACAGTTTTATT
GGCACAAGCAGATGCAAGATTTCTTGTCTATTTGCTGGAAATTTTTGAAA
ATGCATCTAAAGGTGTCGTGACTGAAGAAACAACCTTCACCATCCAAAGG
GTTAATGCTGGCTTGGGATTGTGTTTAACTGCAGGACCTAGAGATAAGGT
TGTTGTAGAAAAGACATTGGATTTCTTGTTCCATGTCTCTGTTTTGAAGCA
CCTTGATCTCTGCATACAGAGTTTACTCTTGAATAGGAGGGGTAAAACCTT
TGGCTGGCAACATGAAGAAGAGGATTACATGCACTTAAGCAGAATGTTAT
CATCTCATTTCAGGAGCAGATGGTTGTCTGTAAAGGTGAAGTCTAAATCT
GTGGATGGCAGTAGTTCCTCTGGCATTAAGACCTCTCCAAAGGTTGGTGC
TTGTTTGGAAACCATATATGAGGATTCAGACACGTCTTCCGTGACAACTCC
GTGCTGTAATTCTATAATGATAGAATGGGCTCACCAGAAACTACCACTTC
CAGTTCACTTTTACCTTAGTCCAATCTCAACAATTTTCCATAGCAAGCGGG
CTGGTACTAAAATTGTTGATGATGTACTACATGATCCCTCTAATCTGCTTG
AAGTCGCCAAATGTGGACTTTTCTTTGTTTTAGGTGTTGAAGCAATGTCCA
TTTTTCATGGCACTGACATTCCTTCTCCTGTTCAACAAGTATCATTGACAT
GGAAGTTACATTCCTTATCTGTCAATTTCCTTGTTGGAATGGAAATACTTG
AACAAGATTGGAGCAGGGATATTTTTGAAGCTTTGCAGGATCTTTATGGT
GAGCTTCTTGATAATGCAAGGTTAAACCAAAGTAAAGAAGTTATTTCAGA
TGATAAAAAGCATCTTGAGTTTCTGAGGTTCCAAACTGAGATTCATGAAA
GTTACTCAACTTTTCTTGAAGAACTTGTGGAGCAGTTTTCTGCTGTTTCTTA
TGGTGATGTTATTTTTGGCCGGCAAGTTTCACTTTATCTACACCGTTGTGTT
GAAACTTCCATTCGACTTGCTGCCTGGAATACACTGTCCAATTCTCGTGTT
CTTGAGCTTTTGCCACCTTTAGAAAAATGCTTCTCTGGTGCTGAAGGATAC
CTTGAACCCGCTGAGGTAAAAAAACATATTCATTCACTACCAGCTATTTTA
CTATGGAACATTATCCGCAGAGGATATTGTTTGAGTACTTGTTCTTTGATA
CATTTTTTCTTTCTATGTTGATGATATTGGGTTGAATTATAAATGTCACCTA
TATTTTATATTTTATAATTTTGTAAATTTTCAAAAAGTGGTTGTTTAAAATG
TGCTTAAAAGCATAAAAGCATATGCTACAAAGTACAGCTAATGTTGCAGA
ATTTTTAATTGTGTTACAAATTTACAATACCTTTGACATTTATTGGTTGTAT
TGTAGCATCTTTCATGAATTTTGAGGAAGCTTTTGAGAGCACAGGAAATA
CCAAACTAAGCCATACTGACACTGGGTCCTTTTATTTTTTATATGATACAT
AACAAAAGTGAAGTTCCTAATTGGAATTTTGGATATAGTCAAGAATCTTA
TACTTCCAACAATATAACAGATAGTCACTTTGGCACAAACATGCACATAT
TTTTTACAATGGCTTGCTTTAAAGCTTGTCTTTAGTAGGGAAGAAATGTAA
GGTTGTATCTTTGATTCTTTGAAGTTAGAAACTATCACGATGGTTTTGTCA
TGTAAGTTACCTCGGAGGAATGAGGATTGTTTTCATCAAGTTTCCTTATCT
AAAATTTTTATTTTCCATTTTTTTGCTAATATCTATAACTTATGTGAATAGA
AACAATTGGAGCTCAATGGGTAGGTGGGATAATTGTTTACTACTGTATTA
TCTGTGGAGGGGTGCACTTGCCACCTGTGTGGTTGCACCAGCAATGCATA
CTAGTCTTTGGCTCAGAATACACCATATATCTATCAACATTAATTGGTGTA
CTGTATCTCTGCAGGATAATGAAGCAATTTTGGAGGCTTATACGAATTTGT
GGGTTTCTGATGCCCTTGACAGGGCTGCAATTCGAGGATCAGTTGCATAT
ACTCTTGTTGTCCATCATCTTTCCTCCTTTATATTTCATGCCTGTCCCACGG
ATAAGTTATTGCTGCGAAACAGACTTGCTAGGTCTCTGTTGCGAGATTATG
CTGGGAAACAGCAACATGAGGTAACCAAAGTCTTGAAAGCTTTATCCTGA
TGGACAAATGGTAGTTTAAGCAAATCTCCATTCAGAATTTGTGCTGCCATT
CATTCTCCAATTAGTTTTAGCCCATCTGATTAAGGAAATCAAATGTTATAT
GTGAATGTGTGCTTGTTCTGTCATCAAGTTGTTTGGAATGTATATCAGCTA
GGACTTTGGCCTACCAAATAAAATTTTAAGAGGGTAGAGTGAGACATCAC
TGTTTAGGTAAAATCCTTTTTATGACCAAGAGTTTGAGGCCATGGAAAAA
GTATTATCTTTTACCTCCTAATTTAATTTGCATTGTTGCATCTGTATTTTCA
ATTATACATCCATAATGTTGGTGTGGCCAGGCATTACTATTTACTAATGGA
ACTCTTGTTGCAGGGTATGTTACTGAACCTCATTCACCATAACAAGCCACC
ACCATCTGTCATGGGAGAGGAGCTGAATGGTATACTTTCTGAAAAGAGTT
GGTTAGAGTCCAGATTAAAAGTATTGGTTGAGGCTTGTGAGGGAAATTCC
TCTATTTTGACAGTAGTAGATAAGTTAAAGGCTGTTGTAAAAAACAGTTC
ATAGTGAGGTAACTAATATTGAATGCGTGTAAATATATTCGAGATATACA
ATGAGCTATACATAAAGTACAACAGAGGCTTATATATAGATTTTTTCAAT
GATGTCTCAAATGTATTAATTGGAGCATGCATGGCCACTGATATTGGTAT
ATTTTGTTAGAATGGAGTCTGCTGGTTTTTTTATCAAGATTCTGGGAACCA
AGTCCTAAGGCATGCAATGTACATAATGAAAGGATACAGGTATTTTAGTT
AGGTTATTATAAAATATACTCTTTCATC
SEQ ID No: 22 RTR1 Oryza sativa ssp. Japonica Os05g04370.1
MGPTTATDTGARMKPTTVASAVHRVQMALYDGAAASREPLLRAAASLLSGP
DYADVVTERSIADACGYPACPNPLPSEDARGKAAPRFRISLREHRVYDLEEA
RKFCSERCLVASAAFGASLPPDRPFGVSPDRLDALVALFEGGGGGGDDGGLA
LGFGASGDGKEVEEGRKVEIMEKEAAGTGEVTLQEWIGPSDAIEGYVPRRDR
VVGGPKKEAKQNDACSAEQSSNINVDSRNASSGESGMVLTENTKAKKKEAT
KTPLKMFKQDEDNDMLSSCISDSIVKQLEDVVLEEKKDKKKNKAAKGTSRV
GKSKPAKRPVGRDGHEVDFTSTIIMGDRGSEMMDHGALGQYNFSSSILANEQ
PSSSQYAAIDSVQAYTEELDELFSNAVNIAKDETSDDSGRCTLRSSLKAVGSK
NAGHSVKWADENGSVLETSRAFVSHSSKSQESMDSSVRRESAEACAAALIEA
AEAISSGTSEVEDAVSKAGIIILPDMVNQQQYNNDYDNDKDAGENEIFEIDRG
VVKWPKKTVLLDTDMFDVDDSWHDTPPEGFSLTLSSFATMWAALFGWVSR
SSLAYVYGLDESSMEDLLIAGGRECPQKRVLNDGHSSEIRRALDTCVCNALP
VLVSNLRMQIPVSKLEITLGYLLDTMSFVDALPSLRSRQWQLMVLVLLDALS
LHRLPALAPIMSDSKLLQKLLNSAQVSREEYDSMIDLLLPFGRSTQSQASLPS
SEQ ID No: 23 RTR1 Zea mays, GRMZM2G065622_T01
MSPPAPAAAAAAAPRTVASAVLRVQMALLDGAAVSSEALIHAAASALLSRA
DYDDVVTERTISDVCGNPACPNPLSSSSAAATGPRFHIALSEHRVYDLEEARK
FCSERCLVASKALAASLPHDRPYGVPLDRLAAVVALVEGAAAGDGSGLGFQ
GLDGNGKVEDGGRKVEIKEKQVAGAGEVLLQDWVGPSDAIEGYVPRHDRS
AHGQKPQVQQNEGAGPELSRTENVDYGAAAPGEDGMTSSPSLVKTHVSSEV
IVERMGSLVLGENTRTPRKKKTKTPSKMLEQEEDNSMLSSCISDSIAKQLEDV
VLEERKGSQKNKMSKASSRAQKGKSTKRPASTNMEENAMNQYNYLSSSVL
VDNHPSSSQSSEKDSTQAYSEQLCEEFSEAVNIGNDETSDEKMRPAWKSSLK
VAGSKSSRQSVTWADENGSVLETSKAYESPSSSIKRPEEGIDNSLRRASAEAC
AAALVEAAEAISSGTAEAEDAVSNAGIIILPDMLNQQEHDNGKNSGGDDDPEI
DRDVIKWPKKPVLLDTDLFEVDDSWHDMPPEGFSLTLSAFGTMWAALFGWI
SSSSLAYVYGLERGSVEELLIANGRECPEKTVLKDGLSLEIRRALDSCVCNAV
PVLISNLRLQIPVSKLEITLGYLIDTMSFVDALPSLRSRQWQAVVLVMLDALS
VHQLPALAPVFSNSKLVQKMLNAAQVSREEYDSMVDLFLPFGRSVQAITPM
SEQ ID No: 24 RTR1 Glycine max, Glyma02g34860.1
MAKDKPVSVKDAVFKLQMSLLEGIQNEDQLFAAGSLMSRSDYEDIVTERSIT
NMCGYPLCSNALPSDRPRKGRYRISLKEHKVYDLQETYMFCSSNCLVSSKTF
AGSLQAERCSGLDLEKLNNVLSLFENLNLEPVETLQKNGDLGLSDLKIQEKTE
RSSGEVSLEQWAGPSNAIEGYVPKPRNRDSKGLRKNVKKECPFIIMFNVRPM
DVYGMTVNEMGFVSTIIMQDEYSVSKVPPGQMDATANHQIKPTATVKQPEK
VDAEVVRKDDDSIQDLSSSFKSSLILSTSEKEEEVTKSCEAVLKFSPGCAIQKK
DVHSISISERQCDVEQNDSARKSVQVKGKTSRVIANDDASTSNLDPANVEEKF
QVEKAGGSLKTKPRSSLKSAGEKKFSRTVTWADEKINSTGSKDLCEFKEFGDI
KKESDSVGNNIDVANDEDILRRASAEACAIALSSASEAVASGDSDVSDAVSE
AGITILPPPHDAAEEGTVEDADILQNDSVTLKWPRKTGISEADFFESDDSWFD
APPEGFSLTLSPFATMWNTLFSWTTSSSLAYIYGRDESFHEEYLSVNGREYPC
KVVLADGRSSEIKQTLASCLARALPALVAVLRLPIPVSIMEQGMACLLETMSF
VDALPAFRTKQWQVVALLFIDALSVCRLPALISYMTDRRASFHRVLSGSQIR
MEEYEVLKDLVVPLGRAPHISSQSGA
SEQ ID No: 25 RTR1 Glycine max, Glyma10g10540.1
MEKDKPVSVKDAVFKLQMSLLEGIQNEDQLFAAGSLMSRSDYEDIVTERSIT
NVCGYPLCSNALPSDRPRKGRYRISLKEHKVYDLHETYMFCCSNCVVSSKAF
AGSLQAERCSGLDLEKLNNILSLFENLNLEPAENLQKNEDFGLSDLKIQEKTE
TSSGEVSLEQWAGPSNAIEGYVPKPRDHDSKGLRKNVKKAEMGFVSTIIMQD
GYSVSKVLPAIVKQLGKVDAKVVRKDDGSIQDLSSSFKSSLILGTSEKEEELA
QSCEAALKSSPDCAIKKKDVYSVSISERQCDVEQNDSAKKSVQKFQVEKAGE
KKLSRTVTWADKKINSTGSKDLCGFKNFGDIRNESDSAGNSIDVANDEDTLR
RASAEACVIALSSASEAVASGDSDVSDAVSEAGIIILPPPHDAGEEGTLEDVDI
LQNDSVTVKWPRKPGISEADFFESDDSWFDAAPEGFSLTLSPFATMWNTLFS
WITSSSLAYIYGRDESFQEEYLSVNGREYPCKVVLADGRSSEIKQTLASCLAR
ALPTLVAVLRLPIPVSTMEQGMACLLETMSFVDALPAFRTKQWQVVALLFID
ALSVCRLPALISYMTDRRASFHRVLSGSQIGMEEYEVLKDLAVPLGRAPHISA
QSGA
SEQ ID No: 26 RTR1 Sorghum bicolor Sb09g002730
MSSPAAAAAAEAPRTVASAVLRIQMALLDGAAASNEALLHAAASALLSRAD
YDDVVTERTIADACGNPACPNPLPSSSSAAAATGPRFHIALSEHRVYDLEEAR
KFCSDRCLVASKALAASLPHDRPYGVPLDRLAAVVALVEGAAAAGDGSGLG
FQGVDGNVKMKDEGRKVEIKEKEVAGAGEVSLQDWIGPSDAIEGYVPRRDR
SAHGQKPQAEQNKVAGSDLSRTKNVDDRTAAPSEDGMTSPLSLVETHMSAE
VMAERMGDLVLGENTKTLSRKKKTKTPSKMMEQEEDDSMLSSCISDSIAKQ
LEDVVLEERKGSKKNKVSKASSRTHKSKSRKRPAGSDGHEVDFTSTIIIGDAS
TNREESAMNQYNYLSSSVLVDNHPSSSQSSAKDSTQAYAEQLCEEFSEAVNI
GNDETTDEKMRPALKPSLKVTGSKSGRQSVTWADENGSVLETSKAYESPSSS
IKQPNEGIDSSLRRASAEACAAALIEAAEAISSGTAETEDAVSKAGIIILPDMLN
QKEYGDAKNNGGDDDPEIDRDVIKWPKKPVLLDTDMFEVDDSWHDTPPEGF
SLTLSAFGTIWAALFGWISRSSLAYVYGLERGSVEELLIANGREYPEKIVLKD
GLSSEIRRALDSCVCNAVPVLISNLRLQIPVSKLEITLGYLIDTMSFVEALPSLR
SRQWQAVVLVMLDALSVHQLPALAPVFSNSKLVQKMLNAAQVSREEYDSM
VDLFLPFGRSVQATTPM
SEQ ID No: 27 RTR1 Brachypodium distachyon Bradi2g38650
MAPHAAAAAAGTTRTTMNVATAVYRVQLALLDGAAASNEPLLHAAAAVLS
RADYDDVVTERSIADACGHPPCASPLPAAAAAAAAPPRFHISLREHRVYDLE
EARKFCSERCLVASAAFAASLPHDRPFGVPPDRLDALVALFEGGGDRPGLGF
REVSSGKDKDEGRKLEIREKEAPGLGEVTLQEWIGPSDAIEGYVPRHHPIPEGP
MPEAKQRKTSRADQSRNKNLDSATSSSVEAPVSSEVIAKKLNDMVLGDNTK
TKKKQVCETPSKMFRPDEHGDMLLSCVTDSIAKQLEDVVLEEKNDMKKERP
TRASSRSRKSKPAKKPAGSDGHEVGFTSTIIMGDHVLAKMDQGPVGQYNFAT
SIADNQPSSSSSLSSSPTQYTARDLTGAYTEQLNKEFSKAVNLGKDEASDEKV
RIVPKSSLKAGGSKNKSQSVTWADENGSLLEISKEYVIHSDDKKHYKEDIDGS
LRRESAEACAAALIEAAGAISLGTSEVEDAVSKAGIIILPDMLHQNQFKSDNG
KNTVEKEISETDNGVVKWPNKPVFLDTDMFEVDDSWHDTPPEGFNLTLSAF
ATMWATLFGWISRSSLAYVYMLDGSSVEELLISSGREYPQKTVSKDSQSSEIK
RTLATCIGNALPVLTSNLRMQIPVSKLETTLGYLIDTMSFVEALPPLRSRQWQ
LMVLVLLDALSVCRLPGLAPVMSDSKLLQKVLNSSQVSREEYDSMVDLFLPF
GRSVQTPPPSQPVQVP
SEQ ID No: 28 RTR1 Oryza sativa ssp. japonica, >0S05G04370.
Gene
CTCTTCGTCTCGACTCTGAACTAAAAACTCAACTCCTCCCAAAATCCCTCG
CCGCCGCCGCCGCCGCCGCCGACGACGACGACATGGGCCCCACCACGGC
CACCGACACCGGCGCGAGGATGAAGCCCACGACCGTCGCGTCGGCGGTG
CACCGCGTCCAGATGGCGCTCTACGACGGCGCCGCGGCGTCGAGGGAGC
CGCTGCTCCGCGCGGCGGCCTCGCTGCTCTCGGGGCCGGACTACGCCGAC
GTCGTCACGGAGCGCTCCATCGCCGACGCCTGCGGGTACCCGGCGTGCCC
CAACCCGCTCCCCTCGGAGGACGCCCGCGGCAAGGCGGCGCCGCGGTTCC
GCATCTCGCTCCGGGAGCACCGCGTGTACGACCTCGAGGAGGCCCGCAAG
TTCTGCTCCGAGCGCTGCCTCGTCGCCTCCGCCGCCTTCGGGGCGTCGCTC
CCGCCCGACCGCCCCTTCGGCGTCTCGCCCGACCGGCTCGACGCCCTCGT
CGCGCTCTTCGAGGGCGGTGGTGGTGGTGGTGATGACGGTGGGTTGGCGC
TAGGGTTTGGGGCGAGCGGCGATGGGAAGGAGGTGGAAGAGGGGAGGA
AGGTGGAGATCATGGAGAAGGAGGCGGCTGGGACGGGGGAGGTGACGCT
GCAGGAGTGGATTGGGCCGTCGGACGCCATCGAGGGCTATGTGCCTCGCC
GTGATCGCGTCGTTGGAGGTGAATCGCCTTTTGCTGACCTCTTCTATTTCT
ATTTGCATTGAGCATTGATTGGTTTTTCTGTTCAAATTATTTACTCGTTTGG
TTTGATTGTGCTGCCATAGGTTAGTAGTACGATGAGTGAGGTTTATGGCG
GTGGCATTCTCTGTTATCCTTTGTGCTCAAATTAGGTAGAGAAAAAACTTT
GGAAGATAGAAAATGGAGGAATTTGTAGTGTTCGTAGTAAATTACTTTGA
GTATCAACAATTGTTGTTGCTAGTAGCAGTTAGGAAAATTCTCTGTAACAT
GTACAACTACAACATTTTGGTGAACTAGTTATAAACTCCTTGTGTAAAATC
TCCCTACCTGATTAGCAAATTAGTAGCATTTGTGTATGACTAGAAACAATC
AACTAGGTAATAATTGAGAGCTATCAAATATGGTAAGGGACAGCTGAAG
AATAGACATAACCGTGGTGCCATATTTTTTGGTTGCAGTCATTCAAATTAC
AAGAGGACAACTAGGCAATTCACTCATTTTGTTTAGACCTACATATTGTTG
TTGATAAGGGACGCTAGAACGAAGATAGGAGATTGAGAGAGTAATTAAT
TTTTGGAAGTACGTTACAGATTTTAATGGGAGGTAGTGACTTCTTTTTTGA
GAGATTAGAGGGGTGAGAGATGAGCTGCTTACTTTGTCAATGAAAAGCGC
TCAGGTTTTGTTCAATATTTCCCCGAAGAAATACAAAAATCATGTCTTTGT
AAGCATCATGTGTTGTTCCTCTTTTCTCACTGCTCACGGAGGTGCCTAAAC
TGCCTAACAAGGTGGCAAACTGGTCCCTAGTGCCTAAACACCAAGCCACC
AAGGCAGTGGTGGTTTGGTGGGAGTGTGGTGGTACTTAGCTCCTACCTCC
TAGGCACTGTTTGTTTTTCTTATATTCAAATAATGTTCTAGAACTAGATGT
GACAATGTTACAAGAGTAACAATAGTTTTACTTGCAGGCACATTTATATA
TATGAAAAGGATATGCTCATTCTTGCTCTTTGTTCGACCTGTCTTAGTTTC
GAACAATTGAAGAAACAATGAGCTTGTGTTCTGTATTTCAGGGCCAAAAA
AAGAGGCTAAACAGAACGATGCTTGTAGTGCTGAGCAGTCCAGTAATATT
AATGTGGATTCTAGGAATGCTTCTTCTGGTGAATCCGGCATGGTTCTTACT
GAGAATACAAAAGCAAAGAAAAAGGAAGCAACCAAAACCCCATTGAAG
ATGTTCAAGCAGGATGAAGATAATGATATGTTGTCGTCTTGCATATCGGA
TTCCATTGTGAAGCAGCTGGAGGATGTAGTTCTCGAAGAGAAAAAGGATA
AGAAGAAAAATAAAGCAGCTAAAGGAACATCGAGGGTAGGTAAGAGTAA
GCCTGCAAAAAGACCAGTTGGGCGTGATGGACATGAAGTGGACTTTACAA
GTACAATTATTATGGGTGATCGTGGTTCAGAAATGATGGATCATGGTGCT
CTGGGTCAATATAATTTCTCAAGTTCTATATTAGCAAATGAGCAGCCTTCA
TCATCTCAATATGCAGCGATAGATTCAGTGCAAGCTTACACTGAAGAACT
AGATGAATTATTTAGTAATGCAGTTAACATTGCAAAAGACGAGACAAGTG
ATGATAGTGGTAGATGTACACTAAGATCTTCATTGAAGGCTGTTGGATCC
AAGAATGCAGGGCATTCTGTGAAATGGGCAGACGAGAATGGAAGTGTGT
TAGAGACAAGCAGAGCATTTGTAAGTCACTCCAGTAAATCTCAAGAAAGC
ATGGACAGTTCAGTAAGGCGTGAATCTGCAGAAGCTTGTGCAGCTGCGCT
TATTGAAGCAGCAGAAGCTATTTCATCTGGCACATCGGAAGTAGAAGATG
CAGGTGAACACTTATTCCTTAACCCTGTGGTGCTTTGACATGCAGCTTTTG
TTTTTGATATGTATTAACCTGTGCCTTTTGGTAACAGTTTCAAAGGCAGGA
ATCATCATACTGCCGGACATGGTTAACCAGCAACAGTACAATAATGATTA
TGACAATGACAAAGATGCAGGGGAAAATGAAATATTTGAAATTGATAGG
GGTGTTGTGAAGTGGCCGAAGAAGACTGTGCTTCTAGACACAGATATGTT
TGATGTCGATGATTCTTGGCATGATACACCACCAGAAGGCTTTAGTCTAA
CTGTAAGAATTCTTGAGAAAAAATAAAGTAGCACTTCTTCTTTTTTTTCCT
TCTGGTTGAATTTGGCATGTCATGCCTTTTTTCCTTTTCAGCTGTCCTCCTT
CGCAACGATGTGGGCTGCATTATTTGGATGGGTATCCCGGTCCTCATTGGC
CTATGTGTATGGGCTTGATGAAAGTTCTATGGAAGATTTGTTGATTGCAGG
TGGAAGAGAATGTCCTCAGAAGAGAGTTTTAAATGATGGCCACTCATCTG
AAATTAGAAGAGCTTTGGATACTTGTGTGTGTAATGCCCTGCCAGTTCTTG
TATCAAACTTGAGGATGCAAATTCCAGTCTCAAAGTTGGAGATTACTCTG
GTATGGATAAAGCTAGTTGAACAAACTAATTAAAAAGTTAAAACATTTTT
TAAAAGAAAAGAATAAGGACTCCGCAATTGGTTAGTGACCTAGTTGAACC
TGGATATGTATTTACTGGCTAGTACATATAGTTTTTCAGCAGCGGCTGTGA
AGAGGCTTTTCGTTGCATTTTGTTCTGGTATCTAGACACCTTTGACTGAGA
TCAATCAAGCTACTAATATCTTGGTCAGTTTAACAATAATTTCTTAGTCAT
TTGGGGTTCTCTCTCATTCATAAGTGTGGGTGATGAGCTGCGCATATCTGG
AAATAGATAAGACGAAGTGGAGGCTGTACGTTACAGTTATTCTGAATTAG
GGCCAGTCTCTTTTTTGTTGATTCTATTGAAGACAGCTTAACTGCTAGATG
TTGCCACCAAGAATTTTATTTGTTAGTTTGATTACAGTTTCAGTAATTAAC
ATTTAGAGCTATAATTAGTGTGCATCTAATGTGTTTTTCCCCTCCCCACTA
GGGATACTTGTTGGACACGATGTCATTTGTTGATGCACTGCCTTCTCTGAG
ATCAAGGCAGTGGCAATTGATGGTTCTCGTGCTGCTTGATGCGCTCTCACT
CCATCGGCTTCCTGCTCTTGCTCCAATAATGTCAGATTCGAAGCTTTTGCA
GAAGGTGATGATGCCTTTCCCTGCCTTTCTGCTATGCAAGTAGACGGATGC
ACATATTCTTTTTAAGAAATCTGATCTTTCTTCCCTTTTGTGTGCTGCAGCT
TTTGAACTCGGCTCAGGTTAGCCGAGAGGAGTATGACTCCATGATTGATC
TCCTCCTCCCTTTTGGAAGATCCACGCAGAGCCAGGCATCCCTGCCAAGTT
AAACCTCAAGCAACACAAGTATCTAAATACATGTTTACACAGCGGAGTAA
ATAGAGAGAGGTTCTACATATCAGCGTCAGCTTGGCAGTCTATTCGGATA
TACTATCATAATGGTGCCTCTTGCGTCTGGTTGTTTAGGTTTCGGAACACG
TCTTGCAAAATATCGGTTGCTCCCAGTTGCTTCAACTATCCCCTTTGCATT
GGCGTCAGACGTACAAGCGGAGCGGGCACTGATTCCTGTCATTCTCAGGA
ATTGTTAGCTTTAGTGAGGAGAGCAAAAGATAACTGCCTCCAGAACAATT
TGGTCCACTATGGACTTTTATCTCTCATTGCTAGCGCAGTTCAGAATTGTA
GTGCATCTTTGGGTTTTTTTTTTACCTCTTTTACCGGTGTGCAGTTTGTCAG
GTTAGTACAGCCGAATTTGACCAGCTAACAACCCACTCGTGAGTCCTAAT
AGGTGAGTGCTAGATATCAAGTGGTATAGTAAACCCAGTGTTAGCATCTA
TAAAATTCTGAATTTTATGCCTGTCAATCTCGC
SEQ ID No: 29 RTR1 Zea mays, >ZM08G20550, Gene
GCTTGAACTCATTTGCAATTGCGAACGCTTCTTCTCCCAGTCCCAGCACGC
CCAACCCCGTCGCCGCCTTCTCGAACCTTCCGAAGATGAGCCCCCCGGCC
CCGGCCGCCGCGGCGGCGGCGGCGCCGCGAACGGTCGCCTCGGCGGTGC
TCCGCGTCCAGATGGCGCTCCTCGACGGCGCCGCGGTGTCCAGCGAGGCC
CTCATCCACGCGGCCGCCTCCGCGCTCCTCTCCCGCGCCGACTACGACGA
CGTCGTCACCGAGCGCACCATCTCGGACGTCTGCGGCAACCCCGCGTGCC
CCAACCCTCTCTCCTCCTCCTCCGCCGCCGCCACGGGGCCCCGCTTCCACA
TCGCCCTCAGCGAGCACCGCGTCTACGACCTCGAGGAGGCGCGCAAGTTC
TGCTCCGAGCGCTGCCTCGTCGCCTCCAAGGCCTTGGCCGCCTCGCTCCCG
CACGACCGGCCCTACGGGGTCCCGCTCGACCGCCTCGCCGCGGTCGTCGC
GCTCGTTGAGGGCGCCGCCGCAGGGGACGGGAGCGGGTTAGGGTTCCAG
GGACTGGATGGGAATGGGAAGGTGGAGGACGGGGGAAGGAAGGTGGAG
ATCAAGGAGAAGCAGGTCGCCGGGGCTGGCGAGGTCTTGCTGCAGGACT
GGGTTGGGCCCTCCGATGCCATTGAGGGTTATGTGCCGCGCCATGACCGC
AGTGCTCATGGTGAGTATCCCATACTTTGTTTGCCCATGATTGCATTTTAT
TATTTTGTAAATATCAATTGATTGAATGACGGAAAGGCGAGAACAGAGCC
CAATGGTGGTGTTAGTGTCTTGTCGTAATGCTGAAATATTGCATGGAGAT
ATGTTGCTTTAGACTTCAGTGTTGCAGTTTATGTATTTGGTCTTATATATTT
CCATAGGTGTTTGCTCATGAATAACTTGGGTAGGTTGAATTGGTTTCCGGA
ATAAGATATGATTCTATGTTGATATTGTTACTTTTAAGTTTTTAACATGTA
AATATAAGTAATCTTTACCTGTGGTTGTGTCGCAATTAGTAGTTTATTTGC
ATCCTGCTAGTGCATCTAGTTAGCAACCTTTTTGTATACATGAAGACTATT
TGGGTGAAAGGAGCCAAACTTGACACCTACTTGCATATTTTCCATGTGAT
GATCGTGTCTATGGCGGTTGACGTGTGCAGCCTGCAGATGTTTGTGTTATT
GTTTAAGTGTGTATGCTAGTAGAAATTAAAGAAAAATAGAACAATAGAG
GTTGTTGCTCAATGGTTTTCTGACTATTACTCCTTTGCACCATGATGTGTTG
AGGTCATTCACCCTTGGAGTTTTAGTCAGTACCCTTGATTTAGGGTCTGTT
TGGTTGGGCTGTGGCTGTGGAAAAAGTTGCTGTGGGCTGTGAGCTGTGGA
AAAAGCTGCTATAGGCTGTGTGCTGTTAAAAAGCTAAAAATCGTTTGGTG
GAAACCACTAAAAGTCGTTAAAAGTTCTTTGATATATGTTTTCACAGTTCC
ATCCAAAAGCCACTAAAAGCAGGTCCAGGGGTGCTTTCAGTTTTGCACTA
CGAGAAAGTCGGCTTTTAGAAAAAGCTGCTTCGTGGATCCAGCCCTTTGG
TTGGCTTTTGGCTTTTAGGGGGCAAAAGCCAAAGCCAAAAGCCAAACCAA
ACACACCCTTAGTATCTCTACTGCTGCATTTTGCACATCATAACTTTGCTA
CTGATGCAGTTCAGCCTGCACTTTGCAGCACACTGACAATTTATAGGGTCC
TGCCTGCAAGCATCTTGGGGTTGATATTGATTTAACACACCAATTTTTTAG
TCACTGGTGTAAGTAAGAATGTTTACCAGAGTAACGGTAGTTTTTCTTGCA
TGCACATTTCTGTACATGAAAGGATCCTTATCTCCATTCTTGCCCTTTGTTC
GACTTGTCTTAAATTTTGAACAATTGAAGATACAGTGAGCTTATGTTCTGT
TGTCAGGACAAAAGCCACAGGTTCAGCAGAACGAAGGTGCTGGACCTGA
ACTGTCCAGAACTGAGAATGTGGATTATGGTGCTGCTGCTCCTGGTGAAG
ATGGCATGACAAGTTCACCTTCATTGGTTAAAACACACGTGAGCTCCGAA
GTAATAGTTGAGAGAATGGGCAGCCTGGTTCTTGGTGAGAATACAAGGAC
GCCTAGAAAGAAGAAAACTAAAACTCCATCAAAGATGTTAGAGCAAGAG
GAAGATAACAGTATGCTGTCATCTTGCATATCTGATTCCATTGCCAAGCA
GCTTGAGGATGTAGTTTTGGAAGAGAGAAAAGGCAGTCAGAAAAATAAA
ATGAGTAAAGCATCATCAAGAGCACAGAAGGGTAAGTCTACAAAAAGGC
CTGCTTCGACAAACATGGAGGAAAATGCTATGAATCAGTATAACTACTTG
TCAAGTTCTGTATTGGTAGACAATCACCCCTCATCATCTCAATCTTCAGAA
AAAGATTCAACACAGGCTTACTCTGAACAACTGTGTGAAGAATTCAGTGA
AGCAGTGAACATTGGAAATGATGAGACAAGTGATGAAAAGATGAGACCT
GCATGGAAGTCTTCGTTGAAAGTTGCCGGGTCTAAGAGCAGTAGGCAGTC
TGTTACATGGGCAGATGAGAATGGAAGTGTCCTAGAAACAAGCAAAGCA
TATGAAAGCCCTTCAAGTAGTATAAAACGACCTGAGGAAGGCATAGACA
ATTCACTAAGGCGTGCATCTGCTGAAGCGTGTGCTGCAGCACTTGTTGAG
GCAGCAGAAGCTATTTCTTCAGGCACAGCAGAAGCAGAAGATGCAGGTG
AGCATGTATTCATTATGTTCCCACGGCTGCTATTCTTTGAGGCTAACAACT
TTTGTTATTAAATGATACTGACGTAAGCCTCTCTAACGTCAGTTTCAAATG
CTGGAATCATCATTCTGCCTGACATGCTTAACCAGCAAGAACATGACAAT
GGCAAAAACAGTGGCGGAGATGATGACCCTGAGATAGATAGGGATGTTA
TCAAGTGGCCTAAGAAACCTGTACTTCTGGATACAGACTTGTTTGAAGTT
GATGATTCTTGGCATGACATGCCTCCAGAAGGTTTTAGTCTAACTGTAAGT
ATTATTAAGAAGGGAAAAAAGAGAGGAGAAATTCCAGTTTTGCTTTTAAT
TCGGTCTCAGCACGATGTACATTTCTTTTCAGCTGTCTGCTTTCGGGACGA
TGTGGGCCGCGCTATTCGGATGGATATCCAGTTCGTCTTTGGCCTATGTGT
ATGGGCTTGAAAGGGGTTCAGTGGAGGAGTTGTTGATTGCCAATGGGAGG
GAATGTCCTGAGAAGACAGTTCTGAAGGATGGGCTCTCATTGGAGATTAG
AAGAGCTCTAGATTCTTGCGTTTGTAACGCCGTGCCAGTACTCATATCAAA
CTTGAGGTTGCAGATACCGGTTTCAAAACTGGAGATTACTCTGGTACGTG
TCAACTTTACCAAGCAGATAATAATATCGTACCTTTTTTAAAATAAGGCTG
CTATAGGTTTAGCTTGATGCTGGTGGCTGACAGTTATATCGGCGGTAACA
CATGATGAAACTACACCGTGTCTGTACCAGGGCTACTTGATTGACACAAT
GTCGTTTGTTGACGCCCTGCCTTCTCTGCGATCGAGGCAGTGGCAGGCTGT
GGTTCTGGTAATGCTTGACGCGCTCTCTGTGCACCAGCTTCCCGCCCTTGC
TCCAGTCTTTTCGAATTCGAAGCTTGTGCAAAAGGCGAGTGACCAGTTTTT
TTGTGGTTAGTTGAATAATATATGTATATCTTATTTTGCTTTGGTGACATCT
GAATTTGTTTCCCCATCAATGTGCGATGCAGATGTTGAACGCTGCTCAGGT
TAGCAGAGAGGAGTATGACTCCATGGTGGACCTGTTTCTACCGTTTGGAA
GATCCGTCCAGGCGATCACGCCCATGTAAACGAGAAGCCGTGTGAATCTG
CATTCCGGAAGCTGCGTGAATCGTAGGGTCTGATCTGATATTTAGTTTTAC
ACAAGTCGCTGTCTAGAACGAGCCATGTATGTATGATTGATTGGTTATCTA
TTGAGCAAGATGCGCTCTGGCAATTTGTGGAAGCTGAAATACTTGCGACC
ATGACGCCTGCCTGTCAGTTTGCGATATTCTTTCAGGTTGTGAGAAATTGA
TTTGCTGTCGTTCTCAGCTATTGTTAGCTTTATGCCCTCTTTGAACTCCTAG
AGCTAATAGTTAGCCAGCTAAACAGCCGAGCTAGCTAATAAACTAACTAA
GCTAACTAATAAACTAATTATTAGTTGTGAGTTAGCTAACAATTAACTGG
ACTATTAGCCTTGGATCTGAACTTTTCCCCCTCTGCTCCTGTTTATCCCTGC
GTTTTTACTTATATACAACGGGCAGTTTCACGTAATTACATGGGCAGTTTC
ACGTAATTACATGACATTTTTGGCATTGTGTAGAACTCATAAAGCTAACG
CTAATAGAGTGTGTTTGGTTTGGTTTGGTTTTAGCTTTTGACAATTAAAAT
TCAAAAGCTAAACCAGAGGGTTCGATCCAGTAAACATTTTTTTTCTCTAAA
AATCGACTTTCTCATAGCACAAGACAGACTGAAAGCATCTCTTCACCTATT
TTTAACGGCTTTTGGATGAAACTGTGAAAATATATATGGAAGAAATTTTA
GCATCTTTTATTGGCTTCCACCAAACCGATTTTTTTGTTGCTTTTTTTACAT
TTCATAGCCCACATTAGTTTTTTATAGCTCACAGCACACAACAACTTTTTT
CATAGCCACAACCCAAACTAAACACACCATAGTTAGCTAGCTAATAATTT
GTTAGCTTGAATAACCTAATTAACTGTTAGCTAGGATCTACTAATTTTTTA
TAGTACCTTGCTAAAAGTAAATGGATTTCACACTCCAAACATAACACAAA
CAACACCAGTCTATTAAAAAACATATAAAA
SEQ ID No: 30 RTR1 Glycine max, >GM02G34860, Gene
TTCAAGCTATTTGGTTCTGAAGCTTTTGTTCAATGGCAAAGGACAAGCCTG
TTTCTGTCAAAGATGCCGTCTTCAAATTGCAAATGTCACTCCTTGAAGGCA
TTCAAAATGAAGACCAGCTGTTTGCTGCCGGGTCTTTGATGTCAAGGAGT
GACTACGAAGACATTGTAACCGAACGATCCATCACAAACATGTGTGGTTA
TCCACTCTGCAGCAATGCTTTGCCATCCGATCGCCCGCGGAAGGGTAGAT
ATAGGATTTCACTGAAGGAGCACAAGGTCTATGACCTACAAGAGACTTAC
ATGTTTTGTTCTTCAAATTGTCTTGTTAGCAGCAAAACTTTTGCTGGGAGC
TTGCAAGCTGAGAGATGCTCTGGTTTAGACCTGGAGAAACTAAACAATGT
TCTTAGCTTGTTTGAGAATTTGAATCTGGAACCGGTGGAGACTTTGCAAA
AGAATGGAGATTTAGGTTTGTCTGATTTGAAAATCCAGGAGAAAACAGAA
AGAAGCAGTGGGGAGGTGTCTTTGGAGCAGTGGGCTGGACCTTCAAATGC
AATTGAGGGATATGTACCAAAACCAAGAAACCGTGATTCTAAGGGTTTGC
GGAAAAATGTTAAAAAAGGTGAGGATTTTTTTGGGTTTTCTAGACAATTG
TGAGAAACTAATCAGTTAGCCATATGCGATTTCTTGCTTAAGAGTGTCCTT
TTATTATCATGTTTAATGTTAGGCCTATGGATGTTTATGGCATGACTGTGA
AGTCTGAGGCTGAGGCTTAATTTATGTGAAATGGAATACTATAATTACTA
TTTCTTATCCTTTTTTCTCTATCCAATCAAGTGAGCTTTGATTCTGCATTGC
AGGGTCCAAAACTGGTCATGGCAAGTCAATTAGTGACATAAATTTAATTA
ACAGTGAGATGGGCTTTGTGAGTACTATAATTATGCAAGATGAGTATAGT
GTTTCAAAAGTACCGCCAGGTCAAATGGATGCAACTGCTAATCATCAAAT
TAAACCAACAGCTACAGTCAAGCAGCCAGAAAAGGTTGATGCTGAAGTG
GTCAGGAAAGATGATGATAGCATTCAAGATTTGTCTTCATCTTTTAAGAG
CAGTTTAATTTTAAGCACCTCAGAAAAAGAGGAGGAAGTAACTAAATCAT
GTGAAGCTGTGCTCAAATTCTCCCCCGGTTGTGCTATTCAAAAGAAAGAT
GTTCATTCAATCTCCATATCAGAAAGACAATGTGATGTGGAACAGAATGA
TTCTGCTAGGAAATCTGTACAAGTCAAAGGGAAAACGAGTAGAGTTATTG
CTAATGATGATGCTTCCACTTCCAATTTAGATCCTGCCAATGTTGAAGAGA
AATTCCAAGTGGAAAAAGCAGGTGGATCATTAAAGACTAAACCCAGATCT
TCCCTTAAATCTGCTGGTGAAAAGAAATTTAGTCGCACTGTTACTTGGGCG
GATGAGAAAATCAACAGCACTGGGAGTAAAGATCTTTGTGAGTTTAAAGA
ATTTGGAGATATTAAAAAAGAATCTGACTCAGTAGGAAATAATATAGATG
TTGCCAATGATGAAGATATATTACGTCGTGCGTCAGCAGAAGCTTGTGCT
ATTGCATTGAGCTCAGCATCAGAAGCAGTTGCCTCTGGAGACTCGGATGT
CAGTGATGCTGGTAATTACTGTTTCTTTTACGAGTTTTCTATTTAAATTAAT
TGGTCTGTAAAAGTTTTCTCGCCTATGAATGAAACTTGTGCAGTTTCTGAA
GCTGGAATCACTATATTGCCACCTCCACATGATGCTGCTGAGGAAGGTAC
TGTGGAGGATGCTGATATACTACAAAATGATTCAGTTACTCTGAAATGGC
CAAGAAAGACTGGAATTTCTGAAGCTGATTTCTTTGAATCTGATGACTCAT
GGTTTGATGCTCCACCAGAGGGTTTCAGTTTGACTGTAAGTTTAGTGAAA
ATTTTAGAAACATTCAAAGCTGTATTAATTTCCACAGTATATTCTATGGCA
TAACTTGTGCTTTCTTTCTTTCCTTTCTTTTTTTTTATAATTTTTTGTTTGTTT
GTGTGTGTAACTTTATTTCAGTTGTCACCTTTTGCAACTATGTGGAATACC
CTCTTTTCATGGACAACATCATCTTCTTTGGCATATATATATGGGAGGGAT
GAAAGTTTTCATGAAGAATATCTATCAGTTAATGGCAGAGAATATCCTTG
CAAAGTTGTCTTGGCAGATGGTCGCTCATCTGAAATAAAACAAACTTTAG
CCAGTTGTCTTGCTCGAGCTTTACCTGCGCTTGTTGCTGTGCTCCGGCTGC
CAATACCAGTATCTATCATGGAGCAAGGGATGGTAGGATCTTGGAGTTAT
CTATTTTTGTAGTTTATTTTGTAGTATAGTTGTCCCAAACTAAGAGTCATT
ACAGATTTATTATACTTCAGCAGGCATGGTCTGGTTTGTTTTACACCATTC
ATTGGGACTTAAAGGAAAATCATTTAATATAAGCATATGCATGTTAATTTT
TGTGAGAAATAGATATGGATCCATTGCTTGAAATAAGAGACACGTGGAAA
TTAAAATATTTGAGCCTGTAGGATAAATACTTCATGCTATTGAAAAGAAA
AGAAATAGAATTTGGGGAGAGGGGGTGGGGTGCTGGTGAGCCCCTTGTAT
CCGTCCCTGCCTGCATAGTCCTTGCTATTTAAATTCCTTAATCATTATGTG
GTTCGATTCAGGGAAAATCTGCAATAATTGCAAGATACCTAGTTGCATTG
GTCTAAGCACTTAAACAAACTAATAGTGACTTCTTTTTGCTGGAAGCAAA
TACTAAAATGATTTCTCCATGTTACATTGATTTAGCATCTGCAACACATTA
ACTGCTTTTGGTTGACAATTAGGAGGATTCTTGGGTTTTAAAATTATGCCT
TATTGTCTGAGAAGACAATATAATTTTTGAGAATCAGTTTAGAAATCAGT
GAAAGTAGGGGAGTCTTGGCACAGGGGTTTCAAGCAATGGCATCAGCTTC
TTGCAGAGACAATGCTGGTTACTACTTCCTACACATATTGGGTCCTACCCT
TCCCTGACCCAACACATAGCTTTATAGCACAAGGTTCCCTATTTTTAAAGT
TTAGAAATTTCTAAATTCATGAGTTTTAGGCTATAAGTTGCAGATGGATTT
AATCTTCATATCTGTATTGAAAAATTCTTTAATATATCCATTTACTTGGTG
CATGGATGGGTTATATGTTAATTCTCGTGACTTTCTATTAGGCATGCTTGC
TGGAGACAATGTCATTTGTGGACGCACTTCCAGCTTTCAGAACAAAACAA
TGGCAAGTGGTTGCTCTTTTGTTCATTGATGCATTGTCCGTATGTAGATTA
CCTGCTCTTATCTCATACATGACGGATAGGAGGGCTTCATTTCATAGGGTG
AGAACCTTTTGTTTATATCATACTAATTTATTATTTTATATACATGATGGAT
AGGAGGAATCTGTTTGCGGTGCATGAAGCATTATATAATGATGTTGGGTT
GTATATATATTCAGGCAAAAAAAGCATAATCCCATATTATGGCTCTGATC
TTTTGGTTAGGGTAGTTCAGTTAACATCTGATATCTCCATTATTTCAATTG
CTAAAATTTTGGTTACTTTTCATCTGCATTTCCTTTATAAATGTTTTGTTGC
CCAATTCTTGATCAAATGGGAGCTTAACAACATAAATCCCACACAACTTA
GCTACAATGTGCTTAATGTTGTTCCCCTTCTATAGCTTTAGACTCGATGGC
CTTATTTTATGATTCTATAGTTTAGTTCATCTAGTGTGGTTCTCCACTTCTC
CTACCATGGCTGTGGACTTTAAATTCTTGTTTGGTTTTCCATCATGTATTTT
CATGCTAATTTTGTTTGAAGTCAGCTAAACCACAAGTCGTGCACATAGATT
GCCATAGGCACAAATTTATGGCACGTGGACATGGAACCCAACTTGGAAAT
TTTTTTCTTCTAATTTGATTCCCTACATATGTGTGTTGGGGTTGCAACAATT
TTGATATGACAGCTGGAATATGGAGGTTAATGATATATCTTATTGTTTTGT
TGTTTTATCTCCTGTTATGTCAGATTTTGATGTTATAATTTTCACTCTTCTTT
TGAACCTGTTAACCATTGTTCTGGTATTGATTTGGCGAAACAGGTTTTGAG
TGGTTCTCAAATACGTATGGAAGAGTATGAGGTTTTGAAGGATCTTGTAG
TACCACTGGGCCGAGCACCTCATATCTCTTCCCAAAGTGGGGCATGACAA
CAAATAAATGTTGGACTTCACCAACGATGAGCGCAATATTATATGCTCTA
ATGCTAGCTAGCGAGTGATAAATGCAAATTGATATTCAAAATAGACATTG
TTCTAAAATATTGTCTTCCTGTTAAATGGCAGTTTGTATGTTTTTATTTTAT
TTTATGCCAAGCAGTCTGTAGAAGGTTCTGAAGATGTCTATTGGATTACTA
ATCTTAGAGGTGCATGTCAGGATGGTTAATACAGAACAAATATCTTCCCG
ATTATAAGATTGATTTTACAGATTTAATTT
SEQ ID No: 31 RTR1 Glycine max, >GM10G10540, Gene
GCTGAAAGGAAAATAGAGGTTTCTCTTTCAAGTTTCACCTCCTCACCACCG
CCAAGCTTCGTCTCCGAGAATGTCACCATCGCACACTTTTTTGTTTCCAAG
CACAGCACCGATGATATTAGCAACCAAACCCTAATCGTGTCCACGCTTTC
TTTCTTCTTTCCTGGCACACTTTCTCCCTCTCCACTTTCAGCACACACTTTT
TCGTTTTCTGGTAAGTCCTAATTTAGAGAACTGTGTGATGGATTTGAAATT
AGGGTTAATGAAAGTGCATTTCTTCTGTCCAAATTAGGGTTATCATGGAA
CCTAATTTTACGGTCTAAATGCGTTAACGTTCAAGGACGACTACGAATGT
GATTATTGTAATATATAATCATCAATGCAATTACTTTTATCTTTCATGTGCT
TTAATAATAGAATTATGATTGTGTCTTGTGACTCTGAAATTTTGTTGGGTT
GAACTGTTGAAGCTATTCTTATTGTGGTGTACTTTGTGCATGATGCAGTTC
TAGTTATTTGGTTCTGAAGCTTTTGTTTCAATGGAAAAGGACAAGCCTGTT
TCTGTCAAAGATGCTGTTTTCAAATTGCAAATGTCGCTCCTTGAAGGCATT
CAAAATGAAGACCAGCTGTTTGCTGCTGGGTCTCTGATGTCAAGGAGTGA
CTACGAAGACATTGTAACCGAACGATCCATTACAAACGTGTGTGGTTATC
CGCTCTGCAGCAATGCTTTGCCATCCGATCGCCCACGGAAGGGTAGATAC
CGGATTTCACTGAAGGAGCACAAGGTCTATGACTTACACGAGACTTACAT
GTTTTGTTGTTCAAATTGTGTTGTTAGCAGCAAAGCTTTTGCTGGGAGCTT
GCAAGCAGAGAGATGCTCAGGTTTAGACCTGGAAAAACTAAACAATATTC
TTAGCTTGTTTGAGAATTTGAATCTGGAACCAGCGGAGAATTTGCAAAAG
AATGAAGATTTCGGTTTGTCTGATTTGAAAATCCAGGAGAAGACAGAAAC
AAGCAGTGGGGAGGTGTCTTTAGAGCAGTGGGCTGGACCTTCAAATGCAA
TTGAGGGTTATGTACCAAAACCAAGAGACCATGATTCTAAGGGTTTGCGG
AAAAATGTTAAAAAAGGTGAGGATTTTTTTTTTGGGTGTTCTAGAGTCTAG
ACAATTGTGAGAAACTATAGATAAATAGATAGCCTTATGCAATTTCTTGC
TTAAGAGTGTCCTGTTTATTATCATGTTTAATGTTAAGCCTATGGATGATT
GTGGCGTGACTGTGAAGTCTGAAGCTGAGGCTTAATTTATGTGAAATGGC
ATACTATAATTACTGTTTCTTATCCTTTTCCACTATCCAACCAAGTGAGCTT
TGATTCTGCATTGCAGGGTCCAAAGCTGGTCATGGCAAGCCAATTAGTGA
CATAAATTTAATTAGCAGTGAGATGGGCTTTGTGAGTACTATAATTATGC
AAGATGGGTATAGTGTTTCAAAAGTACTGCCAGGTCAAAGAGACGCAACC
GCTCATCATCAAATTAAACCAACAGCTATAGTCAAGCAGTTAGGAAAGGT
TGATGCTAAAGTGGTCAGGAAAGATGATGGTAGCATTCAAGATTTGTCTT
CATCTTTTAAGAGCAGTTTAATTTTAGGTACCTCAGAAAAAGAGGAGGAA
TTAGCCCAATCATGTGAAGCTGCGCTCAAATCCTCTCCCGATTGTGCTATT
AAAAAGAAAGATGTTTATTCCGTCTCCATATCAGAAAGACAATGTGATGT
GGAACAGAATGATTCTGCTAAGAAATCTGTACAAGTCAAAGGGAAAATG
AGTAGAGTTACTGCTAATGATGATGCTTCCACTTCCAATTTAGATCCTGCC
AATGTTGAAGAGAAATTCCAAGTGGAAAAAGCAGGTGGATCATTAAACA
CTAAACCCAAATCTTCCCTTAAATCTGCAGGTGAAAAGAAACTTAGTCGC
ACTGTTACTTGGGCAGATAAGAAAATCAACAGCACTGGGAGTAAAGATCT
TTGTGGGTTTAAAAATTTTGGAGATATTAGAAATGAATCTGACTCAGCAG
GAAATAGTATAGATGTTGCCAATGATGAAGATACATTACGTCGCGCGTCA
GCAGAAGCTTGTGTTATTGCATTGAGCTCAGCATCAGAAGCAGTTGCTTCT
GGAGACTCGGATGTCAGTGATGCTGGTATAATTACTGTTTCTCTTACAAGT
TTTCTATTTAAATTGATTGGTTTGTAAAAGGTTTTTTCGCCTATGAATGAA
ACTTGTGCAGTTTCTGAAGCTGGAATCATTATATTGCCACCACCACATGAT
GCTGGTGAGGAAGGTACTCTGGAGGATGTTGATATACTACAAAATGATTC
AGTTACTGTGAAATGGCCTAGAAAGCCTGGAATTTCTGAAGCTGATTTCTT
TGAATCTGATGACTCATGGTTTGATGCTGCACCAGAGGGTTTCAGTTTAAC
TGTAAGTTTAGTGAAAATTTTAGAAACATTCAAACCTGTATTATTTTTCAG
TATATTCTATGGCATAACTTTAAACTTGTGCTTTTTCTTTTCTTTCTTTTTTT
AATTTTTTTGTTTGTTTGTTTGTTTGTGTGTGTGTAACTTTCTTTCAGTTGTC
ACCTTTTGCAACTATGTGGAATACCCTCTTTTCTTGGATAACATCATCTTCT
TTGGCATATATATATGGGAGGGATGAAAGTTTTCAAGAAGAATATCTATC
AGTTAATGGCAGAGAATATCCCTGCAAAGTTGTCTTGGCAGATGGTCGCT
CATCTGAAATAAAACAAACTTTAGCCAGTTGTCTTGCTCGAGCTTTACCTA
CACTTGTTGCTGTGCTCCGGCTGCCAATACCAGTATCTACCATGGAGCAA
GGGATGGTAGGATCTTGGAGTTATCAGTTTTGTAGTTTATGTTGTAGTATA
GTTGTCCCAAATTAAGTCATCAGAGATTTATTATACTTCACCAGGCATGGT
GGGACTTCTCTCATATGTTCATAAGCTCCCCTTCCCACAAAACATGCACAT
AGTCACACATATTCACAAAATATGAGATTGATAAATTTGATGTTAAAAAT
GAACTTTCAAACAGTGGTAAAGGAAAATCAGTTAATATAAGCATATGAAT
GCTACTTTTTGTGAGAATAGAGATGGATCCATTGCTTGAAATAAGAGACA
CATGGAAATTAAAATATTAAGCCTGTAGGATAAATACTTTTTATTGATTGA
AAATAAAAGAAATCGAATGGGAATTCCCCCCCCCCCCCCCCAGAGTAGAT
CCTGCCTGCATAATCCTTATTATTTCAATTCCTTGATTGTCATGTGGTTCAA
TTCAGGGAAAATCTGCAAGAATTGCAAGATCTAGTTGCAAAATTATGCCT
TATTGTCCGAGAAGACAAAATATGTTTTCTGAGAATCAGTTTAGAAATTT
GTGAAAGTAGGGGAGTCTTGGCACAGGGGTTTCAAGCAATGGCATCAGCC
CCTTGCAAAGACAATACTGGCTACTATACTTCCCACACATATTGGGTCCTA
TGCTTCCCTGACCCAGCACATAGCTTTATAGCACAAGGTTCCCTATTTTTA
GAAGTTCAGAAATTTGTAAATTCATGAGTTTTTAGGCTATAAGTTGCAGAT
AGATTTAATCTTCATATCTTTATTGAAAATTCTTTAATATATCCAATTACTT
GGTCTGTGGATGGGTTATATGTTAATTCTCGTGACTTTCTATTAGGCATGC
TTGCTGGAGACAATGTCATTTGTGGACGCACTTCCAGCTTTCAGAACAAA
ACAATGGCAAGTGGTTGCTCTTTTGTTTATTGATGCATTGTCCGTATGTAG
ATTACCTGCTCTTATCTCATACATGACGGATAGGAGGGCTTCATTTCACAG
GGTGAGAACCTTTGTTTATATCATACTGATTTATTATTTTATTTACATGATG
GATAGAAGGAAACTGTTTGCTGTGCATGAAGCATTATATAATGATGTTGG
GTTGTATATAGATCGATGCATTCCCTTATTACGGCACATGATCTTTTGGTT
GGGGTAGTTCAGTTAACATCTGATATCTCCGTTATTTCAATTGCTAAAATT
TTGGTTACTTTTCATCTGCATTTCCTTTATAAATGTTGTCGCCTGATTCTTG
GTCCAAATGGGAGCTTAACAACATAAATCCCACACAACTTAGCTACCATG
TGCTTAATGTTGTTCTCCTTCTATAGCTTTAGACTCGATGGCCTCATCTTAT
AGTTTAGTTCATCTAGTGTGGTTCTGCACTTCTACCATGGCTGTGGACTTG
AAATTCTTGTTTGGTTTTCCATCTTGTATTTTCATGCTAAATTTATTTGAAT
GCAGCTAGACCACAAGTCGTGCACATAGATTGCCATGGCACAAATTTATG
GCACGCGGACACTGAACCCAACTTGGAAATTTTTTCCTTCTAATTTGATTC
CCTACATATATGTGTGTGTTGGGGTTGCAAAAATTCCGATGACAGCTGGA
ATATGGAAGTTCATGATATATCTTATTGTTTTGTTGTTTTATCTCCTATGTC
ATGTCAGATTTTGATGTTCACTCTACTTTTAAATTTGTTAACCATTGTTCTG
TTATTGATTTGGTGAAACAGGTTTTGAGTGGTTCTCAAATAGGTATGGAA
GAATATGAGGTTTTGAAGGATCTTGCAGTGCCACTGGGCCGAGCACCTCA
CATCTCTGCCCAAAGTGGGGCATGACAACAAATAGTGCATTTTTTTTTTAT
TTGGGGAAAATTTACCTGTCAAGCATTGGAGTGCTTGTTTATAAACATAA
AAAATATGCAATGACAATTATGCTAGGCCTCTAGCAACACGGGAACCAGA
TTTCTATATTCTCTAATGCTAGCTAGAGAGTGATGAATGCAAAATTGATAT
TCAAAATAGACATAGTTCTAAAATATTGTCTTCCAGTTAAATAGCAGTTTG
TATTTTTTTTAAAA
SEq ID No. 48 MINIYO promoter
TCATTTACCAAGTTTACAAAGATTATGGTCCAAGTCCTAAAACTGAATGA
ACATCACATACCCGTCTCTTGAGTGATTTAATCATGTTCTTCATTGCACTA
AAGCGACAACTTTTGGTTCAAATATAGACTATGACTATATGGTTTGTTTTG
CACAGGATTAAAGTTGATGTTCCAATTTTATAATTAAAAAGTCAGAAAGG
GGTTTTCTTGTTATTTTTTACTTGTTCTTATAAGCTATCGGGACGACACGG
AGTTTTAAAGAGTTTTCCGTTTTGCTGAGCGGAGGCGAGAGAGGGTTTAG
AGTGATGGAGCAAAGTAGCGGGAGAGTCAATCCGGAACAGCCGAACAAC
GTCTTGGCGAGCCTTGTCGGGAGCATCGTGGAGAAAGGAATATCGGAGA
ATAAGCCTCCAAGCAAGCCGCTTCCCCCAAGGCCCTCCCTTCTTTCCTTCC
CCGTCGCTCGTCATCGTTCTCACGGACCCGTAAGCCAATCCAATCCTCTAG
TGCGTGCTTTTTAGGTTTCCATCTTCCTTTTGTTGCCTTCTTCTAGATTTTA
AGCACCTTCTACTGTTGTTTAGTACTTGGGACTCCACAATTTTTCACCGTG
CCTGACCTTGTAATTCAGCTTTCTGAGACATCTAATTTTTGTTTCTCATGTT
TGATTTTGTAGCATTTGGCTC
SEQ ID No. 49 AtRTR1 promoter
TGAATCATTTCTCAAAAAGAAAATGGGAAAAATGTCATTCAAATAATCAG
TTTACCATTTTCGTTGGTTTTAAACATAAATTTTGGACCTGGTGATTTAAA
TCCTCAATATTATGTTGACTTTCAGTTTAAACACAAATTTTCATTGATTAA
GAGACATCGTTAGAAATTCCCTAGATCACATACCCTTTATCCCAAAACCG
AAAACCGATTTTTGGATTCCCTCTTCTTCTTCGAATTCGAAGTAATCTCTT
GTCTGGAGGTTGACTGATGGCGTAAAAAAAGAAGAATTTGTATCTCAATT
AGTTTAGTTTACAAGAACTCGTGATTAAATTGAAAGTCAAAATAAAAATG
AGAATTTAAATTACCAAATCAAGAGTTTTCATATTTTAAATGGTAAACTG
ATGACATTTTCCCTGTTGAACAATATTGGCCCATAATGTAACCCAATTACT
CGGCCCAATTACGTGAACCGCCTTTCACCTGGCTTAAGGAATAAGTAAGG
ACCATTCATGATCTCATCACTTTTAGCTTTCTGGCTTCTCTGCTTAAGCTCT
CTCGAGTCTGCCTCAAGTGTTTTTGGGGGAAATTGATTTCGTTGAGAAAA
ACCCTAAATTCCGAACTTGAAGCAATTTTTCAATTTCGTTTGCAGAAAAAT
GGCAAAGGATAATGAAGCAATCGCCATTAACGATGCGGTTCACAAGCTTC
AGCTCTATATGCTCGAAAATACCACTGATCAGAACCAGCTCTTCGCGGCG
AGGAAGTTAATGTCTCGATCAGATTACGAAGATGTCGTCACTGAACGAGC
AATCGCTAAGCTCTGTGGTTATACTCTTTGCCAGAGATTTCTCCCTTCCGA
TGTTCTAGAAGAGGGAAGTATCGGATTTCGTTGAAGGACCATAAGGTTTA
CGATTTACAGGAGACGAGCAAGTTTTGCTCCGCTGGTTGTTTAATTGATAG
CAAAACGTTTTCGGGGAGTTTGCAAGAGGCTCGTACATTGGAGTTTGATT
CGGTGAAGTTGAATGAGATTTTGGATTTGTTTGGTGATTCTTTGGAAGTGA
AAGGTTCTTTGGATGTGAATAAGGATTTGGATTTGTCTAAGCTTATGATTA
AGGAGAATTTTGGAGTTAGAGGTGAAGAATTGTCTTTAGAGAAGTGGATG
GGTCCTTCTAATGCTGTTGAAGGTTATGTTCCTTTTGATCGAAGCAAATCA
AGTAATGGTAAGTTCGATGATGAACTATGGTGTGAGCAAAAAATTTCAGT
TAACAAATGTTTTATCGATGTGTAATAATTAAGTTTGGTTTTGGCAGATTC
CAAGGCTACTACTCAAAGTAATCAAGAGAAGCATGAGATGGATTTCACTA
GCACAGTAATTATGCCTGATGTTAATAGTGTTTCAAAGCTTCCACCGCAA
ACCAAGCAAGCTTCTACTGTTGTGGAATCTGTTGATGGCAAAGGGAAAAC
AGTTCTGAAAGAGCAAACTGTAGTTCCTCCCACCAAAAAAGTTTCGAGTA
AGCATTAAGGAGTTTTTAAGAGTAATAGGCCTTATGACCAACATATCTCT
AAGAAATGTAGCTGTATATGTTATTTAGTCTTGCTTAAAGGTATTTGGATG
GTATCATGAATGTTTTGATTTATTCGTCGGAGAGACAGATCTTTTGGTGGT
TATTAGGCCATTTCTACTGATGGGTGAAGCAATAAATGTCGTTGTCCTTGC
TCTCTGTTTATCTGAGTCTTAATGAATTCTAATGTGTGTCTGCAGGATTTC
GTCGTGAGAAAGAAAAGGAGAAGAAGACTTTCGGGGTTGATGGGATGGG
TTGTGCCCAGGAAAAAACTACAGTTCTCCCCAGAAAAATATTGAGTAAGC
ACTTAGGAAGCTGTGAAGATAGTTAGGCCTTACTTTCAAGATATCTCTTAA
AATAATCTGTATATGTTACGTTTTTTTCATTTTGCTGTATTCATTTGGTATC
TCGAATGAGATTCTTTATTCCTTGGGTCTCTAAGTTGTTCTAATGATTGTTA
GGCAGTTTTTGTGCCTGTGTGACTCTGTTTATCTGTCTAACATATGCAGGT
TTTTGTAATGAAATAGAGAAGGATT

The invention is further described by the following numbered paragraphs:

1. An isolated nucleic acid sequence comprising a nucleotide sequence encoding for an amino acid sequence of SEQ ID NO: 5 or an orthologue thereof

2. An isolated nucleic acid sequence according to paragraph 1 wherein said orthologue is at least 30% identical to SEQ ID NO: 5.

3. An isolated nucleic acid sequence according to paragraph 1 or 2 wherein said nucleic acid sequence is SEQ ID No. 1.

4. An isolated nucleic acid sequence comprising a nucleotide sequence encoding for amino acid sequence of SEQ ID NO: 11 or an orthologue thereof

5. An isolated nucleic acid sequence according to paragraph 4 wherein said orthologue is at least 30% identical to SEQ ID NO: 11.

6. An isolated nucleic acid sequence according to paragraph 4 or 5 wherein said nucleic acid sequence is SEQ ID No. 8.

7. An expression vector comprising the isolated nucleic acid sequence as defined in any of claims 1 to 6 characterised in that expression of the nucleic acid sequence is under the control of a promoter sequence.

8. A host cell which comprises the expression vector defined in paragraph 7.

9. A transgenic plant wherein the activity of a MINIYO and/or RTR1 polypeptide is inactivated, repressed or down-regulated.

10. A transgenic plant wherein the expression of a gene encoding a MINIYO and/or RTR1 polypeptide is inactivated, repressed or down-regulated.

11. A transgenic plant according to any of claim 9 or 10 wherein said MINIYO protein is at least 30% identical to the sequences coded by SEQ ID NO:1.

12. A transgenic plant according to paragraph 11 wherein said MINIYO protein comprises SEQ ID No. 5.

13. A transgenic plant according to any of claims 9 to 12 wherein said RTR1 protein is at least 30% identical to the sequences coded by SEQ ID NO:8.

14. A transgenic plant according to paragraph 13 wherein said RTR1 protein comprises SEQ ID No. 11.

15. A transgenic plant according to any of claims 9 to 14 characterised in that in comparison with the wild phenotype plant said plant has a reduction of between 50% and 100% in the expression of an amino acid sequence of the MINIYO and/or RTR1 protein.

16. A transgenic plant according to any of claims 9 to 15 wherein the endogenous MINIYO and/or RTR1 gene carries a functional mutation.

17. A transgenic plant according to any of claims 9 to 16 wherein said plant expresses a transgene said transgene comprising a modified MINIYO and/or or RTR1 nucleic acid sequence when compared to a wild type sequence.

18. A transgenic plant according to paragraph 17 wherein said modification in the MINIYO nucleic acid results in a polypeptide that has a substitution of the second conserved G in the RGG motif.

19. A transgenic plant according to paragraph 17 wherein said modification is a substitution or deletion of one or more residues within one or more of the nuclear localisation signals present in the MINIYO and/or RTR1 protein.

20. A transgenic plant wherein the activity of a MINIYO and/or RTR1 polypeptide is increased or up-regulated.

21. A transgenic plant wherein the expression of a gene encoding a MINIYO and/or RTR1 polypeptide is increased or up-regulated.

22. A transgenic plant according to paragraph 20 or 21 wherein said plant overexpresses a nucleic acid encoding for a MINIYO protein that is at least 30% identical to the sequences coded by SEQ ID NO:1.

23. A transgenic plant according to paragraph 22 wherein said MINIYO protein comprises SEQ ID No. 5.

24. A transgenic plant according to any of claims 20 to 23 wherein said RTR1 protein is at least 30% identical to the sequences coded by SEQ ID NO:8.

25. A transgenic plant according to paragraph 24 wherein said RTR1 protein comprises SEQ ID No. 11.

26. A transgenic plant according to any of claims 20 to 25 wherein said plant expresses a transgene said transgene comprising a modified MINIYO and/or RTR1 nucleic acid sequence when compared to a wild type sequence.

27. A transgenic plant according to paragraph 26 wherein said modification is a substitution or deletion of one or more residues within one or more nuclear export signal present in the MINIYO and/or RTR1 protein.

28. A transgenic plant according to any of claims 9 to 27, characterised in that the plant belongs to the superfamily Viridiplantae.

29. A transgenic plant according to paragraph 28, characterised in that the plant is a crop plant.

30. A product obtained from the transgenic plant defined in any of claims 9 to 29 wherein said product is selected from seed, stem, leaf, flower, root, flour and fruit.

31. Use of an isolated nucleic acid sequence comprising a nucleotide sequence coding for an amino acid sequence which is at least 30% identical to the sequences coded by SEQ ID NO: 1 to control the initiation of cell differentiation in plant apical, root and/or floral meristems.

32. A use according to paragraph 31 wherein said sequences is a modified MINIYO nucleic acid sequence when compared to a wild type sequence.

33. Use of an isolated nucleic acid sequence comprising a nucleotide sequence coding for an amino acid sequence which is at least 30% identical to the sequences coded by SEQ ID NO: 8 to control the initiation of cell differentiation in plant apical, root and/or floral meristems.

34. A use according to paragraph 33 wherein said sequence is a modified RTR1 nucleic acid sequence when compared to a wild type sequence.

35. A method for altering plant architecture by increasing or decreasing activity of the MINIYO and/or RTR1 protein.

36. A method for delaying the onset of cell differentiation and increasing the number of undifferentiated cells in a plant said method comprising decreasing the activity of a MINIYO protein which is at least 30% identical to the sequences encoded by SEQ ID NO: 1 and/or 8.

37. A method for increasing cell differentiation in a plant said method comprising increasing the activity of a MINIYO protein which is at least 30% identical to the sequences encoded by SEQ ID NO: 1 and/or 8.

38. A method for increasing yield of a plant by increasing or decreasing activity of the MINIYO and/or RTR1 protein.

39. An isolated nucleic acid sequence comprising SEQ ID No. 48.

40. An expression construct comprising a nucleic acid sequence according to paragraph 37 operably linked to a gene sequence to direct expression of the target gene in meristems and in cells in the early stages of differentiation.

41. An isolated nucleic acid sequence comprising a nucleotide sequence encoding for an amino acid sequence of SEQ ID NO: SEQ ID No. 49.

42. An expression construct comprising a nucleic acid sequence according to paragraph 39 operably linked to a gene sequence to direct expression of the target gene sites of cell differentiation and proliferation.

43. Use of an isolated nucleic acid sequence as define din claim 39 or 41 or of a vector as defined in claims 42 in directing spatial and temporal expression of a target gene.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An isolated nucleic acid sequence comprising a nucleotide sequence encoding for an amino acid sequence of SEQ ID NO: 5 or SEQ ID NO: 11 or an orthologue thereof.

2. An isolated nucleic acid sequence according to claim 1 wherein said orthologue is at least 30% identical to SEQ ID NO: 5.

3. An isolated nucleic acid sequence according to claim 1 wherein said nucleic acid sequence is SEQ ID No. 1.

4. An isolated nucleic acid sequence according to claim 1 wherein said orthologue is at least 30% identical to SEQ ID NO: 11.

5. An isolated nucleic acid sequence according to claim 1 wherein said nucleic acid sequence is SEQ ID No. 8.

6. An expression vector comprising the isolated nucleic acid sequence as defined in claim 1 characterised in that expression of the nucleic acid sequence is under the control of a promoter sequence.

7. A host cell which comprises the expression vector defined in claim 6.

8. A transgenic plant wherein the activity of a MINIYO and/or RTR1 polypeptide or the expression of a gene encoding a MINIYO and/or RTR1 polypeptide is inactivated, repressed or down-regulated.

9. A transgenic plant according to claim 8 wherein said MINIYO protein is at least 30% identical to the sequences coded by SEQ ID NO:1.

10. A transgenic plant according to claim 9 wherein said MINIYO protein comprises SEQ ID No. 5.

11. A transgenic plant according to claim 8 wherein said RTR1 protein is at least 30% identical to the sequences coded by SEQ ID NO:8.

12. A transgenic plant according to claim 11 wherein said RTR1 protein comprises SEQ ID No. 11.

13. A transgenic plant according to claim 8 characterised in that in comparison with the wild phenotype plant said plant has a reduction of between 50% and 100% in the expression of an amino acid sequence of the MINIYO and/or RTR1 protein.

14. A transgenic plant according to claim 8 wherein the endogenous MINIYO and/or RTR1 gene carries a functional mutation.

15. A transgenic plant according to claim 8 wherein said plant expresses a transgene said transgene comprising a modified MINIYO and/or or RTR1 nucleic acid sequence when compared to a wild type sequence.

16. A transgenic plant according to claim 15 wherein said modification in the MINIYO nucleic acid results in a polypeptide that has a substitution of the second conserved G in the RGG motif.

17. A transgenic plant according to claim 15 wherein said modification is a substitution or deletion of one or more residues within one or more of the nuclear localisation signals present in the MINIYO and/or RTR1 protein.

18. A transgenic plant wherein the activity of a MINIYO and/or RTR1 polypeptide or the expression of a gene encoding a MINIYO and/or RTR1 polypeptide is increased or up-regulated.

19. A transgenic plant according to claim 18 wherein said plant overexpresses a nucleic acid encoding for a MINIYO protein that is at least 30% identical to the sequences coded by SEQ ID NO:1.

20. A transgenic plant according to claim 19 wherein said MINIYO protein comprises SEQ ID No. 5.

21. A transgenic plant according to claim 18 wherein said RTR1 protein is at least 30% identical to the sequences coded by SEQ ID NO:8.

22. A transgenic plant according to claim 21 wherein said RTR1 protein comprises SEQ ID No. 11.

23. A transgenic plant according to claim 18 wherein said plant expresses a transgene said transgene comprising a modified MINIYO and/or RTR1 nucleic acid sequence when compared to a wild type sequence.

24. A transgenic plant according to claim 26 wherein said modification is a substitution or deletion of one or more residues within one or more nuclear export signal present in the MINIYO and/or RTR1 protein.

25. A transgenic plant according to claim 8, characterised in that the plant belongs to the superfamily Viridiplantae.

26. A transgenic plant according to claim 25, characterised in that the plant is a crop plant.

27. A transgenic plant according to claim 18, characterised in that the plant belongs to the superfamily Viridiplantae.

28. A transgenic plant according to claim 27, characterised in that the plant is a crop plant.

29. A product obtained from the transgenic plant of claim 8 wherein said product is selected from seed, stem, leaf, flower, root, flour and fruit.

30. A product obtained from the transgenic plant of claim 18 wherein said product is selected from seed, stem, leaf, flower, root, flour and fruit.

31. An isolated nucleic acid sequence comprising SEQ ID No. 48.

32. An isolated nucleic acid sequence comprising a nucleotide sequence encoding for an amino acid sequence of SEQ ID No. 49.

33. An expression construct comprising a nucleic acid sequence according to claim 31 operably linked to a gene sequence to direct expression of the target gene sites of cell differentiation and proliferation.


SEQUENCE LISTING (54 sequences)

SEQ ID NO: 1 - DNA, 4398 bp, Arabidopsis thaliana
atggagcaaagtagcgggagagtcaatccggaacagccgaacaacgtcttggcgagccttgtcgggagcatcgtggagaaaggaatatcggagaataagcctccaagcaagccgcttcccccaaggccctcccttctttccttccccgtcgctcgtcatcgttctcacggaccccatttggctcctgtgggaagcagcatagcacaacctaaggattacaatgacgatcaggaagaagaagaagcagaagaacgtttcatgaatgcagactccattgctgcttttgctaaaccgcttcaaagaaaagagaagaaagacatggacctcgggaggtggaaagatatggtctctggggatgatcctgcatccacacatgtccctcagcaatcaaggaaacttaagatcattgaaacgagaccgccctatgttgcttcagccgatgcggccactacatccagcaacactttactggctgccagggcatcagaccagagagagtttgtttctgataaagcaccgtttattaaaaatttgggaaccaaggaaagggttcctttaaacgcttctcctcccctagctgtttcgaatggacttgggactcgacacgcgtcttcgtctcttgaaagtgatattgatgttgagaaccatgcaaagttgcagacaatgtcacccgacgagattgctgaggctcaggctgagttattggacaagatggatcctgcactactctccattttgaagaaacgaggtgaggcaaaattgaagaagcgaaagcattctgtgcagggggtttccatcaccgatgaaacagcaaagaattcaagaactgagggtcattttgtcactcctaaagtgatggcaataccgaaagaaaaaagtgtggtgcaaaagccagggatagcccaaggattcgtgtgggatgcatggactgagagggttgaggcagccagagacttgagattttcttttgacgggaatgttgttgaggaagatgttgtctcgccagctgaaactggtggaaagtggtctggtgttgaatctgctgccgaacgtgatttcttgagaaccgagggggatcctggggccgcaggttacactatcaaagaagctattgctcttgcacgaagtgtgattcccgggcagagatgtcttgctttgcatctgcttgcatctgtactcgacaaagctttgaacaaactttgtcaaagcagaataggctacgcaagggaagaaaaagataaatccactgactgggaagccatctgggcttatgcccttggaccggaacctgagcttgtcttagcattgaggatggctcttgatgacaaccatgcctctgttgttatagcatgtgtaaaagtgattcagtgtctactgagctgttctcttaacgagaatttctttaatattctggagaacatgggaccacacgggaaagatatcttcacggcctcggtgttcaggagtaagccggaaattgatcttggcttcctccgtggttgctactggaagtacagcgctaaaccctccaatattgttgcgttccgtgaagaaatcttggatgacgggacagaagatacggatactattcagaaagatgtttttgtagccggacaagatgttgctgctggtctcgtcagaatggatatccttccaagaatttatcaccttctggagacagaaccaacagcagcgcttgaggacagcataatctctgttactattgcgatagcaaggcattctccaaaatgcacaactgcaatcttgaagtatcccaaatttgtgcaaacaattgtgaaaagattccaattgaacaaaagaatggacgttctttcttctcagatcaactctgtccgcctcttaaaggtgttggcccggtatgatcaaagtacttgcatggaatttgtgaagaatgggactttcaatgcggtcacatggcatttgtttcagttcacctcatctcttgactcatgggtgaagctagggaagcagaactgcaagctttcatctaccttgatggttgaacagctccggttttggaaggtctgtatccatagtggctgttgcgtatctcgcttcccagagctattcccagctctgtgtctgtggttgagttgtccatcattcgaaaagctcagggagaaaaatctcatcagcgagtttacttctgtgtcaaacgaggcctacctggtccttgaggcttttgccgagacacttcctaatatgtactcacaaaacattccacggaatgaatctgggacatgggactggagctatgttagccctatgattgattcagcactgagttggataacattggccccgcaattactcaagtgggagaaaggaatcgaaagtgtctctgtatcaactactactctgttgtggttgtattcaggtgtcatgcgtacaatttccaaagtccttgagaaaatctctgcggagggagaggaagaacctctaccatggctaccggagtttgttccaaagattggccttgccattatcaagcacaagcttcttagtttttctgttgcagacgtaagtaggtttggaaaagactcttccaggtgttcctcttttatggagtatttgtgttttctaagagaacgatctcaagatgacgaactagcattagcttctgtgaattgtcttcatgggttaacacggactatcgtgtccatccaaaatctgatagaatctgctagatccaagatgaaagctccgcatcaggtaagtatttccactggagatgaatctgtgcttgcaaatgggatactggcagagtctctggctgagctaacatctgtgtcgtgctcttttagagattctgtttcatcagaatggcccatcgtgcaatcaattgagctacataaacgaggcggattggcccccggcgttggacttggttggggagctagcggtggtgggttttggtcaaccagagttctgttggcacaggctggtgccggtcttctgagtctctttcttaacatctctctgagcgactcgcagaatgatcagggatctgttggctttatggataaagtaaactccgctttagctatgtgtttgattgcaggtccaagggattatttactcgtggaaagagcctttgaatatgtccttagaccgcatgctttagaacacctggcctgctgtatcaagtcaaacaaaaaaaacatatcgtttgaatgggaatgcagcgaaggggactatcatcgtatgagcagtatgcttgcttctcacttcagacatagatggttacagcaaaagggaagatcgatagccgaggaaggggtcagtggggtaaggaagggcacagttggtctggagactattcatgaggacggtgaaatgtcaaatagttcaactcaggataaaaaatcagactcctcgaccatagagtgggctcaccagagaatgcccctacctccacactggtttctcagcgccatctcagcagtccacagtggtaaaacctcaacagggccaccagaatccacagagttgcttgaagttgcaaaagctggagttttctttcttgcaggacttgagtcatcgtctggttttggatcgcttccctctcctgttgtgagtgtaccgttggtttggaagtttcacgctttgtctaccgtattgcttgttggaatggacatcatcgaagacaagaacactaggaacttgtacaattatctgcaggagctttatgggcagtttcttgatgaagcgagactaaatcaccgtgacactgagcttctgaggttcaagtcagacattcatgagaactactctacttttctggagatggtggtggagcagtatgctgcggtgtcatatggtgatgtagtgtatggccggcaggtctcggtttacctgcatcaatgcgtggaacactctgttcggctttcggcatggacagtgctctccaatgcccgtgttctcgagcttctgccgagtctagacaagtgcttgggagaagcggatggttacctcgaacctgttgaggaaaatgaggccgtccttgaggcctacctgaagtcatggacttgtggggcattggacagagctgcgacgcgtggatcagtagcctatacgctggttgtgcatcacttttcatctttagtcttttgcaaccaagccaaggataaagtatccctgcggaataagattgtcaagactcttgtcagggatttatcaagaaagcggcatcgtgaggggatgatgttagatctcctgcggtataagaaagggtctgcgaacgccatggaagaagaagtgatagcagcggagacagagaaaagaatggaggtgttgaaagagggttgcgaagggaactccaccctcctcttggaactggagaagctgaaatcagccgctctctgtggaagaaggtga

SEQ ID NO: 2 - DNA, 4398 bp, Arabidopsis thaliana
atggagcaaagtagcgggagagtcaatccggaacagccgaacaacgtcttggcgagccttgtcgggagcatcgtggagaaaggaatatcggagaataagcctccaagcaagccgcttcccccaaggccctcccttctttccttccccgtcgctcgtcatcgttctcacggaccccatttggctcctgtgggaagcagcatagcacaacctaaggattacaatgacgatcaggaagaagaagaagcagaagaacgtttcatgaatgcagactccattgctgcttttgctaaaccgcttcaaagaaaagagaagaaagacatggacctcgggaggtggaaagatatggtctctggggatgatcctgcatccacacatgtccctcagcaatcaaggaaacttaagatcattgaaacgagaccgccctatgttgcttcagccgatgcggccactacatccagcaacactttactggctgccagggcatcagaccagagagagtttgtttctgataaagcaccgtttattaaaaatttgggaaccaaggaaagggttcctttaaacgcttctcctcccctagctgtttcgaatggacttgggactcgacacgcgtcttcgtctcttgaaagtgatattgatgttgagaaccatgcaaagttgcagacaatgtcacccgacgagattgctgaggctcaggctgagttattggacaagatggatcctgcactactctccattttgaagaaacgaggtgaggcaaaattgaagaagcgaaagcattctgtgcagggggtttccatcaccgatgaaacagcaaagaattcaagaactgagggtcattttgtcactcctaaagtgatggcaataccgaaagaaaaaagtgtggtgcaaaagccagggatagcccaaggattcgtgtgggatgcatggactgagagggttgaggcagccagagacttgagattttcttttgacgggaatgttgttgaggaagatgttgtctcgccagctgaaactggtggaaagtggtctggtgttgaatctgctgccgaacgtgatttcttgagaaccgagggggatcctggggccgcaggttacactatcaaagaagctattgctcttgcacgaagtgtgattcccgggcagagatgtcttgctttgcatctgcttgcatctgtactcgacaaagctttgaacaaactttgtcaaagcagaataggctacgcaagggaagaaaaagataaatccactgactgggaagccatctgggcttatgcccttggaccggaacctgagcttgtcttagcattgaggatggctcttgatgacaaccatgcctctgttgttatagcatgtgtaaaagtgattcagtgtctactgagctgttctcttaacgagaatttctttaatattctggagaacatgggaccacacgggaaagatatcttcacggcctcggtgttcaggagtaagccggaaattgatcttggcttcctccgtggttgctactggaagtacagcgctaaaccctccaatattgttgcgttccgtgaagaaatcttggatgacgggacagaagatacggatactattcagaaagatgtttttgtagccggacaagatgttgctgctggtctcgtcagaatggatatccttccaagaatttatcaccttctggagacagaaccaacagcagcgcttgaggacagcataatctctgttactattgcgatagcaaggcattctccaaaatgcacaactgcaatcttgaagtatcccaaatttgtgcaaacaattgtgaaaagattccaattgaacaaaagaatggacgttctttcttctcagatcaactctgtccgcctcttaaaggtgttggcccggtatgatcaaagtacttgcatggaatttgtgaagaatgggactttcaatgcggtcacatggcatttgtttcagttcacctcatctcttgactcatgggtgaagctagggaagcagaactgcaagctttcatctaccttgatggttgaacagctccggttttggaaggtctgtatccatagtggctgttgcgtatctcgcttcccagagctattcccagctctgtgtctgtggttgagttgtccatcattcgaaaagctcagggagaaaaatctcatcagcgagtttacttctgtgtcaaacgaggcctacctggtccttgaggcttttgccgagacacttcctaatatgtactcacaaaacattccacggaatgaatctgggacatgggactggagctatgttagccctatgattgattcagcactgagttggataacattggccccgcaattactcaagtgggagaaaggaatcgaaagtgtctctgtatcaactactactctgttgtggttgtattcaggtgtcatgcgtacaatttccaaagtccttgagaaaatctctgcggagggagaggaagaacctctaccatggctaccggagtttgttccaaagattggccttgccattatcaagcacaagcttcttagtttttctgttgcagacgtaagtaggtttggaaaagactcttccaggtgttcctcttttatggagtatttgtgttttctaagagaacgatctcaagatgacgaactagcattagcttctgtgaattgtcttcatgggttaacacggactatcgtgtccatccaaaatctgatagaatctgctagatccaagatgaaagctccgcatcaggtaagtatttccactggagatgaatctgtgcttgcaaatgggatactggcagagtctctggctgagctaacatctgtgtcgtgctcttttagagattctgtttcatcagaatggcccatcgtgcaatcaattgagctacataaacgaggcgaattggcccccggcgttggacttggttggggagctagcggtggtgggttttggtcaaccagagttctgttggcacaggctggtgccggtcttctgagtctctttcttaacatctctctgagcgactcgcagaatgatcagggatctgttggctttatggataaagtaaactccgctttagctatgtgtttgattgcaggtccaagggattatttactcgtggaaagagcctttgaatatgtccttagaccgcatgctttagaacacctggcctgctgtatcaagtcaaacaaaaaaaacatatcgtttgaatgggaatgcagcgaaggggactatcatcgtatgagcagtatgcttgcttctcacttcagacatagatggttacagcaaaagggaagatcgatagccgaggaaggggtcagtggggtaaggaagggcacagttggtctggagactattcatgaggacggtgaaatgtcaaatagttcaactcaggataaaaaatcagactcctcgaccatagagtgggctcaccagagaatgcccctacctccacactggtttctcagcgccatctcagcagtccacagtggtaaaacctcaacagggccaccagaatccacagagttgcttgaagttgcaaaagctggagttttctttcttgcaggacttgagtcatcgtctggttttggatcgcttccctctcctgttgtgagtgtaccgttggtttggaagtttcacgctttgtctaccgtattgcttgttggaatggacatcatcgaagacaagaacactaggaacttgtacaattatctgcaggagctttatgggcagtttcttgatgaagcgagactaaatcaccgtgacactgagcttctgaggttcaagtcagacattcatgagaactactctacttttctggagatggtggtggagcagtatgctgcggtgtcatatggtgatgtagtgtatggccggcaggtctcggtttacctgcatcaatgcgtggaacactctgttcggctttcggcatggacagtgctctccaatgcccgtgttctcgagcttctgccgagtctagacaagtgcttgggagaagcggatggttacctcgaacctgttgaggaaaatgaggccgtccttgaggcctacctgaagtcatggacttgtggggcattggacagagctgcgacgcgtggatcagtagcctatacgctggttgtgcatcacttttcatctttagtcttttgcaaccaagccaaggataaagtatccctgcggaataagattgtcaagactcttgtcagggatttatcaagaaagcggcatcgtgaggggatgatgttagatctcctgcggtataagaaagggtctgcgaacgccatggaagaagaagtgatagcagcggagacagagaaaagaatggaggtgttgaaagagggttgcgaagggaactccaccctcctcttggaactggagaagctgaaatcagccgctctctgtggaagaaggtga

SEQ ID NO: 3 - DNA, 4397 bp, Arabidopsis thaliana
  Unsure (273)..(274): In between nucleotide 273 and 274 is a T-DNA insert.  The T-DNA was provided by the Salk Institute and the ID number is Salk_099873
atggagcaaagtagcgggagagtcaatccggaacagccgaacaacgtcttggcgagccttgtcgggagcatcgtggagaaaggaatatcggagaataagcctccaagcaagccgcttcccccaaggccctcccttctttccttccccgtcgctcgtcatcgttctcacggaccccatttggctcctgtgggaagcagcatagcacaacctaaggattacaatgacgatcaggaagaagaagaagcagaagaacgtttcatgaatgcagactccttgctgcttttgctaaaccgcttcaaagaaaagagaagaaagacatggacctcgggaggtggaaagatatggtctctggggatgatcctgcatccacacatgtccctcagcaatcaaggaaacttaagatcattgaaacgagaccgccctatgttgcttcagccgatgcggccactacatccagcaacactttactggctgccagggcatcagaccagagagagtttgtttctgataaagcaccgtttattaaaaatttgggaaccaaggaaagggttcctttaaacgcttctcctcccctagctgtttcgaatggacttgggactcgacacgcgtcttcgtctcttgaaagtgatattgatgttgagaaccatgcaaagttgcagacaatgtcacccgacgagattgctgaggctcaggctgagttattggacaagatggatcctgcactactctccattttgaagaaacgaggtgaggcaaaattgaagaagcgaaagcattctgtgcagggggtttccatcaccgatgaaacagcaaagaattcaagaactgagggtcattttgtcactcctaaagtgatggcaataccgaaagaaaaaagtgtggtgcaaaagccagggatagcccaaggattcgtgtgggatgcatggactgagagggttgaggcagccagagacttgagattttcttttgacgggaatgttgttgaggaagatgttgtctcgccagctgaaactggtggaaagtggtctggtgttgaatctgctgccgaacgtgatttcttgagaaccgagggggatcctggggccgcaggttacactatcaaagaagctattgctcttgcacgaagtgtgattcccgggcagagatgtcttgctttgcatctgcttgcatctgtactcgacaaagctttgaacaaactttgtcaaagcagaataggctacgcaagggaagaaaaagataaatccactgactgggaagccatctgggcttatgcccttggaccggaacctgagcttgtcttagcattgaggatggctcttgatgacaaccatgcctctgttgttatagcatgtgtaaaagtgattcagtgtctactgagctgttctcttaacgagaatttctttaatattctggagaacatgggaccacacgggaaagatatcttcacggcctcggtgttcaggagtaagccggaaattgatcttggcttcctccgtggttgctactggaagtacagcgctaaaccctccaatattgttgcgttccgtgaagaaatcttggatgacgggacagaagatacggatactattcagaaagatgtttttgtagccggacaagatgttgctgctggtctcgtcagaatggatatccttccaagaatttatcaccttctggagacagaaccaacagcagcgcttgaggacagcataatctctgttactattgcgatagcaaggcattctccaaaatgcacaactgcaatcttgaagtatcccaaatttgtgcaaacaattgtgaaaagattccaattgaacaaaagaatggacgttctttcttctcagatcaactctgtccgcctcttaaaggtgttggcccggtatgatcaaagtacttgcatggaatttgtgaagaatgggactttcaatgcggtcacatggcatttgtttcagttcacctcatctcttgactcatgggtgaagctagggaagcagaactgcaagctttcatctaccttgatggttgaacagctccggttttggaaggtctgtatccatagtggctgttgcgtatctcgcttcccagagctattcccagctctgtgtctgtggttgagttgtccatcattcgaaaagctcagggagaaaaatctcatcagcgagtttacttctgtgtcaaacgaggcctacctggtccttgaggcttttgccgagacacttcctaatatgtactcacaaaacattccacggaatgaatctgggacatgggactggagctatgttagccctatgattgattcagcactgagttggataacattggccccgcaattactcaagtgggagaaaggaatcgaaagtgtctctgtatcaactactactctgttgtggttgtattcaggtgtcatgcgtacaatttccaaagtccttgagaaaatctctgcggagggagaggaagaacctctaccatggctaccggagtttgttccaaagattggccttgccattatcaagcacaagcttcttagtttttctgttgcagacgtaagtaggtttggaaaagactcttccaggtgttcctcttttatggagtatttgtgttttctaagagaacgatctcaagatgacgaactagcattagcttctgtgaattgtcttcatgggttaacacggactatcgtgtccatccaaaatctgatagaatctgctagatccaagatgaaagctccgcatcaggtaagtatttccactggagatgaatctgtgcttgcaaatgggatactggcagagtctctggctgagctaacatctgtgtcgtgctcttttagagattctgtttcatcagaatggcccatcgtgcaatcaattgagctacataaacgaggcggattggcccccggcgttggacttggttggggagctagcggtggtgggttttggtcaaccagagttctgttggcacaggctggtgccggtcttctgagtctctttcttaacatctctctgagcgactcgcagaatgatcagggatctgttggctttatggataaagtaaactccgctttagctatgtgtttgattgcaggtccaagggattatttactcgtggaaagagcctttgaatatgtccttagaccgcatgctttagaacacctggcctgctgtatcaagtcaaacaaaaaaaacatatcgtttgaatgggaatgcagcgaaggggactatcatcgtatgagcagtatgcttgcttctcacttcagacatagatggttacagcaaaagggaagatcgatagccgaggaaggggtcagtggggtaaggaagggcacagttggtctggagactattcatgaggacggtgaaatgtcaaatagttcaactcaggataaaaaatcagactcctcgaccatagagtgggctcaccagagaatgcccctacctccacactggtttctcagcgccatctcagcagtccacagtggtaaaacctcaacagggccaccagaatccacagagttgcttgaagttgcaaaagctggagttttctttcttgcaggacttgagtcatcgtctggttttggatcgcttccctctcctgttgtgagtgtaccgttggtttggaagtttcacgctttgtctaccgtattgcttgttggaatggacatcatcgaagacaagaacactaggaacttgtacaattatctgcaggagctttatgggcagtttcttgatgaagcgagactaaatcaccgtgacactgagcttctgaggttcaagtcagacattcatgagaactactctacttttctggagatggtggtggagcagtatgctgcggtgtcatatggtgatgtagtgtatggccggcaggtctcggtttacctgcatcaatgcgtggaacactctgttcggctttcggcatggacagtgctctccaatgcccgtgttctcgagcttctgccgagtctagacaagtgcttgggagaagcggatggttacctcgaacctgttgaggaaaatgaggccgtccttgaggcctacctgaagtcatggacttgtggggcattggacagagctgcgacgcgtggatcagtagcctatacgctggttgtgcatcacttttcatctttagtcttttgcaaccaagccaaggataaagtatccctgcggaataagattgtcaagactcttgtcagggatttatcaagaaagcggcatcgtgaggggatgatgttagatctcctgcggtataagaaagggtctgcgaacgccatggaagaagaagtgatagcagcggagacagagaaaagaatggaggtgttgaaagagggttgcgaagggaactccaccctcctcttggaactggagaagctgaaatcagccgctctctgtggaagaaggtga

SEQ ID NO: 4 - DNA, 4398 bp, Arabidopsis thaliana; identical to SEQ ID NO: 1
  Unsure (1356)..(1357): In between nucleotide 1356 and 1357 is a T-DNA insert.  The T-DNA was provided by the Salk Institute and the ID number is Salk 692_g12

SEQ ID NO: 5 - protein, 1465 aa, Arabidopsis thaliana
MEQSSGRVNPEQPNNVLASLVGSIVEKGISENKPPSKPLPPRPSLLSFPVARHRSHGPHLAPVGSSIAQPKDYNDDQEEEEAEERFMNADSIAAFAKPLQRKEKKDMDLGRWKDMVSGDDPASTHVPQQSRKLKIIETRPPYVASADAATTSSNTLLAARASDQREFVSDKAPFIKNLGTKERVPLNASPPLAVSNGLGTRHASSSLESDIDVENHAKLQTMSPDEIAEAQAELLDKMDPALLSILKKRGEAKLKKRKHSVQGVSITDETAKNSRTEGHFVTPKVMAIPKEKSVVQKPGIAQGFVWDAWTERVEAARDLRFSFDGNVVEEDVVSPAETGGKWSGVESAAERDFLRTEGDPGAAGYTIKEAIALARSVIPGQRCLALHLLASVLDKALNKLCQSRIGYAREEKDKSTDWEAIWAYALGPEPELVLALRMALDDNHASVVIACVKVIQCLLSCSLNENFFNILENMGPHGKDIFTASVFRSKPEIDLGFLRGCYWKYSAKPSNIVAFREEILDDGTEDTDTIQKDVFVAGQDVAAGLVRMDILPRIYHLLETEPTAALEDSIISVTIAIARHSPKCTTAILKYPKFVQTIVKRFQLNKRMDVLSSQINSVRLLKVLARYDQSTCMEFVKNGTFNAVTWHLFQFTSSLDSWVKLGKQNCKLSSTLMVEQLRFWKVCIHSGCCVSRFPELFPALCLWLSCPSFEKLREKNLISEFTSVSNEAYLVLEAFAETLPNMYSQNIPRNESGTWDWSYVSPMIDSALSWITLAPQLLKWEKGIESVSVSTTTLLWLYSGVMRTISKVLEKISAEGEEEPLPWLPEFVPKIGLAIIKHKLLSFSVADVSRFGKDSSRCSSFMEYLCFLRERSQDDELALASVNCLHGLTRTIVSIQNLIESARSKMKAPHQVSISTGDESVLANGILAESLAELTSVSCSFRDSVSSEWPIVQSIELHKRGGLAPGVGLGWGASGGGFWSTRVLLAQAGAGLLSLFLNISLSDSQNDQGSVGFMDKVNSALAMCLIAGPRDYLLVERAFEYVLRPHALEHLACCIKSNKKNISFEWECSEGDYHRMSSMLASHFRHRWLQQKGRSIAEEGVSGVRKGTVGLETIHEDGEMSNSSTQDKKSDSSTIEWAHQRMPLPPHWFLSAISAVHSGKTSTGPPESTELLEVAKAGVFFLAGLESSSGFGSLPSPVVSVPLVWKFHALSTVLLVGMDIIEDKNTRNLYNYLQELYGQFLDEARLNHRDTELLRFKSDIHENYSTFLEMVVEQYAAVSYGDVVYGRQVSVYLHQCVEHSVRLSAWTVLSNARVLELLPSLDKCLGEADGYLEPVEENEAVLEAYLKSWTCGALDRAATRGSVAYTLVVHHFSSLVFCNQAKDKVSLRNKIVKTLVRDLSRKRHREGMMLDLLRYKKGSANAMEEEVIAAETEKRMEVLKEGCEGNSTLLLELEKLKSAALCGRR

SEQ ID NO: 6 - protein, 1465 aa, Arabidopsis thaliana
MEQSSGRVNPEQPNNVLASLVGSIVEKGISENKPPSKPLPPRPSLLSFPVARHRSHGPHLAPVGSSIAQPKDYNDDQEEEEAEERFMNADSIAAFAKPLQRKEKKDMDLGRWKDMVSGDDPASTHVPQQSRKLKIIETRPPYVASADAATTSSNTLLAARASDQREFVSDKAPFIKNLGTKERVPLNASPPLAVSNGLGTRHASSSLESDIDVENHAKLQTMSPDEIAEAQAELLDKMDPALLSILKKRGEAKLKKRKHSVQGVSITDETAKNSRTEGHFVTPKVMAIPKEKSVVQKPGIAQGFVWDAWTERVEAARDLRFSFDGNVVEEDVVSPAETGGKWSGVESAAERDFLRTEGDPGAAGYTIKEAIALARSVIPGQRCLALHLLASVLDKALNKLCQSRIGYAREEKDKSTDWEAIWAYALGPEPELVLALRMALDDNHASVVIACVKVIQCLLSCSLNENFFNILENMGPHGKDIFTASVFRSKPEIDLGFLRGCYWKYSAKPSNIVAFREEILDDGTEDTDTIQKDVFVAGQDVAAGLVRMDILPRIYHLLETEPTAALEDSIISVTIAIARHSPKCTTAILKYPKFVQTIVKRFQLNKRMDVLSSQINSVRLLKVLARYDQSTCMEFVKNGTFNAVTWHLFQFTSSLDSWVKLGKQNCKLSSTLMVEQLRFWKVCIHSGCCVSRFPELFPALCLWLSCPSFEKLREKNLISEFTSVSNEAYLVLEAFAETLPNMYSQNIPRNESGTWDWSYVSPMIDSALSWITLAPQLLKWEKGIESVSVSTTTLLWLYSGVMRTISKVLEKISAEGEEEPLPWLPEFVPKIGLAIIKHKLLSFSVADVSRFGKDSSRCSSFMEYLCFLRERSQDDELALASVNCLHGLTRTIVSIQNLIESARSKMKAPHQVSISTGDESVLANGILAESLAELTSVSCSFRDSVSSEWPIVQSIELHKRGELAPGVGLGWGASGGGFWSTRVLLAQAGAGLLSLFLNISLSDSQNDQGSVGFMDKVNSALAMCLIAGPRDYLLVERAFEYVLRPHALEHLACCIKSNKKNISFEWECSEGDYHRMSSMLASHFRHRWLQQKGRSIAEEGVSGVRKGTVGLETIHEDGEMSNSSTQDKKSDSSTIEWAHQRMPLPPHWFLSAISAVHSGKTSTGPPESTELLEVAKAGVFFLAGLESSSGFGSLPSPVVSVPLVWKFHALSTVLLVGMDIIEDKNTRNLYNYLQELYGQFLDEARLNHRDTELLRFKSDIHENYSTFLEMVVEQYAAVSYGDVVYGRQVSVYLHQCVEHSVRLSAWTVLSNARVLELLPSLDKCLGEADGYLEPVEENEAVLEAYLKSWTCGALDRAATRGSVAYTLVVHHFSSLVFCNQAKDKVSLRNKIVKTLVRDLSRKRHREGMMLDLLRYKKGSANAMEEEVIAAETEKRMEVLKEGCEGNSTLLLELEKLKSAALCGRR

SEQ ID NO: 7 - DNA, 5657 bp, Arabidopsis thaliana
aaagagttttccgttttgctgagcggaggcgagagagggtttagagtgatggagcaaagtagcgggagagtcaatccggaacagccgaacaacgtcttggcgagccttgtcgggagcatcgtggagaaaggaatatcggagaataagcctccaagcaagccgcttcccccaaggccctcccttctttccttccccgtcgctcgtcatcgttctcacggacccgtaagccaatccaatcctctagtgcgtgctttttaggtttccatcttccttttgttgccttcttctagattttaagcaccttctactgttgtttagtacttgggactccacaatttttcaccgtgcctgaccttgtaattcagctttctgagacatctaatttttgtttctcatgtttgattttgtagcatttggctcctgtgggaagcagcatagcacaacctaaggattacaatgacgatcaggaagaagaagaagcagaagaacgtttcatgaatgcagactccattgctgcttttgctaaaccgcttcaaagaaaagagaagaaagacatggacctcgggaggtggaaagatatggtctctggggatgatcctgcatccacacatgtccctcagcaatcaaggaaacttaagatcattgaaacgagaccgccctatgttgcttcagccgatgcggccactacatccagcaacactttactggctgccagggcatcagaccagagagagtttgtttctgataaagcaccgtttattaaaaatttgggaaccaaggaaagggttcctttaaacgcttctcctcccctagctgtttcgaatggacttgggactcgacacgcgtcttcgtctcttgaaagtgatattgatgttgagaaccatgcaaagttgcagacaatgtcacccgacgagattgctgaggctcaggctgagttattggacaagatggatcctgcactactctccattttgaagaaacgaggtgaggcaaaattgaagaagcgaaagcattctgtgcagggggtttccatcaccgatgaaacagcaaagaattcaagaactgagggtcattttgtcactcctaaagtgatggcaataccgaaagaaaaaagtgtggtgcaaaagccagggatagcccaaggattcgtgtgggatgcatggactgagagggttgaggcagccagagacttgagattttcttttgacgggaatgttgttgaggaagatgttgtctcgccagctgaaactggtgagtagaacaatacaactgaaacacatgacaatcttaggttgcttacactttgactgtacaggtggaaagtggtctggtgttgaatctgctgccgaacgtgatttcttgagaaccgagggggatcctggggccgcaggttacactatcaaagaagctattgctcttgcacgaagtgtggtatgtatgattgccacatattttaattttgatgctaattaatggttaaattcttttttccctccattttggctttagctgaacaaaacctgtaggctgagactgcgtttttttcgttatcactgctcattgatttgtatgtattattgatatatatatcagattcccgggcagagatgtcttgctttgcatctgcttgcatctgtactcgacaaagctttgaacaaactttgtcaaagcagaataggctacgcaagggaagaaaaagataaatccactgactgggaagccatctgggcttatgcccttggaccggaacctgagcttgtcttagcattgaggtaatttcctgatgggtgtaattttgagacttatttgtgaagttgtcactcataaatcataaattgtttgttcttatcaatataagtttcttttcttctttaggatggctcttgatgacaaccatgcctctgttgttatagcatgtgtaaaagtgattcagtgtctactgagctgttctcttaacgagaatttctttaatattctggaggtatagttgatttttctcactcctaagaagttatagtcctcatagaacgtgattatacatgttcaaactgataaaacccatttctatttccagaacatgggaccacacgggaaagatatcttcacggcctcggtgttcaggagtaagccggaaattgatcttggcttcctccgtggttgctactggaagtacagcgctaaaccctccaatattgttgcgttccgtgaagaaatcttggatgacgggacagaagatacggatactattcagaaagatgtttttgtagccggacaagatgttgctgctggtctcgtcagaatggatatccttccaagaatttatcaccttctggaggtgagatcactatctatgtgtaactcagcaagtaaaatcattctttttgtgtcgttgcttagttttctggtttttttttaatgttcatgatttcagacagaaccaacagcagcgcttgaggacagcataatctctgttactattgcgatagcaaggcattctccaaaatgcacaactgcaatcttgaagtatcccaaatttgtgcaaacaattgtgaaaagattccaattgaacaaaagaatggacgttctttcttctcagatcaactctgtccgcctcttaaaggtaatactggtccgctcatacaaaattatcttggggtcgttatattcattcgtctttgatgttttttttacagaacctgatgattcgagtttgttaagctatcaattctcagagctattgtaaccttcgttcttctttctctctttttaatttcactaaggtgttggcccggtatgatcaaagtacttgcatggaatttgtgaagaatgggactttcaatgcggtcacatggcatttgtttcagttcacctcatctcttgactcatgggtgaagctagggaagcagaactgcaagctttcatctaccttgatggttgaacagctccggttttggaaggtctgtatccatagtggctgttgcgtatctcgcttcccagagctattcccagctctgtgtctgtggttgagttgtccatcattcgaaaagctcagggagaaaaatctcatcagcgagtttacttctgtgtcaaacgaggcctacctggtccttgaggcttttgccgagacacttcctaatatgtactcacaaaacattccacggaatgaatctgggacatgggactggagctatgttagccctatgattgattcagcactgagttggataacattggccccgcaattactcaagtgggagaaaggaatcgaaagtgtctctgtatcaactactactctgttgtggttgtattcaggtgtcatgcgtacaatttccaaagtccttgagaaaatctctgcggagggagaggaagaacctctaccatggctaccggagtttgttccaaagattggccttgccattatcaagcacaagcttcttagtttttctgttgcagacgtaagtaggtttggaaaagactcttccaggtgttcctcttttatggagtatttgtgttttctaagagaacgatctcaagatgacgaactagcattagcttctgtgaattgtcttcatgggttaacacggactatcgtgtccatccaaaatctgatagaatctgctagatccaagatgaaagctccgcatcaggtaagtatttccactggagatgaatctgtgcttgcaaatgggatactggcagagtctctggctgagctaacatctgtgtcgtgctcttttagagattctgtttcatcagaatggcccatcgtgcaatcaattgagctacataaacgaggcggattggcccccggcgttggacttggttggggagctagcggtggtgggttttggtcaaccagagttctgttggcacaggctggtgccggtcttctgagtctctttcttaacatctctctgagcgactcgcagaatgatcagggatctgttggctttatggataaagtaaactccgctttagctatgtgtttgattgcaggtccaagggattatttactcgtggaaagagcctttgaatatgtccttagaccgcatgctttagaacacctggcctgctgtatcaagtcaaacaaaaaaaacatatcgtttgaatgggaatgcagcgaaggggactatcatcgtatgagcagtatgcttgcttctcacttcagacatagatggttacagcaaaagggaagatcgatagccgaggaaggggtcagtggggtaaggaagggcacagttggtctggagactattcatgaggacggtgaaatgtcaaatagttcaactcaggataaaaaatcagactcctcgaccatagagtgggctcaccagagaatgcccctacctccacactggtttctcagcgccatctcagcagtccacagtggtaaaacctcaacagggccaccagaatccacagagttgcttgaagttgcaaaagctggagttttctttcttgcaggacttgagtcatcgtctggttttggatcgcttccctctcctgttgtgagtgtaccgttggtttggaagtttcacgctttgtctaccgtattgcttgttggaatggacatcatcgaagacaagaacactaggaacttgtacaattatctgcaggagctttatgggcagtttcttgatgaagcgagactaaatcaccgtgacactgagcttctgaggttcaagtcagacattcatgagaactactctacttttctggagatggtggtggagcagtatgctgcggtgtcatatggtgatgtagtgtatggccggcaggtctcggtttacctgcatcaatgcgtggaacactctgttcggctttcggcatggacagtgctctccaatgcccgtgttctcgagcttctgccgagtctagacaagtgcttgggagaagcggatggttacctcgaacctgttgaggtaatttacaaaaataataaaatgttgatagtggtgaataatggcatcttgagcatccaactaagataaaatggtgaattgatattgcaggaaaatgaggccgtccttgaggcctacctgaagtcatggacttgtggggcattggacagagctgcgacgcgtggatcagtagcctatacgctggttgtgcatcacttttcatctttagtcttttgcaaccaagccaaggataaagtatccctgcggaataagattgtcaagactcttgtcagggatttatcaagaaagcggcatcgtgaggtaacttgaaatccctcatctcttgttcaatgtcattctggggactgaggatgataatgaaacgtggaaataatgtttcaggggatgatgttagatctcctgcggtataagaaagggtctgcgaacgccatggaagaagaagtgatagcagcggagacagagaaaagaatggaggtgttgaaagagggttgcgaagggaactccaccctcctcttggaactggagaagctgaaatcagccgctctctgtggaagaaggtgaacgagagaatgagaggaaagaaagtctgtgtgtttctttctctgttttgaggttctcttacagatgaaaagctgtgtaattaaaaatcgatgttcttcttctgttcttgtaagattttggatttttccaatttctgacaaagttcaattaaaaaacttgactgacattttgaaa

SEQ ID NO: 8 - DNA, 2208 bp, Arabidopsis thaliana
atggcaaaggataatgaagcaatcgccattaacgatgcggttcacaagcttcagctctatatgctcgaaaataccactgatcagaaccagctcttcgcggcgaggaagttaatgtctcgatcagattacgaagatgtcgtcactgaacgagcaatcgctaagctctgtggttatactctttgccagagatttctcccttccgatgtttctagaagagggaagtatcggatttcgttgaaggaccataaggtttacgatttacaggagacgagcaagttttgctccgctggttgtttaattgatagcaaaacgttttcggggagtttgcaagaggctcgtacattggagtttgattcggtgaagttgaatgagattttggatttgtttggtgattctttggaagtgaaaggttctttggatgtgaataaggatttggatttgtctaagcttatgattaaggagaattttggagttagaggtgaagaattgtctttagagaagtggatgggtccttctaatgctgttgaaggttatgttccttttgatcgaagcaaatcaagtaatgattccaaggctactactcaaagtaatcaagagaagcatgagatggatttcactagcacagtaattatgcctgatgttaatagtgtttcaaagcttccaccgcaaaccaagcaagcttctactgttgtggaatctgttgatggcaaagggaaaacagttctgaaagagcaaactgtagttcctcccaccaaaaaagtttcgagatttcgtcgtgagaaagaaaaggagaagaagactttcggggttgatgggatgggttgtgcccaggaaaaaactacagttctccccagaaaaatattgagtttttgtaatgaaatagagaaggattttaagaattttgggtttgatgagatgggtcttgcgagttctgctatgatgagtgatggatacggcgtagaatatagtgtgtctaagcagccacaatgttcgatggaagattctcttagttgcaagctaaaaggagatcttcagactttggacgggaaaaataccctatcaggatcctcttctggttctaatacgaagggctcgaagacaaaaccagagaaatcaagaaagaaaattatttctgttgaataccatgctaattcttatgaagatggtgaagaaatccttgcagctgaatcgtatgaaagacataaagctcaggatgtgtgttcatcaagtgaaatcgtcactaaatcatgccttaaaatttctggctcgaagaagcttagtcgttcagttacttgggccgatcagaatgatggccgtggtgatctttgtgaggttagaaacaatgataacgcagcaggtcctagcctgtcttctaatgatatagaggatgtcaatagtttatcacgccttgcattagcagaagcccttgctacggcattgagccaggctgccgaagctgtttcttcgggaaattcagatgcaagtgatgccactgcaaaagctggaatcattttgttgcccagcacacatcaacttgacgaagaggttactgaggaacatagtgaggaggaaatgactgaagaggaaccaactcttctcaagtggccaaataagcccgggattccagattctgatttgtttgaccgtgatcaatcgtggtttgatggacctccagagggcttcaatctcacattatcaaatttcgctgtgatgtgggattcactgtttggctgggtatcatcgtcctctctggcatacatatatgggaaggaagaatctgctcatgaggagttcttattggttaacgggaaggagtacccccggaggattatcatggtagatgggctttcctcagagatcaagcagacaattgctgggtgccttgccagagctttaccgagagtcgtcactcatctcaggctgccaatagcgatatccgagttagaaaagggactgggaagcttgttggagacaatgtcgttgacaggagcagttccatcatttagggtaaaagaatggctagtgattgttcttcttttcttggatgcgttgtctgtatcacgtatccctcggattgcaccttatatatccaacagagacaagattttggaaggaagtggaattggaaatgaagagtatgagacaatgaaggatatcctgttaccacttggccgtgttcctcagtttgctacccgaagcggggcgtag

SEQ ID NO: 9 - DNA, 2208 bp, Arabidopsis thaliana; identical to SEQ ID NO: 8
  Unsure (414)..(415): In between nucleotide 414 and 415 is a T-DNA insert.  The T-DNA was provided by the Salk Institute and the ID number is SALK_012339

SEQ ID NO: 10 - DNA, 2208 bp, Arabidopsis thaliana; identical to SEQ ID NO: 8
  Unsure (865)..(866): In between nucleotide 865 and 866 is a T-DNA insert.  The T-DNA was provided by the Salk Institute and the ID number is SALK_115762

SEQ ID NO: 11 - protein, 735 aa, Arabidopsis thaliana
MAKDNEAIAINDAVHKLQLYMLENTTDQNQLFAARKLMSRSDYEDVVTERAIAKLCGYTLCQRFLPSDVSRRGKYRISLKDHKVYDLQETSKFCSAGCLIDSKTFSGSLQEARTLEFDSVKLNEILDLFGDSLEVKGSLDVNKDLDLSKLMIKENFGVRGEELSLEKWMGPSNAVEGYVPFDRSKSSNDSKATTQSNQEKHEMDFTSTVIMPDVNSVSKLPPQTKQASTVVESVDGKGKTVLKEQTVVPPTKKVSRFRREKEKEKKTFGVDGMGCAQEKTTVLPRKILSFCNEIEKDFKNFGFDEMGLASSAMMSDGYGVEYSVSKQPQCSMEDSLSCKLKGDLQTLDGKNTLSGSSSGSNTKGSKTKPEKSRKKIISVEYHANSYEDGEEILAAESYERHKAQDVCSSSEIVTKSCLKISGSKKLSRSVTWADQNDGRGDLCEVRNNDNAAGPSLSSNDIEDVNSLSRLALAEALATALSQAAEAVSSGNSDASDATAKAGIILLPSTHQLDEEVTEEHSEEEMTEEEPTLLKWPNKPGIPDSDLFDRDQSWFDGPPEGFNLTLSNFAVMWDSLFGWVSSSSLAYIYGKEESAHEEFLLVNGKEYPRRIIMVDGLSSEIKQTIAGCLARALPRVVTHLRLPIAISELEKGLGSLLETMSLTGAVPSFRVKEWLVIVLLFLDALSVSRIPRIAPYISNRDKILEGSGIGNEEYETMKDILLPLGRVPQFATRSGA

SEQ ID NO: 12 - protein, 1544 aa, Oryza sativa
MDDAAERRRRQQQQQQPGAAHPARRKVVEEPFDPSPPPAAAVAPPSSRLVGAIVEKGFSSGAAAAAPSSAPSPTVLPFPVARHRSHGPHWKPAARDAAMAEGEGEEEEGMDVDETDYQPVAAAAGPVKRKEKKGMDFSRWREFVADDAPPKRRQAKPLQPKKQTAQKIDTGVVAATTGGTAQEKRSGGIGMQLEVGNGKEELGGAALMSDVAPRKPMKQVDARDDVRNVELRGEGMESDNGEPSLTAEINAENMARLAGMSAGEIAEAQAEILNRMDPAFVEMLKRRGKEKSGSRKDGGKGKGGGISGPGKISKAMPGEWLSAGEHSGHTWKAWSERVERIRSCRFTLEGDILGFQSCQEQQHVFWYPLHVNLAFPLTGKKAHVETVGERDFLRTEGDPAAVGYTINEAVALSRSMVPGQRVLALQLLALILNRALQNLHKTDLIDNFKESNDDDKFNDWQAVWAYAIGPEPELVLSLRMSLDDNHDSVVLTCAKVINAMLSYEMNEMYFDVLEKVVDQGKDICTAPVFRSKPDQNGGFLEGGFWKYNTKPSNILPHYGENDEEEGDEKHTIQDDVVVSGQDVAAGLVRMGILPRICFLLEMDPHPILEDNLVSILLGLARHSPQSADAILNCPRLVQSVVKLLVKQGSMEIHSSQIKGVNLLKVLSKYNRQTCFNFVNTGVFHQAMWHWYRKAYTLEDWIRSGKEHCKLTSALMVEQLRFWRTCISYGFCITHFTDFFPILCLWLSPSMFQKLSESNVVAEFSSIATESYLVLGALAQRLPLLHSVEQLSKQDMGLSGIQVETWSWSHAVPMVDLALSWLCLNDIPYVCLLISGQSKNILEGSYFALVISSVLGMLDSILERISPDSTHDGKSYCLPWIPDFVPKIGLGVITNGFFNFLDDNAVELEQHTSFHGSSLVQGLFHLRSQGNVDTSLCSISCFQRLLQLSCSIDRVIQNATTNCTEHLKESKTGIAGRILEQGICNFWRNNLLDMLTSLLPMISSQWSILQNIEMFGRGGPAPGVGFGWGAYGGGFWSLNFLLAQLDSHFVLELMKILSTGPEGLVTVNKSVNPIVQEGNNVTDSVAITSERISSVLSVSLMAGPGQISTLEKAFDILFHPSVLKFLKSSVLDSHMKLAKAFEWDITEDEYLHFSSVLNSHFRSRWLVIKKKHSDEFTRNNNGTNVPKIPETLETIQEETELAEAVNPPCSVLAVEWAHQRLPLPVHWILSAVCCIDDPKANLSTSYAVDVSKAGLFFLLGLEAISAAPCLHAPLVWKMHALSASIRSSMDLLLEDRSRDIFHALQELYGLHLDRLCQKYDSAHSVKKEGSASVDEEKVTRTEVLRFQEKIHANYTTFVESLIEQFAAVSYGDALFGRQVAIYLHRSVEPTIRLAAWNALSNAYVLELLPPLDKCVGDVQGYLEPLEDDEGILESYAKSWTSGALDKAFQRDAMSFTVARHHLSGFVFQCSGSGKVRNKLVKSLIRCYGQKRHHEDMLKGFVLQGIAQDSQRNDEVSRRFEIMKDACEMNSSLLAEVRRLKTSIDR

SEQ ID NO: 13 - protein, 1528 aa, Zea mays
MDATTKRRHQPGGAQPTRRKVVEEPFHTAPPTPAAASPSRLVGAIVEKGYSAAAPSSAPRPSVLPFPVARHRSHGPHWVPLVKDAPKDETADNDDEMDMDETDYHPVAAAAAGPVRRKEKKGMDFSRWREFVGDAPPKRRQGKPVQAKKQSDQRIDAGAVASKVGGVAAEGRGLEGGAMRLDSGNASEGPGPVLLVSDVVSKKPMSQVESRDELVNTSEARNLASQAESMDLDGRESSMEAEISAENMARLAGMSAGEIAEAQADIVNKLNPALLEMLRRRGREKSGGTKDVGKDKGLKNSGLQKNKRATPGDWLTAGEHTGHSWKVWSERVERIRSCRFTLDGDILGFQSSHEQQDGKKMPSESVAERDFLRTEGDPAAVGYTINEAVALTRSMVPGQRVLALQLLASILNRALQSLHKTDLMDNVKGMNSKDNIDDWQAVWSYALGPEPELVLSLRMALDDNHDSVVLSCTKVVNVMLSCEFNESYFEFSEKVGNGKDICTAPVFRSKPDLDGGFLEGGFWKYNTKPSNILPHCGDNDEDEADEKHTIQDDVVVSGQDVAAGFVRMGILPRICFLLEMDPSPALEDYLVSVLVALARHSPQSADAILNCPRLIQSVTKLLINQGSMEIRSSQIRGVTLLKVLSKYNRQTCLNFVNHGVFQQALWHWYRKAGTIEDWVRSGKEKCKLSSAMMVEQLRFWRTCISYGFCIAHFADFFPVLCLWLSRPDFKKLSEHNVLVEFSSVARESYLVLAALAQRLPLLHSVEQLANQDLGVSASYIETCSWSHVVPMVDLALSWLHLNDIPYVCSLISEQNRNTEHMLEMSYLILVISSVLGMLNSILERISPDVTPEDKSYSLPWIPDFVPKIGLGIISNGFFSCSTTVAGRNAEHQPFCCASLVQGLCYMRCHGNVDVSLSSISCLQRLVQLSWSVDRVIQGATKCCSECFNESGTGEAGKLLAEGISSLWHNDLLHLLTSLLPMISSQWSISQNIEMFGRGGPAPGVGFGWGTCGGGFWSLKCLLAQLDSQLVVELIKCFSSVQGSPIILDEGVKLDNVTNTVVTASNWISSTLGLSLIAGPGQIYMLEKVFDMIFEPSILKYLKSSIHKFTSDMELLKPFEWDLNEDEYMLFSSVLKSHFRSRWLAIKKKHSDKYAGDNSSTKISKTPEILETIQEETELSEAVNQPCNTLMVEWAHQRLPLPIHWILSAVCCIDDPKGTLSTSANYILDVSRAGLIFLLGLEAISATPCLHAPLIWKIHALSVSIRSSMHLLQEDRSRDIFCALQELYGLHLNRLYQKFCKPNSIEEVKGVVVGTSEEAMEISSLEILRFQEKIHGSYTTFVESLVDQFAAVSYGDFVFGRQVAIYLHRKAEPAVRLAAWNALSSAYVLELLPPLDNCIGNAPGYLEPLEDDEKILESYAKSWTSGVLDKALQRDSMAFTLAKHHLSGFVFQSSDSGTMLRKKLVKSLIRCYAQKRHHEVMLKCFVQQGIAQDSKSSELDRRFEILKDACEMNSNLVGEVQRLKACLGQ

SEQ ID NO: 14 - protein, 1110 aa, Glycine max
MTKNENKVDKSVDWEAVWAFALGPEPELVLSLRICLDDNHNSVVLACTKVVQSVLSYDANENYCDMSEIATCDMDICTAPVFRSRPDINDGFLQGGFWKYSAKPSNILPFSDDSMDNETEGKHTIQDDIVVAAQDFTVGLVRMGILPRLRYLLEKDPTTALEECIISILIAIARHSPTCANAVLKCERLVQTIVNRFTADNFELRSSMTKSVKLLKVFARLDQKTCLEFIKKGYFQAMTWNLYQSPSSVDHWLRLGKEKCKLTSALIVEQMRFWRVCIQYGYCVSYFLEMFPALCFWLNPPSFEKLVENDVLDESTSISREAYLVLESLAGRLPNLFSKQCLNNQLPESAGDTEVWSWNYVGPMVDLAIKWIASRSDPEVSKFFEGQKEGRCDFPFRDLSATPLLWVYAAVTRMLFRVLERMTWGDTISSFETEGHVPWLPEFVPKIGLELIKYWFLGFSASFGAKFGRDSEGESFMKELVYLRQKDDIEMSLASTCCLNGMVKIITTIDNLILSAKAGICSLPRQEQSLSKEGKVLEDGIVNGCLVELRYMLDAFMFSVSSGWHHIQSIESFGRGGPVPGAGIGWGAPSGGFWSATFLLAQIDAKFLVSLLEIFENASKGVVTEETTFIIQRVNAGLGLCLTAGPREKVVVEKALDLLFHVSVLKNLDLCIHNFLFNRRGRTFGWQHEEEDYMHLRRMLSSHFRSRWLSVKVKSKSVDGSSSSGIKTSPKVGACLETIYEDSDMSSMTSPCCNSLMIEWAHQKLPLPVHFYLSPISTIFHSKRAGTKKVDDVLHDPSYLIEVAKCGLFFVLGVEAMSIFHGTDIPSPVEQVSLTWKLHSLSVNFLVGMEILEQDRSRVTFEALQDLYGELLDKARLNQSKEVISNDKKHLEFLRFQTEIHESYSTFLEELVEQFSAVSYGDVIFGRQVSLYLHRYVETSIRLAAWNTLSNARVLELLPPLEKCFSGAEGYLEPAEDNEAILEAYTKSWVSDALDRAAIRGSVAYTLVVHHLSSFIFHACPMDKLLLRNRLARSLLRDYAGKQQHEGMLLNLIHHNKPPPSVMGEELNGGVLSERNWLESRLKVLVEACEGNSSLLIVVEKLKAAVEKSS

SEQ ID NO: 15 - protein, 1387 aa, Glycine max
MKVDTKPLLDNSDGGFINSTTTMEVDTLNKEQNESVPGLDQISSDWMPDYNFGSLDVQRPGQTDLNSSMLEQKSVSLDSEIDAENRARIQQMSAEEIAEAQTEIMEKMSPALLKLLQKRGQNKLKKLKLEVDIGSESVNGHAQSPQDAKHLHTEDGIAQTVIVPPSKEKLDDEKISTKTSTTASSSAWNAWSNRVEAVRELRFSLVGDVVDSERVSVYDNANERDYLRTEGDPGAAGYTIKEAVALTRSVIPGQRTLALHLLSSVLDKALHYICEDRTGHMTKIENKVDKSVDWEAVWAFALGPEPELVLSLRICLDDNHNSVVLACAKVVQCVLSYDANENYCNISEKIATCDMDICTAPVFRSRPDINDGFLQGGFWKYSAKPSNILPFSDDSMDNETEGKHTIQDDIVVAGQDFTVGLVRMGILPRLRYLLETDPTTALEECIISVLIAIARHSPTCANAVLKCERLVQTIANRYTAENFEIRSSMIRSVRLLKVLARSDRKSCLEFIKKGYFQAMTWNLYQSPSSIDHWLRLGKEKCKLTSALIVEQMRFWRVCIQYGYCVSYFSEMFPALCFWLNPPSFEKLVENNVLDESTSISREAYLVLESLAGKLPNLFSKQCLNNQLPESAGDTEVWSWNYVGPMVDLAIKWIASRNDPEVSKFFEGQEEGRYDFTFRDLSATPLLWVYAAVTHMLFRVLERMTWGDTIETEGHVPWLPEFVPKIGLEVIKYWFLGFSASFGAKCGRDSKGESFMKELVYLRQKDDIEMSLASTCCLNGMVKIITAIDNLIQSAKASICSLPCQEQSLSKEGKVLEDGIVKGCWVELRYMLDVFMFSVSSGWHRIQSIESFGRGGLVPGAGIGWGASGGGFWSATVLLAQADARFLVYLLEIFENASKGVVTEETTFTIQRVNAGLGLCLTAGPRDKVVVEKTLDFLFHVSVLKHLDLCIQSLLLNRRGKTFGWQHEEEDYMHLSRMLSSHFRSRWLSVKVKSKSVDGSSSSGIKTSPKVGACLETIYEDSDTSSVTTPCCNSIMIEWAHQKLPLPVHFYLSPISTIFHSKRAGTKIVDDVLHDPSNLLEVAKCGLFFVLGVEAMSIFHGTDIPSPVQQVSLTWKLHSLSVNFLVGMEILEQDWSRDIFEALQDLYGELLDNARLNQSKEVISDDKKHLEFLRFQTEIHESYSTFLEELVEQFSAVSYGDVIFGRQVSLYLHRCVETSIRLAAWNTLSNSRVLELLPPLEKCFSGAEGYLEPAEDNEAILEAYTNLWVSDALDRAAIRGSVAYTLVVHHLSSFIFHACPTDKLLLRNRLARSLLRDYAGKQQHEGMLLNLIHHNKPPPSVMGEELNGILSEKSWLESRLKVLVEACEGNSSILTVVDKLKAVVKNSS

SEQ ID NO: 16 - protein, 1529 aa, Brachypodium distachyon
MLPMDDGTKRKHQPGAHPTRRKVVEEPFDPAPPLSGAATAAASAAAPPPHLVGAIVEKGFSAAAPSSSPRPTVLPFPVARHRSHGPHWNPVTKDAYKEKGEVEDYGMDVDEVDYQPMATVAGPIRRKEKKGMDFSRWREFMADDVPPKRRQAKKNSTQRIDPGIVAEKVDVSVGERALGGDGMELDGGNAKDELGVTTLVSDVLPRKPEKRVDAGDLLMLEGEAGVAEMRGEGMQLDDGEPSVAAEINAENIARLAEMSTEEIAEAQADILNRLDPTLVEILKRRGKEKSGGRKDGVKDKGGEISEPGKTARATPGARLVVGEHNGYSWKAWSERVERIRLCRFTLNGDILGFQSCQEQQDGKNRNAERVAERDFLRTEGDPAAVGYTINEALALTRSTVPGQRVLGLQLLASVLNRAVHNLHEMDLADNLEGANGADKLDDWQAVWAYALGPQPELVLSLRMALDDNHASVVLTCAKVINVMLTYDMNEAYFEFSEKVVHQGKDICTAPVFRSKPDLDGGFLEGGFWKYNTKPSNILPHYGENAEEEGDEEHTIQDDVVVSGQDVAAGLIRMGILPRICSLLEMDPPPILEDYLVSTLVALARHSPQSADAILNCTNLVQSVVKLLVKQGSMEIHSSQIRGVTLLKVLSKYNRQTCSNLVNRGVFQQAMWQWYRKAYTLEDWIRSGKEQCKLSSAMMVEQLRFWRTCISYGFCIGHFTDFFPVLCLWLSPPLFQNLSKSNVLSEFSSISRESYLVLGALAQRLPLLHSMEQLGKQDMGVSGSYIEMWSWSHVVPMVDLALSWLHLNDIPYLCSLINEQSENTAHILEESCLVLLISSVLGMLNSILERISPDGTPDVKSYCLPWIPDFVPKIGLGIITNNFFSFSRDDVVGHEDQLSFCGVSLVQGLCRMRSQGNVDASLSSICCLQRLVQLSFSVDRVIQRVSTKCSEPVKESKTGIAGKILGQGISSLWHHDLLNSLNVMLPLSSSQWPVLKNIETFGRGGLAPGVGFGWGTCGGGFWSLKCLLAQLDSQLVLELIKIFSAVPEVLVTPSKGVNSDNVTNPVAKASGRISPVLGVSLIAGPGQITTLETAFDILFHPSILKCLKSSMQSMASQMELPKTSEWEITEDEYQHFSSVLNSHFRSRWLVIKKKSDKYARDNSGINMPKLSETLDTIQEEVEFTETVNPPCGTLVVEWAHQRLPLPVHWILSSICCIDDAKGTLSVLANHAVDVSRAGLIFLFGLEAISSAPCLDAPLVWKIHALSASLRTNMDLLQEDRSRDIFNALQELYGQHLDMLCHKYYRSHSVKNDEVVGSVTTVEEAKAISSLEILGFKEKIHGSYTTFVESVIDQFAAVSYGDVIFGRQVAIYLHRSVETVVRLAAWNALSNAYVLELLPPLDKCIGDIKGYLEPFEDNEAILEAYAKSWTSGVLDKASQRDSMSFTLVRHHLSGFVFERNASIKVRNKMVKSLIRCYAQKQHHEAMLQGFVLHGTQSSDEVSRRFEILKDACEMNSSLLAEVHRLKTSIDG

SEQ ID NO: 17 - protein, 1549 aa, Sorghum bicolor
MDAPTKRRHQPGGAHPTRRKVVEEPFHPAPPTPAAAAAAAASASPARLVGAIVEKGFSAAAPSSAPRPSVLPFPVARHRSHGPHWGPVAKDAHKDGAADDDDEMDMDETDYHPVAAAAGPVRRKEKKGMDFSRWREFVGDAPPKRRQGKPVQAKKQSDQRIDAGAVASMVGGVAATGRGLEGGAMQLDSGELEGSAMQLDSGNTREGPGAVLSVSDVVSKKPMSQAESRDELVKVGEVRNSTSQAESMDLDGRESSMEAEINAENMARLAGMSAGEIAEAQTDIVNKLNPALVEKLRRRGREKSGGTKDVGKDKGLENSGPQKTKRATPGDWLTPGEHSGHSWKAWSERVERIRSCRFTLDGDILGFQFSHEQQDGKKMHSESVAERDFLRTEGDPAAVGYTIKEAVALTRSMVPGQRVLALQLLASILNRALQNLHKTDLMDNVKEMNSNEKFDDWQAIWSYALGPEPELVLSLRMALDDNHDSVVLSCAKVINVMLSCEFNESYFEFSEKVGNGKDICTAPVFRSKPDLDGDFLEGGFWKYNTKPSNILPHYGENDEDEGDDKHTIQDDVVVSGQDVAAGFVRMGILPRICFLLEMDPSPALEDYLVSVLVALARHSPHSADAILNCPRLIQSVTKLLINQGSMEIRSSQIKGVTLLKVLSKYNRQTCLNFVNHGVFQQALWHWYRKAGTIEDWVRSGKEKCKLSSAMMVEQLRFWRTCISYGFCIAHFADFFPVLCLWLSPPEFKKLNEHNVLVEFSSIARESYLVLAALAQRLPLLHSVEQLANQDRGVSASYIETCSWSHVVPMVDLALSWLHLNDIPYVCSLISGQNRNTKHMVDASYLILVIASVLGMLNSILERISPNVTPEDKSYSLPWIPDFVPKIGLGIISNGFFSCLGTVAVRNAEHQSFCSASLVQGLCYMRCHGNVDVSLSSISCLQRLVQLSWSVDRVIQGAKKSCSECFNESGTGVAGKLLGEGISSLWHNDLLHLLTSLLPMISSQWSISQNIEMFGRGGPAPGVGFGWGACGGGFWSLKCLLAQLDSQLVVELMKCFSSVQGSPVILDEGVKSDNVTNTVVTASNWISSSLGLSLIAGPGQIYMLEKAFDMIFEPSILKYLKSSIHKFASDMVLLKPFEWDINDDEYLLFSSVLNSHFRSRWLAVKKKKHSDKYTGNNSSTKISKTPETLETIQEETELTEAVNQPCNTLVVEWAHQRLPLPIQWILSAVCCIDDPKGTLSTSANYILDVSRAGLIFLLGLEAISATPCLHAPLIWKIHALSVSIRSSMHLLQEDRSRDIFCALQELYGQHLNRLCQKFCKSKSVEEVKGVVVATSEEAMEISNHEILRFQEKIHGSYTTFVESLVDQFAAVSYGDFVFGRQVAIYLHRKVEPAVRLAAWNALSNAYVLELLPPLDKCIGNAQGYLEPLEDDENFLESYAKSWTSGVLDKALQRDSMAFTLVKHHLSGFVFQSSDSGKTLRNKLVKSLIRCYAQKRHHEVMLKSFVLQGIAQDSKSSGNELDRRFEILKDACEMNSSLLGEVQRLRACLGQ

SEQ ID NO: 18 - DNA, 8785 bp, Oryza sativa
atggacgacgcggcggagcggaggcggcggcagcagcagcagcagcagccaggcgccgcccaccccgcgcgccgcaaggtcgtggaggagcccttcgacccctcccctcccccggccgccgccgtggcgccgccttcctcccgcctcgtcggcgccatcgtcgagaagggcttctcctccggcgcggccgccgccgcgccctcctccgccccgagtcccaccgtcctccccttccccgtcgcccgccaccgctcccacggccccgtaagccgcctacgcctcctccgccgccgccctctatctcggaaccctaggtttgatgtggtgctttggttttgtactccctgactgactatctgctcttccgcagcactggaaaccggcggcgagggatgctgccatggcggagggggagggcgaggaggaggaagggatggatgtggacgagacggactaccagcccgtggccgccgcagctgggcccgttaagaggaaggagaagaagggcatggatttcagcaggtggcgggagttcgtcgctgacgatgcgcccccgaagcgaaggcaggcaaagccgttgcagccgaagaaacagactgcgcagaaaattgacaccggggtcgtggctgcaacgacgggtggcaccgcacaggagaagcgctccgggggaattggtatgcagctggaagttggaaatggtaaggaagaattgggtggagctgctttgatgtctgatgtggcgccaaggaagccgatgaaacaggttgatgctagagatgatgtgaggaatgtggaattgcgaggagagggtatggaatcggataatggggaaccatctcttaccgcagagattaatgcggagaacatggctaggctggcagggatgtcagctggggagattgcagaggcacaggcagagatcctgaataggatggacccggcatttgtggagatgctgaaacgacgggggaaggagaagtctgggagcaggaaagatgggggaaagggcaagggtggggggatttcaggcccagggaagatctcgaaggctatgcctggagaatggttgtcagctggtgagcatagtggacacacttggaaggcatggagtgagagagtagagcggatcaggtcttgtaggttcacattggaaggagatattttggggtttcaatcttgtcaggagcaacaacatggtaaatcatttttctttgctcttgtgttgcttttagcttctagtgttctggtacccgttacatgtcaacctggctttcccacttacaggcaagaaagcacatgtggaaactgtaggtgagcgtgattttcttcgaacagagggagatcccgcagctgttgggtacacaattaatgaagcagtggcacttagcaggagcatggtttgttactttcttttgttgttcagtgtgaaaatggacttttgagtaaagttcaagggctaaaaatggactttacccaatgcaatacaacatctagtttctctttagtatttttagagatattaacctttgcatgtgaatggattttgttgtttttttttttaaatgtcatatcatgcataactgagatacaccatcatcgcttaatgtttttcttacatcatttctaaagtgtgcctccaaaatattgcaagataaataaatgtaataattcagtttttacgttcaaaccataggttcctggacagcgcgtgcttgcgcttcagctccttgctttgattcttaatagggccttgcagaacctacataagacggatctaattgataactttaaagaatcaaatgatgatgacaagtttaatgactggcaagcggtttgggcatatgccatcggacctgaacctgagttggttctctctctaaggtaaactggttgttttcaaactataataataattttaatttggatttgcttattccatgcaagagtttattagttaccaaagtgaagagtacttaactgaaatataccggacaattctattaagactaaataatcaagaaaccttaacacatgctttagtgtctgcacctatgcttaaaattgtatgcctgaaaatttgggctgccacatcttgtattttagtcatgaattcatgacagcaggcaagcattaacagcataaggtcatcttgtccattagctatcagttagcacttgtaagaagatgtatccagttaacttaggccatgtctagtgtaggtgaagctaaggttgtcacatcacagccatgtgatacacagggtggataatttttcttctctagtgtgctagcatcgattttagtttttgagtggactccactagaaattttaaaaactggctttcctagtgtatgcaaataaaacattttgcttcgcatcaaaccatatatgcatcgattttgttcctagcatctcctcttctagtgcctagaatcattactaagccttcaaatccgcttgctagcatcggtcaaggtgtgcactagacatgcccttattgtctttctttttgcagtcacgttggctgtttcatgtataataagttaggaaaattccatctatagcacaaagttttcataggtactaataaataccacaattttcaacccttccagaaatgccacaatgtgacaccacacgtaaaaaataccacaaaattttacagaactgaacgtatgacaaattgaactattaccatggacaaaattgcccctggcctttcttcaatctgcattcagtagtctccccgtgattccactcgtctcctctctctctctctctctctctctctctctctctctctctctcggcgagatgaagtgaagctggtggccacgaattccggcaatgaagcacggtcctacctacaatccgtcctcgaggactagcgaccgccgccagccgcggaggccgccagcgccgaggccgctgaggcgttggctctcgaggtgaagtcttccgcactcctcccgtaaccgatgaaggtctggcaaccatggccatgcctctgacctgctctagcgtgatccaatcacttggtctcacaggtctccatgatgctcttattgccggcggccattgcaacatccacacatgcatccgaatcatatgtccccaaatccgaacgcctaaatccatttcctccttgtgttattctcttggccacagaaaacacctcgttttggcaggatttgctgcgaatcaagccaaataatccattgccttcgtggtaatctgggcattttctttgtaaaatggcactgtccatccccgccttgttctgttctgagttgtggtatttctgaaagggtggaaaaatccatggtattattaggtaccagtgaaaactttgtgctatagatggaattttccctaataagttataatctcagaaaactgcggtgtgacctaattaactgttgtctataggttttttttatttagttctctcatatgccatctaattagtttctttgcttatatgagttaattgcaccagtgttagaccttaaaatggcctatgcttttttcattgtttattctacttatgtgtcatcactaactggaatatctgcaggatgtcattggatgataaccatgattctgtagttttgacttgtgctaaagttatcaatgctatgctgagctatgaaatgaatgagatgtatttcgatgttttagaggtaactttactaatttatcttctaatttgtttttcgttgagaactttatccttaaatggctgattttcaacattacagaaagtagtagatcaagggaaggatatctgtacagctcctgttttccgtagcaaacctgatcagaatgggggttttcttgaaggaggtttctggaaatacaacacaaaaccatccaatatactcccacattatggtgagaatgatgaggaagaaggtgatgagaaacataccattcaagatgatgtggttgtatcaggtcaagatgttgctgctggtcttgttagaatgggaatacttccacggatctgcttccttttggaggtgagggtctggccatctgctctccctcatcccctatctgccattgtttctttcagaatcccactgccatgcatgcacccagaatcccaccgccatgcatgcacaatatgggtgcattaggtcatcttttgtttactatatccgtgaattgtctggttccttgatgttcaatcctgtctagtacttgccatccatccttgaaatggcagtgaaatgcctccttacctggttgacacatcacttgttttcaccaggaaatctggtttgcagcagaatttgtggctggctttgattgttcttttaccatcggaaaattacaattttgatattcaacaaccaaactatgataaattgtgtatagcctttcatttttagcaatattttttatttcctgaagctatgggccttatccatttgcagttttaagtattttgtagttctatgatgtcatgtcatctgatacccgtaatctttcaaacttcttatctggttattggactgtctcgtagattgttctgttcacccttggtgctcttgcttgttgactgtttgttttatagtgatagctttatattgcagttctattacttcactagtaaacatcatttgagctagttggtcttctttttatttatgttttcatttcctattctacgagaaagtgatggtcatgattacggatcttaatggtcaagtttttgttgattcagatggacccacatccaattctagaagataatcttgtttcaattcttttgggattagcgagacactctccacaatctgctgatgctatcttgaactgcccaaggctcgttcaaagtgttgttaagttgttagtcaagcaaggatcaatggaaattcactcctcgcaaattaaaggagtcaatctcttgaaggtatcttctggtgatataaataatcttttattataggagtatgctgattttgtgtagttttggaagttgcagattatttattatatagtttttcttctatatcattggttgataataacttgatctagttatccttcagaaggattccttctgaaggataactagatcaagtctgccagtaataccacttgcaattgctgattgtacatctgaaatggtttccttttgttggtaattagctaccaacagcatcattatgagttttttttttgtttgctgcttctgatatcttaacacatagcaattgtattagccatgcctgctgttatctgaatcaatcacactccttgcacaatgccttcactgtcaacacacaactgtttggtccccccccctcctgcgggcactttagttgccccattaattcattattatggtaagatgagaaacaccatagtcataagatttgaatgttcagattatgctaaaaaaaatcattacttctatttctaaatttgtcaaagatttttttttcactagcatctaatttcttgttccttttgcctaggttttgtccaagtacaacaggcaaacatgcttcaattttgtgaacactggagtttttcatcaggctatgtggcactggtacagaaaagcctatactcttgaggattggataagatctggaaaggagcactgcaagcttacttcagcattaatggttgagcagctgcggttttggaggacctgtatctcttacgggttttgtataacgcatttcacagatttctttcctattttatgcctgtggcttagcccttcgatgtttcagaagctaagtgaaagcaatgttgtagctgaatttagttccattgcaacagagagttatcttgtattaggagctctggctcaaaggcttccacttcttcattcagttgagcagcttagcaagcaagacatgggactttctggtatccaagttgagacatggtcgtggagccatgcagttccaatggtagatttggcgctatcttggctatgcctgaatgatattccttatgtgtgtttgctaatcagtgggcaaagtaagaatatactagagggaagctactttgctttggttatttcttctgtgctaggcatgcttgattcaatactagaaaggatatctccagatagtacacatgatggtaaaagttactgcttgccttggatacctgactttgtacccaaaattggcctgggagttattactaatggatttttcaacttcttggatgataatgccgttgaacttgagcaacatacatctttccatggttcatcgttggtgcagggactttttcatttgagatcccagggtaatgttgacacatcattgtgttcaattagttgcttccaaaggttattgcagctatcttgctctattgacagagtaatccagaacgccacaacaaattgtacagagcatctgaaagaatcaaaaacagggatagctggcaggatactagaacaaggtatttgcaatttctggcgtaataacttgttggacatgctaacttcattgttgccaatgatttcctcacagtggtccatattacaaaacatagagatgtttggtagaggaggaccagcacctggtgttggttttggttggggagcatatggtggaggattttggtctttgaatttccttcttgcacaattggattcacattttgttctagaattgatgaaaatcttgtccacggggccagaaggccttgtcaccgtcaataaaagtgtgaatccaattgtgcaagaaggaaataatgtgactgattcagttgccatcacttcagagagaatcagttctgtcctcagtgtatctttgatggcaggacctgggcagatatctacactagagaaagcctttgatatcctcttccacccttctgttctgaagtttctcaaatcttcagtactagactcacacatgaaattagcaaaagcttttgaatgggacataactgaagatgagtatctccattttagcagtgtactgaattcacatttcagatccagatggttggtcatcaagaagaagcattcagatgaatttacaagaaataacaatggcacaaatgtgccaaaaataccagagacattggagacgattcaagaagaaacagagttagcagaagctgtaaatccaccttgcagtgtgttagctgtagagtgggcacaccagagattgccccttcctgtgcactggattctaagtgcagtctgttgcattgatgatccaaaagccaacctatcaaccagttatgctgttgatgtttcaaaagctggtctcttctttcttttaggtctggaagccatttcagcggctccatgtcttcatgctcctttggtttggaagatgcatgcactttctgcctctatccgcagtagcatggatttgcttctagaagacagaagtagggatatttttcatgctttgcaagaactgtatggcctgcatttggatagattatgccagaaatatgacagtgctcactctgtcaagaaagaaggatcagcctctgtggacgaagaaaaggtgaccaggactgaagttctcagatttcaggagaaaatccatgcaaactatactacttttgttgagagccttattgagcaatttgcagctgtctcatatggtgatgctctttttggtcgacaagtagccatttatttgcaccggagtgttgaacccacaattcggcttgcggcctggaacgctctgtctaatgcttatgtgcttgaactgttacctccactagacaaatgcgtcggtgatgttcaagggtacttggagcctcttgaggtattgtctctttagtttcttgtatgcttgtcttttggttggcattttgtagtaacaatgtaaatttgggtctagaagtttgctttatactactgcttttggggttgcacattacatagagtatgcaaagagtaaacaatgcatgctatttttcttaactatttgatttcggtacatacatgagcacatgccatgattctttgtaagcatgttcccacctgcttgtactttgttgcatctgtgtgataaaacagtttcagattttctgattattattttcacaataattttgtaggatgacgaaggaattttggagtcttatgctaaatcatggacttctggtgcccttgacaaagcttttcagcgggatgcaatgtctttcacagtagcaaggcatcacctttctggcttcgtcttccagtgcagcggttctggcaaagtgcggaataagctggttaaatcgcttatccggtgctatggccagaagcgccatcacgaggtaattccctcgtgctttccggaatattagtcatatatttcataaatcatgaaccatccttgatgtttggaaaattctataccgcaggatatgctcaagggttttgttctacaaggcattgcgcaagattcacaacgtaatgatgaagttagccgacgatttgaaatcatgaaggatgcttgtgagatgaattcttctcttttagctgaggttcggagactgaagacatcaattgatagataaatggccccaaaagcatttcacatgcactcagagaaggagcaatttttttggtgattgtaaatagtaacatgtgtcaatgacaaatggctagagaatagttgcattgttcgagtttactttgcattacaaaccaaccaaaggaaaacatcagtgtgtgtaggagtgctcccggttgtggcttgtgggttacattttaaaggacaatgaaacagactggtgttacagagatgctaactccaaccttatgctccagtatccaacattttctcc

SEQ ID NO: 19 - DNA, 12446 bp, Zea Mays
atggacgcgacgacgaagcggcggcaccagcccggcggcgcgcagcccacacgccgtaaggtcgtggaggaacccttccacaccgcaccccctacacctgccgcggcgtccccctcccgcctcgtcggcgccatcgtcgagaagggctactccgccgccgcaccctcctcggcgccccgaccttccgtcctccccttccccgtcgcccgccaccgctcccacggccccgtaagcagacgccgccctcccaaacaaaccctaggctcctaacccgctaacctcgatttgcttcgccgctgaccaactactacttcccgtttcgcagcactgggttcccttggtgaaggacgcccccaaggatgagaccgcggacaacgacgacgagatggacatggatgagacagactaccatcctgtggcagctgctgcagctggtcctgtgaggaggaaggagaagaagggcatggattttagccggtggcgtgagtttgttggcgatgctccccccaagcggaggcaggggaagccagtgcaggccaagaaacagagcgatcagagaattgatgctggggctgtagcttctaaggtaggtggtgtggcagctgaggggagaggattggagggaggtgctatgcggcttgacagtggaaatgctagcgaagggccaggtcccgtgcttttggtttctgatgtcgtgtccaagaagccaatgagtcaggttgaatcgagagatgaattggtgaacacaagtgaggctaggaatttggcatcacaagcagagagtatggacctcgacggcagggagtcatctatggaagcagagatcagcgcagagaacatggctaggttggctgggatgtctgcaggggagattgcagaggcccaagcagatattgtaaataagctgaaccctgcattgctggagatgctgaggcggcggggaagggagaagtctggaggcacgaaggatgtgggtaaggacaagggtctgaaaaactcagggctgcagaagaataaaagggctacaccaggggattggttgactgctggtgaacatactgggcactcctggaaggtgtggagcgaaagagtggagcggatcaggtcttgtaggttcacattagatggagacatattggggttccaatcttctcacgagcaacaagatggtataccacatggttttgctcatatgagctacatttgcttctgttggtgtggtatcctttagatgacaacctatatctcccactttcaggcaagaagatgccttcagaaagtgttgctgagcgtgatttccttagaacagaaggtgatcctgcagctgttgggtacacaatcaatgaggcagtggcacttaccaggagcatggtttgttttttttatacaccctccatttcgaattttatttgtccatttacttgggaattgattttcctgtcgaatacgaatgagagctgtttcattgcatttagaagaaaaaccagaatacaatgcacccacaatatgtgccacatccacacaccaatctcttagttacaatcgcttcagttacataaagaacatgagccgacctgaacactatcaagacaactatgagaaagttgttgaagtgtataagtggattggctaccttctcccatcagcttaagcttttgggttgaactggttagtgcgtccattctaacatggtatcaaagccagaggtctcgagttcgaatcctggcagaggctttatttgtgcctccacccatttatttccacgtttgcgcctttctctctggctgcgttcgcgcctttctctctggctgcacgtgagtgggggtgttgaagtgtataagtggattggctaccttctcccatcagcttaagcttttgggttgaactggttagtgcgtccattctaacaaaagtaaccaggcaatgcgttcaacctgccttagcccttagcagcagctgcattgcatatatttatatgggcacatggccattgttacagactaggagttgaccatttagggatgaaaatggatacttattcggatatcaattttttgatcttttttctttgattgtgaataaataggatataaaatctgttatgtaaattcatattcttgtttttagcattgagcttgtaaagttacataaaatcttaaaaagtaaacctcaaatttatcatatatcttctcaagtgatagatataaaatttggatacaattcagatttggacaccaataattttttttaccttttttgttgtagagagcaaataatgtataaaacagttcatgcaaaattttattcttatctgtaataatgtgcttgataacacaaaaaaagattaacatcaaggtgctaggcgaggtaatgagtttttttcccacaaatttggtttgtatagagcgttagctattgaattggctatatggcaagttcaatattctagtctattttatacgtatttggtgaaggtagtaaatagtaaccgttttttctcgaacgcgcaagaaaaatgcacatcattaattcattatattaagaagatgtagaaaaaggtccatgaggaccagtacaaagtcaggcaccccttacggtggccaaaaacaggaatacacaaaagaatccacaaacattctagcaaacgaaactctactcaagaccagggattggggctgttaggaggtcagaggagtatccgaggaagacacagtgagtggagcggggggctagtttatgaggagcagtggaggcgagattagggtagcaagcgcacccgaaaacacaaagatggttataggaggggtggatgccgaaaagagcgaagtaaggagtggggtgggtaaccgccttggtgggaagacagttgaggaggtaggtggcagtggcgagggcctcagcccagtagtgagcaggaagggaagcctgaaagagtagagagcgcacaacatcgttcgtcgtgcgaatcatacgttcagccctgctgttttgaggagacgtgtaggggcatgacatgcagaggtggatgccgcgagagaggaagaagtcacgggacacattgttatagaactcacgctcgttgtcacactggatgctccggatggtgcgaccgaactgagtagagacccaggcgaagaagtgagacagggtgggaaaagtgtccgacttctgacgtaaaggaaaagtccacaaataatgctagaagtcatcgagaatgagtaaataatactcatagccagaaatgctggttacatgggatgtcaatacatcacagtgtatcagatcaaaaatgcctgctgctctagaggaggaggtggggaatgggagcctaacatggtgacctaactgacaagcatgatagaggtgctcaaaagatcccctacaaccagaaacagaagtactactggagagcttggacatcacatcacgcccggggtggccaagacgacgatgccaagtcgcagaagaggcggtcgctgcaagaacatgtggggcagaggtcgaagtagtgggagcaggtagacgaagcgtgtagaggggcccggggctgtcacaccgagcgagaagagtcctggtggcaagatccttcacagaaagaccaaacgggtcaaactccatgggacaagagttgtcagtagtgaactgacgtacgaaaagaagattttgaatgatgtggggggcaacaaggacgttggttaagcggaaaggaccgggaaggaccgcgtcacctactgaggtgacgggaagggcggacccgtttccgacaatgatggaagagggaagagaagggtggacgggggaggtggaagatagaatacatgcgtccggggtggtgtggtaggaggcgccggaatccgccacccagtcagagactgaggtgggcggggccagcgtcatcatggagaaggagctggcgagggattgcgcgtcccacccgttagtccaaggcccccacatggactgagccggagaccccgggagtggcagaagaccttgctgaggctggggaggagccgagggtgccgccgggggtgcggcggcaaagaagaactgctgagaggcggtggggcaaggggccgaggcacccgtggaccgcccgagccacatgtgaatggtgccagtccacgggttgtagaaggatggccactgagagccgcccgaggaaccaccacggcctcccgacgggccatcgccactgcgtccgcccttgcgaccacggccgcgaccgcggctgcgagcccccccgaagccagcagtctgtcgcgaagctccagagggcagcggggctggagtgcggggagcaggaaccccccccggagtcggagggggcagcctgaggtgcgctgtagagggccgtggcgggagcggtggttgcagcggcggagaggcgaagctcctcgagaagcagatcgttcttagcctccgcaaaggtgggaaacggcttcatgcgggtgaggatgggggccacgcgatcgaagcgagtactcagaccacagaggaggttgagcacgaggctctcatcggtcatgggagccccgagggtgcggagagtgtctgccatcgtcttcatctgacggcagtactcgtccatagagagatcgccctgaacaagctggcggaaggcggtctctaggtacaggatccgagtctggcggttgtcgaggaactgagcctcaagggcaccccagatacggtgagcagtatcgtccggcactcgaacaatgtcttgaagttcggcggtgagggtgccatggatccaagagagcaccacttcgtccatcaggagccaatcttccgtcggtgcagcgaccgtggggaggacgtggtcggcgagggcgtagcggcggagggtctgcaggacctggtcgcgccaccgaggatactgagtggagtccggcgcgagaacgtcggtgacggttgcccggatgctggtgagggccgccgcctgggcgtgaagagaggcgcgaagggtcgaggttgggacgggcgcgggctcgaaagtgtcggcgctgtgacgagcggacatgggagaaccatctgagacgcccttgccagcgaggcgacgataggtctcggcgtactggcgctcgatggcgtcgacaaccgcctgttcctgctcaagggcacgagcggcgtcctgggcccactggcgagccgccgcaacggcagcacgggtggcagcgaggatggctgcagcgtcatccgaaggggaaggagcacgcagtgtagaggtgatagcggacgcctggctatagaccgtcgtgccaatggtgccactggaaaaaaccaggggcaacaacacggcgtcgtcgtggccgagcacggtggtctgggacgcgtgaggaagggagatcggcgcgggacagacaccggcaaagccggagccggccggactcggcagcggcacgggcgaaggaagcgccggcggcgcagggggcgcaggaggaggggcagcgaggccgggggcgggcagacccggcggcagaggtgtggccgacacgggctgtgcgccggcggggccggagccggagtcgctggagggcggtggcggcgcggggctcccggccatgagggccagcccggcgggtggtggcgcggccgacgcgggcccgatccagcggagggaggccggcggcacgggatgcccggccagagtcgcgccgcgcgcgggaggagagggcgcggccaacgcagccctgtaggagagagggagaggagggaagagctcgccggaggaggggagagggggccggccagccatggggcggcggctggaggaagggaggggtggcggcggcggctggaacagtcaaggctctggtaccatgtgggaatgtctgctgttacagaaaccctggctacagtgcggctgcggctacagtactagtaaaccctagctacagtattagtgacctaatgggccaggcccatgtacatatatctgtaacaagttctattgttccttattctcttgggtactacagcaaaaagtctaggagcaagatcctcaatcctttttctttgaagccagcggtcagtccagaacaaggttgaggagccattaccaatttcagtgattacagcaatggaaaagaatgctttcactgtttcagggagctgcatagggaacaagatccagggttttgaaggatcagtttttgctaaccacagccatctcactcttagagcccagccaagttctttttgggtttcaaagaatacatttttaccatcatctgttaaaatgtacttctaaatttttagaagttgcaatatcatgacatatgatataattcagcttcctcaaattataggttccggggcagcgtgtgcttgcactgcaacttcttgcttctattctgaatagggcattgcagagccttcataagaccgatctaatggataatgttaaaggaatgaattccaaggataacattgatgactggcaagcagtttggtcctatgcccttgggcctgaaccagaattggtactctccctaaggtaaatttacctttcttaaataccccttttattatcaatgcaaacccattttcttgggagttagcttcacaattttatctactgaggaagtgagtggagctattgcatggtttctactctttaccgatatctagtgctgacactggcattctatctactgagtgtttgtgctatgtgagtttgtgatctaaacgtcattttttatttattggatttagtgcttccttgttacatgcacggagtaatattatgcattcagatttacaatgcatataaatgaagagaaccataaatggaacatttgaatagttttgatcagtagtaaacatgtaaatacattcatggacctctcatatttgtgttgtcccttagctgtgtattttaaatttaaaggtggcatgtgtgttggtgtttgtgtgtgcatccaccttgtaatctaaaatacatggttgcattttttttgtttgacccaaagacaactacttggatttgagaccttgtgatgccattttcttttctttcctattttctactctgcactctgtcactaactgatgcatttgcaggatggctttggatgataaccatgattctgtagttttgagttgtactaaagtagttaatgttatgctgagctgtgagttcaatgaatcctattttgaattttcagaggtaattttgtcctttttgggcagttctagtctattttcgttgctagtttgagcctcattttgtcattgttatacctgcagaaagtaggtaatggaaaagatatctgcacagctcctgtgtttcgtagcaaacctgacttagatggaggctttcttgaaggtgggttctggaaatacaacacaaaaccttcaaatatactcccacattgtggtgataatgacgaagacgaagctgatgagaagcatacaattcaggatgatgttgttgtgtctggccaagatgttgctgctggttttgttaggatgggaatacttccacgaatctgttttctgttggaggtcagtttgccttattctttgatttgttgtcgtttctttgaaaatacttcaaacatgcggaagttacattgacttttgtggtttcttatgttcaataatacataacccactcgatgttatttgcacttactcatgcccccacactattgagtgatgtcaggggtggtttgaaagtttgactaatttttaatggccaatgccgctattgggccacctggtcctcaccagcaaatatcattttggtcacaccatgtcaatatttgttgctagtgaaagtgtaataagataattatgattaacttttgaatttttagcaccaaccttgggaattgggaaattttagaacatattgtggtgctaggtgtcctataattatgactccgttatcaattactcactcgtgtacagctttgttgttgttgcttgccccatcctgtggggctagtatctagggcatttctattcctcttaatttatgctcggatcccctactatttttcttttgaaaaataattgccatataattttaactggatgtacctattaatttattatatcttttatccaacatctgcatataattttgaatgtataccctggcttgtgtgctgttctcttgaactgtcttgatatttgttccactatgtattttgtagtgataactgataacggtgcactgcacttgtacatttctagtgagccctgtatcagctaatgactagttgactcatttttttgctaatatttttgttcttttcattcaaaaaactgtattcattatgggtgttaaactgaaccttttcttttccattggttcagatggatccatctcctgctttagaagattatcttgtatcggttcttgtagcactagccaggcactccccacaatctgctgatgcaatcttgaattgtccaagacttattcaaagtgttactaagctgttgataaatcaaggatcaatggaaattcgctcctcacagatcagaggggttactctcttgaaggtatttttttggttcttttgacaccccccgtgttatagagttatcatgatagatagttggaatcatcgattgacatatattgcacacatgttgatagtttgatgctgaataattcctgtacgtgcagattattattagacagatgcttctcttggtcatccttgctattttactgaccgggagatgttttaatattcccagtaatgtgtggtactccgtactcgtctgaattattagttgagctcaaatgattttctttgtatgcaagtgcattaatagttcacattgaattgccatatgatatacatgttccatgaatcggttcctttctagttcaggatgcttgctccttgtctccctcaatcacactgttctccacattttatgtacccacacatactaattatgtgttttgatattttatagtctacatattcacttatatgttccagtattattattatttgaaaatacaagcatcttgaaagctactgcaattgagtcatattccagaaaacatgtgcaattttctttgtttttctgccaagcaggacttcttttgaccatctaaagccacctattctgttttgatcaggttttgtccaaatacaacagacaaacctgcttgaattttgtgaatcatggagttttccagcaggcattgtggcactggtacagaaaagctggtactattgaggactgggtaagatctggaaaggaaaaatgcaagcttagttcagcaatgatggttgagcagctgcggttttggagaacctgcatctcctatgggttttgtatagctcactttgctgatttctttcctgttttgtgtctgtggcttagtcgtcctgattttaagaaactaagtgaacacaatgttcttgttgagtttagttccgttgctagagagtcatatcttgtcttagctgctctggcacaaaggttaccacttcttcattcagtggagcagcttgccaatcaagatctgggagtttctgccagttatattgagacatgttcttggagccatgttgtcccgatggttgacttagctttatcttggttacatcttaatgatattccctatgtatgttcactaatcagcgagcagaataggaatacagagcacatgttagagatgagttatctgattttggtgatttcttctgtgctaggcatgcttaattcaattttggaaagaatatcaccagatgtcactcctgaggataaaagttacagcttgccctggatacctgattttgtccccaaaattggcctgggcataattagtaatggttttttcagctgttcgaccactgttgctggtagaaatgcagaacatcagcctttttgctgtgcatctttggtgcagggactttgttatatgagatgccatggtaatgttgatgtatcattgtcttccattagctgccttcaaagattggtgcagctatcctggtctgtcgacagagtgatccagggagccacaaaatgttgttccgagtgtttcaatgagtctggaacaggtgaagctggcaaactactagctgaaggtatctccagtttatggcataatgatttgctacacttgctgacttcgcttttgccaatgatttcatcacaatggtccatatcacagaacatagagatgtttggtagaggaggaccagctcctggtgttgggtttggctgggggacatgtggtggagggttttggtctcttaaatgcctacttgcacaattggattcacaattggttgtagaattgatcaaatgtttctcttcagttcaaggaagtcctatcatcctcgatgaaggtgtgaagttagataatgtgactaacacagttgtgacagcttcaaattggatcagttctaccctagggttgtctctgattgctggacctggacaaatctatatgttggagaaggttttcgatatgatttttgaaccttccattctgaagtatctcaaatcatctatacataaatttacctctgacatggaattactgaaaccttttgaatgggacttaaatgaagatgaatatatgctcttcagcagtgttctcaaatcacatttcagatccagatggttagccatcaagaagaagcattcagataaatatgcaggagataatagcagcaccaagatttcaaaaacaccagagatattggagacaattcaagaagaaacagagttgtcagaagctgtaaatcaaccttgcaacacattaatggtagagtgggcgcatcagagactgcctcttcctatccactggattctaagtgcagtttgctgcattgatgatccaaaaggcacactctcaacatcagccaactatattcttgatgtctcaagggctggtcttatcttccttttaggtctggaggccatttcagctaccccgtgccttcatgctcctttgatctggaaaattcatgcactttcggtctctatccgctctagcatgcatttgctacaggaagacagaagtagggatattttctgtgctttacaggaactgtatggcctgcatctgaacaggttataccaaaaattctgtaaaccaaactctatcgaggaagttaagggcgttgtagtgggcacttcagaggaagcgatggagatcagtagccttgaaattctcaggtttcaggagaaaattcatggaagctatactacttttgttgagagcctggttgatcaatttgcagctgtctcatatggagattttgtttttggtcggcaagtggccatttatctgcatagaaaggctgagccagcagtacgtcttgcagcatggaatgcactgtctagtgcgtatgtgcttgaactgttacccccgctagacaattgcattggcaacgccccaggatacttggagcctcttgaggtacattttctttaatttattttgcatttctcttccagagaaaccttttcatggagtaactatgtgagtgatttattttgcaacttgaccatgtactcttgtctctgtgtgttgtagagcctagaatatgcaataggcacatcatgatgcattactattttgctgacatttattttggtttctttatgtatgaaatccatccaccatttagtatgatgaacttcttttgttggctgcttttccatgttcaaaggacagtctcactttgtcaaatttttacaatacttctgcaggatgatgaaaaaattttggaatcttatgctaaatcatggacgtctggtgtcctggacaaagctttacagcgtgattccatggccttcacattggcaaagcatcacctttcaggctttgtcttccagtccagcgattctggcacaatgctgcgaaaaaaactggtcaaatcgcttatccggtgctatgcacagaagcggcatcatgaggtagttggtcgctcatgtttctttgtttgcttggtccacggcaattccttccacgccactgtctgagtgtctgttgaattctgtacaacaggttatgcttaagtgcttcgttcagcaaggcatcgcacaggattccaagagcagtgagcttgaccggagatttgaaatcttgaaggatgcttgtgagatgaactccaacctcgtaggtgaagtccagagactgaaggcatgtcttggccaatgaagccagatatttaagtgtgtcattaacttggcagcgttagttgttgggaaagctcgacaaagtggcccaaaatatgcaactgaggaaactcacggtcggggctagtggttacttttaattttgatgaagggaacgccgagcacgcagcaaccctgagtctaacctggctatggttaggctgctcttagcagagcatgcatatggtcatgtaaacagtagaccacactgtctattgagtgaagtttgaaatagactttatatatagcattaggcttcctttgattcc

SEQ ID NO: 20 - DNA, 6718 bp, Glycine max
attcctggacaaagagcccttgcattgcatctcctttcgtctgtgcttgataaggcattacactatatttgcaaagacagaacagggtatatgacaaaaaatgagaacaaagttgacaaatcagttgactgggaagctgtttgggcttttgcactgggcccagaacctgagcttgtgttgtcacttaggtaaagtttgctttttggtccaagctgttggtattataaactacctgctataactgagtgaagatgcatggttttctttttcctcttttaagaaaatgaataatgttgtttatattgctgttaatcattaagcatacaagttgattggtttggatgtaagtaaaattccagctaggttttatgtgtgtaattttaatatcacttccatctggcgtgtgacatatatctggttccattactgagtatttacatgtcttgcaccctagaagccagccttatttgtggtaaaatgttttataatattattatgccttagaatactttagagtatcttagttaatctcgagagtggtttccatatttaattatttttgatgattttttaattattaggaatttaggatttggaaccttgtcttaaggatgagggttagtgctttgtcttttgtaaaatatcattacacaccaatgaagataaaattctctcctattctctttatttatttaaatccccataatatgaacctggttccgaagtgatcccatcatctgtgatttgaccccatcacttgctgccttgttctctaaacgggtgctcatctttgtttctgtgtttttttttttcttttaatttttggtcgctgtgtccttagtccttgaggattcttatcctcttatatttgtatctttctcttctatcttaatatattctttgcttatataagctttgttgattattatgctttgggatgagctcttgtgtatactggctttttctaatctgtgccctttttttgtggtcttcatcctaaaactgtgatgatcatcatatgttaaaatggatgctggttggcccttggacccttagggaattttacttcctgcataactgtttctaaaattcaattggaagagtgggaatctagattgcatacttgactcacaccttctgtcctcttgtatttaataatatggagtgttggtgaggctataaagattttttattatgaattataaattactaaatgcaaataaatgtcatcccaaatagatggctatatatcttgtcaatatgtttgaattttgcctatagatggtattattatgttgtaatccatgtggtattttgagttgcttatgataaatatactgatgaatactttaattctttcaaattgtattagagtttaatgcccatgcattggtagtgtaaaaaagtttgacactgtcatccaataacaaatcaatgtttgaattactttaaaataattattttataagtcaaaaaacttaccattcattgtgggttgtgattggataactgtaaaactttttacactgtcagtacataatcctttttctcattgtattatcataaatttatttttttcctcattatatgggcatcttttgacggcagttgtgctctaattttgtaggatatgtcttgatgataaccacaattctgtagttctggcctgcacaaaagttgttcaatctgtattgagttatgatgcaaatgagaactactgtgatatgtcagaggtaactggatttttattatcttttaatttgacaatattattatgtttaaaattagattcatctgattttttattttaattccagaagatagcaacttgtgacatggatatttgcacggctccagtttttaggagcagaccagatattaatgatggattccttcaagggggtttctggaagtacagtgccaaaccttctaatattcttcctttcagcgatgattcaatggataatgagactgaaggaaaacatactattcaagacgatatagtggttgctgcgcaagattttactgtaggtctagttcgcatgggaatccttcctaggcttcgttatcttttggaggtaaaacataattaatcattgatcatgctgaaacatttgatgtcatgagactctatctacaaagattaaggatatttttgttttatgatgtttgaacttccttgtcaaattcattattctctaatggttgcagaaagatcctacaacagctttagaagaatgtatcatttctatactgattgctatagcgaggcattcacctacatgtgctaatgctgtactgaaatgtgaaagacttgttcagacaattgtgaatagatttactgctgacaattttgaacttcgatcttctatgactaaatctgtaaaactcttgaaggtgagtggtaattttccattttattttataggaagtgatttctacatcagcagccagagaatcattcctaatgtttcatttgattttggaaaaggtgtttgctcggttagaccagaaaacttgtttagagtttataaagaaagggtattttcaggctatgacttggaatttatatcaaagtccttcctccgttgaccactggctaaggctagggaaggaaaaatgtaaactcacatctgctctgattgttgaacaaatgcgtttctggagggtctgcattcaatatggatattgtgtgtcttacttcttggaaatgttccctgccttgtgtttttggttgaatccaccttcatttgaaaaacttgttgaaaacgatgttttggatgaatctacttccatctctagggaggcataccttgttctggagtctttggctggaaggcttcctaatctattttcaaagcagtgtctaaacaaccaacttccagagtctgctggtgacaccgaggtatggtcttggaattatgttggtccaatggttgacttagccataaagtggatagcaagcagaagtgatccagaagtatccaagttctttgagggacagaaagaaggaagatgtgactttcctttccgagatctttctgcaactcctttgttgtgggtgtatgctgctgtgactcgcatgcttttcagagtgcttgaaagaatgacatggggggatactatcagctcttttgaaactgaagggcatgtgccatggcttccagaatttgtacctaagattggacttgagttgatcaaatattggtttttgggcttttctgcatcctttggggcaaaatttggaagagattctgaaggtgaatcttttatgaaggaactagtttatttgaggcagaaggatgatattgaaatgtctttagcttccacctgttgtctaaatggaatggtcaagattattactacaattgataatctgatactgtctgccaaagctggcatctgtagtctcccacgccaagaacaaagtctctcaaaagaaggaaaagtgcttgaggacggcattgttaatgggtgtttggttgagttaaggtatatgcttgatgctttcatgttttcagtttcttcagggtggcaccacatacagtccattgagtcatttggcagaggaggaccagttccaggggcaggaattggatggggtgccccaagtggaggattttggtcagcaacatttttattggcacaaatagatgcaaaatttctcgtctctttgctggaaatttttgaaaatgcgtctaaaggtgttgtgactgaagaaacaaccttcattatccaaagggtgaatgctggcttgggattgtgtttaactgcaggacctagagaaaaggttgttgtagaaaaggcattggatctcttgttccatgtctctgttttgaagaaccttgatctctgcatacataattttctcttcaatagaaggggtagaacttttggctggcaacatgaagaagaggattacatgcacttaagaagaatgttatcatctcatttcaggagcagatggttgtctgtaaaggtgaagtctaaatctgtggatggcagtagttcctctggcattaagacctctccaaaggttggtgcttgtttggaaaccatatatgaggattcagacatgtcttccatgacaagtccgtgctgtaattctttaatgatagaatgggctcaccagaaactaccacttccagttcacttttaccttagtccaatctcaacaattttccatagcaagcgggctggtactaaaaaagttgatgatgtactacatgatccatcttatctgattgaagttgccaaatgtggacttttctttgttttaggtgttgaagcaatgtccatttttcatggcactgacattccttctcctgttgaacaagtatcattgacatggaagttacattccttatctgtcaatttccttgttggaatggagatacttgaacaagatcggagcagggttacttttgaagctttgcaggatctttatggtgagcttcttgataaggcaaggttaaaccaaagtaaagaagttatttcaaatgataaaaagcatcttgagtttctgaggttccaaactgagattcatgaaagttactcaacttttcttgaagaacttgtggagcagttttctgctgtttcttatggtgatgttatttttggccggcaagtttcactttatctacaccgttatgttgaaacttccattcgacttgctgcctggaatacactgtccaatgctcgtgttcttgagcttttgccacctttagaaaaatgcttttctggtgctgaaggataccttgaacctgctgaggtaaaaaaaataatcattaactaccagctattttactctggagcattatttgcagaggatattgattgagtacttgttcttttgatacttttttcctttctatgttgatgatattgggttgaatatataaatgtcacttatattttatattttattataattttgtaaattttcaaaaagtggttgtttaaaatgcgcttaaaaacagaaaagcgtatgctacaaagtacagctaatgttgcagagtttaactgtgttacaaaatttacaatacctttgacatttattggttgtattgtagcatctgtcatgaattttgaggaagcttttgagagcacaggaaataccaaaataagccatactgatactgggtccttttattttttatatgatgcataataaagtgaagttcctaattggaattttggatatagtgaaggatcttatacttccagcaatataacagatagggtcacttagcaccaaacatgcacatattttttacaatgacatgtttttaaagcttgtctttagtagggaagaaatgtaaggttgtatctttgatactttgaagttagaaactatcacgatggctttgttgtgtaagttaccgtgaggaatgaggattgttgttcatcaagtttccttatctaaatttttttattttccattttttgttaatatctataacttctgtgaatagcaacaattggaaatgggtaggtgataattgtttactactgtattatctgtggaggggtgcacttgccacctgtgtggttgcaccagcaatgcatactagtcattggctcagaatacaccatatatctatcaacattaatggtgtactgtatctctgcaggataatgaagccattttggaggcttatacgaagtcgtgggtttctgatgcccttgacagggctgcaattcgaggatcagttgcatatactcttgttgtccatcatctttcctcctttatatttcatgcctgtcccatggataagttattgctgcgaaacagacttgctaggtctctgttgcgagattatgctgggaaacagcaacacgaggtaaccaaagtcttaaaagcttttatcctgatggacaaatggtagtttaagcaaatcaccattcagagcttgtgctgccattcattctccaattagttttatcccatctgattatggaaatcaaatgttatatgtgaatgtgtgctgtgcacgtgtgtgcttgttctgtcatcacgttgtttggaatgtatcagctaggactttggcctacctaataaaattttaagagtgtgcagtgagacatcactgtttaggtaaatcctagttgcagtatttttattaccaagaatttgaggccatggaagaagtataagattttgtcctatctttttcatcctaatttaatttgcattgttgcgtctgtatttacgattatatagccataatgttggtgtagccagacattactaatggaacccttgttgcagggtatgttactgaacctcattcaccataacaagccgccaccatctgtcatgggagaggagctgaatggtggtgtactttctgaaaggaattggttagagtccagattaaaagtattggttgaggcttgtgagggaaattcctctcttttgatagtagtagagaagttaaaggctgctgtagaaaagagttcatagtgaggtaactaatatatcgaatgcttgtaaatatattcgagatatacgatgagtacaactaaggtttatatatagatttttgcaatggggtctcaaatgcattaattggagcatacatggccactgatattgatattttttgttagaatggagtctgctgattttttaatcgagtcctaaggcatgcaatgtacataatgaaggaatacaagtattttagtcgggttattataaaatacactgtttcactcc

SEQ ID NO: 21 - DNA, 9771 bp, Glycine max
gagtttgaaaaagtttcagcctttgccaagccggtacagaggaggaggaaaaagggtttggattttagaaaatggaaagagatcactcgggatgatagttcttccttcgggaaggaatcagagaaggatgtgtcaagctttagccaaactactgggaaaaagaagaatgaaaagggcagtaagagcacatacaagaaaacctcatctttggatgataatgtcatttctccaatgaaagtggatacaaaaccactgttagataactcagatggtgggtttatcaattcaactaccactatggaagtagatacattaaataaggtagatcatgaggaaaaagttaaacacgccagaatttatgatgacaaggagcaaaatgaatctgtgcctggattggaccaaatttcttctgattggatgcctgattacaattttggatccctggatgtgcaaaggccagggcaaactgacttgaattcaagcatgctgtcttgttctagttccaatagtattagaagtgaacaaaagtccgtgtctcttgatagtgaaattgatgctgagaatcgagctcggattcagcaaatgtcagctgaggagattgcagaagcccagactgagataatggagaagatgagccctgcattactaaaattactgcagaagagggggcagaataaattgaagaaactaaaattagaagtggatattggctcagaatctgtgaatggacatgctcagagtcctcaggatgcaaaacatctacacacagaggatgggatcgctcaaacagtgattgtgccaccatccaaagaaaagctagatgatgagaaaattagcacgaagacttcaaccaccgctagtagtagtgcatggaatgcttggagcaatagagttgaggctgttagggagctacgattttccttggttggggatgttgttgattctgaacgtgtatcagtttatggtatgcgtttctattcttcttatgtttctttactatcttctggttctaatatcaagatgatgctttgtatgatccttgcatatgtatatatattagagtatctccttgtaccattctggactctgtagttaactaacaagtgccggctaagaattctgttattagtcagttgtaactgacagattaatttcagctaggctatctctatatatctcagacatacctcttctatttgcgatcaatgaataaacattttatcagtaatcactctgaatatctcagtgctctctttctctctctgctcgatgaatcctaacaatatatgggttcttctgtatttagtgttttgcatatgtaggtgcttggttagtgatagtaatagtattgtcttcttaatgtagatatatgctcttctgtaatttttttttctttattgggtatgtggtaaagctagtgatagtttgggttgctaatgtttgaatactttgcttcatgaaacatcaaccattgtttatagattgtgatttgtattcaatattatacattatactacctccagactaaaatataagcaacaaaaaatcaatgtgtttgggttaaaatataaacaaattttaactaactctctcctatttaatgataatatctccaaaatacccttcatttaattagagttttatttccaataaacttctcttttttgtcctatgcaattaatgtaaatggtactttaggaaataaattaactttttttattgagactaacaaaattaaataatgctaactaattttattgatgagtgtgaattagttttttttgcttatatttcagtccggagggagtagtattttttgttcccagcaattgatgatctttttttggttgactacatttctaaatgcacatgaaccaaaaaaaacacttactgtttgtttaacttatttggagaaataatcacattttttctggtttctgagagagattttgaaaaattggcgattattaatccaaatttaatctttatcaaacattttagcatttccatgtgtcatgtaggcaatatattgctggtaaatacaaatacaggaaagttaagatcaatgatgttgctatatcatggcttgtctgatagcactgagtagattatgctccctgcccccctgcatttgccacaaattacaataatgttctggttcatgcctagactgaaatttctgtatggtttcctttgcttccaaatgtttccaagttcatagacttgattagatggcttctgttgtatattcagcatattagatgttagcatttgcatataacagaacggaggtgcaaatattgatttaaataatgaatattcatggatatccattgatatcctgattattctgttagcactgatatccatcaaatcttagcatttgttgtatattactatattcattgatatcctgtttattttggaaataggatggaggtttaaatataaattttgaattttctttatgtgacaattctttgtcatgatacagacaatgccaatgaacgtgactatctacggactgagggagatcctggtgctgccggttatacaattaaagaagcagtggcactcactagaagtgtggtatgttatttgttgtaactgatgtatatttcagttggatgcctggacctggaacagtttggatggctttacaattatgaatataaaatatataaatttatagcattcccctttagtaacacagcgtctgccaaatgttttgtttttttagattcctggacaaaggacccttgcattgcatctcctttcatctgtgcttgataaggcattacactatatttgcgaagacagaacagggcatatgacaaaaattgagaacaaagttgacaaatcagttgactgggaggctgtttgggcttttgcactcggcccagaacctgagcttgtgttgtcacttaggtaaagtttactttttggtccaagctgttggtattataaactacctgctatgactgagtgaagatgcatgggtttctttttcttcttttaagaaaacgaatattgttgtttatattgctgttaatcattaatcatacaagttgataggtttggatgtaagtaaaattccagcttggttatgtgtgtaattttaatatcactttcatctggcatgtgagatggactcaatgaagcttgagatcacatatctatctgtttacattactgattatttacatgtcttgcactcttgaagccagccttttttgtggtaaaatgtttaatattattatgccttagaatactttagagtatcttagttaatctcttgagagtggtttccatatttaattatttttcatgatttcttaattaattaggatttggaatcttttcttaaggatgaggattagtgctttgtcttttgtcacatatcattacacaccgatgaagatagcattctctcctattctctttatttaattaaatccctataatattaacctggtttcaaagtggtcccatcctctgtgatttgaccccatcacctgctgcctagttctctaaactggtgctcatctttgtttctgtggtttttttcttccttccagttagctgtctttccttctgccattgtctatcttttcttctttgtatgtaaagattttgatctgtcaaatttgatgcctaattgccttatctcctagttactgcatgatgtccagattgagctatggttctggttctcatttgttcttacattgcccagcagtttcctttctctggaataatttgtttagtgttttcatagaatatggggttcgtccctagaatcttcagcacttcctgtatgttaattttggaggatttggtactagctgagaatccagaaagttttggtgttcttctctgtgtgaagttctttgggtatattcagttagaaatcatcttctgtggatgatagtcttctaatgagttcattcgggttcagctagtttttctagcttctttgtggtgttctgcttatggtctttatatataagattttatttttgtttgattgattgtgtgtttcttaatctttgtttttttcctttaatttttggttgtcgtgtctttagtccttgaggattcttatcctcttatatttgtatctttctcttctatcttaataaattctttgcttatataagctttgttgattgttatgttttgggatgagctcttatgtatactggcttttcctaatctgtgcccttttttgtggtttcttcctaaaactgtgatgagcatcatatgttaaaacagaagctggttggcccttggacccttgggtaattttactacctgcatagctgtttctaaaattcaattggaagagtgggaatgtagattgcatacttgattgactcataataccttctgtcctcttgtatttaataacatggagtgttggtgaggctagaaagatattttattttgaattataaattactaaatgcaaataaatgtcatcccaaatagatggctatatatcttgtcaatatgtttgaattttgccaatagatggtattattatgttgtgacttgtgatccttgtgctattttgactagcttatgataaatatactgatgaatactttaattctttcaaattgtatttttataatttttttcctcattatatggccatcttttgacagcagtattgtgctctgattttttgtaggatatgtcttgatgataaccacaattctgtagttctggcctgtgcaaaagttgttcaatgtgtattgagttatgatgcaaatgagaactattgtaatatctcagaggtaactggatttttattatcttttaatttgacaatattgttgcgtttaaaattagattcatctggttttttattttaattccagaagatagcaacttgtgacatggatatttgcacggctccagtttttaggagcagacctgatattaatgatggattccttcaagggggtttctggaagtacagtgccaaaccttctaatattcttcctttcagtgatgattcaatggataatgagaccgaaggaaaacatactattcaagacgatatagtggttgctgggcaagattttactgtaggtctagttcgcatgggaatccttcctaggcttcgttatcttttggaggtaaaacataattaatcattcatcatgctgaaacatttgatgtcatgagattctgtctacaaagattaaggatatttttgttttacaaggtttgaactttcatgtcaaattcattattctctaatggttgcagacagatcctacaacagctttagaagaatgtattatttccgtactgattgctatagcgaggcattcacctacatgtgctaatgctgtactgaaatgtgaaagacttgttcagacaattgcaaatagatatactgctgaaaattttgaaattcgatcttctatgattagatctgtaagactcttgaaggtgagtggtaattttccattttattttacaggaagttatttctgcatcatcagctagagaatcattcctaatgtttcatttgattttggaaaaggttttagctcggtcggaccggaaatcttgtttagagtttataaagaaagggtattttcaggctatgacttggaatttatatcaaagcccttcctccattgaccactggctaaggttagggaaggaaaaatgtaaactcacatctgctctgattgttgaacaaatgcgtttctggagggtctgcattcaatatggatattgtgtgtcttacttctcggaaatgttccctgccttgtgtttttggttgaatccgccttcatttgaaaaacttgttgaaaacaatgttttggatgaatctacttccatctctagggaggcttaccttgttctggagtctttggctggaaaacttccaaacctattttcaaagcagtgcctaaacaatcaacttccagagtctgctggtgacacagaggtatggtcttggaattatgttggtccaatggttgacttagccataaagtggatagcaagcagaaatgatccagaagtatctaagttctttgagggacaggaagaaggaagatatgactttactttccgagatctttctgcaactcctttgttgtgggtgtatgctgctgtgactcacatgcttttcagagtgcttgaaaggatgacatggggggatactattgaaactgaagggcatgtgccatggcttccagaatttgtacctaagattggacttgaggtaatcaaatattggtttttgggcttttctgcatcttttggggcaaaatgtggaagagattctaaaggcgaatcttttatgaaggaactagtttatttgaggcagaaggatgatattgaaatgtctttagcttccacctgttgtctaaatggaatggttaagattattactgcaattgataatctgatacagtctgccaaggctagcatctgtagtctcccatgccaagaacaaagtctctcaaaagaaggaaaagtgcttgaggatggcatcgttaaagggtgttgggttgaattaaggtatatgcttgatgttttcatgttttcagtttcttcagggtggcaccgcatacagtccattgagtcatttggcagaggaggactggttccaggggcaggaattggatggggtgcctcaggtggaggattttggtcagcaacagttttattggcacaagcagatgcaagatttcttgtctatttgctggaaatttttgaaaatgcatctaaaggtgtcgtgactgaagaaacaaccttcaccatccaaagggttaatgctggcttgggattgtgtttaactgcaggacctagagataaggttgttgtagaaaagacattggatttcttgttccatgtctctgttttgaagcaccttgatctctgcatacagagtttactcttgaataggaggggtaaaacctttggctggcaacatgaagaagaggattacatgcacttaagcagaatgttatcatctcatttcaggagcagatggttgtctgtaaaggtgaagtctaaatctgtggatggcagtagttcctctggcattaagacctctccaaaggttggtgcttgtttggaaaccatatatgaggattcagacacgtcttccgtgacaactccgtgctgtaattctataatgatagaatgggctcaccagaaactaccacttccagttcacttttaccttagtccaatctcaacaattttccatagcaagcgggctggtactaaaattgttgatgatgtactacatgatccctctaatctgcttgaagtcgccaaatgtggacttttctttgttttaggtgttgaagcaatgtccatttttcatggcactgacattccttctcctgttcaacaagtatcattgacatggaagttacattccttatctgtcaatttccttgttggaatggaaatacttgaacaagattggagcagggatatttttgaagctttgcaggatctttatggtgagcttcttgataatgcaaggttaaaccaaagtaaagaagttatttcagatgataaaaagcatcttgagtttctgaggttccaaactgagattcatgaaagttactcaacttttcttgaagaacttgtggagcagttttctgctgtttcttatggtgatgttatttttggccggcaagtttcactttatctacaccgttgtgttgaaacttccattcgacttgctgcctggaatacactgtccaattctcgtgttcttgagcttttgccacctttagaaaaatgcttctctggtgctgaaggataccttgaacccgctgaggtaaaaaaacatattcattcactaccagctattttactatggaacattatccgcagaggatattgtttgagtacttgttctttgatacattttttctttctatgttgatgatattgggttgaattataaatgtcacctatattttatattttataattttgtaaattttcaaaaagtggttgtttaaaatgtgcttaaaagcataaaagcatatgctacaaagtacagctaatgttgcagaatttttaattgtgttacaaatttacaatacctttgacatttattggttgtattgtagcatctttcatgaattttgaggaagcttttgagagcacaggaaataccaaactaagccatactgacactgggtccttttattttttatatgatacataacaaaagtgaagttcctaattggaattttggatatagtcaagaatcttatacttccaacaatataacagatagtcactttggcacaaacatgcacatattttttacaatggcttgctttaaagcttgtctttagtagggaagaaatgtaaggttgtatctttgattctttgaagttagaaactatcacgatggttttgtcatgtaagttacctcggaggaatgaggattgttttcatcaagtttccttatctaaaatttttattttccatttttttgctaatatctataacttatgtgaatagaaacaattggagctcaatgggtaggtgggataattgtttactactgtattatctgtggaggggtgcacttgccacctgtgtggttgcaccagcaatgcatactagtctttggctcagaatacaccatatatctatcaacattaattggtgtactgtatctctgcaggataatgaagcaattttggaggcttatacgaatttgtgggtttctgatgcccttgacagggctgcaattcgaggatcagttgcatatactcttgttgtccatcatctttcctcctttatatttcatgcctgtcccacggataagttattgctgcgaaacagacttgctaggtctctgttgcgagattatgctgggaaacagcaacatgaggtaaccaaagtcttgaaagctttatcctgatggacaaatggtagtttaagcaaatctccattcagaatttgtgctgccattcattctccaattagttttagcccatctgattaaggaaatcaaatgttatatgtgaatgtgtgcttgttctgtcatcaagttgtttggaatgtatatcagctaggactttggcctaccaaataaaattttaagagggtagagtgagacatcactgtttaggtaaaatcctttttatgaccaagagtttgaggccatggaaaaagtattatcttttacctcctaatttaatttgcattgttgcatctgtattttcaattatacatccataatgttggtgtggccaggcattactatttactaatggaactcttgttgcagggtatgttactgaacctcattcaccataacaagccaccaccatctgtcatgggagaggagctgaatggtatactttctgaaaagagttggttagagtccagattaaaagtattggttgaggcttgtgagggaaattcctctattttgacagtagtagataagttaaaggctgttgtaaaaaacagttcatagtgaggtaactaatattgaatgcgtgtaaatatattcgagatatacaatgagctatacataaagtacaacagaggcttatatatagattttttcaatgatgtctcaaatgtattaattggagcatgcatggccactgatattggtatattttgttagaatggagtctgctggtttttttatcaagattctgggaaccaagtcctaaggcatgcaatgtacataatgaaaggatacaggtattttagttaggttattataaaatatactctttcatc

SEQ ID NO: 22 - protein, 726 aa, Oryza sativa
MGPTTATDTGARMKPTTVASAVHRVQMALYDGAAASREPLLRAAASLLSGPDYADVVTERSIADACGYPACPNPLPSEDARGKAAPRFRISLREHRVYDLEEARKFCSERCLVASAAFGASLPPDRPFGVSPDRLDALVALFEGGGGGGDDGGLALGFGASGDGKEVEEGRKVEIMEKEAAGTGEVTLQEWIGPSDAIEGYVPRRDRVVGGPKKEAKQNDACSAEQSSNINVDSRNASSGESGMVLTENTKAKKKEATKTPLKMFKQDEDNDMLSSCISDSIVKQLEDVVLEEKKDKKKNKAAKGTSRVGKSKPAKRPVGRDGHEVDFTSTIIMGDRGSEMMDHGALGQYNFSSSILANEQPSSSQYAAIDSVQAYTEELDELFSNAVNIAKDETSDDSGRCTLRSSLKAVGSKNAGHSVKWADENGSVLETSRAFVSHSSKSQESMDSSVRRESAEACAAALIEAAEAISSGTSEVEDAVSKAGIIILPDMVNQQQYNNDYDNDKDAGENEIFEIDRGVVKWPKKTVLLDTDMFDVDDSWHDTPPEGFSLTLSSFATMWAALFGWVSRSSLAYVYGLDESSMEDLLIAGGRECPQKRVLNDGHSSEIRRALDTCVCNALPVLVSNLRMQIPVSKLEITLGYLLDTMSFVDALPSLRSRQWQLMVLVLLDALSLHRLPALAPIMSDSKLLQKLLNSAQVSREEYDSMIDLLLPFGRSTQSQASLPS

SEQ ID NO: 23 - protein, 725 aa, Zea mays
MSPPAPAAAAAAAPRTVASAVLRVQMALLDGAAVSSEALIHAAASALLSRADYDDVVTERTISDVCGNPACPNPLSSSSAAATGPRFHIALSEHRVYDLEEARKFCSERCLVASKALAASLPHDRPYGVPLDRLAAVVALVEGAAAGDGSGLGFQGLDGNGKVEDGGRKVEIKEKQVAGAGEVLLQDWVGPSDAIEGYVPRHDRSAHGQKPQVQQNEGAGPELSRTENVDYGAAAPGEDGMTSSPSLVKTHVSSEVIVERMGSLVLGENTRTPRKKKTKTPSKMLEQEEDNSMLSSCISDSIAKQLEDVVLEERKGSQKNKMSKASSRAQKGKSTKRPASTNMEENAMNQYNYLSSSVLVDNHPSSSQSSEKDSTQAYSEQLCEEFSEAVNIGNDETSDEKMRPAWKSSLKVAGSKSSRQSVTWADENGSVLETSKAYESPSSSIKRPEEGIDNSLRRASAEACAAALVEAAEAISSGTAEAEDAVSNAGIIILPDMLNQQEHDNGKNSGGDDDPEIDRDVIKWPKKPVLLDTDLFEVDDSWHDMPPEGFSLTLSAFGTMWAALFGWISSSSLAYVYGLERGSVEELLIANGRECPEKTVLKDGLSLEIRRALDSCVCNAVPVLISNLRLQIPVSKLEITLGYLIDTMSFVDALPSLRSRQWQAVVLVMLDALSVHQLPALAPVFSNSKLVQKMLNAAQVSREEYDSMVDLFLPFGRSVQAITPM

SEQ ID NO: 24 - protein, 709 aa, Glycine max
MAKDKPVSVKDAVFKLQMSLLEGIQNEDQLFAAGSLMSRSDYEDIVTERSITNMCGYPLCSNALPSDRPRKGRYRISLKEHKVYDLQETYMFCSSNCLVSSKTFAGSLQAERCSGLDLEKLNNVLSLFENLNLEPVETLQKNGDLGLSDLKIQEKTERSSGEVSLEQWAGPSNAIEGYVPKPRNRDSKGLRKNVKKECPFIIMFNVRPMDVYGMTVNEMGFVSTIIMQDEYSVSKVPPGQMDATANHQIKPTATVKQPEKVDAEVVRKDDDSIQDLSSSFKSSLILSTSEKEEEVTKSCEAVLKFSPGCAIQKKDVHSISISERQCDVEQNDSARKSVQVKGKTSRVIANDDASTSNLDPANVEEKFQVEKAGGSLKTKPRSSLKSAGEKKFSRTVTWADEKINSTGSKDLCEFKEFGDIKKESDSVGNNIDVANDEDILRRASAEACAIALSSASEAVASGDSDVSDAVSEAGITILPPPHDAAEEGTVEDADILQNDSVTLKWPRKTGISEADFFESDDSWFDAPPEGFSLTLSPFATMWNTLFSWTTSSSLAYIYGRDESFHEEYLSVNGREYPCKVVLADGRSSEIKQTLASCLARALPALVAVLRLPIPVSIMEQGMACLLETMSFVDALPAFRTKQWQVVALLFIDALSVCRLPALISYMTDRRASFHRVLSGSQIRMEEYEVLKDLVVPLGRAPHISSQSGA

SEQ ID NO: 25 - protein, 634 aa, Glycine max
MEKDKPVSVKDAVFKLQMSLLEGIQNEDQLFAAGSLMSRSDYEDIVTERSITNVCGYPLCSNALPSDRPRKGRYRISLKEHKVYDLHETYMFCCSNCVVSSKAFAGSLQAERCSGLDLEKLNNILSLFENLNLEPAENLQKNEDFGLSDLKIQEKTETSSGEVSLEQWAGPSNAIEGYVPKPRDHDSKGLRKNVKKAEMGFVSTIIMQDGYSVSKVLPAIVKQLGKVDAKVVRKDDGSIQDLSSSFKSSLILGTSEKEEELAQSCEAALKSSPDCAIKKKDVYSVSISERQCDVEQNDSAKKSVQKFQVEKAGEKKLSRTVTWADKKINSTGSKDLCGFKNFGDIRNESDSAGNSIDVANDEDTLRRASAEACVIALSSASEAVASGDSDVSDAVSEAGIIILPPPHDAGEEGTLEDVDILQNDSVTVKWPRKPGISEADFFESDDSWFDAAPEGFSLTLSPFATMWNTLFSWITSSSLAYIYGRDESFQEEYLSVNGREYPCKVVLADGRSSEIKQTLASCLARALPTLVAVLRLPIPVSTMEQGMACLLETMSFVDALPAFRTKQWQVVALLFIDALSVCRLPALISYMTDRRASFHRVLSGSQIGMEEYEVLKDLAVPLGRAPHISAQSGA

SEQ ID NO: 26 - protein, 746 aa, Sorghum bicolor
MSSPAAAAAAEAPRTVASAVLRIQMALLDGAAASNEALLHAAASALLSRADYDDVVTERTIADACGNPACPNPLPSSSSAAAATGPRFHIALSEHRVYDLEEARKFCSDRCLVASKALAASLPHDRPYGVPLDRLAAVVALVEGAAAAGDGSGLGFQGVDGNVKMKDEGRKVEIKEKEVAGAGEVSLQDWIGPSDAIEGYVPRRDRSAHGQKPQAEQNKVAGSDLSRTKNVDDRTAAPSEDGMTSPLSLVETHMSAEVMAERMGDLVLGENTKTLSRKKKTKTPSKMMEQEEDDSMLSSCISDSIAKQLEDVVLEERKGSKKNKVSKASSRTHKSKSRKRPAGSDGHEVDFTSTIIIGDASTNREESAMNQYNYLSSSVLVDNHPSSSQSSAKDSTQAYAEQLCEEFSEAVNIGNDETTDEKMRPALKPSLKVTGSKSGRQSVTWADENGSVLETSKAYESPSSSIKQPNEGIDSSLRRASAEACAAALIEAAEAISSGTAETEDAVSKAGIIILPDMLNQKEYGDAKNNGGDDDPEIDRDVIKWPKKPVLLDTDMFEVDDSWHDTPPEGFSLTLSAFGTIWAALFGWISRSSLAYVYGLERGSVEELLIANGREYPEKIVLKDGLSSEIRRALDSCVCNAVPVLISNLRLQIPVSKLEITLGYLIDTMSFVEALPSLRSRQWQAVVLVMLDALSVHQLPALAPVFSNSKLVQKMLNAAQVSREEYDSMVDLFLPFGRSVQATTPM

SEQ ID NO: 27 - protein, 746 aa, Brachypodium distachyon
MAPHAAAAAAGTTRTTMNVATAVYRVQLALLDGAAASNEPLLHAAAAVLSRADYDDVVTERSIADACGHPPCASPLPAAAAAAAAPPRFHISLREHRVYDLEEARKFCSERCLVASAAFAASLPHDRPFGVPPDRLDALVALFEGGGDRPGLGFREVSSGKDKDEGRKLEIREKEAPGLGEVTLQEWIGPSDAIEGYVPRHHPIPEGPMPEAKQRKTSRADQSRNKNLDSATSSSVEAPVSSEVIAKKLNDMVLGDNTKTKKKQVCETPSKMFRPDEHGDMLLSCVTDSIAKQLEDVVLEEKNDMKKERPTRASSRSRKSKPAKKPAGSDGHEVGFTSTIIMGDHVLAKMDQGPVGQYNFATSIADNQPSSSSSLSSSPTQYTARDLTGAYTEQLNKEFSKAVNLGKDEASDEKVRIVPKSSLKAGGSKNKSQSVTWADENGSLLEISKEYVIHSDDKKHYKEDIDGSLRRESAEACAAALIEAAGAISLGTSEVEDAVSKAGIIILPDMLHQNQFKSDNGKNTVEKEISETDNGVVKWPNKPVFLDTDMFEVDDSWHDTPPEGFNLTLSAFATMWATLFGWISRSSLAYVYMLDGSSVEELLISSGREYPQKTVSKDSQSSEIKRTLATCIGNALPVLTSNLRMQIPVSKLETTLGYLIDTMSFVEALPPLRSRQWQLMVLVLLDALSVCRLPGLAPVMSDSKLLQKVLNSSQVSREEYDSMVDLFLPFGRSVQTPPPSQPVQVP

SEQ ID NO: 28 - DNA, 4665 bp, Oryza sativa
ctcttcgtctcgactctgaactaaaaactcaactcctcccaaaatccctcgccgccgccgccgccgccgccgacgacgacgacatgggccccaccacggccaccgacaccggcgcgaggatgaagcccacgaccgtcgcgtcggcggtgcaccgcgtccagatggcgctctacgacggcgccgcggcgtcgagggagccgctgctccgcgcggcggcctcgctgctctcggggccggactacgccgacgtcgtcacggagcgctccatcgccgacgcctgcgggtacccggcgtgccccaacccgctcccctcggaggacgcccgcggcaaggcggcgccgcggttccgcatctcgctccgggagcaccgcgtgtacgacctcgaggaggcccgcaagttctgctccgagcgctgcctcgtcgcctccgccgccttcggggcgtcgctcccgcccgaccgccccttcggcgtctcgcccgaccggctcgacgccctcgtcgcgctcttcgagggcggtggtggtggtggtgatgacggtgggttggcgctagggtttggggcgagcggcgatgggaaggaggtggaagaggggaggaaggtggagatcatggagaaggaggcggctgggacgggggaggtgacgctgcaggagtggattgggccgtcggacgccatcgagggctatgtgcctcgccgtgatcgcgtcgttggaggtgaatcgccttttgctgacctcttctatttctatttgcattgagcattgattggtttttctgttcaaattatttactcgtttggtttgattgtgctgccataggttagtagtacgatgagtgaggtttatggcggtggcattctctgttatcctttgtgctcaaattaggtagagaaaaaactttggaagatagaaaatggaggaatttgtagtgttcgtagtaaattactttgagtatcaacaattgttgttgctagtagcagttaggaaaattctctgtaacatgtacaactacaacattttggtgaactagttataaactccttgtgtaaaatctccctacctgattagcaaattagtagcatttgtgtatgactagaaacaatcaactaggtaataattgagagctatcaaatatggtaagggacagctgaagaatagacataaccgtggtgccatattttttggttgcagtcattcaaattacaagaggacaactaggcaattcactcattttgtttagacctacatattgttgttgataagggacgctagaacgaagataggagattgagagagtaattaatttttggaagtacgttacagattttaatgggaggtagtgacttcttttttgagagattagaggggtgagagatgagctgcttactttgtcaatgaaaagcgctcaggttttgttcaatatttccccgaagaaatacaaaaatcatgtctttgtaagcatcatgtgttgttcctcttttctcactgctcacggaggtgcctaaactgcctaacaaggtggcaaactggtccctagtgcctaaacaccaagccaccaaggcagtggtggtttggtgggagtgtggtggtacttagctcctacctcctaggcactgtttgtttttcttatattcaaataatgttctagaactagatgtgacaatgttacaagagtaacaatagttttacttgcaggcacatttatatatatgaaaaggatatgctcattcttgctctttgttcgacctgtcttagtttcgaacaattgaagaaacaatgagcttgtgttctgtatttcagggccaaaaaaagaggctaaacagaacgatgcttgtagtgctgagcagtccagtaatattaatgtggattctaggaatgcttcttctggtgaatccggcatggttcttactgagaatacaaaagcaaagaaaaaggaagcaaccaaaaccccattgaagatgttcaagcaggatgaagataatgatatgttgtcgtcttgcatatcggattccattgtgaagcagctggaggatgtagttctcgaagagaaaaaggataagaagaaaaataaagcagctaaaggaacatcgagggtaggtaagagtaagcctgcaaaaagaccagttgggcgtgatggacatgaagtggactttacaagtacaattattatgggtgatcgtggttcagaaatgatggatcatggtgctctgggtcaatataatttctcaagttctatattagcaaatgagcagccttcatcatctcaatatgcagcgatagattcagtgcaagcttacactgaagaactagatgaattatttagtaatgcagttaacattgcaaaagacgagacaagtgatgatagtggtagatgtacactaagatcttcattgaaggctgttggatccaagaatgcagggcattctgtgaaatgggcagacgagaatggaagtgtgttagagacaagcagagcatttgtaagtcactccagtaaatctcaagaaagcatggacagttcagtaaggcgtgaatctgcagaagcttgtgcagctgcgcttattgaagcagcagaagctatttcatctggcacatcggaagtagaagatgcaggtgaacacttattccttaaccctgtggtgctttgacatgcagcttttgtttttgatatgtattaacctgtgccttttggtaacagtttcaaaggcaggaatcatcatactgccggacatggttaaccagcaacagtacaataatgattatgacaatgacaaagatgcaggggaaaatgaaatatttgaaattgataggggtgttgtgaagtggccgaagaagactgtgcttctagacacagatatgtttgatgtcgatgattcttggcatgatacaccaccagaaggctttagtctaactgtaagaattcttgagaaaaaataaagtagcacttcttcttttttttccttctggttgaatttggcatgtcatgccttttttccttttcagctgtcctccttcgcaacgatgtgggctgcattatttggatgggtatcccggtcctcattggcctatgtgtatgggcttgatgaaagttctatggaagatttgttgattgcaggtggaagagaatgtcctcagaagagagttttaaatgatggccactcatctgaaattagaagagctttggatacttgtgtgtgtaatgccctgccagttcttgtatcaaacttgaggatgcaaattccagtctcaaagttggagattactctggtatggataaagctagttgaacaaactaattaaaaagttaaaacattttttaaaagaaaagaataaggactccgcaattggttagtgacctagttgaacctggatatgtatttactggctagtacatatagtttttcagcagcggctgtgaagaggcttttcgttgcattttgttctggtatctagacacctttgactgagatcaatcaagctactaatatcttggtcagtttaacaataatttcttagtcatttggggttctctctcattcataagtgtgggtgatgagctgcgcatatctggaaatagataagacgaagtggaggctgtacgttacagttattctgaattagggccagtctcttttttgttgattctattgaagacagcttaactgctagatgttgccaccaagaattttatttgttagtttgattacagtttcagtaattaacatttagagctataattagtgtgcatctaatgtgtttttcccctccccactagggatacttgttggacacgatgtcatttgttgatgcactgccttctctgagatcaaggcagtggcaattgatggttctcgtgctgcttgatgcgctctcactccatcggcttcctgctcttgctccaataatgtcagattcgaagcttttgcagaaggtgatgatgcctttccctgcctttctgctatgcaagtagacggatgcacatattctttttaagaaatctgatctttcttcccttttgtgtgctgcagcttttgaactcggctcaggttagccgagaggagtatgactccatgattgatctcctcctcccttttggaagatccacgcagagccaggcatccctgccaagttaaacctcaagcaacacaagtatctaaatacatgtttacacagcggagtaaatagagagaggttctacatatcagcgtcagcttggcagtctattcggatatactatcataatggtgcctcttgcgtctggttgtttaggtttcggaacacgtcttgcaaaatatcggttgctcccagttgcttcaactatcccctttgcattggcgtcagacgtacaagcggagcgggcactgattcctgtcattctcaggaattgttagctttagtgaggagagcaaaagataactgcctccagaacaatttggtccactatggacttttatctctcattgctagcgcagttcagaattgtagtgcatctttgggttttttttttacctcttttaccggtgtgcagtttgtcaggttagtacagccgaatttgaccagctaacaacccactcgtgagtcctaataggtgagtgctagatatcaagtggtatagtaaacccagtgttagcatctataaaattctgaattttatgcctgtcaatctcgc

SEQ ID NO: 29 - DNA, 4966 bp, Zea mays
gcttgaactcatttgcaattgcgaacgcttcttctcccagtcccagcacgcccaaccccgtcgccgccttctcgaaccttccgaagatgagccccccggccccggccgccgcggcggcggcggcgccgcgaacggtcgcctcggcggtgctccgcgtccagatggcgctcctcgacggcgccgcggtgtccagcgaggccctcatccacgcggccgcctccgcgctcctctcccgcgccgactacgacgacgtcgtcaccgagcgcaccatctcggacgtctgcggcaaccccgcgtgccccaaccctctctcctcctcctccgccgccgccacggggccccgcttccacatcgccctcagcgagcaccgcgtctacgacctcgaggaggcgcgcaagttctgctccgagcgctgcctcgtcgcctccaaggccttggccgcctcgctcccgcacgaccggccctacggggtcccgctcgaccgcctcgccgcggtcgtcgcgctcgttgagggcgccgccgcaggggacgggagcgggttagggttccagggactggatgggaatgggaaggtggaggacgggggaaggaaggtggagatcaaggagaagcaggtcgccggggctggcgaggtcttgctgcaggactgggttgggccctccgatgccattgagggttatgtgccgcgccatgaccgcagtgctcatggtgagtatcccatactttgtttgcccatgattgcattttattattttgtaaatatcaattgattgaatgacggaaaggcgagaacagagcccaatggtggtgttagtgtcttgtcgtaatgctgaaatattgcatggagatatgttgctttagacttcagtgttgcagtttatgtatttggtcttatatatttccataggtgtttgctcatgaataacttgggtaggttgaattggtttccggaataagatatgattctatgttgatattgttacttttaagtttttaacatgtaaatataagtaatctttacctgtggttgtgtcgcaattagtagtttatttgcatcctgctagtgcatctagttagcaacctttttgtatacatgaagactatttgggtgaaaggagccaaacttgacacctacttgcatattttccatgtgatgatcgtgtctatggcggttgacgtgtgcagcctgcagatgtttgtgttattgtttaagtgtgtatgctagtagaaattaaagaaaaatagaacaatagaggttgttgctcaatggttttctgactattactcctttgcaccatgatgtgttgaggtcattcacccttggagttttagtcagtacccttgatttagggtctgtttggttgggctgtggctgtggaaaaagttgctgtgggctgtgagctgtggaaaaagctgctataggctgtgtgctgttaaaaagctaaaaatcgtttggtggaaaccactaaaagtcgttaaaagttctttgatatatgttttcacagttccatccaaaagccactaaaagcaggtccaggggtgctttcagttttgcactacgagaaagtcggcttttagaaaaagctgcttcgtggatccagccctttggttggcttttggcttttagggggcaaaagccaaagccaaaagccaaaccaaacacacccttagtatctctactgctgcattttgcacatcataactttgctactgatgcagttcagcctgcactttgcagcacactgacaatttatagggtcctgcctgcaagcatcttggggttgatattgatttaacacaccaattttttagtcactggtgtaagtaagaatgtttaccagagtaacggtagtttttcttgcatgcacatttctgtacatgaaaggatccttatctccattcttgccctttgttcgacttgtcttaaattttgaacaattgaagatacagtgagcttatgttctgttgtcaggacaaaagccacaggttcagcagaacgaaggtgctggacctgaactgtccagaactgagaatgtggattatggtgctgctgctcctggtgaagatggcatgacaagttcaccttcattggttaaaacacacgtgagctccgaagtaatagttgagagaatgggcagcctggttcttggtgagaatacaaggacgcctagaaagaagaaaactaaaactccatcaaagatgttagagcaagaggaagataacagtatgctgtcatcttgcatatctgattccattgccaagcagcttgaggatgtagttttggaagagagaaaaggcagtcagaaaaataaaatgagtaaagcatcatcaagagcacagaagggtaagtctacaaaaaggcctgcttcgacaaacatggaggaaaatgctatgaatcagtataactacttgtcaagttctgtattggtagacaatcacccctcatcatctcaatcttcagaaaaagattcaacacaggcttactctgaacaactgtgtgaagaattcagtgaagcagtgaacattggaaatgatgagacaagtgatgaaaagatgagacctgcatggaagtcttcgttgaaagttgccgggtctaagagcagtaggcagtctgttacatgggcagatgagaatggaagtgtcctagaaacaagcaaagcatatgaaagcccttcaagtagtataaaacgacctgaggaaggcatagacaattcactaaggcgtgcatctgctgaagcgtgtgctgcagcacttgttgaggcagcagaagctatttcttcaggcacagcagaagcagaagatgcaggtgagcatgtattcattatgttcccacggctgctattctttgaggctaacaacttttgttattaaatgatactgacgtaagcctctctaacgtcagtttcaaatgctggaatcatcattctgcctgacatgcttaaccagcaagaacatgacaatggcaaaaacagtggcggagatgatgaccctgagatagatagggatgttatcaagtggcctaagaaacctgtacttctggatacagacttgtttgaagttgatgattcttggcatgacatgcctccagaaggttttagtctaactgtaagtattattaagaagggaaaaaagagaggagaaattccagttttgcttttaattcggtctcagcacgatgtacatttcttttcagctgtctgctttcgggacgatgtgggccgcgctattcggatggatatccagttcgtctttggcctatgtgtatgggcttgaaaggggttcagtggaggagttgttgattgccaatgggagggaatgtcctgagaagacagttctgaaggatgggctctcattggagattagaagagctctagattcttgcgtttgtaacgccgtgccagtactcatatcaaacttgaggttgcagataccggtttcaaaactggagattactctggtacgtgtcaactttaccaagcagataataatatcgtaccttttttaaaataaggctgctataggtttagcttgatgctggtggctgacagttatatcggcggtaacacatgatgaaactacaccgtgtctgtaccagggctacttgattgacacaatgtcgtttgttgacgccctgccttctctgcgatcgaggcagtggcaggctgtggttctggtaatgcttgacgcgctctctgtgcaccagcttcccgcccttgctccagtcttttcgaattcgaagcttgtgcaaaaggcgagtgaccagtttttttgtggttagttgaataatatatgtatatcttattttgctttggtgacatctgaatttgtttccccatcaatgtgcgatgcagatgttgaacgctgctcaggttagcagagaggagtatgactccatggtggacctgtttctaccgtttggaagatccgtccaggcgatcacgcccatgtaaacgagaagccgtgtgaatctgcattccggaagctgcgtgaatcgtagggtctgatctgatatttagttttacacaagtcgctgtctagaacgagccatgtatgtatgattgattggttatctattgagcaagatgcgctctggcaatttgtggaagctgaaatacttgcgaccatgacgcctgcctgtcagtttgcgatattctttcaggttgtgagaaattgatttgctgtcgttctcagctattgttagctttatgccctctttgaactcctagagctaatagttagccagctaaacagccgagctagctaataaactaactaagctaactaataaactaattattagttgtgagttagctaacaattaactggactattagccttggatctgaacttttccccctctgctcctgtttatccctgcgtttttacttatatacaacgggcagtttcacgtaattacatgggcagtttcacgtaattacatgacatttttggcattgtgtagaactcataaagctaacgctaatagagtgtgtttggtttggtttggttttagcttttgacaattaaaattcaaaagctaaaccagagggttcgatccagtaaacattttttttctctaaaaatcgactttctcatagcacaagacagactgaaagcatctcttcacctatttttaacggcttttggatgaaactgtgaaaatatatatggaagaaattttagcatcttttattggcttccaccaaaccgatttttttgttgctttttttacatttcatagcccacattagttttttatagctcacagcacacaacaacttttttcatagccacaacccaaactaaacacaccatagttagctagctaataatttgttagcttgaataacctaattaactgttagctaggatctactaattttttatagtaccttgctaaaagtaaatggatttcacactccaaacataacacaaacaacaccagtctattaaaaaacatataaaa

SEQ ID NO: 30 - DNA, 4584 bp, Glycine max
ttcaagctatttggttctgaagcttttgttcaatggcaaaggacaagcctgtttctgtcaaagatgccgtcttcaaattgcaaatgtcactccttgaaggcattcaaaatgaagaccagctgtttgctgccgggtctttgatgtcaaggagtgactacgaagacattgtaaccgaacgatccatcacaaacatgtgtggttatccactctgcagcaatgctttgccatccgatcgcccgcggaagggtagatataggatttcactgaaggagcacaaggtctatgacctacaagagacttacatgttttgttcttcaaattgtcttgttagcagcaaaacttttgctgggagcttgcaagctgagagatgctctggtttagacctggagaaactaaacaatgttcttagcttgtttgagaatttgaatctggaaccggtggagactttgcaaaagaatggagatttaggtttgtctgatttgaaaatccaggagaaaacagaaagaagcagtggggaggtgtctttggagcagtgggctggaccttcaaatgcaattgagggatatgtaccaaaaccaagaaaccgtgattctaagggtttgcggaaaaatgttaaaaaaggtgaggatttttttgggttttctagacaattgtgagaaactaatcagttagccatatgcgatttcttgcttaagagtgtccttttattatcatgtttaatgttaggcctatggatgtttatggcatgactgtgaagtctgaggctgaggcttaatttatgtgaaatggaatactataattactatttcttatccttttttctctatccaatcaagtgagctttgattctgcattgcagggtccaaaactggtcatggcaagtcaattagtgacataaatttaattaacagtgagatgggctttgtgagtactataattatgcaagatgagtatagtgtttcaaaagtaccgccaggtcaaatggatgcaactgctaatcatcaaattaaaccaacagctacagtcaagcagccagaaaaggttgatgctgaagtggtcaggaaagatgatgatagcattcaagatttgtcttcatcttttaagagcagtttaattttaagcacctcagaaaaagaggaggaagtaactaaatcatgtgaagctgtgctcaaattctcccccggttgtgctattcaaaagaaagatgttcattcaatctccatatcagaaagacaatgtgatgtggaacagaatgattctgctaggaaatctgtacaagtcaaagggaaaacgagtagagttattgctaatgatgatgcttccacttccaatttagatcctgccaatgttgaagagaaattccaagtggaaaaagcaggtggatcattaaagactaaacccagatcttcccttaaatctgctggtgaaaagaaatttagtcgcactgttacttgggcggatgagaaaatcaacagcactgggagtaaagatctttgtgagtttaaagaatttggagatattaaaaaagaatctgactcagtaggaaataatatagatgttgccaatgatgaagatatattacgtcgtgcgtcagcagaagcttgtgctattgcattgagctcagcatcagaagcagttgcctctggagactcggatgtcagtgatgctggtaattactgtttcttttacgagttttctatttaaattaattggtctgtaaaagttttctcgcctatgaatgaaacttgtgcagtttctgaagctggaatcactatattgccacctccacatgatgctgctgaggaaggtactgtggaggatgctgatatactacaaaatgattcagttactctgaaatggccaagaaagactggaatttctgaagctgatttctttgaatctgatgactcatggtttgatgctccaccagagggtttcagtttgactgtaagtttagtgaaaattttagaaacattcaaagctgtattaatttccacagtatattctatggcataacttgtgctttctttctttcctttctttttttttataattttttgtttgtttgtgtgtgtaactttatttcagttgtcaccttttgcaactatgtggaataccctcttttcatggacaacatcatcttctttggcatatatatatgggagggatgaaagttttcatgaagaatatctatcagttaatggcagagaatatccttgcaaagttgtcttggcagatggtcgctcatctgaaataaaacaaactttagccagttgtcttgctcgagctttacctgcgcttgttgctgtgctccggctgccaataccagtatctatcatggagcaagggatggtaggatcttggagttatctatttttgtagtttattttgtagtatagttgtcccaaactaagagtcattacagatttattatacttcagcaggcatggtctggtttgttttacaccattcattgggacttaaaggaaaatcatttaatataagcatatgcatgttaatttttgtgagaaatagatatggatccattgcttgaaataagagacacgtggaaattaaaatatttgagcctgtaggataaatacttcatgctattgaaaagaaaagaaatagaatttggggagagggggtggggtgctgctgagccccttgtatccgtccctgcctgcatagtccttgctatttaaattccttaatcattatgtggttcgattcagggaaaatctgcaataattgcaagatacctagttgcattggtctaagcacttaaacaaactaatagtgacttctttttgctggaagcaaatactaaaatgatttctccatgttacattgatttagcatctgcaacacattaactgcttttggttgacaattaggaggattcttgggttttaaaattatgccttattgtctgagaagacaatataatttttgagaatcagtttagaaatcagtgaaagtaggggagtcttggcacaggggtttcaagcaatggcatcagcttcttgcagagacaatgctggttactacttcctacacatattgggtcctacccttccctgacccaacacatagctttatagcacaaggttccctatttttaaagtttagaaatttctaaattcatgagttttaggctataagttgcagatggatttaatcttcatatctgtattgaaaaattctttaatatatccatttacttggtgcatggatgggttatatgttaattctcgtgactttctattaggcatgcttgctggagacaatgtcatttgtggacgcacttccagctttcagaacaaaacaatggcaagtggttgctcttttgttcattgatgcattgtccgtatgtagattacctgctcttatctcatacatgacggataggagggcttcatttcatagggtgagaaccttttgtttatatcatactaatttattattttatatacatgatggataggaggaatctgtttgcggtgcatgaagcattatataatgatgttgggttgtatatatattcaggcaaaaaaagcataatcccatattatggctctgatcttttggttagggtagttcagttaacatctgatatctccattatttcaattgctaaaattttggttacttttcatctgcatttcctttataaatgttttgttgcccaattcttgatcaaatgggagcttaacaacataaatcccacacaacttagctacaatgtgcttaatgttgttccccttctatagctttagactcgatggccttattttatgattctatagtttagttcatctagtgtggttctccacttctcctaccatggctgtggactttaaattcttgtttggttttccatcatgtattttcatgctaattttgtttgaagtcagctaaaccacaagtcgtgcacatagattgccataggcacaaatttatggcacgtggacatggaacccaacttggaaatttttttcttctaatttgattccctacatatgtgtgttggggttgcaacaattttgatatgacagctggaatatggaggttaatgatatatcttattgttttgttgttttatctcctgttatgtcagattttgatgttataattttcactcttcttttgaacctgttaaccattgttctggtattgatttggcgaaacaggttttgagtggttctcaaatacgtatggaagagtatgaggttttgaaggatcttgtagtaccactgggccgagcacctcatatctcttcccaaagtggggcatgacaacaaataaatgttggacttcaccaacgatgagcgcaatattatatgctctaatgctagctagcgagtgataaatgcaaattgatattcaaaatagacattgttctaaaatattgtcttcctgttaaatggcagtttgtatgtttttattttattttatgccaagcagtctgtagaaggttctgaagatgtctattggattactaatcttagaggtgcatgtcaggatggttaatacagaacaaatatcttcccgattataagattgattttacagatttaattt

SEQ ID NO: 31 - DNA, 4979 bp, Glycine max
gctgaaaggaaaatagaggtttctctttcaagtttcacctcctcaccaccgccaagcttcgtctccgagaatgtcaccatcgcacacttttttgtttccaagcacagcaccgatgatattagcaaccaaaccctaatcgtgtccacgctttctttcttctttcctggcacactttctccctctccactttcagcacacactttttcgttttctggtaagtcctaatttagagaactgtgtgatggatttgaaattagggttaatgaaagtgcatttcttctgtccaaattagggttatcatggaacctaattttacggtctaaatgcgttaacgttcaaggacgactacgaatgtgattattgtaatatataatcatcaatgcaattacttttatctttcatgtgctttaataatagaattatgattgtgtcttgtgactctgaaattttgttgggttgaactgttgaagctattcttattgtggtgtactttgtgcatgatgcagttctagttatttggttctgaagcttttgtttcaatggaaaaggacaagcctgtttctgtcaaagatgctgttttcaaattgcaaatgtcgctccttgaaggcattcaaaatgaagaccagctgtttgctgctgggtctctgatgtcaaggagtgactacgaagacattgtaaccgaacgatccattacaaacgtgtgtggttatccgctctgcagcaatgctttgccatccgatcgcccacggaagggtagataccggatttcactgaaggagcacaaggtctatgacttacacgagacttacatgttttgttgttcaaattgtgttgttagcagcaaagcttttgctgggagcttgcaagcagagagatgctcaggtttagacctggaaaaactaaacaatattcttagcttgtttgagaatttgaatctggaaccagcggagaatttgcaaaagaatgaagatttcggtttgtctgatttgaaaatccaggagaagacagaaacaagcagtggggaggtgtctttagagcagtgggctggaccttcaaatgcaattgagggttatgtaccaaaaccaagagaccatgattctaagggtttgcggaaaaatgttaaaaaaggtgaggattttttttttgggtgttctagagtctagacaattgtgagaaactatagataaatagatagccttatgcaatttcttgcttaagagtgtcctgtttattatcatgtttaatgttaagcctatggatgattgtggcgtgactgtgaagtctgaagctgaggcttaatttatgtgaaatggcatactataattactgtttcttatccttttccactatccaaccaagtgagctttgattctgcattgcagggtccaaagctggtcatggcaagccaattagtgacataaatttaattagcagtgagatgggctttgtgagtactataattatgcaagatgggtatagtgtttcaaaagtactgccaggtcaaagagacgcaaccgctcatcatcaaattaaaccaacagctatagtcaagcagttaggaaaggttgatgctaaagtggtcaggaaagatgatggtagcattcaagatttgtcttcatcttttaagagcagtttaattttaggtacctcagaaaaagaggaggaattagcccaatcatgtgaagctgcgctcaaatcctctcccgattgtgctattaaaaagaaagatgtttattccgtctccatatcagaaagacaatgtgatgtggaacagaatgattctgctaagaaatctgtacaagtcaaagggaaaatgagtagagttactgctaatgatgatgcttccacttccaatttagatcctgccaatgttgaagagaaattccaagtggaaaaagcaggtggatcattaaacactaaacccaaatcttcccttaaatctgcaggtgaaaagaaacttagtcgcactgttacttgggcagataagaaaatcaacagcactgggagtaaagatctttgtgggtttaaaaattttggagatattagaaatgaatctgactcagcaggaaatagtatagatgttgccaatgatgaagatacattacgtcgcgcgtcagcagaagcttgtgttattgcattgagctcagcatcagaagcagttgcttctggagactcggatgtcagtgatgctggtataattactgtttctcttacaagttttctatttaaattgattggtttgtaaaaggttttttcgcctatgaatgaaacttgtgcagtttctgaagctggaatcattatattgccaccaccacatgatgctggtgaggaaggtactctggaggatgttgatatactacaaaatgattcagttactgtgaaatggcctagaaagcctggaatttctgaagctgatttctttgaatctgatgactcatggtttgatgctgcaccagagggtttcagtttaactgtaagtttagtgaaaattttagaaacattcaaacctgtattatttttcagtatattctatggcataactttaaacttgtgctttttcttttctttctttttttaatttttttgtttgtttgtttgtttgtgtgtgtgtaactttctttcagttgtcaccttttgcaactatgtggaataccctcttttcttggataacatcatcttctttggcatatatatatgggagggatgaaagttttcaagaagaatatctatcagttaatggcagagaatatccctgcaaagttgtcttggcagatggtcgctcatctgaaataaaacaaactttagccagttgtcttgctcgagctttacctacacttgttgctgtgctccggctgccaataccagtatctaccatggagcaagggatggtaggatcttggagttatcagttttgtagtttatgttgtagtatagttgtcccaaattaagtcatcagagatttattatacttcaccaggcatggtgggacttctctcatatgttcataagctccccttcccacaaaacatgcacatagtcacacatattcacaaaatatgagattgataaatttgatgttaaaaatgaactttcaaacagtggtaaaggaaaatcagttaatataagcatatgaatgctactttttgtgagaatagagatggatccattgcttgaaataagagacacatggaaattaaaatattaagcctgtaggataaatactttttattgattgaaaataaaagaaatcgaatgggaattccccccccccccccccagagtagatcctgcctgcataatccttattatttcaattccttgattgtcatgtggttcaattcagggaaaatctgcaagaattgcaagatctagttgcaaaattatgccttattgtccgagaagacaaaatatgttttctgagaatcagtttagaaatttgtgaaagtaggggagtcttggcacaggggtttcaagcaatggcatcagccccttgcaaagacaatactggctactatacttcccacacatattgggtcctatgcttccctgacccagcacatagctttatagcacaaggttccctatttttagaagttcagaaatttgtaaattcatgagtttttaggctataagttgcagatagatttaatcttcatatctttattgaaaattctttaatatatccaattacttggtctgtggatgggttatatgttaattctcgtgactttctattaggcatgcttgctggagacaatgtcatttgtggacgcacttccagctttcagaacaaaacaatggcaagtggttgctcttttgtttattgatgcattgtccgtatgtagattacctgctcttatctcatacatgacggataggagggcttcatttcacagggtgagaacctttgtttatatcatactgatttattattttatttacatgatggatagaaggaaactgtttgctgtgcatgaagcattatataatgatgttgggttgtatatagatcgatgcattcccttattacggcacatgatcttttggttggggtagttcagttaacatctgatatctccgttatttcaattgctaaaattttggttacttttcatctgcatttcctttataaatgttgtcgcctgattcttggtccaaatgggagcttaacaacataaatcccacacaacttagctaccatgtgcttaatgttgttctccttctatagctttagactcgatggcctcatcttatagtttagttcatctagtgtggttctgcacttctaccatggctgtggacttgaaattcttgtttggttttccatcttgtattttcatgctaaatttatttgaatgcagctagaccacaagtcgtgcacatagattgccatggcacaaatttatggcacgcggacactgaacccaacttggaaattttttccttctaatttgattccctacatatatgtgtgtgttggggttgcaaaaattccgatgacagctggaatatggaagttcatgatatatcttattgttttgttgttttatctcctatgtcatgtcagattttgatgttcactctacttttaaatttgttaaccattgttctgttattgatttggtgaaacaggttttgagtggttctcaaataggtatggaagaatatgaggttttgaaggatcttgcagtgccactgggccgagcacctcacatctctgcccaaagtggggcatgacaacaaatagtgcattttttttttatttggggaaaatttacctgtcaagcattggagtgcttgtttataaacataaaaaatatgcaatgacaattatgctaggcctctagcaacacgggaaccagatttctatattctctaatgctagctagagagtgatgaatgcaaaattgatattcaaaatagacatagttctaaaatattgtcttccagttaaatagcagtttgtattttttttaaaa

SEQ ID NO: 32 - protein, 21 aa, Arabidopsis sp.
RGGLAPGVGLGWGASGGGFWS

SEQ ID NO: 33 - protein, 47 aa, Arabidopsis sp.
SDIDVENHAKLQTMSPDEIAEAQAELLDKMDPALLSILKKRGEAKLK

SEQ ID NO: 34 - protein, 80 aa, Arabidopsis sp.
RDLRFSFDGNVVEEDVVSPAETGGKWSGVESAAERDFLRTEGDPGAAGYTIKEAIALARSVIPGQRCLALHLLASVLDKA

SEQ ID NO: 35 - protein, 40 aa, Arabidopsis sp.
ERDFLRTEGDPGAAGYTIKEAIALARSVIPGQRCLALHLL

SEQ ID NO: 36 - protein, 21 aa, Arabidopsis sp.
DWEAIWAYALGPEPELVLALR

SEQ ID NO: 37 - protein, 32 aa, Arabidopsis sp.
TIQKDVFVAGQDVAAGLVRMDILPRIYHLLEE

SEQ ID NO: 38 - protein, 68 aa, Arabidopsis sp.
TIQKDVFVAGQDVAAGLVRMDILPRIYHLLEEPTAALEDSIISVTIAIARHSPKCTTAILKYPKFVQT

SEQ ID NO: 39 - protein, 10 aa, Arabidopsis sp.
EWAHQRMPLP

SEQ ID NO: 40 - protein, 273 aa, Arabidopsis sp.
LPPHWFLSAISAVHSGKTSTGPPESTELLEVAKAGVFFLAGLESSSGFGSLPSPVVSVPLVWKFHALSTVLLVGMDIIEDKNTRNLYNYLQELYGQFLDEARLNHRDTELLRFKSDIHENYSTFLEMVVEQYAAVSYGDVVYGRQVSVYLHQCVEHSVRLSAWTVLSNARVLELLPSLDKCLGEADGYLEPVEENEAVLEAYLKSWTCGALDRAATRGSVAYTLVVHHFSSLVFCNQAKDKVSLRNKIVKTLVRDLSRKRHREGMMLDLLRYK

SEQ ID NO: 41 - protein, 54 aa, Arabidopsis sp.
DVVTERAIAKLCGYTLCQRFLPSDVSRRGKYRISLKDHKVYDLQETSKFCSAGC

SEQ ID NO: 42 - protein, 54 aa, Arabidopsis sp.; identical to SEQ ID NO: 41

SEQ ID NO: 43 - protein, 23 aa, Arabidopsis sp.
SRSDYEDVVTERAIAKLCGYTLC

SEQ ID NO: 44 - protein, 13 aa, Arabidopsis sp.
ISLKDHKVYDLQE

SEQ ID NO: 45 - protein, 7 aa, Arabidopsis sp.
SVTWADQ

SEQ ID NO: 46 - protein, 35 aa, Arabidopsis sp.
AEALATALSQAAEAVSSGNSDASDATAKAGIILLP

SEQ ID NO: 47 - protein, 38 aa, Arabidopsis sp.
SWFDGPPEGFNLTLSNFAVMWDSLFGWVSSSSLAYIYG

SEQ ID NO: 48 - DNA, 679 bp, Artificial
  MINIYO promoter
tcatttaccaagtttacaaagattatggtccaagtcctaaaactgaatgaacatcacatacccgtctcttgagtgatttaatcatgttcttcattgcactaaagcgacaacttttggttcaaatatagactatgactatatggtttgttttgcacaggattaaagttgatgttccaattttataattaaaaagtcagaaaggggttttcttgttattttttacttgttcttataagctatcgggacgacacggagttttaaagagttttccgttttgctgagcggaggcgagagagggtttagagtgatggagcaaagtagcgggagagtcaatccggaacagccgaacaacgtcttggcgagccttgtcgggagcatcgtggagaaaggaatatcggagaataagcctccaagcaagccgcttcccccaaggccctcccttctttccttccccgtcgctcgtcatcgttctcacggacccgtaagccaatccaatcctctagtgcgtgctttttaggtttccatcttccttttgttgccttcttctagattttaagcaccttctactgttgtttagtacttgggactccacaatttttcaccgtgcctgaccttgtaattcagctttctgagacatctaatttttgtttctcatgtttgattttgtagcatttggctc

SEQ ID NO: 49 - DNA, 2097 bp, Artificial
  AtRTR1 promoter
tgaatcatttctcaaaaagaaaatgggaaaaatgtcattcaaataatcagtttaccattttcgttggttttaaacataaattttggacctggtgatttaaatcctcaatattatgttgactttcagtttaaacacaaattttcattgattaagagacatcgttagaaattccctagatcacataccctttatcccaaaaccgaaaaccgatttttggattccctcttcttcttcgaattcgaagtaatctcttgtctggaggttgactgatggcgtaaaaaaagaagaatttgtatctcaattagtttagtttacaagaactcgtgattaaattgaaagtcaaaataaaaatgagaatttaaattaccaaatcaagagttttcatattttaaatggtaaactgatgacattttccctgttgaacaatattggcccataatgtaacccaattactcggcccaattacgtgaaccgcctttcacctggcttaaggaataagtaaggaccattcatgatctcatcacttttagctttctggcttctctgcttaagctctctcgagtctgcctcaagtgtttttgggggaaattgatttcgttgagaaaaaccctaaattccgaacttgaagcaatttttcaatttcgtttgcagaaaaatggcaaaggataatgaagcaatcgccattaacgatgcggttcacaagcttcagctctatatgctcgaaaataccactgatcagaaccagctcttcgcggcgaggaagttaatgtctcgatcagattacgaagatgtcgtcactgaacgagcaatcgctaagctctgtggttatactctttgccagagatttctcccttccgatgttctagaagagggaagtatcggatttcgttgaaggaccataaggtttacgatttacaggagacgagcaagttttgctccgctggttgtttaattgatagcaaaacgttttcggggagtttgcaagaggctcgtacattggagtttgattcggtgaagttgaatgagattttggatttgtttggtgattctttggaagtgaaaggttctttggatgtgaataaggatttggatttgtctaagcttatgattaaggagaattttggagttagaggtgaagaattgtctttagagaagtggatgggtccttctaatgctgttgaaggttatgttccttttgatcgaagcaaatcaagtaatggtaagttcgatgatgaactatggtgtgagcaaaaaatttcagttaacaaatgttttatcgatgtgtaataattaagtttggttttggcagattccaaggctactactcaaagtaatcaagagaagcatgagatggatttcactagcacagtaattatgcctgatgttaatagtgtttcaaagcttccaccgcaaaccaagcaagcttctactgttgtggaatctgttgatggcaaagggaaaacagttctgaaagagcaaactgtagttcctcccaccaaaaaagtttcgagtaagcattaaggagtttttaagagtaataggccttatgaccaacatatctctaagaaatgtagctgtatatgttatttagtcttgcttaaaggtatttggatggtatcatgaatgttttgatttattcgtcggagagacagatcttttggtggttattaggccatttctactgatgggtgaagcaataaatgtcgttgtccttgctctctgtttatctgagtcttaatgaattctaatgtgtgtctgcaggatttcgtcgtgagaaagaaaaggagaagaagactttcggggttgatgggatgggttgtgcccaggaaaaaactacagttctccccagaaaaatattgagtaagcacttaggaagctgtgaagatagttaggccttactttcaagatatctcttaaaataatctgtatatgttacgtttttttcattttgctgtattcatttggtatctcgaatgagattctttattccttgggtctctaagttgttctaatgattgttaggcagtttttgtgcctgtgtgactctgtttatctgtctaacatatgcaggtttttgtaatgaaatagagaaggatt

SEQ ID NO: 50 - protein, 13 aa, Arabidopsis sp.
GEAKLKKRKHSVQ

SEQ ID NO: 51 - protein, 24 aa, Arabidopsis sp.
RDLSRKRHREGMMLDLLRYKKGSA

SEQ ID NO: 52 - protein, 29 aa, Arabidopsis sp.
LKGDLQTLDGKNTLSGSSSGSNTKGSKTK

SEQ ID NO: 53 - protein, 9 aa, Arabidopsis sp.
LVLALRMAL

SEQ ID NO: 54 - protein, 10 aa, Arabidopsis sp.
LKGDLQTLDG